(19) United States
(12) Patent Application Publication
(10) Pub. No.: US 2026/0151450 A1
(43) Pub. Date: Jun. 4, 2026

(54) FORMULATIONS AND METHODS

(71) Applicant: Flagship Pioneering Innovations VII, LLC, Cambridge, MA (US)

(72) Inventor: Hari Ahlad ATTLURI, Ashland, MA (US)

(21) Appl. No.: 19/406,916

(22) Filed: Dec. 2, 2025

Related U.S. Application Data
(60) Provisional application No. 63/727,059, filed on Dec. 2, 2024.

(51) Int. Cl. A61K 38/08; A61K 38/10; A61K 47/10; A61K 47/22; A61K 47/24

(52) U.S. Cl. CPC A61K 38/08; A61K 38/10; A61K 47/10; A61K 47/22; A61K 47/24

(57) ABSTRACT

The disclosure provides, inter alia, formulations for drug delivery, such as extended release of peptide therapeutics.

### CROSS-REFERENCE TO RELATED APPLICATIONS

This application claims priority to and the benefit of U.S. Provisional Patent Application No. 63/727,059, filed Dec. 2, 2024, the contents of which are incorporated herein by reference in their entireties.

### FIELD

The present disclosure relates to, inter alia, formulations and methods of making and using the same, e.g., for peptide therapeutics.

### BACKGROUND

Formulation of therapeutic compounds, such as peptide therapeutics, requires significant development to optimize PK and therapeutic efficacy, for example by extending residency time and release of the therapeutic compound or active pharmaceutical ingredient (API). Accordingly, a need exists for improved formulation compositions as well as methods of making and using the same.

### SUMMARY

The disclosure provides, inter alia, improved formulation compositions as well as methods of making and using the same, e.g., for peptide therapeutics, such as GPCR modulators, such as agonists of GPCRs, such as melanocortin receptors.

In embodiments, there is provided a composition comprising:
(a) about: 20-60% (W/W) total phospholipids, wherein the total phospholipids comprise a first and a second species of phospholipid in a ratio of about: 80:20, 75:25, 70:30, 60:40, 50:50, or 40:60 of the first species to the second species, wherein the second species is a phospholipid comprising a lipid with a dioleoyl and glycero group and/or a phosphoethanolamine group;
(b) about: 10-60% (W/W) of a slow diffusing solvent;
(c) about: 5-30% (W/W) of a fast diffusing solvent or solvent that is highly miscible with water such as an alcohol (such as ethanol), NMP, DMSO, or a combination thereof; and
(d) optionally an active pharmaceutical ingredient (API), optionally wherein the API is a peptide, optionally wherein the peptide comprises one or more non-canonical amino acids.

In embodiments, the second species of phospholipid is 1,2-dioleoyl-sn-glycero-3-phosphoethanolamine (DOPE).

In embodiments, the first species of phospholipid is phosphatidylcholine, optionally wherein the first species of phospholipid is Phospholipon® 90 G or variants thereof (e.g., LIPOID S 100, LIPOID E PC S, LIPOID P 100-3), optionally wherein the phosphatidylcholine is soy bean phosphatidylcholine, egg yolk phosphatidylcholine, sunflower phosphatidylcholine, or synthetic phosphatidylcholine.

In embodiments, the first species of phospholipid is phosphatidyl choline, the second species of phospholipid is DOPE, and the first and second species are in a ratio of about: 80:20, 75:25, 70:30, 60:40, 50:50, or 40:60; optionally wherein the composition comprises at least about: 30, 35, 36, 37, 38, 39, 40, 41, 42, 43, 44, 45, 46, 47, 48, 49, 50, 51, 52, 53, 54, 55, or 60% (W/W) total phospholipids, e.g., about: 40-60% (W/W).

In embodiments, the slow diffusing solvent comprises one or more of medium or long chain triglycerides, Miglyol® 812 N and variants thereof (e.g., Miglyol® 810 N, Miglyol® 840), capric triglycerides, caprylic triglycerides, caproic triglycerides, lauric triglycerides, MYRITOL® 318 and variants thereof (e.g., MYRITOL® 312, MYRITOL® 331 N), NEOBEE® Caprylic Triglyceride (e.g., NEOBEE® 1053 MB), CAPTEX® medium chain triglycerides (e.g., CAPTEX® 200P, CAPTEX® 300 EP/NF, CAPTEX® 8000), medium chain triglycerides oil, sesame oil, castor oil, polyoxyl 35 castor oil, soybean oil, PEG-60 hydrogenated castor oil, peanut oil, cottonseed oil, corn oil, coconut oil, glycerin, monothioglycerol, glyceryl palmitostearate, glycerol dioleate, including combinations of the foregoing.

In embodiments, the slow diffusing solvent is a medium chain triglyceride.

In embodiments, the slow diffusing solvent is present at a concentration of about: 14, 15, 16, 17, 20, 25, 28, 30, 31, 33, 34, 35, 37, 38, 39, 40, 41, 42, 43, 44, 52, 53, 55, 58, or 60% (W/W).

In embodiments, the fast diffusing solvent is ethanol, optionally at a concentration of about: 6, 7, 8, 9, 10, 11, 12, 13, 14, 15, 16, 20, 25, 26, 27, 28, 29, or 30% (W/W).

In embodiments, the composition comprises an API.

In embodiments, the API is present at a concentration of at least about: 0.1, 0.5, 1, 2, 3, 4, 5, 6, 7, 8, 9, 10, 12, 15% (W/W), or more.

In embodiments, the API is a peptide comprising at least about: 5, 6, 7, 8, 9, or 10 amino acids.

In embodiments, the API is a peptide comprising one or more non-canonical amino acids.

In embodiments, the API is a G protein coupled receptor (GPCR) modulator, such as an agonist, antagonist, or biased signaling molecule; e.g., wherein the GPCR is a melanocortin receptor, such as a MC4R, optionally wherein the API is a MC4R agonist peptide or a pharmaceutically acceptable salt thereof.

In embodiments, the API is a peptide or a salt thereof

In embodiments, the API is a peptide or a salt thereof, wherein the peptide comprises the amino acid sequence of formula (I):

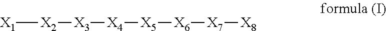

wherein in formula (I):
   X₃ is 3-Aminooxetane-3-carboxylic acid (Aib(O-cyclic));
   X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
   X₁, X₂, X₅, X₆, X₇, and X₈ are each independently a canonical or non-canonical amino acid.

In embodiments, X₄ is Gln.

In embodiments, X₅ is selected from 4-fluoro-D-phenylalanine (D-Phe(4-F)), D-phenylalanine (D-Phe), and 4-methyl-D-phenylalanine (D-Phe(4-Me)), optionally wherein X₅ is D-Phe(4-F).

In embodiments, X₆ is arginine (Arg).

In embodiments, X₇ is 6-fluoro-L-tryptophan (Trp(6-F)).

In embodiments, X₈ is penicillamine (Pen) or cysteine (Cys), optionally wherein X₈ is penicillamine (Pen).

In embodiments, X₁ is selected from D-norarginine (D-Nar) and beta-homo-L-arginine (Beta-homoArg), optionally wherein X₁ is D-norarginine (D-Nar).

In embodiments, X₂ is Cys.

In embodiments, the peptide is a cyclic peptide, optionally wherein the cyclic peptide comprises a disulfide bridge or a lactam bridge.

In embodiments, the peptide is a cyclic peptide and comprises a disulfide bridge.

In embodiments, the cyclic peptide has the amino acid sequence of formula (II):

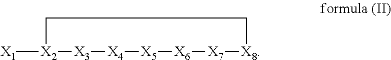

In embodiments,

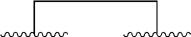 [SMILES: *CC*]

represents a disulfide bridge.

In embodiments, the peptide is capped with N-terminal acetyl and/or C-terminal amide groups, optionally wherein the peptide is capped with N-terminal acetyl.

In embodiments, the peptide comprises the amino acid sequence as set forth in formula (III):

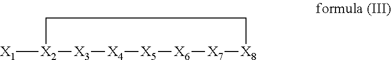

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

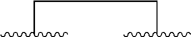 [SMILES: *CC*]

disulfide bridge, and the peptide is capped with N-terminal acetyl.

represents a

In embodiments, the peptide of formula (I) is a peptide of any one of formula (Ia), formula (Ib), formula (Ic), formula (Id), formula (Ie), or formula (If):

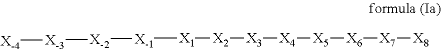

wherein in formula (Ia):
   X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker;

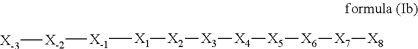

wherein in formula (Ib):
   X₋₁, X₋₂, X₋₃, X₁, X₂, X₃, X₄, X₅, X₆, X₇, and X₈ are each independently an amino acid selected from Table 1, Table 2, Table 3, Table A1, Table AlA, Table A2, and Table A2A or a linker;

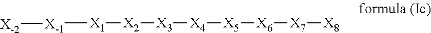

wherein in formula (Ic):
   X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker;

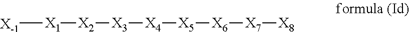

wherein in formula (Id):
   X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker;

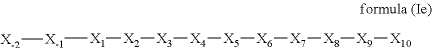

wherein in formula (Ie):
   X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker;

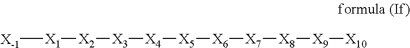

wherein in formula (If):
   X₋₁, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

In embodiments, the cyclic peptide of formula (II) is a cyclic peptide of any one of formula (IIa), formula (IIb), formula (IIc), formula (IId), formula (IIe), or formula (IIf):

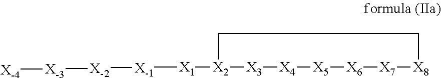

wherein in formula (IIa):
   X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker;

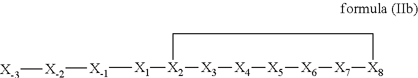

wherein in formula (IIb):
   X₋₁, X₋₂, X₋₃, X₁, X₂, X₃, X₄, X₅, X₆, X₇, and X₈ are each independently an amino acid selected from Table 1, Table 2, Table 3, Table A1, Table A1A, Table A2, and Table A2A or a linker:

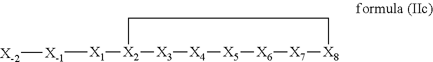

wherein in formula (IIc):
   X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker;

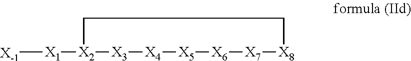

wherein in formula (IId):
   X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker:

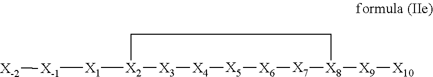

wherein in formula (IIe):
   X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker:

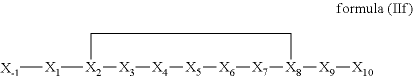

wherein in formula (IIf):
   X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

In embodiments, the peptide is selected from Table A1, Table A1A, Table A2, Table A2A, Table 1, and Table 2.

In embodiments, the API is a salt of a peptide.

In embodiments, the salt is an acetate salt, a trifluoroacetate salt, a phosphate salt, a phosphite salt, a propionate salt, a chloride salt, a fumarate salt, a citrate salt, a tartrate salt, an oxalate salt, a succinate salt, a mandelate salt, a methanesulfonate salt, a p-toluenesulfonate salt, a bromide salt, an iodide salt, a hydroxide salt, a sulfate salt, a sulfite salt, a nitrate salt, a malate salt, a maleate salt, an aspartate salt, a glutamate salt, a lactate salt, a gluconate salt, a benzoate salt, a salicylate salt, an ethanesulfonate salt, a naphthalenesulfonate salt, or a camphorsulfonate salt.

In embodiments, the salt is a pharmaceutically acceptable salt, optionally wherein the pharmaceutically acceptable salt is selected from a sulfate salt, a citrate salt, an acetate salt, a oxalate salt, a chloride salt, a bromide salt, an iodide salt, a nitrate salt, a bisulfate salt, a phosphate salt, an acid phosphate salt, an isonicotinate salt, a lactate salt, a salicylate salt, an acid citrate salt, a tartrate salt, an oleate salt, a tannate salt, a pantothenate salt, a bitartrate salt, an ascorbate salt, a succinate salt, a maleate salt, a gentisinate salt, a fumarate salt, a gluconate salt, a glucuronate salt, a saccharate salt, a formate salt, a benzoate salt, a glutamate salt, a methanesulfonate “mesylate” salt, an ethanesulfonate salt, a benzenesulfonate salt, a p-toluenesulfonate salt, and a pamoate salt (i.e., 1, l′-methylene-bis-(2-hydroxy-3-naphthoate)).

In embodiments, the salt is an acetate salt or a trifluoroacetate salt.

In embodiments, the salt is an acetate salt.

In embodiments, the salt is a trifluoroacetate salt.

In embodiments, the peptide comprises one charged atom, and the salt comprises one counterion; or the peptide comprises two charged atoms, and the salt comprises two counterions; or the peptide comprises three charged atoms, and the salt comprises three counterions; or the peptide comprises four charged atoms, and the salt comprises four counterions, optionally wherein the counterion is acetate, optionally wherein the counterion is trifluoroacetate.

In embodiments, each counterion is independently selected from (i) an acetate ion, a trifluoroacetate ion, a phosphate ion, a phosphite ion, a propionate ion, a chloride ion, a fumarate ion, a citrate ion, a tartrate ion, an oxalate ion, a succinate ion, a mandelate ion, a methanesulfonate ion, a p-toluenesulfonate ion, a bromide ion, a iodide ion, a hydroxide ion, a sulfate ion, a sulfite ion, a nitrate ion, a malate ion, a maleate ion, a aspartate ion, a glutamate ion, a lactate ion, a gluconate ion, a benzoate ion, a salicylate ion, a ethanesulfonate ion, a naphthalenesulfonate ion, and a camphorsulfonate ion, optionally wherein each counterion is an acetate ion, optionally wherein each counterion is a trifluoroacetate ion; or (ii) from a sulfate ion, a citrate ion, an acetate ion, an oxalate ion, a chloride ion, a bromide ion, an iodide ion, a nitrate ion, a bisulfate ion, a phosphate ion, an acid phosphate ion, an isonicotinate ion, a lactate ion, a salicylate ion, an acid citrate ion, a tartrate ion, an oleate ion, a tannate ion, a pantothenate ion, a bitartrate ion, a ascorbate ion, a succinate ion, a maleate ion, a gentisinate ion, a fumarate ion, a gluconate ion, a glucuronate ion, a saccharate ion, a formate ion, a benzoate ion, a glutamate ion, a methanesulfonate “mesylate” ion, an ethanesulfonate ion, a benzenesulfonate ion, a p-toluenesulfonate ion, and a pamoate (i.e., 1,1′-methylene-bis-(2-hydroxy-3-naphthoate)) ion.

In embodiments, the API is an acetate salt of a peptide comprising the amino acid sequence as set forth in formula (III):

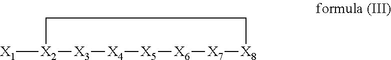

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

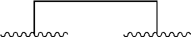 [SMILES: *CC*]

represents a disulfide bridge, and the peptide is capped with N-terminal acetyl, optionally wherein the peptide.

In embodiments, the API is a trifluoroacetate salt of a peptide comprising the amino acid sequence as set forth in formula (III):

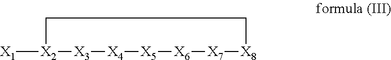

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

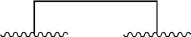 [SMILES: *CC*]

represents a disulfide bridge, and the peptide is capped with N-terminal acetyl.

In embodiments, the API is a bistrifluoroacetate salt of a peptide comprising the amino acid sequence as set forth in formula (III):

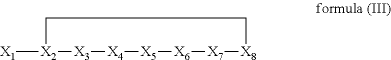

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

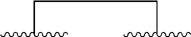 [SMILES: *CC*]

represents a disulfide bridge, and the peptide is capped with N-terminal acetyl.

In embodiments, the API is an acetate salt of a peptide consisting of the amino acid sequence as set forth in formula (III):

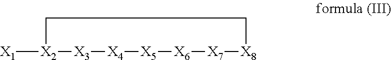

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

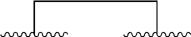 [SMILES: *CC*]

represents a disulfide bridge, and the peptide is capped with N-terminal acetyl, optionally wherein the peptide.

In embodiments, the API is a trifluoroacetate salt of a peptide consisting of the amino acid sequence as set forth in formula (III):

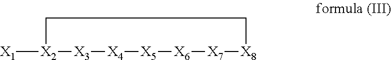

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

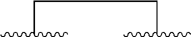 [SMILES: *CC*]

represents a disulfide bridge, and the peptide is capped with N-terminal acetyl.

In embodiments, the API is a bistrifluoroacetate salt of a peptide consisting of the amino acid sequence as set forth in formula (III):

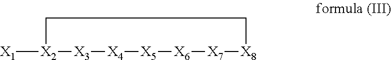

wherein in formula (III):
   X₁ is D-norarginine (D-Nar);
   X₂ is cysteine (Cys);
   X₃ is 3-Aminooxetane-3-carboxylic acid (Aib(O-cyclic));
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

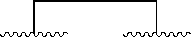 [SMILES: *CC*]

represents a disulfide bridge, and the peptide is capped with N-terminal acetyl.

In embodiments, the peptide is peptide 1158, optionally wherein the salt is an acetate salt of peptide 1158, optionally wherein the salt is a trifluoroacetate salt of peptide 1158, optionally wherein the salt is a bistrifluoroacetate salt of peptide 1158:

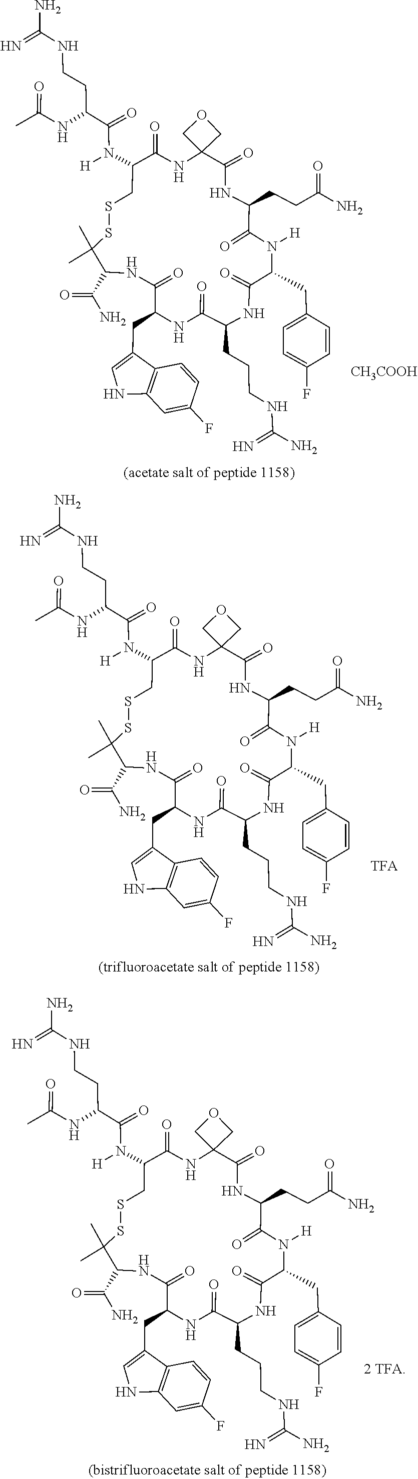

In embodiments, the salt demonstrates one or more of (a)-(h):
(a) increased selectivity for MC4R over MC1R when administered to a subject compared to a control;
(b) increased selectivity for MC4R over MC1R when administered to a subject as measured by an in vitro, ex vivo, or in vivo assay when compared to a control;
(c) an increased ratio of MC4R intracellular signaling to MC1R intracellular signaling when administered to a subject compared to a control;
(d) increased selectivity for MC4R intracellular signaling to MC1R intracellular signaling as measured by an in vitro, ex vivo, or in vivo assay when compared to a control;
(e) enhanced melanocortin 4 receptor (MC4R) function in a subject when compared to before the salt is administered or to a pre-treatment or non-treatment state, or a subject treated with control;
(f) decreased melanocortin 1 receptor (MC1R) function in a subject when compared to before the salt is administered or to a pre-treatment or non-treatment state, or a subject treated with control;
(g) enhanced melanocortin 4 receptor (MC4R) function as measured by an in vitro, ex vivo, or in vivo assay when compared to a control; and
(h) decreased melanocortin 1 receptor (MC1R) function as measured by an in vitro, ex vivo, or in vivo assay when compared to a control.

In embodiments, the composition comprises an API and, upon administration to a mammalian subject (e.g., by subcutaneous injection), exhibits an extended release of the API, relative to a control composition, such as a control composition not comprising the second phospholipid species, as evaluated by, for example maximum serum concentration (Cmax), steady-state concentration (Css), or flat exposure of the API, e.g., at up to 24, 36, 48, 60, 72, 84, or 96 hours or more, optionally wherein the API is a peptide or a pharmaceutically acceptable salt thereof.

In embodiments, the composition is an extended release composition.

In embodiments, there is provided a composition comprising:
(a) about: 20-60% (W/W) total phospholipids, wherein the total phospholipids comprise Phospholipon® 90 G or soybean phosphatidylcholine and 1,2-dioleoyl-sn-glycero-3-phosphoethanolamine (DOPE) in a ratio of about: 80:20, 75:25, 70:30, 60:40, 50:50, or 40:60 by weight;
(b) about: 10-60% (W/W) of a slow diffusing solvent, wherein the slow diffusing solvent is Miglyol® 812 N or caprylic acid triglycerides and/or capric acid triglycerides (e.g., a mixture of caprylic acid triglycerides and capric acid triglycerides);
(c) about: 5-30% (W/W) of a fast diffusing solvent or solvent that is highly miscible with water, wherein the fast diffusing solvent or solvent that is highly miscible with water is ethanol; and
(d) about: 1-15% (W/W) of an active pharmaceutical ingredient (API), wherein the API is a peptide or a pharmaceutically acceptable salt thereof, and wherein the peptide comprises one or more non-canonical amino acids.

In embodiments, there is provided a composition comprising:
(a) about 55% (W/W) total phospholipids, wherein the total phospholipids comprise Phospholipon® 90 G or soybean phosphatidylcholine and 1,2-dioleoyl-sn-glycero-3-phosphoethanolamine (DOPE) in a ratio of about 50:50 by weight;
(b) about: 40% (W/W) of a slow diffusing solvent, wherein the slow diffusing solvent is Miglyol® 812 N or caprylic acid triglycerides and/or capric acid triglycerides (e.g., a mixture of caprylic acid triglycerides and capric acid triglycerides);
(c) about: 12% (W/W) of a fast diffusing solvent or solvent that is highly miscible with water, wherein the fast diffusing solvent or solvent that is highly miscible with water is ethanol; and
(d) about: 3% (W/W) of an active pharmaceutical ingredient (API), wherein the API is a peptide or a pharmaceutically acceptable salt thereof, and wherein the peptide comprises one or more non-canonical amino acids.

In embodiments, there is provided a composition selected from any one of Table BBB, Table CCC, or Table DDD.

In embodiments, there is provided an article of manufacture comprising the compositions of present disclosure.

In embodiments, the article comprises a vial or a prefilled medical device, such as a syringe, optionally made of Glass, COP, or COC and optionally with a closed stopper or plunger.

In embodiments, there is provided a method comprising administering an effective amount of the compositions of present disclosure, optionally via the articles of present disclosure.

In embodiments, there is provided a method of making a composition, the composition comprising:
(a) about: 20-60% (W/W) total phospholipids, wherein the total phospholipids comprise a first and a second species of phospholipid in a ratio of about: 80:20, 75:25, 70:30, 60:40, 50:50, or 40:60 of the first species to the second species, wherein the second species is a phospholipid comprising a lipid with a dioleoyl and glycero group and/or a phosphoethanolamine group;
(b) about: 10-60% (W/W) of a slow diffusing solvent;
(c) about: 5-30% (W/W) of a fast diffusing solvent or solvent that is highly miscible with water such as an alcohol (such as ethanol), NMP, DMSO, or a combination thereof; and
(d) optionally an active pharmaceutical ingredient (API), optionally wherein the API is a peptide, optionally wherein the peptide comprises one or more non-canonical amino acids,
wherein the composition is made by a method substantially similar to a method disclosed herein, including all equivalents and variants thereof.

In embodiments, the method comprises the steps of:
(a) adding the fast diffusing solvent to the slow diffusing solvent and mixing, optionally mixing for about 5-15 minutes, to obtain a solution;
(b) adding the first species of phospholipid to the solution of (a) while mixing the solution;
(c) mixing the solution of (b) following the addition of the first species of phospholipid, optionally mixing for about 15-90 minutes;
(d) adding the second species of phospholipid to the solution of (c) while mixing the solution;
(e) mixing the solution of (d) following the addition of the second species of phospholipid, optionally mixing for about 20 minutes to about 8 hours;
(f) optionally adding the API to the solution of (e) while mixing the solution; and
(g) optionally mixing the solution of (e) following the addition of the API, optionally mixing for about 5 minutes to about 2 hours.

In embodiments, the method comprises the steps of:
(a) adding the fast diffusing solvent to the slow diffusing solvent and mixing, optionally mixing for about 5-15 minutes, to obtain a solution;
(b) adding the second species of phospholipid to the solution of (a) while mixing the solution;
(c) mixing the solution of (b) following the addition of the second species of phospholipid, optionally mixing for about 15-90 minutes;
(d) adding the first species of phospholipid to the solution of (c) while mixing the solution;
(e) mixing the solution of (d) following the addition of the first species of phospholipid, optionally mixing for about 20 minutes to about 8 hours;
(f) adding the API to the solution of (e) while mixing the solution; and
(g) mixing the solution of (e) following the addition of the API, optionally mixing for about 5 minutes to about 2 hours.

In embodiments, there is provided a method of preparing the compositions of present disclosure, comprising the steps of:
(a) adding the fast diffusing solvent to the slow diffusing solvent and mixing, optionally mixing for about 5-15 minutes, to obtain a solution;
(b) adding the first species of phospholipid to the solution of (a) while mixing the solution;
(c) mixing the solution of (b) following the addition of the first species of phospholipid, optionally mixing for about 15-90 minutes;
(d) adding the second species of phospholipid to the solution of (c) while mixing the solution;
(e) mixing the solution of (d) following the addition of the second species of phospholipid, optionally mixing for about 20 minutes to about 8 hours;
(f) optionally adding the API to the solution of (e) while mixing the solution; and
(g) optionally mixing the solution of (e) following the addition of the API, optionally mixing for about 5 minutes to about 2 hours.

In embodiments, there is provided a method of preparing the compositions of present disclosure, comprising the steps of:
(a) adding the fast diffusing solvent to the slow diffusing solvent and mixing, optionally mixing for about 5-15 minutes, to obtain a solution;
(b) adding the second species of phospholipid to the solution of (a) while mixing the solution;
(c) mixing the solution of (b) following the addition of the second species of phospholipid, optionally mixing for about 15-90 minutes;
(d) adding the first species of phospholipid to the solution of (c) while mixing the solution;
(e) mixing the solution of (d) following the addition of the first species of phospholipid, optionally mixing for about 20 minutes to about 8 hours;
(f) adding the API to the solution of (e) while mixing the solution; and
(g) mixing the solution of (e) following the addition of the API, optionally mixing for about 5 minutes to about 2 hours.

In embodiments, one or more of steps (a)-(g) are performed at a temperature of about 30-35° C., optionally wherein steps (b)-(g) are performed at a temperature of about 30-35° C., optionally wherein steps (d)-(g) are performed at a temperature of about 30-35° C., optionally wherein steps (f)-(g) are performed at a temperature of about 30-35° C.

In embodiments, one or more of steps (a)-(g) are performed at a temperature of about 15-25° C. (e.g., room temperature), optionally wherein steps (a), and (d)-(g) are performed at a temperature of about 15-25° C., optionally wherein steps (a)-(c) and (f)-(g) are performed at a temperature of about 15-25° C., optionally wherein steps (a), and (f)-(g) are performed at a temperature of about 15-25° C.

In embodiments, the method further comprises the step of:
(h) filtering the solution of (g) through a sterile filter, optionally wherein the sterile filter is a 0.22 μm filter, or a 0.8/0.22 μm filter, or a 0.4/0.22 μm filter, or a 0.22/0.4 μm filter.

In embodiments, the method further comprises the step of:
(i) titrating the solution of (h) to a target weight of the composition using the fast diffusing solvent, optionally wherein the fast diffusing solvent is filtered through a sterile filter, optionally wherein the sterile filter is a 0.22 μm filter, or a 0.8/0.22 μm filter, or a 0.4/0.22 μm filter, or a 0.22/0.4 μm filter.

In embodiments, the method further comprises the step of:
(j) filling an amount of the solution of (i) into a vial, a pre-syringe, or a cartridge, optionally wherein the amount is about 1-10 mL, optionally wherein the vial, pre-syringe, or cartridge is sterile.

In embodiments, one or more of (i)-(v) apply:
(i) the first species of phospholipid is Phospholipon® 90 G or soybean phosphatidylcholine;
(ii) the second species of phospholipid is 1,2-dioleoyl-sn-glycero-3-phosphoethanolamine (DOPE);
(iii) the slow diffusing solvent is Miglyol® 812 N or caprylic acid triglycerides and/or capric acid triglycerides (e.g., a mixture of caprylic acid triglycerides and capric acid triglycerides);
(iv) the fast diffusing solvent is ethanol; and
(v) the API is a peptide or a pharmaceutically acceptable salt thereof, optionally wherein the API is a MC4R agonist peptide or a pharmaceutically acceptable salt thereof, optionally wherein the API is peptide 1158 or a pharmaceutically acceptable salt thereof.

### BRIEF DESCRIPTION OF THE DRAWINGS

FIG. 1 is a line graph summarizing rat in vivo mean concentration of API for different formulations for a peptide API.

FIG. 2 is a line graph summarizing mini pig in vivo mean concentration of API for different formulations for a peptide API.

FIG. 3 is a line graph summarizing dog in vivo mean concentration of API for different methods of preparing compositions of present disclosure comprising a peptide API.

FIG. 4 is a line graph summarizing mini pig in vivo mean concentration of API for different compositions of present disclosure with three different concentrations of a peptide API.

FIG. 5 shows the results of a weight loss study in mice administered with various nonlipidated peptides. The graph represents a comparison of the percentage of weight loss over three days in diet induced obese (DIO) mice with treatment of exemplary peptides.

FIG. 6 is a bar graph of the amount of food intake in DIO mice after treatment with exemplary peptides.

FIG. 7 is a graph of the amount of weight loss in DIO mice after treatment with 10 mg/kg of a MC4R agonist peptide (peptide 1158) of the disclosure dosed once daily.

FIG. 8 is a table listing various exemplary peptides and their related results when assessing the selectivity of MC4R versus MC1R as well as the bias of MC4R B-arrestin versus MC4R cAMP. When comparing the selectivity of MC4R v MC1R, larger values indicate selectivity towards MC4R. When comparing the bias of MC4R B-arrestin v MC4R CAMP, larger values indicate bias towards B-arrestin. * denotes MC4R vs MC1R selectivity having a range of 0.01 to <1.00, ** denotes MC4R vs MC1R selectivity having a range of ≥1.00 to ≤7.40, *** denotes MC4R vs MC1R selectivity of about >7.40. + denotes MC4R B-arrestin v MC4R cAMP bias having a range of >0.00 to ≤2.00, ++ denotes MC4R B-arrestin v MC4R CAMP bias having a range of 2.00 to ≤10.00, +++ denotes MC4R B-arrestin v MC4R cAMP bias having a value of >10.00. Grey color denotes peptides with a lipidated state. N/A denotes peptides with no data collected. IA denotes an inactive state.

### DETAILED DESCRIPTION

The present disclosure provides, inter alia, improved formulations for drug delivery and methods of preparing and uses thereof.

### Compositions and Uses Thereof

In aspects, the present disclosure provides compositions for drug delivery.

In embodiments, the compositions are for peptide drug delivery. In embodiments, the compositions are for extended release delivery (e.g., of peptide drugs) commonly known as long acting injectable products.

In embodiments, the disclosure is based, at least in part, on Applicant's discovery that certain phospholipids with dioleoyl and glycero group and/or phosphoethanolamine groups, such as 1,2-dioleoyl-sn-glycero-3-phosphoethanolamine (DOPE, PubChem CID 10350317), can substantially improve formulation and PK properties. Phospholipids with dioleoyl and glycero group and/or phosphoethanolamine groups are known. Exemplary, non-limiting examples include DOPE and compounds described in Zhang et al. 2024 (http://dx.doi.org/10.2139/ssrn.5003728) or with the descriptor “dioleoyl” identifiable on known providers' websites, such as AvantiResearch, the contents of which are incorporated herein by reference in their entireties.

In aspects, the disclosure provides a composition comprising:
(a) about: 20-60% (W/W) total phospholipids, wherein the total phospholipids comprise a first and a second species of phospholipid in a ratio of about: 80:20, 75:25, 70:30, 60:40, 50:50, or 40:60 of the first species to the second species, wherein the second species is a phospholipid comprising a lipid with dioleoyl and glycero group and/or phosphoethanolamine group;
(b) about: 10-60% (W/W) of a slow diffusing solvent;
(c) about: 5-30% (W/W) of a fast diffusing solvent or solvent that is highly miscible with water such as an alcohol (such as ethanol), NMP, DMSO, or a combination thereof; and
(d) optionally an active pharmaceutical ingredient (API), optionally wherein the API is a peptide, optionally wherein the peptide comprises one or more non-canonical amino acids.

### Phospholipids

In aspects, the compositions of the disclosure comprise phospholipids.

In embodiments, the composition comprises about 20% (W/W) to about 60% (W/W) total phospholipids. In embodiments, the composition comprises about 20% (W/W), or about 25% (W/W), or about 30% (W/W), or about 35% (W/W), or about 40% (W/W), or about 45% (W/W), about 50% (W/W), or about 60% (W/W) total phospholipids.

In embodiments, the composition consists of about 20% (W/W) to about 60% (W/W) total phospholipids. In embodiments, the composition consists of about 20% (W/W), or about 25% (W/W), or about 30% (W/W), or about 35% (W/W), or about 40% (W/W), or about 45% (W/W), about 50% (W/W), or about 60% (W/W) total phospholipids.

In embodiments, the total phospholipids comprise a first species of phospholipid and a second species in a ratio of about 80:20, or about 75:25, or about 70:30, or about 60:40, or about 50:50, or about 40:60. In embodiments, the total phospholipids consist of a first species of phospholipid and a second species in a ratio of about 80:20, or about 75:25, or about 70:30, or about 60:40, or about 50:50, or about 40:60. In embodiments, the ratio is a ratio of weight between the first species of phospholipid and the second species of phospholipid.

In embodiments, each phospholipid is independently selected from phosphatidylethanolamine, phosphatidylcholine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

In embodiments, the first species of phospholipid is selected from phosphatidylethanolamine, phosphatidylcholine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. In embodiments, the first species of phospholipid is phosphatidylcholine. In embodiments, the phosphatidylcholine is selected from 1-palmitoyl-2-oleoyl-sn-glycero-3-phosphocholine (POPC). 1,2-dioleoyl-sn-glycero-3-phosphocholine (DOPC), dipalmitoylphosphatidylcholine (DPPC), and egg yolk phosphatidylcholine (EggPC), or any combinations thereof. In embodiments, the first species of phospholipid is Phospholipon® 90 G (PL 90 G). PL 90 G comprises phosphatidylcholine. In embodiments, the first species of phospholipid is phosphatidylcholine.

In embodiments, the first species of phospholipid is PhospholiponR 90 G or variants thereof (e.g., LIPOID S 100, LIPOID E PC S. LIPOID P 100-3). In embodiments, the first species of phospholipid is phosphatidylcholine selected from soybean phosphatidylcholine, egg yolk phosphatidylcholine, sunflower phosphatidylcholine, and synthetic phosphatidylcholine.

In embodiments, the first species of phospholipid comprises Phospholipon® 90 G or soy bean phosphatidylcholine. In embodiments, the first species of phospholipid consists of Phospholipon® 90 G or soy bean phosphatidylcholine.

In embodiments, the second species of phospholipid is a phospholipid comprising a lipid with dioleoyl group and glycero group. In embodiments, the second species of phospholipid is a phospholipid comprising a lipid with dioleoyl group and glycero group and/or phosphoethanolamine group. In embodiments, the second species of phospholipid is a phospholipid comprising a lipid with dioleoyl group, glycero group, and phosphoethanolamine group. In embodiments, the second species of phospholipid is a phospholipid comprising a lipid with dioleoyl group and phosphoethanolamine group. In embodiments, the second species of phospholipid is a phospholipid comprising a lipid with dioleoyl and glycero group or phosphoethanolamine group.

In embodiments, the second species of phospholipid is phosphatidylethanolamine.

In embodiments, the second species of phospholipid is selected from 1,2-dioleoyl-sn-glycero-3-phosphoethanolamine (DOPE), 1,2-dielaidoyl-sn-glycero-3-phosphoethanolamine, 1,2-dimyristoyl-sn-glycero-3-phosphoethanolamine (DMPE), 1,2-dipalmitoyl-sn-glycero-3-phosphoethanolamine (DPPE), 1,2-distearoyl-sn-glycero-3-phosphoethanolamine (DSPE), 1,2-dihexadecanoyl-sn-glycero-phosphoethanolamine (DHPE), 1,2-dioleoyl-sn-glycero-3-phosphocholine (DOPC), 1,2-dioleoyl-sn-glycero-3-phospho-L-serine (DOPS), 1,2-dioleoyl-sn-glycero-3-phosphate (DOPA), 1,2-dioleoyl-sn-glycero-3-phospho-(1′-rac-glycerol) (DOPG), and 1,2-dioleoyl-sn-glycero-3-phospho-(l′-myo-inositol), or any combinations thereof.

In embodiments, the second species of phospholipid is 1,2-dioleoyl-sn-glycero-3-phosphoethanolamine (DOPE).

In embodiments, the second species of phospholipid is 1,2-dielaidoyl-sn-glycero-3-phosphoethanolamine.

In embodiments, the first species of phospholipid is phosphatidylcholine (e.g., PL 90 G), and the second species of phospholipid is DOPE. In embodiments, the first species of phospholipid is Phospholipon® 90 G (PL 90 G), and the second species of phospholipid is DOPE. In embodiments, the first species of phospholipid (e.g., PL 90) G) and the second species of phospholipid (e.g., DOPE) are in a ratio of about 80:20, or about 75:25, or about 70:30, or about 60:40, or about 50:50, or about 40:60 by weight. In embodiments, the first species of phospholipid is PL 90 G, the second species of phospholipid is DOPE, and the first and second species are in a ratio of about 80:20, or about 75:25, or about 70:30, or about 60:40, or about 50:50, or about 40:60 by weight.

In embodiments, the composition comprises at least about 30%, or at least about 35%, or at least about 36%, or at least about 37%, or at least about 38%, or at least about 39%, or at least about 40%, or at least about 41%, or at least about 42%, or at least about 43%, or at least about 44%, or at least about 45%, or at least about 46%, or at least about 47%, or at least about 48%, or at least about 49%, or at least about 50%, or at least about 51%, or at least about 52%, or at least about 53%, or at least about 54%, or at least about 55%, or at least about 60% (W/W) total phospholipids. In embodiments, the composition comprises about 40% to about 60% (W/W) total phospholipids.

In embodiments, the total phospholipids comprise a first species of phospholipid (e.g., phosphatidylcholine) and second species of phospholipid (e.g., DOPE), and the composition comprises at least about 30%, or at least about 35%, or at least about 36%, or at least about 37%, or at least about 38%, or at least about 39%, or at least about 40%, or at least about 41%, or at least about 42%, or at least about 43%, or at least about 44%, or at least about 45%, or at least about 46%, or at least about 47%, or at least about 48%, or at least about 49%, or at least about 50%, or at least about 51%, or at least about 52%, or at least about 53%, or at least about 54%, or at least about 55%, or at least about 60% (W/W) total phospholipids. In embodiments, the total phospholipids comprise a first species of phospholipid (e.g., phosphatidylcholine) and second species of phospholipid (e.g., DOPE), and the composition comprises about 40% to about 60% (W/W) total phospholipids. In embodiments, the first species of phospholipid is phosphatidylcholine and the second species of phospholipid is DOPE. In embodiments, the first species of phospholipid is Phospholipon® 90 G (PL 90 G) and the second species of phospholipid is DOPE.

### Slow Diffusing Solvents

In aspects, the compositions of the disclosure comprise a slow diffusing solvent. In embodiments, the slow diffusing solvents are viscous solvents (such as triglycerides), non-polar, water-immiscible solvents (such as oils), or a combination of polar, water-miscible solvents and non-polar, water-immiscible solvents.

In embodiments, the composition comprises the slow diffusing solvent at a concentration of about 10%-60% (W/W). In embodiments, the composition comprises the slow diffusing solvent at a concentration of about 10% to about 45%, about 10% to about 40%, about 20% to about 45%, about 25% to about 30%, about 25% to about 45%, about 35% to about 45%, about 35% to about 40%, or about 40% to about 45% (W/W).

In embodiments, the composition comprises the slow diffusing solvent at a concentration of about 6%, or about 7%, or about 8%, or about 9%, or about 10%, or about 11%, or about 12%, or about 13%, or about 14%, or about 15%, or about 16%, or about 20%, or about 25%, or about 26%, or about 27%, or about 28%, or about 29%, or about 30% (W/W).

In embodiments, the slow diffusing solvents is a viscous solvent. Slow diffusing/viscous solvents include triglycerides (e.g., medium chain triglycerides or long chain triglycerides) including combinations thereof. Exemplary slow diffusing solvents include, without limitation, e.g., Miglyol® 812 N, sesame oil, castor oil, polyoxyl 35 castor oil, soybean oil, PEG-60 hydrogenated castor oil, peanut oil, cottonseed oil, corn oil, glycerin, monothioglycerol, glyceryl palmitostearate, glycerol dioleate, including combinations of the foregoing.

In embodiments, the slow diffusing solvent comprises a medium or a long chain triglyceride. In embodiments, the slow diffusing solvent comprises a medium chain triglyceride. In embodiments, the slow diffusing solvent comprises a mixture of more than one medium chain triglycerides (e.g., Miglyol® 812 N). In embodiments, the slow diffusing solvent consists of a mixture of more than one medium chain triglycerides (e.g., Miglyol® 812 N). In embodiments, the slow diffusing solvent comprises Miglyol® 812 N. In embodiments, the slow diffusing solvent consists of Miglyol® 812 N.

In embodiments, the slow diffusing solvent comprises one or more of medium or long chain triglycerides, Miglyol® 812 N and variants thereof (e.g., Miglyol® 810 N, Miglyol® 840), capric triglycerides, caprylic triglycerides, caproic triglycerides, lauric triglycerides, MYRITOL® 318 and variants thereof (e.g., MYRITOL® 312, MYRITOL® 331 N), NEOBEE® Caprylic Triglyceride (e.g., NEOBEE® 1053 MB), CAPTEX® medium chain triglycerides (e.g., CAPTEX® 200P, CAPTEX® 300 EP/NF, CAPTEX® 8000), medium chain triglycerides oil, sesame oil, castor oil, polyoxyl 35 castor oil, soybean oil, PEG-60 hydrogenated castor oil, peanut oil, cottonseed oil, corn oil, coconut oil, glycerin, monothioglycerol, glyceryl palmitostearate, glycerol dioleate, including combinations of the foregoing.

In embodiments, the slow diffusing solvent consists of one or more of medium or long chain triglycerides, Miglyol® 812 N and variants thereof (e.g., Miglyol® 810 N, Miglyol® 840), capric triglycerides, caprylic triglycerides, caproic triglycerides, lauric triglycerides, MYRITOL® 318 and variants thereof (e.g., MYRITOL® 312, MYRITOL® 331 N), NEOBEE® Caprylic Triglyceride (e.g., NEOBEE® 1053 MB), CAPTEX® medium chain triglycerides (e.g., CAPTEX® 200P, CAPTEX® 300 EP/NF, CAPTEX® 8000), medium chain triglycerides oil, sesame oil, castor oil, polyoxyl 35 castor oil, soy bean oil, PEG-60 hydrogenated castor oil, peanut oil, cottonseed oil, corn oil, coconut oil, glycerin, monothioglycerol, glyceryl palmitostearate, glycerol dioleate, including combinations of the foregoing.

In embodiments, the slow diffusing solvent comprises Miglyol® 812 N or caprylic acid triglycerides and/or capric acid triglycerides (e.g., a mixture of caprylic acid triglycerides and capric acid triglycerides). In embodiments, the slow diffusing solvent consists of Miglyol® 812 N or caprylic acid triglycerides and/or capric acid triglycerides (e.g., a mixture of caprylic acid triglycerides and capric acid triglycerides).

### Fast Diffusing Solvent

In aspects, the compositions of the disclosure comprise a fast diffusing solvent. In embodiments, the fast diffusing solvents are polar, water-miscible solvents.

In embodiments, the composition comprises the fast diffusing solvent at a concentration of about 5% to about 30% (W/W). In embodiments, the composition comprises the fast diffusing solvent at a concentration of about 10% to about 15%, about 10% to about 25%, or about 25% to about 30% (W/W).

In embodiments, the composition comprises the fast diffusing solvent at a concentration of about 6%, or about 7%, or about 8%, or about 9%, or about 10%, or about 11%, or about 12%, or about 13%, or about 14%, or about 15%, or about 16%, or about 20%, or about 25%, or about 26%, or about 27%, or about 28%, or about 29%, or about 30% (W/W).

In embodiments, the fast diffusing solvent is a solvent that is highly miscible with water. In embodiments, the fast diffusing solvent comprises one or more of alcohol (e.g., ethanol), N-methyl-2-pyrrolidone (NMP), dimethyl sulfoxide (DMSO), or any combinations thereof. In embodiments, the fast diffusing solvent comprises ethanol. In embodiments, the fast diffusing solvent is ethanol.

### Active Pharmaceutical Ingredient (API)

In aspects, the compositions of the disclosure comprise an active pharmaceutical ingredient (API).

In embodiments, the API is present in the composition at a concentration of about 0.1% to about 15% (W/W). In embodiments, the API is present in the composition at a concentration of about 2% to about 6% (W/W).

In embodiments, the API is present in the composition at a concentration of at least about 0.1%, or at least about 0.5%, or at least about 1%, or at least about 2%, or at least about 3%, or at least about 4%, or at least about 5%, or at least about 6%, or at least about 7%, or at least about 8%, or at least about 9%, or at least about 10%, or at least about 12%, or at least about 15% or at least about 15% (W/W), or more.

Active Pharmaceutical ingredients (API) s can be any API and in certain embodiments include peptide APIs, including GPCR modulator peptides.

In embodiments, the API comprises a peptide. In embodiments, the API is a peptide, or a pharmaceutically acceptable salt thereof. In embodiments, the pharmaceutically acceptable salt of a peptide is an acetate salt, a trifluoroacetate salt, a phosphate salt, a phosphite salt, a propionate salt, a chloride salt, a fumarate salt, a citrate salt, a tartrate salt, an oxalate salt, a succinate salt, a mandelate salt, a methanesulfonate salt, a p-toluenesulfonate salt, a bromide salt, an iodide salt, a hydroxide salt, a sulfate salt, a sulfite salt, a nitrate salt, a malate salt, a maleate salt, an aspartate salt, a glutamate salt, a lactate salt, a gluconate salt, a benzoate salt, a salicylate salt, an ethanesulfonate salt, a naphthalenesulfonate salt, or a camphorsulfonate salt.

In embodiments, the peptide comprises one or more non-canonical amino acids.

In embodiments, the peptide comprises at least about 5, 6, 7, 8, 9, or 10 amino acids.

In embodiments, the API is a G protein coupled receptor (GPCR) modulator, such as an agonist, antagonist, or biased signaling molecule. In embodiments, the API comprises a GPCR modulator peptide or a pharmaceutically acceptable salt thereof. In embodiments, the API is a GPCR modulator peptide or a pharmaceutically acceptable salt thereof.

In embodiments, the GPCR is a melanocortin receptor, such as a MC4R. In embodiments, the API is a MC4R agonist. In embodiments, the API is or comprises a MC4R agonist peptide or a pharmaceutically acceptable salt thereof. In embodiments, the API is or comprises a MC4R agonist peptide or a pharmaceutically acceptable salt thereof. In embodiments, the API is or comprises a MC4R agonist peptide comprising one or more non-canonical amino acids. Methods of designing and preparing MC4R agonist peptides can be found in PCT/US2025/030832, the contents of which are hereby incorporated by reference herein in their entirety.

In embodiments, the API are for the treating and/or prevention of diseases or disorders associated with upregulation of MC4R. In embodiments, the API described herein, demonstrate enhanced MC4R function. In embodiments, the API described herein are MC4R agonistic peptides or salts thereof that display superior selectivity towards MC4R as compared with the other melanocortin receptors (such as MC1R). In embodiments, the API described herein display varying activity on G-protein coupled pathways stemming from the MC4R, namely one or more of Gs-coupled (e.g, cAMP), Gq-coupled, and B-arrestin dependent signaling pathways. In embodiments, the API of the present disclosure have increased in vitro selectivity and potency, in vivo effectiveness, pharmacokinetic attributes, and/or stability when compared to other APIs, e.g., other melanocortin receptor binding peptides or salts thereof.

In embodiments, the API is a peptide, or a salt thereof, comprising the amino acid sequence of formula (I):

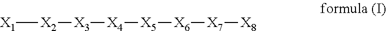

wherein in formula (I):
X₃ is 3-aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

In embodiments, the API is a peptide, or a salt thereof, comprising the amino acid sequence of formula (I):

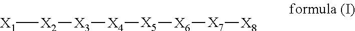

wherein in formula (I): X₁, X₂, X₃, X₄, X₅, X₆, X₇, and X₈ are each independently an amino acid selected from Table 1.

In embodiments, the API is a peptide, or a salt thereof, comprising the amino acid sequence of formula (I):

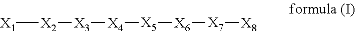

wherein in formula (I): X₁, X₂, X₃, X₄, X₅, X₆, X₇, and X₈ are each independently an amino acid selected from Table 1, Table 2, Table 3, Table A1, Table A1A, Table A2, and Table A2A.

In embodiments, the API is a peptide, or a salt thereof, consists of the amino acid sequence as set forth in formula (I).

In embodiments, the API is a peptide, or a salt thereof, of Table 1, Table 2, Table 3. Table A1, Table A1A, Table A2, and Table A2A are not limited to N-terminal functional group, C-terminal functional group, and/or status or type of cyclic function.

In embodiments, the salt of a peptide comprising or consisting of formula (I) is an acetate salt, a trifluoroacetate salt, a phosphate salt, a phosphite salt, a propionate salt, a chloride salt, a fumarate salt, a citrate salt, a tartrate salt, an oxalate salt, a succinate salt, a mandelate salt, a methanesulfonate salt, a p-toluenesulfonate salt, a bromide salt, an iodide salt, a hydroxide salt, a sulfate salt, a sulfite salt, a nitrate salt, a malate salt, a maleate salt, an aspartate salt, a glutamate salt, a lactate salt, a gluconate salt, a benzoate salt, a salicylate salt, an ethanesulfonate salt, a naphthalenesulfonate salt, or a camphorsulfonate salt.

In embodiments, the salt of a peptide comprising or consisting of formula (I) is a pharmaceutically acceptable salt. In embodiments, the pharmaceutically acceptable salt is selected from a sulfate salt, a citrate salt, an acetate salt, a oxalate salt, a chloride salt, a bromide salt, an iodide salt, a nitrate salt, a bisulfate salt, a phosphate salt, an acid phosphate salt, an isonicotinate salt, a lactate salt, a salicylate salt, an acid citrate salt, a tartrate salt, an oleate salt, a tannate salt, a pantothenate salt, a bitartrate salt, an ascorbate salt, a succinate salt, a maleate salt, a gentisinate salt, a fumarate salt, a gluconate salt, a glucuronate salt, a saccharate salt, a formate salt, a benzoate salt, a glutamate salt, a methanesulfonate “mesylate” salt, an ethanesulfonate salt, a benzenesulfonate salt, a p-toluenesulfonate salt, and a pamoate salt (i.e., 1, l′-methylene-bis-(2-hydroxy-3-naphthoate)).

In embodiments, the salts of peptides of formula (I) are acetate salts. In embodiments, the peptide of formula (I) comprises one charged atom, and the salt comprises one acetate counterion. In embodiments, the peptide of formula (I) comprises two charged atoms, and the salt comprises two acetate counterions (e.g. bisacetate salt). In embodiments, the peptide of formula (I) comprises three charged atoms, and the salt comprises three acetate counterions (e.g. trisacetate salt). In embodiments, the peptide of formula (I) comprises four charged atoms, and the salt comprises four acetate counterions (e.g. tetraacetate salt).

In embodiments, the salts of peptides of formula (I) of the disclosure are trifluoroacetate salts. In embodiments, the peptide of formula (I) comprises one charged atom, and the salt comprises one trifluoroacetate counterion. In embodiments, the peptide of formula (I) comprises two charged atoms, and the salt comprises two trifluoroacetate counterions (e.g. bistrifluoroacetate salt). In embodiments, the peptide of formula (I) comprises three charged atoms, and the salt comprises three trifluoroacetate counterions (e.g. tristrifluoroacetate salt). In embodiments, the peptide of formula (I) comprises four charged atoms, and the salt comprises four trifluoroacetate counterions (e.g. tetrafluoroacetate salt).

In embodiments, the salt of a peptide comprising or consisting of formula (I) is an acetate salt. In embodiments, the salt of a peptide comprising or consisting of formula (I) is a trifluoroacetate salt.

In embodiments, the API is a peptide, or a salt thereof, wherein the peptide is a cyclic peptide.

In embodiments, the cyclic peptide comprises a disulfide bridge or a lactam bridge.

In embodiments, the cyclic peptide has the formula (II):

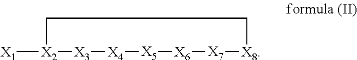

In embodiments, the cyclic peptide of formula (II) is a cyclic peptide of any one of:

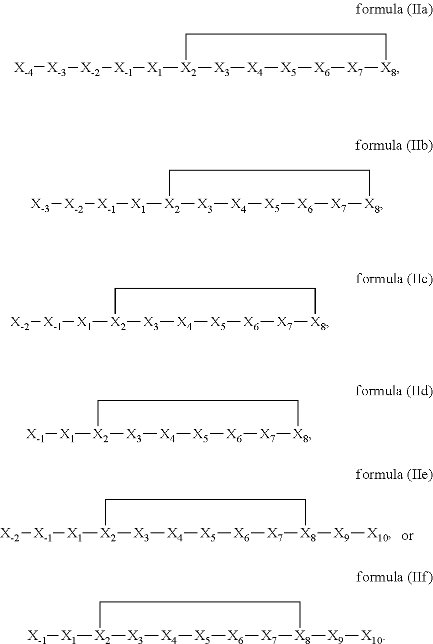

wherein X₋₁, X₋₂, X₋₃, X₋₄, X₁, X₂, X₃, X₄, X₅, X₆, X₇, X₈, X₉, and X₁₀ are each independently an amino acid selected from Table 1 and Table 2.

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3.

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the API is a peptide, or a salt thereof, wherein the peptide consists of the amino acid sequence as set forth in formula (II). In embodiments, the peptide consists of the amino acid sequence as set forth in formula (IIa). In embodiments, the peptide consists of the amino acid sequence as set forth in formula (IIb). In embodiments, the peptide consists of the amino acid sequence as set forth in formula (IIc). In embodiments, the peptide consists of the amino acid sequence as set forth in formula (IId). In embodiments, the peptide consists of the amino acid sequence as set forth in formula (IIe). In embodiments, the peptide consists of the amino acid sequence as set forth in formula (IIf).

In embodiments, the cyclic peptide has a formula selected from formula (BII), formula (CII), formula (DII), formula (EII), and formula (FII).

In embodiments, the cyclic peptide of formula (II) is a cyclic peptide of any one of formula (IIa), formula (IIb), formula (IIc), formula (IId), formula (IIe), or formula (IIf).

In embodiments, the cyclic peptide of formula (BII) is a cyclic peptide of any one of formula (BIIa), formula (BIIb), formula (BIIc), formula (BIId), formula (BIIe), or formula (BIIf).

In embodiments, the cyclic peptide of formula (CII) is a cyclic peptide of any one of formula (CIIa), formula (CIIb), formula (CIIc), formula (CIId), formula (CIIe), or formula (CIIf).

In embodiments, the cyclic peptide of formula (DII) is a cyclic peptide of any one of formula (DIIa), formula (DIIb), formula (DIIc), formula (DIId), formula (IIe), or formula (DIIf).

In embodiments, the cyclic peptide of formula (EII) is a cyclic peptide of any one of formula (EIIa), formula (EIIb), formula (EIIc), formula (EIId), formula (EIIe), or formula (EIIf).

In embodiments, the cyclic peptide of formula (FII) is a cyclic peptide of any one of formula (FIIa), formula (FIIb), formula (FIIc), formula (FIId), formula (FIIe), or formula (IFIf).

In embodiments, the API is a peptide, or a salt thereof, wherein the peptide consists of the amino acid sequence as set forth in formula (BII). In embodiments, the peptide consists of the amino acid sequence as set forth in formula (BIIa). In embodiments, the peptide consists of the amino acid sequence as set forth in formula (BIIb). In embodiments, the peptide consists of the amino acid sequence as set forth in formula (BIIc). In embodiments, the peptide consists of the amino acid sequence as set forth in formula (BIId). In embodiments, the peptide consists of the amino acid sequence as set forth in formula (BIle). In embodiments, the peptide consists of the amino acid sequence as set forth in formula (BIIf).

In embodiments, the API is a peptide, or a salt thereof, wherein the peptide consists of the amino acid sequence as set forth in formula (CII). In embodiments, the peptide consists of the amino acid sequence as set forth in formula (CIIa). In embodiments, the peptide consists of the amino acid sequence as set forth in formula (CIIb). In embodiments, the peptide consists of the amino acid sequence as set forth in formula (CIIc). In embodiments, the peptide consists of the amino acid sequence as set forth in formula (CIId). In embodiments, the peptide consists of the amino acid sequence as set forth in formula (CIIe). In embodiments, the peptide consists of the amino acid sequence as set forth in formula (CIIf).

In embodiments, the API is a peptide, or a salt thereof, wherein the peptide consists of the amino acid sequence as set forth in formula (DII). In embodiments, the peptide consists of the amino acid sequence as set forth in formula (DIIa). In embodiments, the peptide consists of the amino acid sequence as set forth in formula (DIIb). In embodiments, the peptide consists of the amino acid sequence as set forth in formula (DIIc). In embodiments, the peptide consists of the amino acid sequence as set forth in formula (DIId). In embodiments, the peptide consists of the amino acid sequence as set forth in formula (DIIe). In embodiments, the peptide consists of the amino acid sequence as set forth in formula (DIIf).

In embodiments, the API is a peptide, or a salt thereof, wherein the peptide consists of the amino acid sequence as set forth in formula (EII). In embodiments, the peptide consists of the amino acid sequence as set forth in formula (EIIa). In embodiments, the peptide consists of the amino acid sequence as set forth in formula (EIIb). In embodiments, the peptide consists of the amino acid sequence as set forth in formula (EIIc). In embodiments, the peptide consists of the amino acid sequence as set forth in formula (EIId). In embodiments, the peptide consists of the amino acid sequence as set forth in formula (EIIe). In embodiments, the peptide consists of the amino acid sequence as set forth in formula (EIIf).

In embodiments, the API is a peptide, or a salt thereof, wherein the peptide consists of the amino acid sequence as set forth in formula (FII). In embodiments, the peptide consists of the amino acid sequence as set forth in formula (FIIa). In embodiments, the peptide consists of the amino acid sequence as set forth in formula (FIIb). In embodiments, the peptide consists of the amino acid sequence as set forth in formula (FIIc). In embodiments, the peptide consists of the amino acid sequence as set forth in formula (FIId). In embodiments, the peptide consists of the amino acid sequence as set forth in formula (FIIe). In embodiments, the peptide consists of the amino acid sequence as set forth in formula (FIIf).

In embodiments, in formula (BIIa), formula (BIIb), formula (BIIc), formula (BIId), formula (BIIe), or formula (BIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (CIIa), formula (CIIb), formula (CIIc), formula (CIId), formula (CIIe), or formula (CIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (DIIa), formula (DIIb), formula (DIIc), formula (DIId), formula (DIIe), or formula (DIIf), X₋₁, X₋₂, X₋₃, X₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (EIIa), formula (EIIb), formula (EIIc), formula (EIId), formula (EIIe), or formula (EIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (FIIa), formula (FIIb), formula (FIIc), formula (FIId), formula (FIIe), or formula (FIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (II) is selected from Table 2. Table A2 and Table A2A.

In embodiments, the salt of a peptide comprising or consisting of formula (II) is an acetate salt, a trifluoroacetate salt, a phosphate salt, a phosphite salt, a propionate salt, a chloride salt, a fumarate salt, a citrate salt, a tartrate salt, an oxalate salt, a succinate salt, a mandelate salt, a methanesulfonate salt, a p-toluenesulfonate salt, a bromide salt, an iodide salt, a hydroxide salt, a sulfate salt, a sulfite salt, a nitrate salt, a malate salt, a maleate salt, an aspartate salt, a glutamate salt, a lactate salt, a gluconate salt, a benzoate salt, a salicylate salt, an ethanesulfonate salt, a naphthalenesulfonate salt, or a camphorsulfonate salt.

In embodiments, the salt of a peptide comprising or consisting of formula (II) is a pharmaceutically acceptable salt. In embodiments, the pharmaceutically acceptable salt is selected from a sulfate salt, a citrate salt, an acetate salt, a oxalate salt, a chloride salt, a bromide salt, an iodide salt, a nitrate salt, a bisulfate salt, a phosphate salt, an acid phosphate salt, an isonicotinate salt, a lactate salt, a salicylate salt, an acid citrate salt, a tartrate salt, an oleate salt, a tannate salt, a pantothenate salt, a bitartrate salt, an ascorbate salt, a succinate salt, a maleate salt, a gentisinate salt, a fumarate salt, a gluconate salt, a glucuronate salt, a saccharate salt, a formate salt, a benzoate salt, a glutamate salt, a methanesulfonate “mesylate” salt, an ethanesulfonate salt, a benzenesulfonate salt, a p-toluenesulfonate salt, and a pamoate salt (i.e., 1,1′-methylene-bis-(2-hydroxy-3-naphthoate)).

In embodiments, the salts of peptides of formula (II) are acetate salts. In embodiments, the peptide of formula (I) comprises one charged atom, and the salt comprises one acetate counterion. In embodiments, the peptide of formula (I) comprises two charged atoms, and the salt comprises two acetate counterions (e.g. bisacetate salt). In embodiments, the peptide of formula (II) comprises three charged atoms, and the salt comprises three acetate counterions (e.g. trisacetate salt). In embodiments, the peptide of formula (I) comprises four charged atoms, and the salt comprises four acetate counterions (e.g. tetraacetate salt).

In embodiments, the salts of peptides of formula (II) of the disclosure are trifluoroacetate salts. In embodiments, the peptide of formula (II) comprises one charged atom, and the salt comprises one trifluoroacetate counterion. In embodiments, the peptide of formula (II) comprises two charged atoms, and the salt comprises two trifluoroacetate counterions (e.g. bistrifluoroacetate salt). In embodiments, the peptide of formula (II) comprises three charged atoms, and the salt comprises three trifluoroacetate counterions (e.g. tristrifluoroacetate salt). In embodiments, the peptide of formula (II) comprises four charged atoms, and the salt comprises four trifluoroacetate counterions (e.g. tetrafluoroacetate salt).

In embodiments, the salt of a peptide comprising or consisting of formula (II) is an acetate salt. In embodiments, the salt of a peptide comprising or consisting of formula (II) is a trifluoroacetate salt.

In embodiments, the peptide of formula (I) or formula (II) is selected from Table 1, Table 2, Table A1, Table A1A, Table A2, and Table A2A.

In embodiments, the peptides of formula (I) or formula (II) comprise a N-terminal functional group, a C-terminal functional group, and/or a cyclic function.

In embodiments, the peptide of formula (I) or formula (II) is selected from Table 1, Table 2, Table A1, Table A1A, Table A2, and Table A2A, wherein the N-terminal, C-terminal and/or cyclic structure are optional features.

In embodiments, the peptide is a cyclic peptide (e.g., Table 1). In embodiments, the cyclic peptide comprises a disulfide bridge or a lactam bridge.

In embodiments, the peptide of formula (I) or formula (II) is lipidated (e.g., Table 2).

In embodiments, the peptide of formula (I) is selected from Table A1, Table A1A, Table A2 and Table A2A.

In embodiments, the peptides of Table 1, Table 2, Table A1, Table A1A, Table A2 and Table A2A are not limited to N-terminal functional group, C-terminal functional group, and/or status or type of cyclic function.

TABLE A1
Exemplary peptides.

| Molecule Name | X₁ | X₂ | X₃ | X₄ | X₅ | X₆ | X₇ | X₈ |
|---|---|---|---|---|---|---|---|---|
| 1093-1 | D-Nar | Cys | Aib(O- | Gln | D-Phe(4-F) | Arg | Trp(6-F) | Cys |
| | | | cyclic) | | | | | |
| 1092-1 | D-Nar | Cys | Aib(O- | Gln | D-Phe | Arg | Trp(6-F) | Cys |
| | | | cyclic) | | | | | |
| 1107-1 | D-Nar | Cys | Aib(O- | hCit | D-Phe(4-F) | Arg | Trp(6-F) | Cys |
| | | | cyclic) | | | | | |
| 1106-1 | D-Nar | Cys | Aib(O- | Cit | D-Phe(4-F) | Arg | Trp(6-F) | Cys |
| | | | cyclic) | | | | | |
| 1103-1 | D-Nar | Cys | Aib(O- | Cit | D-Phe | Arg | Trp(6-F) | Cys |
| | | | cyclic) | | | | | |
| 1105-1 | D-Nar | Cys | Aib(O- | Cit | D-Phe(4-Me) | Arg | Trp(6-F) | Cys |
| | | | cyclic) | | | | | |
| 1095-1 | D-Nar | Cys | Aib(O- | Gln | D-Phe(4-Me) | Arg | Trp(6-F) | Cys |
| | | | cyclic) | | | | | |
| 1122 -1 | D-Nar | Cys | Aib(O- | 3-Pal | D-Phe(4-F) | Arg | Trp(6-F) | Cys |
| | | | cyclic) | | | | | |
| 1102-1 | D-Nar | Cys | Aib(O- | hGln | D-Phe | Arg | Trp(6-F) | Cys |
| | | | cyclic) | | | | | |
| 1058-1 | D-Nar | Cys | Aib(O- | His | D-Phe | Arg | Trp(6-F) | Cys |
| | | | cyclic) | | | | | |
| 1123-1 | D-Nar | Cys | Aib(O- | Orn | D-Phe(4-F) | Arg | Trp(6-F) | Cys |
| | | | cyclic) | | | | | |
| 1158-1 | D-Nar | Cys | Aib(O- | Gln | D-Phe(4-F) | Arg | Trp(6-F) | Per |
| | | | cyclic) | | | | | |

In embodiments, the peptide of formula (I) is selected from Table B1, Table BIA, Table B2 and Table B2A.

In embodiments, the peptides of Table B1, Table B1A, Table B2 and Table B2A are not limited to N-terminal functional group, C-terminal functional group, and/or status or type of cyclic function.

TABLE B1
Exemplary peptide.

| Molecule Name | X₁ | X₂ | X₃ | X₄ | X₅ | X₆ | X₇ | X₈ |
|---|---|---|---|---|---|---|---|---|
| 1119-1 | D-Nar | Cys | Phg | 3-Pal | D-Phe(4-F) | Arg | Trp(6-F) | Cys |

In embodiments, the peptide of formula (I) is selected from Table B1A.

In embodiments, the peptide of formula (I) is selected from Table BIA, wherein the N-terminal, C-terminal and/or cyclic structure are optional feature.

TABLE B1A
Exemplary peptide with N-terminal, C-terminal and/or cyclic structure as optional feature.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1119 | D-Nar | Cys | Phg | 3-Pal | D-Phe(4-F) | Arg | Trp(6-F) | Cys | Ac | NH₂ | Disulfide |

In embodiments, the peptide of formula (I) is selected from Table B2.

TABLE B2
Exemplary lipidated peptides.

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1146-2 | Lys* | D- | Gly | D-Arg | D-Nar | Cys | Phg | 3- | D- | Arg | Trp(6- | Cys |
| | | Arg | | | | | | Pal | Phe(4- | | F) | |
| | | | | | | | | | F) | | | |
| 1139-2 | | Lys* | Gly | D-Arg | D-Nar | Cys | Phg | 3- | D- | Arg | Trp(6- | Cys |
| | | | | | | | | Pal | Phe(4- | | F) | |
| | | | | | | | | | F) | | | |
| 1145-2 | | Lys* | PEG1 | PEG1 | D-Nar | Cys | Phg | 3- | D- | Arg | Trp(6- | Cys |
| | | | | | | | | Pal | Phe(4- | | F) | |
| 1147-2 | Lys* | D- | PEG1 | D-Arg | Beta- | Cys | Phg | 3- | D- | Arg | Trp(6- | Cys |
| | | Arg | | | homoArg | | | Pal | Phe(4- | | F) | |
| | | | | | | | | | F) | | | |

In embodiments, the peptide of formula (II) is selected from Table B2A.

In embodiments, the peptide of formula (II) is selected from Table B2A, wherein the N-terminal, C-terminal and/or cyclic structure are optional feature.

TABLE B2A
Exemplary lipidated peptides.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1146 | Lys* | D- | Gly | D- | D- | Cys | Phg | 3- | D- | Arg | Trp(6- | Cys | Ac | NH₂ | Disulfide |
| | | Arg | | Arg | Nar | | | Pal | Phe(4- | | F) | | | | |
| | | | | | | | | | F) | | | | | | |
| 1139 | | Lys* | Gly | D- | D- | Cys | Phg | 3- | D- | Arg | Trp(6- | Cys | Ac | NH₂ | Disulfide |
| | | | | Arg | Nar | | | Pal | Phe(4- | | F) | | | | |
| | | | | | | | | | F) | | | | | | |
| 1145 | | Lys* | PEG1 | PEG1 | D- | Cys | Phg | 3- | D- | Arg | Trp(6- | Cys | Ac | NH₂ | Disulfide |
| | | | | | Nar | | | Pal | Phe(4- | | F) | | | | |
| | | | | | | | | | F) | | | | | | |
| 1147 | Lys* | D- | PEG1 | D- | Beta- | Cys | Phg | 3- | D- | Arg | Trp(6- | Cys | Ac | NH₂ | Disulfide |
| | | Arg | | Arg | homoArg | | | Pal | Phe(4- | | F) | | | | |
| | | | | | | | | | F) | | | | | | |

In embodiments, the peptide of formula (I) is selected from Table C1, Table C1A, Table C2 and Table C2A.

In embodiments, the peptides of Table C1, Table CIA, Table C2 and Table C2A are not limited to N-terminal functional group, C-terminal functional group, and/or status or type of cyclic function.

TABLE C1
Exemplary peptide.

| Molecule | | | | | | | | |
|---|---|---|---|---|---|---|---|---|
| Name | X₁ | X₂ | X₃ | X₄ | X₅ | X₆ | X₇ | X₈ |
| 1094-1 | D- | Cys | D- | Gln | D- | Arg | Trp(6- | Cys |
| | Nar | | aMeOrn | | Phe | | F) | |

In embodiments, the peptide of formula (I) is selected from Table CIA.

In embodiments, the peptide of formula (I) is selected from Table CIA, wherein the N-terminal, C-terminal and/or cyclic structure are optional feature.

TABLE C1A
Exemplary peptides.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1094 | D- | Cys | D- | Gln | D- | Arg | Trp(6- | Cys | Ac | NH2 | Disulfide |
| | Nar | | aMeOrn | | Phe | | F) | | | | |

In embodiments, the peptide of formula (II) is selected from Table C2.

TABLE C2
Exemplary lipidated peptides

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1148-2 | Lys* | D- | Gly | D- | D- | Cys | D- | Gln | D- | Arg | Trp(6- | Cys |
| | | Arg | | Arg | Nar | | aMeOrn | | Phe | | F) | |
| 1149-2 | | Lys* | Gly | D- | D- | Cys | D- | Gln | D- | Arg | Trp(6- | Cys |
| | | | | Arg | Nar | | aMeOrn | | Phe | | F) | |
| 1137-2 | | Lys* | PEG1 | PEG1 | D- | Cys | D- | Gln | D- | Arg | Trp(6- | Cys |
| | | | | | Nar | | aMeOrn | | Phe | | F) | |
| 1136-2 | Lys* | D- | PEG1 | D- | Beta- | Cys | D- | Gln | D- | Arg | Trp(6- | Cys |
| | | Arg | | Arg | homoArg | | aMeOrn | | Phe | | F) | |

In embodiments, the peptide of formula (II) is selected from Table C2A.

In embodiments, the peptide of formula (II) is selected from Table C2A, wherein the N-terminal, C-terminal and/or cyclic structure are optional feature.

TABLE C2A
Exemplary lipidated peptides

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1148 | Lys* | D- | Gly | D- | D- | Cys | D- | Gln | D- | Arg | Trp(6- | Cys | Ac | NH₂ | Disulfide |
| | | Arg | | Arg | Nar | | aMeOrn | | Phe | | F) | | | | |
| 1149 | | Lys* | Gly | D- | D- | Cys | D- | Gln | D- | Arg | Trp(6- | Cys | Ac | NH₂ | Disulfide |
| | | | | Arg | Nar | | aMeOrn | | Phe | | F) | | | | |
| 1137 | | Lys* | PEG1 | PEG1 | D- | Cys | D- | Gln | D- | Arg | Trp(6- | Cys | Ac | NH₂ | Disulfide |
| | | | | | Nar | | aMeOrn | | Phe | | F) | | | | |
| 1136 | Lys* | D- | PEG1 | D- | Beta- | Cys | D- | Gln | D- | Arg | Trp(6- | Cys | Ac | NH₂ | Disulfide |
| | | Arg | | Arg | homoArg | | aMeOrn | | Phe | | F) | | | | |

In embodiments, the peptides of Table D1, Table DIA, Table D2 and Table D2A are not limited to N-terminal functional group, C-terminal functional group, and/or status or type of cyclic function.

In embodiments, the peptide of formula (I) is selected from Table DI.

TABLE D1
Exemplary peptides.

| Molecule | | | | | | | | |
|---|---|---|---|---|---|---|---|---|
| Name | X₁ | X₂ | X₃ | X₄ | X₅ | X₆ | X₇ | X₈ |
| 1119-1 | D-Nar | Cys | Phg | 3-Pal | D-Phe(4-F) | Arg | Trp(6-F) | Cys |
| 1094-1 | D-Nar | Cys | D-aMeOrn | Gln | D-Phe | Arg | Trp(6-F) | Cys |
| 1093-1 | D-Nar | Cys | Aib(O-cyclic) | Gln | D-Phe(4-F) | Arg | Trp(6-F) | Cys |
| 1092-1 | D-Nar | Cys | Aib(O-cyclic) | Gln | D-Phe | Arg | Trp(6-F) | Cys |
| 1107-1 | D-Nar | Cys | Aib(O-cyclic) | hCit | D-Phe(4-F) | Arg | Trp(6-F) | Cys |
| 1106-1 | D-Nar | Cys | Aib(O-cyclic) | Cit | D-Phe(4-F) | Arg | Trp(6-F | Cys |
| 1015-1 | D-Nar | Cys | L-aMeGlu | His | D-Phe | Arg | Trp(6-Me) | Cys |
| 1035-1 | D-Nar | Cys | L-aMeGlu | His | D-Phe | Arg | Trp(6-F) | Cys |
| 1091-1 | D-Nar | Cys | hGlu | Gln | D-Phe | Arg | Trp(6-F) | Cys |
| 1096-1 | D-Nar | Cys | D-aMeSer | Gln | D-Phe | Arg | Trp(6-F) | Cys |
| 1043-1 | D-Nar | Cys | L-aMeGlu | His | D-Phe | Arg | Trp(5-Me) | Cys |
| 1012-1 | Arg | Cys | Cyclo-Leu | Gln | D-Phe | Arg | Trp | Cys |
| 1049-1 | D-Nar | Cys | hGlu | His | D-Phe | Arg | Trp(6-Me) | Cys |
| 1041-1 | D-Nar | Cys | L-aMeGlu | His | D-Phe(4-F) | Arg | Trp(6-Me) | Cys |
| 1099-1 | D-Nar | Cys | Ala(2-Me) | Gln | D-Phe(4-F) | Arg | Trp(6-F) | Cys |
| 1030-1 | Beta- | Cys | L-aMeGlu | His | D-Phe(3- | Arg | Trp | Cys |
| | homoArg | | | | CF3) | | | |
| 1121-1 | D-Nar | Glu | L-aMeAsp | His | D-Phe(4-F) | Arg | Trp(6-F) | Dap |
| 1042-1 | D-Nar | Cys | L-aMeAsp | His | D-Phe | Arg | Trp(6-F) | Cys |
| 1024-1 | Beta- | Cys | L-aMeGlu | His | D-Phe | Arg | Trp(6-Me) | Cys |
| | homoArg | | | | | | | |
| 1064-1 | D-Nar | Cys | L-aMeAsp | His | D-Phe | Arg | Trp(6-Me) | Cys |
| 1037-1 | D-Nar | Cys | Ala(2-Me) | His | D-Phe | Arg | Trp(6-F) | Cys |
| 1019-1 | D-Nar | Cys | L-aMeGlu | His | D-Phe | Arg | Trp | Cys |
| 1085-1 | D-Nar | Cys | L-aMeGlu | His | D-Phe(3-F) | Arg | Trp(6-Me) | Cys |
| 1016-1 | D-Arg | Cys | L-aMeGlu | His | D-Phe | Arg | Trp(6-Me) | Cys |
| 1111-1 | D-Nar | Cys | Phg | His | D-Phe(4-F) | Arg | Trp(6-F) | Cys |
| 1108-1 | D-Nar | Cys | Cyclo-Leu | 3-Pal | D-Phe | Arg | Trp(6-F) | Cys |
| 1050-1 | D-Nar | Cys | L-aMeGlu | His | D-Phe(3- | Arg | Trp(6-Me) | Cys |
| | | | | | CF3) | | | |
| 1044-1 | D-Nar | Cys | D-bhGlu | His | D-Phe | Arg | Trp(6-Me) | Cys |
| 1040-1 | D-Nar | Cys | L-aMeGlu | His | D-Phe(3-F) | Arg | Trp(6-F) | Cys |
| 1039-1 | D-Nar | Cys | D-aMeSer | His | D-Phe | Arg | Trp(6-Me) | Cys |
| 1033-1 | Beta- | Cys | bhGlu | His | D-Phe | Arg | Trp | Cys |
| | homoArg | | | | | | | |
| 1013-1 | Arg | Cys | Ala(2-Me) | Gln | D-Phe | Arg | Trp | Cys |
| 1124-1 | D-Nar | Cys | D-aMeOrn | 3-Pal | D-Phe(4-F) | Arg | Trp(6-F) | Cys |
| 1122-1 | D-Nar | Cys | Aib(O-cyclic) | 3-Pal | D-Phe(4-F) | Arg | Trp(6-F) | Cys |
| 1126-1 | D-Nar | Cys | D-aMeOrn | 3-Pal | D-Phe | Arg | Trp(6-F) | Cys |
| 1158-1 | D-Nar | Cys | Aib(O-cyclic) | Gln | D-Phe(4-F) | Arg | Trp(6-F) | Pen |

In embodiments, the peptide of formula (I) is selected from Table DIA.

In embodiments, the peptide of formula (I) is selected from Table DIA, wherein the N-terminal. C-terminal and/or cyclic structure are optional feature.

TABLE D1A
Exemplary peptides

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1119 | D-Nar | Cys | Phg | 3-Pal | D-Phe(4- | Arg | Trp(6-F) | Cys | Ac | NH₂ | Disulfide |
| 1094 | D-Nar | Cys | D- | Gln | D-Phe | Arg | Trp(6-F) | Cys | Ac | NH₂ | Disulfide |
| | | | aMeOrn | | | | | | | | |
| 1093 | D-Nar | Cys | Aib(O- | Gln | D-Phe(4- | Arg | Trp(6-F) | Cys | Ac | NH₂ | Disulfide |
| | | | cyclic) | | F) | | | | | | |
| 1092 | D-Nar | Cys | Aib(O- | Gln | D-Phe | Arg | Trp(6-F) | Cys | Ac | NH₂ | Disulfide |
| | | | cyclic) | | | | | | | | |
| 1107 | D-Nar | Cys | Aib(O- | hCit | D-Phe(4- | Arg | Trp(6-F) | Cys | Ac | NH₂ | Disulfide |
| | | | cyclic) | | F) | | | | | | |
| 1106 | D-Nar | Cys | Aib(O- | Cit | D-Phe(4- | Arg | Trp(6-F) | Cys | Ac | NH₂ | Disulfide |
| | | | cyclic) | | F) | | | | | | |
| 1015 | D-Nar | Cys | L- | His | D-Phe | Arg | Trp(6- | Cys | Ac | NH₂ | Disulfide |
| | | | aMeGlu | | | | Me) | | | | |
| 1035 | D-Nar | Cys | L- | His | D-Phe | Arg | Trp(6-F) | Cys | Ac | NH₂ | Disulfide |
| | | | aMeGlu | | | | | | | | |
| 1091 | D-Nar | Cys | hGlu | Gln | D-Phe | Arg | Trp(6-F) | Cys | Ac | NH₂ | Disulfide |
| 1096 | D-Nar | Cys | D- | Gln | D-Phe | Arg | Trp(6-F) | Cys | Ac | NH₂ | Disulfide |
| | | | aMeSer | | | | | | | | |
| 1043 | D-Nar | Cys | L- | His | D-Phe | Arg | Trp(5- | Cys | Ac | NH₂ | Disulfide |
| | | | aMeGlu | | | | Me) | | | | |
| 1012 | Arg | Cys | Cyclo- | Gln | D-Phe | Arg | Trp | Cys | Ac | NH₂ | Disulfide |
| | | | Leu | | | | | | | | |
| 1049 | D-Nar | Cys | hGlu | His | D-Phe | Arg | Trp(6- | Cys | Ac | NH₂ | Disulfide |
| | | | | | | | Me) | | | | |
| 1041 | D-Nar | Cys | L- | His | D-Phe(4- | Arg | Trp(6- | Cys | Ac | NH₂ | Disulfide |
| | | | aMeGlu | | F) | | Me) | | | | |
| 1099 | D-Nar | Cys | Ala(2- | Gln | D-Phe(4- | Arg | Trp(6-F) | Cys | Ac | NH₂ | Disulfide |
| | | | Me) | | F) | | | | | | |
| 1030 | Beta- | Cys | L- | His | D-Phe(3- | Arg | Trp | Cys | Ac | NH₂ | Disulfide |
| | homoArg | | aMeGlu | | CF3) | | | | | | |
| 1121 | D-Nar | Glu | L- | His | D-Phe(4- | Arg | Trp(6-F) | Dap | Ac | NH₂ | Lactam |
| | | | aMeAsp | | F) | | | | | | |
| 1042 | D-Nar | Cys | L- | His | D-Phe | Arg | Trp(6-F) | Cys | Ac | NH₂ | Disulfide |
| | | | aMeAsp | | | | | | | | |
| 1024 | Beta- | Cys | L- | His | D-Phe | Arg | Trp(6- | Cys | Ac | NH₂ | Disulfide |
| | homoArg | | aMeGlu | | | | Me) | | | | |
| 1064 | D-Nar | Cys | L- | His | D-Phe | Arg | Trp(6- | Cys | Ac | NH₂ | Disulfide |
| | | | aMeAsp | | | | Me) | | | | |
| 1037 | D-Nar | Cys | Ala(2- | His | D-Phe | Arg | Trp(6-F) | Cys | Ac | NH₂ | Disulfide |
| | | | Me) | | | | | | | | |
| 1019 | D-Nar | Cys | L- | His | D-Phe | Arg | Trp | Cys | Ac | NH₂ | Disulfide |
| | | | aMeGlu | | | | | | | | |
| 1085 | D-Nar | Cys | L- | His | D-Phe(3- | Arg | Trp(6- | Cys | Ac | NH₂ | Disulfide |
| | | | aMeGlu | | F) | | Me) | | | | |
| 1016 | D-Arg | Cys | L- | His | D-Phe | Arg | Trp(6- | Cys | Ac | NH₂ | Disulfide |
| | | | aMeGlu | | | | Me) | | | | |
| 1111 | D-Nar | Cys | Phg | His | D-Phe(4- | Arg | Trp(6-F) | Cys | Ac | NH₂ | Disulfide |
| | | | | | F) | | | | | | |
| 1108 | D-Nar | Cys | Cyclo- | 3-Pal | D-Phe | Arg | Trp(6-F) | Cys | Ac | NH₂ | Disulfide |
| | | | Leu | | | | | | | | |
| 1050 | D-Nar | Cys | L- | His | D-Phe(3- | Arg | Trp(6- | Cys | Ac | NH₂ | Disulfide |
| | | | aMeGlu | | CF3) | | Me) | | | | |
| 1044 | D-Nar | Cys | D-bhGlu | His | D-Phe | Arg | Trp(6- | Cys | Ac | NH₂ | Disulfide |
| | | | | | | | Me) | | | | |
| 1040 | D-Nar | Cys | L- | His | D-Phe(3- | Arg | Trp(6-F) | Cys | Ac | NH₂ | Disulfide |
| | | | aMeGlu | | F) | | | | | | |
| 1039 | D-Nar | Cys | D- | His | D-Phe | Arg | Trp(6- | Cys | Ac | NH₂ | Disulfide |
| | | | aMeSer | | | | Me) | | | | |
| 1033 | Beta- | Cys | bhGlu | His | D-Phe | Arg | Trp | Cys | Ac | NH₂ | Disulfide |
| | homoArg | | | | | | | | | | |
| 1013 | Arg | Cys | Ala(2- | Gln | D-Phe | Arg | Trp | Cys | Ac | NH₂ | Disulfide |
| | | | Me) | | | | | | | | |
| 1124 | D-Nar | Cys | D- | 3-Pal | D-Phe(4- | Arg | Trp(6-F) | Cys | Ac | NH₂ | Disulfide |
| | | | aMeOrn | | F) | | | | | | |
| 1122 | D-Nar | Cys | Aib(O- | 3-Pal | D-Phe(4- | Arg | Trp(6-F) | Cys | Ac | NH₂ | Disulfide |
| | | | cyclic) | | F | | | | | | |
| 1126 | D-Nar | Cys | D- | 3-Pal | D-Phe | Arg | Trp(6-F) | Cys | Ac | NH₂ | Disulfide |
| | | | aMeOrn | | | | | | | | |
| 1158 | D-Nar | Cys | Aib(O- | Gln | D-Phe(4- | Arg | Trp(6-F) | Pen | Ac | NH₂ | Disulfide |
| | | | cyclic) | | F) | | | | | | |

In embodiments, the peptide of formula (II) is selected from Table D2.

TABLE D2
Exemplary lapidated peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | X₉ | X₁₀ | X₁₁ |
| 1129-2 | | Lys* | Gly | D- | D- | Cys | L- | His | D- | Arg | Trp(6- | Cys | | | |
| | | | | Arg | Nar | | aMeGlu | | Phe | | Me) | | | | |
| 1128-2 | | Lys* | Glu | PRO | D- | Cys | L- | His | D- | Arg | Trp(6- | Cys | | | |
| | | | | | Nar | | aMeGlu | | Phe | | Me) | | | | |
| 1131-2 | | Lys* | PEG1 | PEG1 | D- | Cys | L- | His | D- | Arg | Trp(6- | Cys | | | |
| | | | | | Nar | | aMeAsp | | Phe | | Me) | | | | |
| 1130-2 | | Lys* | Gly | Gly | beta- | Cys | L- | His | D- | Arg | Trp(6- | Cys | | | |
| | | | | | homoArg | | aMeAsp | | Phe | | Me) | | | | |
| 1146-2 | Lys* | D- | Gly | D- | D- | Cys | Phg | 3- | D- | Arg | Trp(6- | Cys | | | |
| | | Arg | | Arg | Nar | | | Pal | Phe(4- | | F) | | | | |
| | | | | | | | | | F) | | | | | | |
| 1139-2 | | Lys* | Gly | D- | D- | Cys | Phg | 3- | D- | Arg | Trp(6- | Cys | | | |
| | | | | Arg | Nar | | | Pal | Phe(4- | | F) | | | | |
| | | | | | | | | | F) | | | | | | |
| 1145-2 | | Lys* | PEG1 | PEG1 | D- | Cys | Phg | 3- | D- | Arg | Trp(6- | Cys | | | |
| | | | | | Nar | | | Pal | Phe(4- | | F) | | | | |
| | | | | | | | | | F) | | | | | | |
| 1147-2 | Lys* | D- | PEG1 | D- | Beta- | Cys | Phg | 3- | D- | Arg | Trp(6- | Cys | | | |
| | | Arg | | Arg | homoArg | | | Pal | Phe(4- | | F) | | | | |
| | | | | | | | | | F) | | | | | | |
| 1148-2 | Lys* | D- | Gly | D- | D- | Cys | D- | Gln | D- | Arg | Trp(6- | Cys | | | |
| | | Arg | | Arg | Nar | | aMeOrn | | Phe | | F) | | | | |
| 1149-2 | | Lys* | Gly | D- | D- | Cys | D- | Gln | D- | Arg | Trp(6- | Cys | | | |
| | | | | Arg | Nar | | aMeOrn | | Phe | | F) | | | | |
| 1137-2 | | Lys* | PEG1 | PEG1 | D- | Cys | D- | Gln | D- | Arg | Trp(6- | Cys | | | |
| | | | | | Nar | | aMeOrn | | Phe | | F) | | | | |
| 1136-2 | Lys* | D- | PEG1 | D- | Beta- | Cys | D- | Gln | D- | Arg | Trp(6- | Cys | | | |
| | | Arg | | Arg | homoArg | | aMeOrn | | Phe | | F) | | | | |
| 1150-2 | Lys* | D- | Gly | D- | D- | Cys | Aib(O- | Gln | D- | Arg | Trp(6- | Cys | | | |
| | | Arg | | Arg | Nar | | cyclic) | | Phe(4- | | F) | | | | |
| | | | | | | | | | F) | | | | | | |
| 1142-2 | | Lys* | Gly | D- | D- | Cys | Aib(O- | Gln | D- | Arg | Trp(6- | Cys | | | |
| | | | | Arg | Nar | | cyclic) | | Phe(4- | | F) | | | | |
| | | | | | | | | | F) | | | | | | |
| 1144-2 | | Lys* | PEG1 | PEG1 | D- | Cys | Aib(O- | Gln | D- | Arg | Trp(6- | Cys | | | |
| | | | | | Nar | | cyclic) | | Phe(4- | | F) | | | | |
| | | | | | | | | | F) | | | | | | |
| 1151-2 | Lys* | D- | PEG1 | D- | Beta- | Cys | Aib(O- | Gln | D- | Arg | Trp(6- | Cys | | | |
| | | Arg | | Arg | homoArg | | cyclic) | | Phe(4- | | F) | | | | |
| | | | | | | | | | F) | | | | | | |
| 1152-2 | Lys* | D- | Gly | D- | D- | Cys | Aib(O- | 3- | D- | Arg | Trp(6- | Cys | | | |
| | | Arg | | Arg | Nar | | cyclic) | Pal | Phe(4- | | F) | | | | |
| | | | | | | | | | F) | | | | | | |
| 1154-2 | Lys* | D- | Gly | D- | D- | Cys | D- | 3- | D- | Arg | Trp(6- | Cys | | | |
| | | Arg | | Arg | Nar | | aMeOrn | Pal | Phe(4- | | F) | | | | |
| | | | | | | | | | F) | | | | | | |
| 1156-2 | Lys* | D- | Gly | D- | D- | Cys | D- | 3- | D- | Arg | Trp(6- | Cys | | | |
| | | Arg | | Arg | Nar | | aMeOrn | Pal | Phe | | F) | | | | |
| 1200-2 | | | | | D- | Cys | L- | His | D- | Arg | Trp(6- | Cys | Gly | Gly | Lys* |
| | | | | | Arg | | aMeAsp | | Phe | | Me) | | | | |

In embodiments, the peptide of formula (II) is selected from Table D2A.

In embodiments, the peptide of formula (II) is selected from Table D2A, wherein the N-terminal. C-terminal and/or cyclic structure are an optional feature.

TABLE D2A
Exemplary lipidated peptides.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1129 | | Lys* | Gly | D- | D- | Cys | L- | His | D- | Arg | Trp(6- | Cys | Ac | NH₂ | Disulfide |
| | | | | Arg | Nar | | aMeGlu | | Phe | | Me) | | | | |
| 1128 | | Lys* | Glu | PRO | D- | Cys | L- | His | D- | Arg | Trp(6- | Cys | Ac | NH₂ | Disulfide |
| | | | | | Nar | | aMeGlu | | Phe | | Me) | | | | |
| 1131 | | Lys* | PEG1 | PEG1 | D- | Cys | L- | His | D- | Arg | Trp(6- | Cys | Ac | NH₂ | Disulfide |
| | | | | | Nar | | aMeAsp | | Phe | | Me) | | | | |
| 1130 | | Lys* | Gly | Gly | beta- | Cys | L- | His | D- | Arg | Trp(6- | Cys | Ac | NH₂ | Disulfide |
| | | | | | homoArg | | aMeAsp | | Phe | | Me) | | | | |
| 1146 | Lys* | D- | Gly | D- | D- | Cys | Phg | 3- | D- | Arg | Trp(6- | Cys | Ac | NH₂ | Disulfide |
| | | Arg | | Arg | Nar | | | Pal | Phe(4- | | F) | | | | |
| | | | | | | | | | F) | | | | | | |
| 1139 | | Lys* | Gly | D- | D- | Cys | Phg | 3- | D- | Arg | Trp(6- | Cys | Ac | NH₂ | Disulfide |
| | | | | Arg | Nar | | | Pal | Phe(4- | | F) | | | | |
| | | | | | | | | | F) | | | | | | |
| 1145 | | Lys* | PEG1 | PEG1 | D- | Cys | Phg | 3- | D- | Arg | Trp(6- | Cys | Ac | NH₂ | Disulfide |
| | | | | | Nar | | | Pal | Phe(4- | | F) | | | | |
| | | | | | | | | | F) | | | | | | |
| 1147 | Lys* | D- | PEG1 | D- | Beta- | Cys | Phg | 3- | D- | Arg | Trp(6- | Cys | Ac | NH₂ | Disulfide |
| | | Arg | | Arg | homoArg | | | Pal | Phe(4- | | F) | | | | |
| | | | | | | | | | F) | | | | | | |
| 1148 | Lys* | D- | Gly | D- | D- | Cys | D- | Gln | D- | Arg | Trp(6- | Cys | Ac | NH₂ | Disulfide |
| | | Arg | | Arg | Nar | | aMeOrn | | Phe | | F) | | | | |
| 1149 | | Lys* | Gly | D- | D- | Cys | D- | Gln | D- | Arg | Trp(6- | Cys | Ac | NH₂ | Disulfide |
| | | | | Arg | Nar | | aMeOrn | | Phe | | F) | | | | |
| 1137 | | Lys* | PEG1 | PEG1 | D- | Cys | D- | Gln | D- | Arg | Trp(6- | Cys | Ac | NH₂ | Disulfide |
| | | | | | Nar | | aMeOrn | | Phe | | F) | | | | |
| 1136 | Lys* | D- | PEG1 | D- | Beta- | Cys | D- | Gln | D- | Arg | Trp(6- | Cys | Ac | NH₂ | Disulfide |
| | | Arg | | Arg | homoArg | | aMeOrn | | Phe | | F) | | | | |
| 1150 | Lys* | D- | Gly | D- | D- | Cys | Aib(O- | Gln | D- | Arg | Trp(6- | Cys | Ac | NH₂ | Disulfide |
| | | Arg | | Arg | Nar | | cyclic) | | Phe(4- | | F) | | | | |
| | | | | | | | | | F) | | | | | | |
| 1142 | | Lys* | Gly | D- | D- | Cys | Aib(O- | Gln | D- | Arg | Trp(6- | Cys | Ac | NH₂ | Disulfide |
| | | | | Arg | Nar | | cyclic) | | Phe(4- | | F) | | | | |
| | | | | | | | | | F) | | | | | | |
| 1144 | | Lys* | PEG1 | PEG1 | D- | Cys | Aib(O- | Gln | D- | Arg | Trp(6- | Cys | Ac | NH₂ | Disulfide |
| | | | | | Nar | | cyclic) | | Phe(4- | | F) | | | | |
| | | | | | | | | | F) | | | | | | |
| 1151 | Lys* | D- | PEG1 | D- | Beta- | Cys | Aib(O- | Gln | D- | Arg | Trp(6- | Cys | Ac | NH₂ | Disulfide |
| | | Arg | | Arg | homoArg | | cyclic) | | Phe(4- | | F) | | | | |
| | | | | | | | | | F) | | | | | | |
| 1152 | Lys* | D- | Gly | D- | D- | Cys | Aib(O- | 3- | D- | Arg | Trp(6- | Cys | Ac | NH₂ | Disulfide |
| | | Arg | | Arg | Nar | | cyclic) | Pal | Phe(4- | | F) | | | | |
| | | | | | | | | | F) | | | | | | |
| 1154 | Lys* | D- | Gly | D- | D- | Cys | D- | 3- | D- | Arg | Trp(6- | Cys | Ac | NH₂ | Disulfide |
| | | Arg | | Arg | Nar | | aMeOrn | Pal | Phe(4- | | F) | | | | |
| | | | | | | | | | F) | | | | | | |
| 1156 | Lys* | D- | Gly | D- | D- | Cys | D- | 3- | D- | Arg | Trp(6- | Cys | Ac | NH₂ | Disulfide |
| | | Arg | | Arg | Nar | | aMeOrn | Pal | Phe | | F) | | | | |

| Molecule | | | | | | | | | | | | N- | C- | |
|---|---|---|---|---|---|---|---|---|---|---|---|---|---|---|
| Name | X₁ | X₂ | X₃ | X₄ | X₅ | X₆ | X₇ | X₈ | X₉ | X₁₀ | X₁₁ | term | term | Cyclic |
| 1200 | D- | Cys | L- | His | D- | Arg | Trp(6- | Cys | Gly | Gly | Lys* | Ac | NH₂ | Disulfide |
| | Arg | | aMeAsp | | Phe | | Me | | | | | | | |

In embodiments, the peptides of Table E1, Table E1A, Table E2 and Table E2A are not limited to N-terminal functional group, C-terminal functional group, and/or status or type of cyclic function.

In embodiments, the peptide of formula (I) is selected from Table E1.

TABLE E1
Exemplary peptides.

| Molecule | | | | | | | | |
|---|---|---|---|---|---|---|---|---|
| Name | X₁ | X₂ | X₃ | X₄ | X₅ | X₆ | X₇ | X₈ |
| 1001-1 | Arg | Cys | Cyclo-Leu | His | D-Phe | Arg | Trp | Cys |
| 1002-1 | Arg | Cys | D-Ala | His | D-Phe(3,4- | Arg | Trp | Cys |
| | | | | | diMe) | | | |
| 1003-1 | Arg | Cys | L-aMeGlu | His | D-Phe | Arg | Trp | Cys |
| 1004-1 | Arg | Cys | L-aMeAsp | His | D-Phe | Arg | Trp | Cys |
| 1005-1 | D-Arg | Cys | Cyclo-Leu | His | D-Phe | Arg | Trp | Cys |
| 1006-1 | Beta- | Cys | Cyclo-Leu | His | D-Phe | Arg | Trp | Cys |
| | homoArg | | | | | | | |
| 1007-1 | D-Arg | Cys | L-aMeGlu | His | D-Phe | Arg | Trp | Cys |
| 1008-1 | Beta- | Cys | Cyclo-Leu | His | D-Phe | Arg | Trp(6- | Cys |
| | homoArg | | | | | | Me) | |
| 1009-1 | D-Arg | Cys | Cyclo-Leu | His | D-Phe | Arg | Trp(6- | Cys |
| | | | | | | | Me) | |
| 1010-1 | Arg | Cys | D-aMeOrn | His | D-Phe | Arg | Trp | Cys |
| 1011-1 | Arg | Cys | D-Ala | Gln | D-Phe | Arg | Trp | Cys |
| 1012-1 | Arg | Cys | Cyclo-Leu | Gln | D-Phe | Arg | Trp | Cys |
| 1013-1 | Arg | Cys | Ala(2-Me) | Gln | D-Phe | Arg | Trp | Cys |
| 1014-1 | Beta- | Cys | D-Dab | Gln | D-Phe | Arg | Trp | Cys |
| | homoArg | | | | | | | |
| 1015-1 | D-Arg | Cys | Cyclo-Leu | His | D-Phe(4-Me) | Arg | Trp(6- | Cys |
| | | | | | | | Me) | |
| 1016-1 | D-Arg | Cys | L-aMeGlu | His | D-Phe | Arg | Trp(6- | Cys |
| | | | | | | | Me) | |
| 1017-1 | D-Arg | Cys | L-aMeGlu | His | D-Phe(4-Me) | Arg | Trp | Cys |
| 1018-1 | D-Arg | Cys | L-aMeGlu | His | D-Phe | Arg | Trp | Cys |
| 1019-1 | D-Nar | Cys | L-aMeGlu | His | D-Phe | Arg | Trp | Cys |
| 1020-1 | D-Nar | Cys | L-aMeGlu | His | D-Phe | Arg | Trp(6- | Cys |
| | | | | | | | Me) | |
| 1021-1 | D-Nar | Cys | L-aMeGlu | His | D-Phe(4-Me) | Arg | Trp | Cys |
| 1022-1 | Beta- | Cys | L-aMeGlu | His | D-Phe | Arg | Trp | Cys |
| | homoArg | | | | | | | |
| 1023-1 | Beta- | Cys | L-aMeGlu | His | D-Phe(4-Me) | Arg | Trp | Cys |
| | homoArg | | | | | | | |
| 1024-1 | Beta- | Cys | L-aMeGlu | His | D-Phe | Arg | Trp(6- | Cys |
| | homoArg | | | | | | Me) | |
| 1025-1 | Beta- | Cys | L-aMeGlu | His | D-Phe | Arg | Trp | Cys |
| | homoArg | | | | | | | |
| 1026-1 | Beta- | Cys | L-aMeGlu | His | D-Phe(4-Cl) | Arg | Trp(6- | Cys |
| | homoArg | | | | | | Me) | |
| 1027-1 | Beta- | Cys | Cyclo-Leu | His | D-Phe(4-Cl) | Arg | TRP | Cys |
| | homoArg | | | | | | | |
| 1028-1 | Beta- | Cys | L-aMeGlu | His | D-Phe | Arg | Trp(6-F) | Cys |
| | homoArg | | | | | | | |
| 1029-1 | L-hArg | Cys | L-aMeGlu | His | D-Phe | Arg | Trp(6- | Cys |
| | | | | | | | Me) | |
| 1030-1 | Beta- | Cys | L-aMeGlu | His | D-Phe(3-CF3) | Arg | Trp | Cys |
| | homoArg | | | | | | | |
| 1031-1 | Beta- | Cys | L-aMeGlu | His | D-Phe(3-Cl) | Arg | TRP | Cys |
| | homoArg | | | | | | | |
| 1032-1 | Beta- | Cys | L-aMeGlu | His | D-Phe | Arg | Trp(6-CI) | Cys |
| | homoArg | | | | | | | |
| 1033-1 | Beta- | Cys | bhGlu | His | D-Phe | Arg | Trp | Cys |
| | homoArg | | | | | | | |
| 1034-1 | Beta- | Cys | Phg | His | D-Phe | Arg | Trp | Cys |
| | homoArg | | | | | | | |
| 1035-1 | D-Nar | Cys | L-aMeGlu | His | D-Phe | Arg | Trp(6-F) | Cys |
| 1036-1 | D-Nar | Cys | D-aMeOrn | His | D-Phe | Arg | Trp(6-F) | Cys |
| 1037-1 | D-Nar | Cys | Ala(2-Me) | His | D-Phe | Arg | Trp(6-F) | Cys |
| 1038-1 | D-Nar | Cys | L-aMeGlu | His | D-Phe(4-F) | Arg | Trp(6-F) | Cys |
| 1039-1 | D-Nar | Cys | D-aMeSer | His | D-Phe | Arg | Trp(6- | Cys |
| | | | | | | | Me) | |
| 1040-1 | D-Nar | Cys | L-aMeGlu | His | D-Phe(3-F) | Arg | Trp(6-F) | Cys |
| 1041-1 | D-Nar | Cys | L-aMeGlu | His | D-Phe(4-F) | Arg | Trp(6- | Cys |
| | | | | | | | Me) | |
| 1042-1 | D-Nar | Cys | L-aMeAsp | His | D-Phe | Arg | Trp(6-F) | Cys |
| 1043-1 | D-Nar | Cys | L-aMeGlu | His | D-Phe | Arg | Trp(5- | Cys |
| | | | | | | | Me) | |
| 1044-1 | D-Nar | Cys | D-bhGlu | His | D-Phe | Arg | Trp(6- | Cys |
| | | | | | | | Me) | |
| 1045-1 | D-Nar | Cys | L-aMeGlu | His | D-Phe(4-F) | Arg | Trp(6-CI) | Cys |
| 1046-1 | D-Nar | Cys | L-aMeGlu | His | D-Phe(3,4,5- | Arg | Trp(6- | Cys |
| | | | | | triF) | | Me) | |
| 1047-1 | D-Nar | Cys | L-aMeGlu | His | D-Phe(3,4,5- | Arg | Trp(6-F) | Cys |
| | | | | | triF) | | | |
| 1048-1 | D-Nar | Cys | L-aMeGlu | His | D-Phe(3-Cl) | Arg | Trp(6- | Cys |
| | | | | | | | Me) | |
| 1049-1 | D-Nar | Cys | hGlu | His | D-Phe | Arg | Trp(6- | Cys |
| | | | | | | | Me) | |
| 1050-1 | D-Nar | Cys | L-aMeGlu | His | D-Phe(3-CF3) | Arg | Trp(6- | Cys |
| | | | | | | | Me) | |
| 1051-1 | D-Nar | Cys | L-aMeGlu | His | D-Phe(4-Cl) | Arg | Trp(6-F) | Cys |
| 1052-1 | Beta- | Cys | Cyclo-Leu | His | D-Phe | Arg | Trp(6-F) | Cys |
| | homoArg | | | | | | | |
| 1053-1 | Arg | Cys | L-aMeGlu | His | D-Phe(4-F) | Arg | Trp(6-F) | Cys |
| 1054-1 | L-hArg | Cys | L-aMeGlu | His | D-Phe(4-F) | Arg | Trp(6-F) | Cys |
| 1055-1 | D-Arg | Cys | D-aMeOrn | His | D-Phe | Arg | Trp(6 | Cys |
| | | | | | | | Me) | |
| 1056-1 | Arg | Cys | L-aMeGlu | His | D-Phe(4-F) | Arg | Trp(6- | Cys |
| | | | | | | | Me) | |
| 1057-1 | Arg | Cys | L-aMeGlu | His | D-Phe(3-F) | Arg | Trp(6-F) | Cys |
| 1058-1 | D-Nar | Cys | Aib(O- | His | D-Phe | Arg | Trp(6-F) | Cys |
| | | | cyclic) | | | | | |
| 1059-1 | Beta- | Cys | L-aMeGlu | His | D-Phe(4-F) | Arg | Trp(6- | Cys |
| | homoArg | | | | | | Me) | |
| 1060-1 | D-Arg | Cys | L-aMeGlu | His | D-Phe(4-F) | Arg | Trp(6- | Cys |
| | | | | | | | Me) | |
| 1061-1 | L-hArg | Cys | L-aMeGlu | His | D-Phe(4-F) | Arg | Trp(6- | Cys |
| | | | | | | | Me) | |
| 1062-1 | D-Nar | Cys | L-aMeAsp | His | D-Phe(4-F) | Arg | Trp(6- | Cys |
| | | | | | | | Me) | |
| 1063-1 | D-Arg | Cys | L-aMeAsp | His | D-Phe(4-F) | Arg | Trp(6- | Cys |
| | | | | | | | Me) | |
| 1064-1 | D-Nar | Cys | L-aMeAsp | His | D-Phe | Arg | Trp(6- | Cys |
| | | | | | | | Me) | |
| 1065-1 | Beta- | Cys | L-aMeAsp | His | D-Phe | Arg | Trp(6- | Cys |
| | homoArg | | | | | | Me) | |
| 1066-1 | D-Nar | Glu | L-aMeGlu | His | D-Phe | Arg | Trp(6- | Dap |
| | | | | | | | Me) | |
| 1067-1 | D-Nar | Asp | L-aMeGlu | His | D-Phe | Arg | Trp(6- | Dap |
| | | | | | | | Me) | |
| 1068-1 | D-Nar | Glu | L-aMeAsp | His | D-Phe(4-F) | Arg | Trp(6-F) | Dap |
| 1069-1 | D-Nar | Glu | L-aMeGlu | His | D-Phe | Arg | Trp(6- | Dap |
| | | | | | | | Me) | |
| 1070-1 | Arg | Cys | D-Ala | Gln | D-Phe | Arg | Trp | Cys |
| 1071-1 | D-Nar | Cys | L-aMeGlu | His | D-Phe(3-F,4- | Arg | Trp(6-F) | Cys |
| | | | | | Me) | | | |
| 1072-1 | D-Nar | Cys | L-aMeGlu | His | D-Phe(4-CF3) | Arg | Trp(6-F) | Cys |
| 1073-1 | D-Nar | Cys | L-aMeGlu | His | D-Phe(2-F,4- | Arg | Trp(6-F) | Cys |
| | | | | | CI) | | | |
| 1074-1 | D-Nar | Cys | L-aMeGlu | His | D-Phe(3,4- | Arg | Trp(6-F) | Cys |
| | | | | | diF) | | | |
| 1075-1 | D-Nar | Cys | L-aMeGlu | His | D-Phe | Arg | Trp(6- | Cys |
| | | | | | | | CF3) | |
| 1076-1 | D-Nar | Cys | L-aMeGlu | His | D-Phe | Arg | Trp(4-F) | Cys |
| 1077-1 | D-Nar | Cys | L-aMeGlu | His | D-Phe | Arg | Trp(5-F) | Cys |
| 1078-1 | D-Nar | Cys | L-aMeGlu | His | D-Phe | Arg | Trp(7-F) | Cys |
| 1079-1 | D-Nar | Cys | L-aMeGlu | His | D-Phe | Arg | Trp(5-CI) | Cys |
| 1080-1 | D-Nar | Cys | L-aMeGlu | His | D-Phe | Arg | Trp(6- | Cys |
| | | | | | | | Br) | |
| 1081-1 | D-Nar | Cys | L-aMeGlu | His | D-Phe(3-F) | Arg | Trp(5-F) | Cys |
| 1082-1 | D-Nar | Cys | L-aMeGlu | His | D-Phe(2,4- | Arg | Trp(6-F) | Cys |
| | | | | | diCI) | | | |
| 1083-1 | D-Nar | Cys | L-aMeGlu | His | D-Phe(2,3- | Arg | Trp(6-F) | Cys |
| | | | | | diF) | | | |
| 1084-1 | D-Nar | Cys | L-aMeGlu | His | D-Phe(3-Cl) | Arg | Trp(6-F) | Cys |
| 1085-1 | D-Nar | Cys | L-aMeGlu | His | D-Phe(3-F) | Arg | Trp(6- | Cys |
| | | | | | | | Me) | |
| 1086-1 | D-Nar | Cys | L-aMeGlu | His | D-Phe(3-Me) | Arg | Trp(6-F) | Cys |
| 1087-1 | D-Nar | Cys | L-aMeGlu | His | D-Phe(2,4- | Arg | Trp(6-F) | Cys |
| | | | | | diF) | | | |
| 1088-1 | D-Nar | Cys | L-aMeGlu | His | D-Phe(2,4,5- | Arg | Trp(6-F) | Cys |
| | | | | | triF) | | | |
| 1089-1 | D-Nar | Cys | L-aMeAsp | His | D-Phe(3-CF3) | Arg | Trp(6-F) | Cys |
| 1090-1 | D-Nar | Cys | L-aMeGlu | Gln | D-Phe | Arg | Trp(6-F) | Cys |
| 1091-1 | D-Nar | Cys | hGlu | Gln | D-Phe | Arg | Trp(6-F) | Cys |
| 1092-1 | D-Nar | Cys | Aib(O- | Gln | D-Phe | Arg | Trp(6-F) | Cys |
| | | | cyclic) | | | | | |
| 1093-1 | D-Nar | Cys | Aib(O- | Gln | D-Phe(4-F) | Arg | Trp(6-F) | Cys |
| | | | cyclic) | | | | | |
| 1094-1 | D-Nar | Cys | D-aMeOrn | Gln | D-Phe | Arg | Trp(6-F) | Cys |
| 1095-1 | D-Nar | Cys | Aib(O- | Gln | D-Phe(4-Me) | Arg | Trp(6-F) | Cys |
| | | | cyclic) | | | | | |
| 1096-1 | D-Nar | Cys | D-aMeSer | Gln | D-Phe | Arg | Trp(6-F) | Cys |
| 1097-1 | D-Nar | Cys | D-aMeSer | Gln | D-Phe(4-F) | Arg | Trp(6-F) | Cys |
| 1098-1 | D-Nar | Cys | bhGlu | Gln | D-Phe | Arg | Trp(6-F) | Cys |
| 1099-1 | D-Nar | Cys | Ala(2-Me) | Gln | D-Phe(4-F) | Arg | Trp(6-F) | Cys |
| 1100-1 | D-Nar | Cys | D-aMeOrn | hGIn | D-Phe | Arg | Trp(6-F) | Cys |
| 1101-1 | D-Nar | Cys | D-aMeOrn | hGln | D-Phe(4-F) | Arg | Trp(6-F) | Cys |
| 1102-1 | D-Nar | Cys | Aib(O- | hGln | D-Phe | Arg | Trp(6-F) | Cys |
| | | | cyclic) | | | | | |
| 1103-1 | D-Nar | Cys | Aib(O- | Cit | D-Phe | Arg | Trp(6-F) | Cys |
| | | | cyclic) | | | | | |
| 1104-1 | D-Nar | Cys | L-aMeGlu | Cit | D-Phe | Arg | Trp(6-F) | Cys |
| 1105-1 | D-Nar | Cys | Aib(O- | Cit | D-Phe(4-Me) | Arg | Trp(6-F) | Cys |
| | | | cyclic) | | | | | |
| 1106-1 | D-Nar | Cys | Aib(O- | Cit | D-Phe(4-F) | Arg | Trp(6-F) | Cys |
| | | | cyclic) | | | | | |
| 1107-1 | D-Nar | Cys | Aib(O- | hCit | D-Phe(4-F) | Arg | Trp(6-F) | Cys |
| | | | cyclic) | | | | | |
| 1108-1 | D-Nar | Cys | Cyclo-Leu | 3-Pal | D-Phe | Arg | Trp(6-F) | Cys |
| 1109-1 | D-Nar | Cys | D-aMeOrn | 4-Pal | D-Phe | Arg | Trp(6-F) | Cys |
| 1110-1 | D-Nar | Cys | Phg | His | D-Phe | Arg | Trp(6-F) | Cys |
| 1111-1 | D-Nar | Cys | Phg | His | D-Phe(4-F) | Arg | Trp(6-F) | Cys |
| 1112-1 | D-Nar | Cys | Phg | His | D-Phe(4-F) | Arg | Trp(5- | Cys |
| | | | | | | | Me) | |
| 1113-1 | D-Nar | Cys | Phg | His | D-Phe(4-F) | Arg | Trp(6- | Cys |
| | | | | | | | Me) | |
| 1114-1 | Nar | Cys | L-aMeGlu | His | D-Phe | Arg | Trp(6-F) | Cys |
| 1115-1 | Nar | Cys | L-aMeGlu | His | D-Phe(4-F) | Arg | Trp(6-F) | Cys |
| 1116-1 | D-Nar | Cys | L-aMeGlu | 3-Pal | D-Phe | Arg | Trp(6- | Cys |
| | | | | | | | Me) | |
| 1117-1 | D-Nar | Cys | L-aMeGlu | 3-Pal | D-Phe | Arg | Trp(6- | Cys |
| | | | | | | | Me) | |
| 1118-1 | D-Nar | Cys | L-aMeGlu | 4-Pal | D-Phe | Arg | Trp(6- | Cys |
| | | | | | | | Me) | |
| 1119-1 | D-Nar | Cys | Phg | 3-Pal | D-Phe(4-F) | Arg | Trp(6-F) | Cys |
| 1120-1 | D-Nar | Glu | L-aMeAsp | His | D-Phe | Arg | Trp(6-F) | Dap |
| 1121-1 | D-Nar | Glu | L-aMeAsp | His | D-Phe(4-F) | Arg | Trp(6-F) | Dap |
| 1122-1 | D-Nar | Cys | Aib(O- | 3-Pal | D-Phe(4-F) | Arg | Trp(6-F) | Cys |
| | | | cyclic) | | | | | |
| 1123-1 | D-Nar | Cys | Aib(O- | Orn | D-Phe(4-F) | Arg | Trp(6-F) | Cys |
| | | | cyclic) | | | | | |
| 1124-1 | D-Nar | Cys | D-aMeOrn | 3-Pal | D-Phe(4-F) | Arg | Trp(6-F) | Cys |
| 1125-1 | D-Nar | Cys | D-aMeOrn | Orn | D-Phe(4-F) | Arg | Trp(6-F) | Cys |
| 1126-1 | D-Nar | Cys | D-aMeOrn | 3-Pal | D-Phe | Arg | Trp(6-F) | Cys |
| 1127-1 | D-Nar | Cys | D-aMeOrn | Orn | D-Phe | Arg | Trp(6-F) | Cys |
| 1158-1 | D-Nar | Cys | Aib(O- | Gln | D-Phe(4-F) | Arg | Trp(6-F) | Pen |
| | | | cyclic) | | | | | |

In embodiments, the peptide of formula (I) is selected from Table E1A.

In embodiments, the peptide of formula (I) is selected from Table E1A, wherein the N-terminal, C-terminal and/or cyclic structure are an optional feature.

TABLE E1A
Exemplary peptides.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1001 | Arg | Cys | Cyclo- | His | D-Phe | Arg | Trp | Cys | Ac | NH₂ | Disulfide |
| | | | Leu | | | | | | | | |
| 1002 | Arg | Cys | D-Ala | His | D- | Arg | Trp | Cys | Ac | NH₂ | Disulfide |
| | | | | | Phe(3,4- | | | | | | |
| | | | | | diMe) | | | | | | |
| 1003 | Arg | Cys | L- | His | D-Phe | Arg | Trp | Cys | Ac | NH₂ | Disulfide |
| | | | aMeGlu | | | | | | | | |
| 1004 | Arg | Cys | L- | His | D-Phe | Arg | Trp | Cys | Ac | NH₂ | Disulfide |
| | | | aMeAsp | | | | | | | | |
| 1005 | D-Arg | Cys | Cyclo- | His | D-Phe | Arg | Trp | Cys | Ac | NH₂ | Disulfide |
| | | | Leu | | | | | | | | |
| 1006 | Beta- | Cys | Cyclo- | His | D-Phe | Arg | Trp | Cys | Ac | NH₂ | Disulfide |
| | homoArg | | Leu | | | | | | | | |
| 1007 | D-Arg | Cys | L- | His | D-Phe | Arg | Trp | Cys | Ac | NH₂ | Disulfide |
| | | | aMeGlu | | | | | | | | |
| 1008 | Beta- | Cys | Cyclo- | His | D-Phe | Arg | Trp(6- | Cys | Ac | NH₂ | Disulfide |
| | homoArg | | Leu | | | | Me) | | | | |
| 1009 | D-Arg | Cys | Cyclo- | His | D-Phe | Arg | Trp(6- | Cys | Ac | NH₂ | Disulfide |
| | | | Leu | | | | Me) | | | | |
| 1010 | Arg | Cys | D- | His | D-Phe | Arg | Trp | Cys | Ac | NH₂ | Disulfide |
| | | | aMeOrn | | | | | | | | |
| 1011 | Arg | Cys | D-Ala | Gln | D-Phe | Arg | Trp | Cys | Ac | NH₂ | Disulfide |
| 1012 | Arg | Cys | Cyclo- | Gln | D-Phe | Arg | Trp | Cys | Ac | NH₂ | Disulfide |
| | | | Leu | | | | | | | | |
| 1013 | Arg | Cys | Ala(2- | Gln | D-Phe | Arg | Trp | Cys | Ac | NH₂ | Disulfide |
| | | | Me) | | | | | | | | |
| 1014 | Beta- | Cys | D-Dab | Gln | D-Phe | Arg | Trp | Cys | Ac | NH₂ | Disulfide |
| | homoArg | | | | | | | | | | |
| 1015 | D-Arg | Cys | Cyclo- | His | D-Phe(4- | Arg | Trp(6- | Cys | Ac | NH₂ | Disulfide |
| | | | Leu | | Me) | | Me) | | | | |
| 1016 | D-Arg | Cys | L- | His | D-Phe | Arg | Trp(6- | Cys | Ac | NH₂ | Disulfide |
| | | | aMeGlu | | | | Me) | | | | |
| 1017 | D-Arg | Cys | L- | His | D-Phe(4- | Arg | Trp | Cys | Ac | NH₂ | Disulfide |
| | | | aMeGlu | | Me) | | | | | | |
| 1018 | D-Arg | Cys | L- | His | D-Phe | Arg | Trp | Cys | Ac | NH₂ | Disulfide |
| | | | aMeGlu | | | | | | | | |
| 1019 | D-Nar | Cys | L- | His | D-Phe | Arg | Trp | Cys | Ac | NH₂ | Disulfide |
| | | | aMeGlu | | | | | | | | |
| 1020 | D-Nar | Cys | L- | His | D-Phe | Arg | Trp(6- | Cys | Ac | NH₂ | Disulfide |
| | | | aMeGlu | | | | Me) | | | | |
| 1021 | D-Nar | Cys | L- | His | D-Phe(4- | Arg | Trp | Cys | Ac | NH₂ | Disulfide |
| | | | aMeGlu | | Me) | | | | | | |
| 1022 | Beta- | Cys | L- | His | D-Phe | Arg | Trp | Cys | Ac | NH₂ | Disulfide |
| | homoArg | | aMeGlu | | | | | | | | |
| 1023 | Beta- | Cys | L- | His | D-Phe(4- | Arg | Trp | Cys | Ac | NH₂ | Disulfide |
| | homoArg | | aMeGlu | | Me) | | | | | | |
| 1024 | Beta- | Cys | L- | His | D-Phe | Arg | Trp(6- | Cys | Ac | NH₂ | Disulfide |
| | homoArg | | aMeGlu | | | | Me) | | | | |
| 1025 | Beta- | Cys | L- | His | D-Phe | Arg | Trp | Cys | Ac | NH₂ | Disulfide |
| | homoArg | | aMeGlu | | | | | | | | |
| 1026 | Beta- | Cys | L- | His | D-Phe(4- | Arg | Trp(6 | Cys | Ac | NH₂ | Disulfide |
| | homoArg | | aMeGlu | | Cl) | | Me) | | | | |
| 1027 | Beta- | Cys | Cyclo- | His | D-Phe(4- | Arg | TRP | Cys | Ac | NH₂ | Disulfide |
| | homoArg | | Leu | | Cl) | | | | | | |
| 1028 | Beta- | Cys | L- | His | D-Phe | Arg | Trp(6- | Cys | Ac | NH₂ | Disulfide |
| | homoArg | | aMeGlu | | | | F) | | | | |
| 1029 | L-hArg | Cys | L- | His | D-Phe | Arg | Trp(6- | Cys | Ac | NH₂ | Disulfide |
| | | | aMeGlu | | | | Me) | | | | |
| 1030 | Beta- | Cys | L- | His | D-Phe(3- | Arg | Trp | Cys | Ac | NH₂ | Disulfide |
| | homoArg | | aMeGlu | | CF3) | | | | | | |
| 1031 | Beta- | Cys | L- | His | D-Phe(3- | Arg | TRP | Cys | Ac | NH₂ | Disulfide |
| | homoArg | | aMeGlu | | Cl) | | | | | | |
| 1032 | Beta- | Cys | L- | His | D-Phe | Arg | Trp(6- | Cys | Ac | NH₂ | Disulfide |
| | homoArg | | aMeGlu | | | | CI) | | | | |
| 1033 | Beta- | Cys | bhGlu | His | D-Phe | Arg | Trp | Cys | Ac | NH₂ | Disulfide |
| | homoArg | | | | | | | | | | |
| 1034 | Beta- | Cys | Phg | His | D-Phe | Arg | Trp | Cys | Ac | NH₂ | Disulfide |
| | homoArg | | | | | | | | | | |
| 1035 | D-Nar | Cys | L- | His | D-Phe | Arg | Trp(6- | Cys | Ac | NH₂ | Disulfide |
| | | | aMeGlu | | | | F) | | | | |
| 1036 | D-Nar | Cys | D- | His | D-Phe | Arg | Trp(6- | Cys | Ac | NH₂ | Disulfide |
| | | | aMeOrn | | | | F) | | | | |
| 1037 | D-Nar | Cys | Ala(2- | His | D-Phe | Arg | Trp(6- | Cys | Ac | NH₂ | Disulfide |
| | | | Me) | | | | F) | | | | |
| 1038 | D-Nar | Cys | L- | His | D-Phe(4- | Arg | Trp(6- | Cys | Ac | NH₂ | Disulfide |
| | | | aMeGlu | | F) | | F) | | | | |
| 1039 | D-Nar | Cys | D- | His | D-Phe | Arg | Trp(6- | Cys | Ac | NH₂ | Disulfide |
| | | | aMeSer | | | | Me) | | | | |
| 1040 | D-Nar | Cys | L- | His | D-Phe(3- | Arg | Trp(6 | Cys | Ac | NH₂ | Disulfide |
| | | | aMeGlu | | F) | | F) | | | | |
| 1041 | D-Nar | Cys | L- | His | D-Phe(4- | Arg | Trp(6- | Cys | Ac | NH₂ | Disulfide |
| | | | aMeGlu | | F) | | Me) | | | | |
| 1042 | D-Nar | Cys | L- | His | D-Phe | Arg | Trp(6 | Cys | Ac | NH₂ | Disulfide |
| | | | aMeAsp | | | | F) | | | | |
| 1043 | D-Nar | Cys | L- | His | D-Phe | Arg | Trp(5- | Cys | Ac | NH₂ | Disulfide |
| | | | aMeGlu | | | | Me) | | | | |
| 1044 | D-Nar | Cys | D- | His | D-Phe | Arg | Trp(6- | Cys | Ac | NH₂ | Disulfide |
| | | | bhGlu | | | | Me) | | | | |
| 1045 | D-Nar | Cys | L- | His | D-Phe(4- | Arg | Trp(6 | Cys | Ac | NH₂ | Disulfide |
| | | | aMeGlu | | F) | | CI) | | | | |
| 1046 | D-Nar | Cys | L- | His | D- | Arg | Trp(6- | Cys | Ac | NH₂ | Disulfide |
| | | | aMeGlu | | Phe(3,4,5- | | Me) | | | | |
| | | | | | triF) | | | | | | |
| 1047 | D-Nar | Cys | L- | His | D- | Arg | Trp(6- | Cys | Ac | NH₂ | Disulfide |
| | | | aMeGlu | | Phe(3,4,5- | | F) | | | | |
| | | | | | triF) | | | | | | |
| 1048 | D-Nar | Cys | L- | His | D-Phe(3- | Arg | Trp(6- | Cys | Ac | NH₂ | Disulfide |
| | | | aMeGlu | | Cl) | | Me) | | | | |
| 1049 | D-Nar | Cys | hGlu | His | D-Phe | Arg | Trp(6- | Cys | Ac | NH₂ | Disulfide |
| | | | | | | | Me) | | | | |
| 1050 | D-Nar | Cys | L- | His | D-Phe(3- | Arg | Trp(6- | Cys | Ac | NH₂ | Disulfide |
| | | | aMeGlu | | CF3) | | Me) | | | | |
| 1051 | D-Nar | Cys | L- | His | D-Phe(4- | Arg | Trp(6- | Cys | Ac | NH₂ | Disulfide |
| | | | aMeGlu | | Cl) | | F) | | | | |
| 1052 | Beta- | Cys | Cyclo- | His | D-Phe | Arg | Trp(6 | Cys | Ac | NH₂ | Disulfide |
| | homoArg | | Leu | | | | F) | | | | |
| 1053 | Arg | Cys | L- | His | D-Phe(4- | Arg | Trp(6- | Cys | Ac | NH₂ | Disulfide |
| | | | aMeGlu | | F) | | F) | | | | |
| 1054 | L-hArg | Cys | L- | His | D-Phe(4- | Arg | Trp(6- | Cys | Ac | NH₂ | Disulfide |
| | | | aMeGlu | | F) | | F) | | | | |
| 1055 | D-Arg | Cys | D- | His | D-Phe | Arg | Trp(6- | Cys | Ac | NH₂ | Disulfide |
| | | | aMeOrn | | | | Me) | | | | |
| 1056 | Arg | Cys | L- | His | D-Phe(4- | Arg | Trp(6 | Cys | Ac | NH₂ | Disulfide |
| | | | aMeGlu | | F) | | Me) | | | | |
| 1057 | Arg | Cys | L- | His | D-Phe(3- | Arg | Trp(6 | Cys | Ac | NH₂ | Disulfide |
| | | | aMeGlu | | F) | | F) | | | | |
| 1058 | D-Nar | Cys | Aib(O- | His | D-Phe | Arg | Trp(6- | Cys | Ac | NH₂ | Disulfide |
| | | | cyclic) | | | | F) | | | | |
| 1059 | Beta- | Cys | L- | His | D-Phe(4- | Arg | Trp(6- | Cys | Ac | NH₂ | Disulfide |
| | homoArg | | aMeGlu | | F) | | Me) | | | | |
| 1060 | D-Arg | Cys | L- | His | D-Phe(4- | Arg | Trp(6- | Cys | Ac | NH₂ | Disulfide |
| | | | aMeGlu | | F) | | Me) | | | | |
| 1061 | L-hArg | Cys | L- | His | D-Phe(4- | Arg | Trp(6- | Cys | Ac | NH₂ | Disulfide |
| | | | aMeGlu | | F) | | Me) | | | | |
| 1062 | D-Nar | Cys | L- | His | D-Phe(4- | Arg | Trp(6- | Cys | Ac | NH₂ | Disulfide |
| | | | aMeAsp | | F) | | Me) | | | | |
| 1063 | D-Arg | Cys | L- | His | D-Phe(4- | Arg | Trp(6- | Cys | Ac | NH₂ | Disulfide |
| | | | aMeAsp | | F) | | Me) | | | | |
| 1064 | D-Nar | Cys | L- | His | D-Phe | Arg | Trp(6- | Cys | Ac | NH₂ | Disulfide |
| | | | aMeAsp | | | | Me) | | | | |
| 1065 | Beta- | Cys | L- | His | D-Phe | Arg | Trp(6- | Cys | Ac | NH₂ | Disulfide |
| | homoArg | | aMeAsp | | | | Me) | | | | |
| 1066 | D-Nar | Glu | L- | His | D-Phe | Arg | Trp(6- | Dap | Ac | NH₂ | Lactam |
| | | | aMeGlu | | | | Me) | | | | |
| 1067 | D-Nar | Asp | L- | His | D-Phe | Arg | Trp(6- | Dap | Ac | NH₂ | Lactam |
| | | | aMeGlu | | | | Me) | | | | |
| 1068 | D-Nar | Glu | L- | His | D-Phe(4- | Arg | Trp(6- | Dap | Ac | NH₂ | Lactam |
| | | | aMeAsp | | F) | | F) | | | | |
| 1069 | D-Nar | Glu | L- | His | D-Phe | Arg | Trp(6- | Dap | Ac | NH₂ | Lactam |
| | | | aMeGlu | | | | Me) | | | | |
| 1070 | Arg | Cys | D-Ala | Gln | D-Phe | Arg | Trp | Cys | Ac | NH₂ | Disulfide |
| 1071 | D-Nar | Cys | L- | His | D-Phe(3- | Arg | Trp(6 | Cys | Ac | NH₂ | Disulfide |
| | | | aMeGlu | | F,4-Me) | | F) | | | | |
| 1072 | D-Nar | Cys | L- | His | D-Phe(4- | Arg | Trp(6- | Cys | Ac | NH₂ | Disulfide |
| | | | aMeGlu | | CF3) | | F) | | | | |
| 1073 | D-Nar | Cys | L- | His | D-Phe(2- | Arg | Trp(6- | Cys | Ac | NH₂ | Disulfide |
| | | | aMeGlu | | F,4-Cl) | | F) | | | | |
| 1074 | D-Nar | Cys | L- | His | D- | Arg | Trp(6- | Cys | Ac | NH₂ | Disulfide |
| | | | aMeGlu | | Phe(3,4- | | F) | | | | |
| | | | | | diF) | | | | | | |
| 1075 | D-Nar | Cys | L- | His | D-Phe | Arg | Trp(6- | Cys | Ac | NH₂ | Disulfide |
| | | | aMeGlu | | | | CF3) | | | | |
| 1076 | D-Nar | Cys | L- | His | D-Phe | Arg | Trp(4- | Cys | Ac | NH₂ | Disulfide |
| | | | aMeGlu | | | | F) | | | | |
| 1077 | D-Nar | Cys | L- | His | D-Phe | Arg | Trp(5- | Cys | Ac | NH₂ | Disulfide |
| | | | aMeGlu | | | | F) | | | | |
| 1078 | D-Nar | Cys | L- | His | D-Phe | Arg | Trp(7- | Cys | Ac | NH₂ | Disulfide |
| | | | aMeGlu | | | | F) | | | | |
| 1079 | D-Nar | Cys | L- | His | D-Phe | Arg | Trp(5- | Cys | Ac | NH₂ | Disulfide |
| | | | aMeGlu | | | | CI) | | | | |
| 1080 | D-Nar | Cys | L- | His | D-Phe | Arg | Trp(6- | Cys | Ac | NH₂ | Disulfide |
| | | | aMeGlu | | | | Br) | | | | |
| 1081 | D-Nar | Cys | L- | His | D-Phe(3- | Arg | Trp(5- | Cys | Ac | NH₂ | Disulfide |
| | | | aMeGlu | | F) | | F) | | | | |
| 1082 | D-Nar | Cys | L- | His | D- | Arg | Trp(6- | Cys | Ac | NH₂ | Disulfide |
| | | | aMeGlu | | Phe(2,4- | | F) | | | | |
| | | | | | diCl) | | | | | | |
| 1083 | D-Nar | Cys | L- | His | D- | Arg | Trp(6- | Cys | Ac | NH₂ | Disulfide |
| | | | aMeGlu | | Phe(2,3- | | F) | | | | |
| | | | | | diF) | | | | | | |
| 1084 | D-Nar | Cys | L- | His | D-Phe(3- | Arg | Trp(6- | Cys | Ac | NH₂ | Disulfide |
| | | | aMeGlu | | Cl) | | F) | | | | |
| 1085 | D-Nar | Cys | L- | His | D-Phe(3- | Arg | Trp(6- | Cys | Ac | NH₂ | Disulfide |
| | | | aMeGlu | | F) | | Me) | | | | |
| 1086 | D-Nar | Cys | L- | His | D-Phe(3- | Arg | Trp(6- | Cys | Ac | NH₂ | Disulfide |
| | | | aMeGlu | | Me) | | F) | | | | |
| 1087 | D-Nar | Cys | L- | His | D- | Arg | Trp(6- | Cys | Ac | NH₂ | Disulfide |
| | | | aMeGlu | | Phe(2,4- | | F) | | | | |
| | | | | | diF) | | | | | | |
| 1088 | D-Nar | Cys | L- | His | D- | Arg | Trp(6- | Cys | Ac | NH₂ | Disulfide |
| | | | aMeGlu | | Phe(2,4,5- | | F) | | | | |
| | | | | | triF) | | | | | | |
| 1089 | D-Nar | Cys | L- | His | D-Phe(3- | Arg | Trp(6- | Cys | Ac | NH₂ | Disulfide |
| | | | aMeAsp | | CF3) | | F) | | | | |
| 1090 | D-Nar | Cys | L- | Gln | D-Phe | Arg | Trp(6- | Cys | Ac | NH₂ | Disulfide |
| | | | aMeGlu | | | | F) | | | | |
| 1091 | D-Nar | Cys | hGlu | Gln | D-Phe | Arg | Trp(6- | Cys | Ac | NH₂ | Disulfide |
| | | | | | | | F) | | | | |
| 1092 | D-Nar | Cys | Aib(O- | Gln | D-Phe | Arg | Trp(6- | Cys | Ac | NH₂ | Disulfide |
| | | | cyclic) | | | | F) | | | | |
| 1093 | D-Nar | Cys | Aib(O- | Gln | D-Phe(4- | Arg | Trp(6- | Cys | Ac | NH₂ | Disulfide |
| | | | cyclic) | | F) | | F) | | | | |
| 1094 | D-Nar | Cys | D- | Gln | D-Phe | Arg | Trp(6- | Cys | Ac | NH₂ | Disulfide |
| | | | aMeOrn | | | | F) | | | | |
| 1095 | D-Nar | Cys | Aib(O- | Gln | D-Phe(4- | Arg | Trp(6- | Cys | Ac | NH₂ | Disulfide |
| | | | cyclic) | | Me) | | F) | | | | |
| 1096 | D-Nar | Cys | D- | Gln | D-Phe | Arg | Trp(6- | Cys | Ac | NH₂ | Disulfide |
| | | | aMeSer | | | | F) | | | | |
| 1097 | D-Nar | Cys | D- | Gln | D-Phe(4- | Arg | Trp(6- | Cys | Ac | NH₂ | Disulfide |
| | | | aMeSer | | F) | | F) | | | | |
| 1098 | D-Nar | Cys | bhGlu | Gln | D-Phe | Arg | Trp(6- | Cys | Ac | NH₂ | Disulfide |
| | | | | | | | F) | | | | |
| 1099 | D-Nar | Cys | Ala(2- | Gln | D-Phe(4- | Arg | Trp(6- | Cys | Ac | NH₂ | Disulfide |
| | | | Me) | | | | F) | | | | |
| 1100 | D-Nar | Cys | D- | hGln | D-Phe | Arg | Trp(6- | Cys | Ac | NH₂ | Disulfide |
| | | | aMeOrn | | | | F) | | | | |
| 1101 | D-Nar | Cys | D- | hGln | D-Phe(4- | Arg | Trp(6- | Cys | Ac | NH₂ | Disulfide |
| | | | aMeOrn | | F) | | F) | | | | |
| 1102 | D-Nar | Cys | Aib(O- | hGIn | D-Phe | Arg | Trp(6- | Cys | Ac | NH₂ | Disulfide |
| | | | cyclic) | | | | F) | | | | |
| 1103 | D-Nar | Cys | Aib(O- | Cit | D-Phe | Arg | Trp(6- | Cys | Ac | NH₂ | Disulfide |
| | | | cyclic) | | | | F) | | | | |
| 1104 | D-Nar | Cys | L- | Cit | D-Phe | Arg | Trp(6- | Cys | Ac | NH₂ | Disulfide |
| | | | aMeGlu | | | | F) | | | | |
| 1105 | D-Nar | Cys | Aib(O- | Cit | D-Phe(4- | Arg | Trp(6- | Cys | Ac | NH₂ | Disulfide |
| | | | cyclic) | | Me) | | F) | | | | |
| 1106 | D-Nar | Cys | Aib(O- | Cit | D-Phe(4- | Arg | Trp(6- | Cys | Ac | NH₂ | Disulfide |
| | | | cyclic) | | F) | | F) | | | | |
| 1107 | D-Nar | Cys | Aib(O- | hCit | D-Phe(4- | Arg | Trp(6- | Cys | Ac | NH₂ | Disulfide |
| | | | cyclic) | | F) | | F) | | | | |
| 1108 | D-Nar | Cys | Cyclo- | 3- | D-Phe | Arg | Trp(6- | Cys | Ac | NH₂ | Disulfide |
| | | | Leu | Pal | | | F) | | | | |
| 1109 | D-Nar | Cys | D- | 4- | D-Phe | Arg | Trp(6- | Cys | Ac | NH₂ | Disulfide |
| | | | aMeOrn | Pal | | | F) | | | | |
| 1110 | D-Nar | Cys | Phg | His | D-Phe | Arg | Trp(6- | Cys | Ac | NH₂ | Disulfide |
| | | | | | | | F) | | | | |
| 1111 | D-Nar | Cys | Phg | His | D-Phe(4- | Arg | Trp(6- | Cys | Ac | NH₂ | Disulfide |
| | | | | | F) | | F) | | | | |
| 1112 | D-Nar | Cys | Phg | His | D-Phe(4- | Arg | Trp(5- | Cys | Ac | NH₂ | Disulfide |
| | | | | | F) | | Me) | | | | |
| 1113 | D-Nar | Cys | Phg | His | D-Phe(4- | Arg | Trp(6 | Cys | Ac | NH₂ | Disulfide |
| | | | | | F) | | Me) | | | | |
| 1114 | Nar | Cys | L- | His | D-Phe | Arg | Trp(6- | Cys | Ac | NH₂ | Disulfide |
| | | | aMeGlu | | | | F) | | | | |
| 1115 | Nar | Cys | L- | His | D-Phe(4- | Arg | Trp(6- | Cys | Ac | NH₂ | Disulfide |
| | | | aMeGlu | | F) | | F) | | | | |
| 1116 | D-Nar | Cys | L- | 3- | D-Phe | Arg | Trp(6- | Cys | Ac | NH₂ | Disulfide |
| | | | aMeGlu | Pal | | | Me) | | | | |
| 1117 | D-Nar | Cys | L- | 3- | D-Phe | Arg | Trp(6- | Cys | Ac | NH₂ | Disulfide |
| | | | aMeGlu | Pal | | | Me) | | | | |
| 1118 | D-Nar | Cys | L- | 4- | D-Phe | Arg | Trp(6- | Cys | Ac | NH₂ | Disulfide |
| | | | aMeGlu | Pal | | | Me) | | | | |
| 1119 | D-Nar | Cys | Phg | 3- | D-Phe(4- | Arg | Trp(6- | Cys | Ac | NH₂ | Disulfide |
| | | | | Pal | F) | | F) | | | | |
| 1120 | D-Nar | Glu | L- | His | D-Phe | Arg | Trp(6- | Dap | Ac | NH₂ | Lactam |
| | | | aMeAsp | | | | F) | | | | |
| 1121 | D-Nar | Glu | L- | His | D-Phe(4- | Arg | Trp(6- | Dap | Ac | NH₂ | Lactam |
| | | | aMeAsp | | F) | | F) | | | | |
| 1122 | D-Nar | Cys | Aib(O- | 3- | D-Phe(4- | Arg | Trp(6- | Cys | Ac | NH₂ | Disulfide |
| | | | cyclic) | Pal | F) | | F) | | | | |
| 1123 | D-Nar | Cys | Aib(O- | Orn | D-Phe(4- | Arg | Trp(6- | Cys | Ac | NH₂ | Disulfide |
| | | | cyclic) | | F) | | F | | | | |
| 1124 | D-Nar | Cys | D- | 3- | D-Phe(4- | Arg | Trp(6- | Cys | Ac | NH₂ | Disulfide |
| | | | aMeOrn | Pal | F) | | F) | | | | |
| 1125 | D-Nar | Cys | D- | Orn | D-Phe(4- | Arg | Trp(6- | Cys | Ac | NH₂ | Disulfide |
| | | | aMeOrn | | F) | | F) | | | | |
| 1126 | D-Nar | Cys | D- | 3- | D-Phe | Arg | Trp(6- | Cys | Ac | NH₂ | Disulfide |
| | | | aMeOrn | Pal | | | F) | | | | |
| 1127 | D-Nar | Cys | D- | Orn | D-Phe | Arg | Trp(6- | Cys | Ac | NH₂ | Disulfide |
| | | | aMeOrn | | | | F) | | | | |
| 1158 | D-Nar | Cys | Aib(O- | Gln | D-Phe(4- | Arg | Trp(6- | Pen | Ac | NH₂ | Disulfide |
| | | | cyclic) | | F) | | F | | | | |

In embodiments, the peptide of formula (II) is selected from Table E2.

TABLE E2
Exemplary peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | X₉ | X₁₀ | X₁₁ |
| 1128-2 | | Lys* | Glu | Pro | D- | Cys | L- | His | D- | Arg | Trp(6- | Cys | | | |
| | | | | | Nar | | aMeGlu | | Phe | | Me) | | | | |
| 1129-2 | | Lys* | Gly | D- | D- | Cys | L- | His | D- | Arg | Trp(6- | Cy | | | |
| | | | | Arg | Nar | | aMeGlu | | Phe | | Me) | S | | | |
| 1130-2 | | Lys* | Gly | Gly | beta- | Cys | L- | His | D- | Arg | Trp(6- | Cys | | | |
| | | | | | homoArg | | aMeAsp | | Phe | | Me) | | | | |
| 1131-2 | | Lys* | PEG1 | PEG1 | D- | Cys | L- | His | D- | Arg | Trp(6- | Cys | | | |
| | | | | | Nar | | aMeAsp | | Phe | | Me) | | | | |
| 1132-2 | Lys* | Glu | PEG1 | PEG1 | D- | Cys | L- | His | D- | Arg | Trp(6- | Cys | | | |
| | | | | | Nar | | aMeGlu | | Phe(3,4,5- | | Me) | | | | |
| | | | | | | | | | triF) | | | | | | |
| 1133-2 | | Lys* | Gly | gGlu | D- | Cys | L- | His | D- | Arg | Trp(6- | Cys | | | |
| | | | | | Nar | | aMeGlu | | Phe | | Me) | | | | |
| 1134-2 | Lys* | Glu | Glu | Pro | D- | Cys | L- | His | D- | Arg | Trp(6- | Cys | | | |
| | | | | | Nar | | aMeGlu | | Phe(3,4,5- | | Me) | | | | |
| | | | | | | | | | triF) | | | | | | |
| 1135-2 | | Lys* | Gly | Gly | D- | Cys | L- | His | D- | Arg | Trp(6- | Cys | | | |
| | | | | | Arg | | aMeAsp | | Phe | | Me) | | | | |
| 1136-2 | Lys* | D- | PEG1 | D- | Beta- | Cys | D- | Gln | D- | Arg | Trp(6- | Cys | | | |
| | | Arg | | Arg | homoArg | | aMeOrn | | Phe | | F) | | | | |
| 1137-2 | | Lys* | PEG1 | PEG1 | D- | Cys | D- | Gln | D- | Arg | Trp(6- | Cys | | | |
| | | | | | Nar | | aMeOrn | | Phe | | F) | | | | |
| 1138-2 | | Lys* | Gly | D- | D- | Cys | L- | His | D- | Arg | Trp(6- | Cys | | | |
| | | | | Arg | Nar | | aMeGlu | | Phe | | F) | | | | |
| 1139-2 | | Lys* | Gly | D- | D- | Cys | Phg | 3- | D- | Arg | Trp(6- | Cys | | | |
| | | | | Arg | Nar | | | Pal | Phe(4- | | F) | | | | |
| | | | | | | | | | F) | | | | | | |
| 1140-2 | | Lys* | D- | Y- | D- | Cys | L- | His | D- | Arg | Trp(6- | Cys | | | |
| | | | Arg | Glu | Nar | | aMeGlu | | Phe | | F) | | | | |
| 1141-2 | Lys* | Ser | Glu | Pro | D- | Cys | L- | His | D- | Arg | Trp(6- | Cys | | | |
| | | | | | Nar | | aMeGlu | | Phe | | F) | | | | |
| 1142-2 | | Lys* | Gly | D- | D- | Cys | Aib(O- | Gln | D- | Arg | Trp(6- | Cys | | | |
| | | | | Arg | Nar | | cyclic) | | Phe(4- | | F) | | | | |
| | | | | | | | | | F) | | | | | | |
| 1143-2 | Lys* | Gly | Gly | Y- | D- | Cys | L- | His | D- | Arg | Trp(6- | Cys | | | |
| | | | | Glu | Nar | | aMeGlu | | Phe | | F) | | | | |
| 1144-2 | | Lys* | PEG1 | PEG1 | D- | Cys | Aib(O- | Gln | D- | Arg | Trp(6- | Cys | | | |
| | | | | | Nar | | cyclic) | | Phe(4- | | F) | | | | |
| | | | | | | | | | F) | | | | | | |
| 1145-2 | | Lys* | PEG1 | PEG1 | D- | Cys | Phg | 3- | D- | Arg | Trp(6- | Cys | | | |
| | | | | | Nar | | | Pal | Phe(4- | | F) | | | | |
| | | | | | | | | | F) | | | | | | |
| 1146-2 | Lys* | D- | Gly | D- | D- | Cys | Phg | 3- | D- | Arg | Trp(6- | Cys | | | |
| | | Arg | | Arg | Nar | | | Pal | Phe(4- | | F) | | | | |
| | | | | | | | | | F) | | | | | | |
| 1147-2 | Lys* | D- | PEG1 | D- | Beta- | Cys | Phg | 3- | D- | Arg | Trp(6- | Cys | | | |
| | | Arg | | Arg | homoArg | | | Pal | Phe(4- | | F) | | | | |
| | | | | | | | | | F) | | | | | | |
| 1148-2 | Lys* | D- | Gly | D- | D- | Cys | D- | Gln | D- | Arg | Trp(6- | Cys | | | |
| | | Arg | | Arg | Nar | | aMeOrn | | Phe | | F) | | | | |
| 1149-2 | | Lys* | Gly | D- | D- | Cys | D- | Gln | D- | Arg | Trp(6- | Cys | | | |
| | | | | Arg | Nar | | aMeOrn | | Phe | | F) | | | | |
| 1150-2 | Lys* | D- | Gly | D- | D- | Cys | Aib(O- | Gln | D- | Arg | Trp(6- | Cys | | | |
| | | Arg | | Arg | Nar | | cyclic) | | Phe(4- | | F) | | | | |
| | | | | | | | | | F) | | | | | | |
| 1151-2 | Lys* | D- | PEG1 | D- | Beta- | Cys | Aib(O- | Gln | D- | Arg | Trp(6- | Cys | | | |
| | | Arg | | Arg | homoArg | | cyclic) | | Phe(4- | | F) | | | | |
| | | | | | | | | | F) | | | | | | |
| 1152-2 | Lys* | D- | Gly | D- | D- | Cys | Aib(O- | 3- | D- | Arg | Trp(6- | Cys | | | |
| | | Arg | | Arg | Nar | | cyclic) | Pal | Phe(4- | | F) | | | | |
| | | | | | | | | | F) | | | | | | |
| 1153-2 | Lys* | D- | Gly | D- | D- | Cys | Aib(O- | Orn | D- | Arg | Trp(6- | Cys | | | |
| | | Arg | | Arg | Nar | | cyclic) | | Phe(4- | | F) | | | | |
| | | | | | | | | | F) | | | | | | |
| 1154-2 | Lys* | D- | Gly | D- | D- | Cys | D- | 3- | D- | Arg | Trp(6- | Cys | | | |
| | | Arg | | Arg | Nar | | aMeOrn | Pal | Phe(4- | | F) | | | | |
| | | | | | | | | | F) | | | | | | |
| 1155-2 | Lys* | D- | Gly | D- | D- | Cys | D- | Orn | D- | Arg | Trp(6- | Cys | | | |
| | | Arg | | Arg | Nar | | aMeOrn | | Phe(4- | | F) | | | | |
| | | | | | | | | | F) | | | | | | |
| 1156-2 | Lys* | D- | Gly | D- | D- | Cys | D- | 3- | D- | Arg | Trp(6- | Cys | | | |
| | | Arg | | Arg | Nar | | aMeOrn | Pal | Phe | | F) | | | | |
| 1157-2 | Lys* | D- | Gly | D- | D- | Cys | D- | Orn | D- | Arg | Trp(6- | Cys | | | |
| | | Arg | | Arg | Nar | | aMeOrn | | Phe | | F) | | | | |
| 1200-2 | | | | | D- | Cys | L- | His | D- | Arg | Trp(6- | Cys | Gly | Gly | Lys* |
| | | | | | Arg | | aMeAsp | | Phe | | Me) | | | | |
| 1201-2 | | | | | D- | Cys | L- | His | D- | Arg | Trp(6- | Cys | Gly | Gly | Lys* |
| | | | | | Nar | | aMeAsp | | Phe | | Me) | | | | |

In embodiments, the peptide of formula (II) is selected from Table E2A.

In embodiments, the peptide of formula (II) is selected from Table E2A, wherein the N-terminal. C-terminal and/or cyclic structure are optional feature.

TABLE E2A
Exemplary lipidated molecules.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1128 | | Lys* | Glu | Pro | D- | Cys | L- | His | D- | Arg | Trp(6- | Cys | Ac | NH₂ | Disulfide |
| | | | | | Nar | | aMeGlu | | Phe | | Me) | | | | |
| 1129 | | Lys* | Gly | D- | D- | Cys | L- | His | D- | Arg | Trp(6- | Cys | Ac | NH₂ | Disulfide |
| | | | | Arg | Nar | | aMeGlu | | Phe | | Me) | | | | |
| 1130 | | Lys* | Gly | Gly | beta- | Cys | L- | His | D- | Arg | Trp(6- | Cys | Ac | NH₂ | Disulfide |
| | | | | | homoArg | | aMeAsp | | Phe | | Me) | | | | |
| 1131 | | Lys* | PEG1 | PEG1 | D- | Cys | L- | His | D- | Arg | Trp(6- | Cys | Ac | NH₂ | Disulfide |
| | | | | | Nar | | aMeAsp | | Phe | | Me) | | | | |
| 1132 | Lys* | Glu | PEG1 | PEG1 | D- | Cys | L- | His | D- | Arg | Trp(6- | Cys | Ac | NH₂ | Disulfide |
| | | | | | Nar | | aMeGlu | | Phe(3,4,5- | | Me) | | | | |
| | | | | | | | | | triF) | | | | | | |
| 1133 | | Lys* | Gly | gGlu | D- | Cys | L- | His | D- | Arg | Trp(6- | Cys | Ac | NH₂ | Disulfide |
| | | | | | Nar | | aMeGlu | | Phe | | Me) | | | | |
| 1134 | Lys* | Glu | Glu | Pro | D- | Cys | L- | His | D- | Arg | Trp(6- | Cys | Ac | NH₂ | Disulfide |
| | | | | | Nar | | aMeGlu | | Phe(3,4,5- | | Me) | | | | |
| | | | | | | | | | triF) | | | | | | |
| 1135 | | Lys* | Gly | Gly | D- | Cys | L- | His | D- | Arg | Trp(6- | Cys | Ac | NH₂ | Disulfide |
| | | | | | Arg | | aMeAsp | | Phe | | Me) | | | | |
| 1136 | Lys* | D- | PEG1 | D- | Beta- | Cys | D- | Gln | D- | Arg | Trp(6- | Cys | Ac | NH₂ | Disulfide |
| | | Arg | | Arg | homoArg | | aMeOrn | | Phe | | F) | | | | |
| 1137 | | Lys* | PEG1 | PEG1 | D- | Cys | D- | Gln | D- | Arg | Trp(6- | Cys | Ac | NH₂ | Disulfide |
| | | | | | Nar | | aMeOrn | | Phe | | F) | | | | |
| 1138 | | Lys* | Gly | D- | D- | Cys | L- | His | D- | Arg | Trp(6- | Cys | Ac | NH₂ | Disulfide |
| | | | | Arg | Nar | | aMeGlu | | Phe | | F) | | | | |
| 1139 | | Lys* | Gly | D- | D- | Cys | Phg | 3- | D- | Arg | Trp(6- | Cys | Ac | NH₂ | Disulfide |
| | | | | Arg | Nar | | | Pal | Phe(4- | | F) | | | | |
| | | | | | | | | | F) | | | | | | |
| 1140 | | Lys* | D- | Y- | D- | Cys | L- | His | D- | Arg | Trp(6- | Cys | Ac | NH₂ | Disulfide |
| | | | Arg | Glu | Nar | | aMeGlu | | Phe | | F) | | | | |
| 1141 | Lys* | Ser | Glu | Pro | D- | Cys | L- | His | D- | Arg | Trp(6- | Cys | Ac | NH₂ | Disulfide |
| | | | | | Nar | | aMeGlu | | Phe | | F) | | | | |
| 1142 | | Lys* | Gly | D- | D- | Cys | Aib(O- | Gln | D- | Arg | Trp(6- | Cys | Ac | NH₂ | Disulfide |
| | | | | Arg | Nar | | cyclic) | | Phe(4- | | F) | | | | |
| | | | | | | | | | F) | | | | | | |
| 1143 | Lys* | Gly | Gly | Y- | D- | Cys | L- | His | D- | Arg | Trp(6- | Cys | Ac | NH₂ | Disulfide |
| | | | | Glu | Nar | | aMeGlu | | Phe | | F) | | | | |
| 1144 | | Lys* | PEG1 | PEG1 | D- | Cys | Aib(O- | Gln | D- | Arg | Trp(6- | Cys | Ac | NH₂ | Disulfide |
| | | | | | Nar | | cyclic) | | Phe(4- | | F) | | | | |
| | | | | | | | | | F) | | | | | | |
| 1145 | | Lys* | PEG1 | PEG1 | D- | Cys | Phg | 3- | D- | Arg | Trp(6- | Cys | Ac | NH₂ | Disulfide |
| | | | | | Nar | | | Pal | Phe(4- | | F) | | | | |
| | | | | | | | | | F) | | | | | | |
| 1146 | Lys* | D- | Gly | D- | D- | Cys | Phg | 3- | D- | Arg | Trp(6- | Cys | Ac | NH₂ | Disulfide |
| | | Arg | | Arg | Nar | | | Pal | Phe(4- | | F) | | | | |
| | | | | | | | | | F) | | | | | | |
| 1147 | Lys* | D- | PEG1 | D- | Beta- | Cys | Phg | 3- | D- | Arg | Trp(6- | Cys | Ac | NH₂ | Disulfide |
| | | Arg | | Arg | homoArg | | | Pal | Phe(4- | | F) | | | | |
| | | | | | | | | | F) | | | | | | |
| 1148 | Lys* | D- | Gly | D- | D- | Cys | D- | Gln | D- | Arg | Trp(6- | Cys | Ac | NH₂ | Disulfide |
| | | Arg | | Arg | Nar | | aMeOrn | | Phe | | F) | | | | |
| 1149 | | Lys* | Gly | D- | D- | Cys | D- | Gln | D- | Arg | Trp(6- | Cys | Ac | NH₂ | Disulfide |
| | | | | Arg | Nar | | aMeOrn | | Phe | | F) | | | | |
| 1150 | Lys* | D- | Gly | D- | D- | Cys | Aib(O- | Gln | D- | Arg | Trp(6- | Cys | Ac | NH₂ | Disulfide |
| | | Arg | | Arg | Nar | | cyclic) | | Phe(4- | | F) | | | | |
| | | | | | | | | | F) | | | | | | |
| 1151 | Lys* | D- | PEG1 | D- | Beta- | Cys | Aib(O- | Gln | D- | Arg | Trp(6- | Cys | Ac | NH₂ | Disulfide |
| | | Arg | | Arg | homoArg | | cyclic) | | Phe(4- | | F) | | | | |
| | | | | | | | | | F) | | | | | | |
| 1152 | Lys* | D- | Gly | D- | D- | Cys | Aib(O- | 3- | D- | Arg | Trp(6- | Cys | Ac | NH₂ | Disulfide |
| | | Arg | | Arg | Nar | | cyclic) | Pal | Phe(4- | | F) | | | | |
| | | | | | | | | | F) | | | | | | |
| 1153 | Lys* | D- | Gly | D- | D- | Cys | Aib(O- | Orn | D- | Arg | Trp(6- | Cys | Ac | NH₂ | Disulfide |
| | | Arg | | Arg | Nar | | cyclic) | | Phe(4- | | F) | | | | |
| | | | | | | | | | F) | | | | | | |
| 1154 | Lys* | D- | Gly | D- | D- | Cys | D- | 3- | D- | Arg | Trp(6- | Cys | Ac | NH₂ | Disulfide |
| | | Arg | | Arg | Nar | | aMeOrn | Pal | Phe(4- | | F) | | | | |
| | | | | | | | | | F) | | | | | | |
| 1155 | Lys* | D- | Gly | D- | D- | Cys | D- | Orn | D- | Arg | Trp(6- | Cys | Ac | NH₂ | Disulfide |
| | | Arg | | Arg | Nar | | aMeOrn | | Phe(4- | | F) | | | | |
| | | | | | | | | | F) | | | | | | |
| 1156 | Lys* | D- | Gly | D- | D- | Cys | D- | 3- | D- | Arg | Trp(6- | Cys | Ac | NH₂ | Disulfide |
| | | Arg | | Arg | Nar | | aMeOrn | Pal | Phe | | F) | | | | |
| 1157 | Lys* | D- | Gly | D- | D- | Cys | D- | Orn | D- | Arg | Trp(6- | Cys | Ac | NH₂ | Disulfide |
| | | Arg | | Arg | Nar | | aMeOrn | | Phe | | F) | | | | |

| Molecule | | | | | | | | | | | | N- | C- | |
|---|---|---|---|---|---|---|---|---|---|---|---|---|---|---|
| Name | X₁ | X₂ | X₃ | X₄ | X₅ | X₆ | X₇ | X₈ | X₉ | X₁₀ | X₁₁ | term | term | Cyclic |
| 1200 | D- | Cys | L- | His | D- | Arg | Trp(6- | Cys | Gly | Gly | Lys* | Ac | NH₂ | Disulfide |
| | Arg | | aMeAsp | | Phe | | Me) | | | | | | | |
| 1201 | D- | Cys | L- | His | D- | Arg | Trp(6- | Cys | Gly | Gly | Lys* | Ac | NH₂ | Disulfide |
| | Nar | | aMeAsp | | Phe | | Me) | | | | | | | |

In embodiments, the peptide of formula (I) is selected from Table F1 or Table F2.

In embodiments, the peptides of Table F1 and Table F2 are not limited to N-terminal functional group, C-terminal functional group, and/or status or type of cyclic function.

In embodiments, the peptide of formula (I) is selected from Table F1.

TABLE F1
Exemplary peptide.

| Molecule | | | | | | | | |
|---|---|---|---|---|---|---|---|---|
| Name | X₁ | X₂ | X₃ | X₄ | X₅ | X₆ | X₇ | X₈ |
| 1108-1 | D- | Cys | Cyclo- | 3Pal | D- | Arg | Trp(6- | Cys |
| | Nar | | Leu | | Phe | | F) | |

In embodiments, the peptide of formula (I) is selected from Table F2.

In embodiments, the peptide of formula (I) is selected from Table F2, wherein the N-terminal. C-terminal and/or cyclic structure are optional feature.

TABLE F2
Exemplary peptide.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1108 | D- | Cys | Cyclo- | 3Pal | D- | Arg | Trp(6- | Cys | Ac | NH2 | Disulfide |
| | Nar | | Leu | | Phe | | F) | | | | |

TABLE 1
Exemplary peptides. Cyclic peptides include bridge (e.g. disulfide) between X₂ and X₈.

| Molecule | | | | | | | |
|---|---|---|---|---|---|---|---|
| Name | X₋₄ | X₋₃ | X₋₂ | X₋₁ | X₁ | X₂ | X₃ |
| 1146 | Lys* | D-Arg | Gly | D-Arg | D-Nar | Cys | Phg |
| 1139 | | Lys* | Gly | D-Arg | D-Nar | Cys | Phg |
| 1145 | | Lys* | PEG1 | PEG1 | D-Nar | Cys | Phg |
| 1147 | Lys* | D-Arg | PEG1 | D-Arg | Beta-homoArg | Cys | Phg |
| 1148 | Lys* | D-Arg | Gly | D-Arg | D-Nar | Cys | D-aMeOrn |
| 1149 | | Lys* | Gly | D-Arg | D-Nar | Cys | D-aMeOrn |
| 1137 | | Lys* | PEG1 | PEG1 | D-Nar | Cys | D-aMeOrn |
| 1136 | Lys* | D-Arg | PEG1 | D-Arg | Beta-homoArg | Cys | D-aMeOrn |
| 1150 | Lys* | D-Arg | Gly | D-Arg | D-Nar | Cys | Aib(O-cyclic) |
| 1142 | | Lys* | Gly | D-Arg | D-Nar | Cys | Aib(O-cyclic) |
| 1144 | | Lys* | PEG1 | PEG1 | D-Nar | Cys | Aib(O-cyclic) |
| 1151 | Lys* | D-Arg | PEG1 | D-Arg | Beta-homoArg | Cys | Aib(O-cyclic) |
| 1152 | Lys* | D-Arg | Gly | D-Arg | D-Nar | Cys | Aib(O-cyclic) |
| 1153 | Lys* | D-Arg | Gly | D-Arg | D-Nar | Cys | Aib(O-cyclic) |
| 1154 | Lys* | D-Arg | Gly | D-Arg | D-Nar | Cys | D-aMeOrn |
| 1155 | Lys* | D-Arg | Gly | D-Arg | D-Nar | Cys | D-aMeOrn |
| 1156 | Lys* | D-Arg | Gly | D-Arg | D-Nar | Cys | D-aMeOrn |
| 1157 | Lys* | D-Arg | Gly | D-Arg | D-Nar | Cys | D-aMeOrn |
| 1122 | | | | | D-Nar | Cys | Aib(O-cyclic) |
| 1123 | | | | | D-Nar | Cys | Aib(O-cyclic) |
| 1124 | | | | | D-Nar | Cys | D-aMeOrn |
| 1125 | | | | | D-Nar | Cys | D-aMeOrn |
| 1126 | | | | | D-Nar | Cys | D-aMeOrn |
| 1127 | | | | | D-Nar | Cys | D-aMeOrn |
| 25 | | | | | D-Nar | Glu | Aib(O-cyclic) |
| 26 | | | | | D-Nar | Glu | D-aMeOrn |
| 27 | | | | | D-Nar | Glu | Phg |
| 28 | | | | | Beta-homoArg | Cys | Aib(O-cyclic) |
| 29 | | | | | Beta-homoArg | Cys | D-aMeOrn |
| 30 | | | | | Beta-homoArg | Cys | Phg |
| 31 | | | | | Beta-homoArg | Cys | Phg |
| 32 | | | | | D-Nar | Cys | Phg |
| 33 | | | | | D-Nar | Cys | Phg |
| 34 | | | | | D-Nar | Cys | Phg |
| 35 | | | | | D-Nar | Cys | Phe |
| 36 | | | | | D-Nar | Cys | Tyr |
| 37 | | | | | D-Nar | Cys | Phe |
| 38 | | | | | D-Nar | Cys | Tyr |
| 39 | | | | | D-Nar | Cys | D-Phe |
| 1158 | | | | | D-Nar | Cys | Aib(O-cyclic) |
| 41 | | | | | D-Nar | Cys | D-aMeOrn |
| 42 | | | | | D-Nar | Cys | Phg |
| 43 | | | | | D-Nar | hCys | Aib(O-cyclic) |
| 44 | | | | | D-Nar | hCys | D-aMeOrn |
| 45 | | | | | D-Nar | hCys | Phg |
| 46 | | | | | D-Nar | Cys | Aib(O-cyclic) |
| 47 | | | | | D-Nar | Cys | D-aMeOrn |
| 48 | | | | | D-Nar | Cys | Phg |
| 49 | | | | | Arg | Cys | Phg |
| 50 | | | | | Arg | Cys | Phg |
| 51 | | | | | Arg | Cys | Aib(O-cyclic) |
| 52 | | | | | Arg | Cys | D-aMeOrn |
| 53 | | | | | Arg | Cys | D-aMeOrn |
| 54 | | | | | D-Nar | Cys | Phg |
| 55 | | | | | D-Nar | Cys | D-Phg |
| 56 | | | | | D-Nar | Cys | D-Phg |
| 57 | | | | | D-Nar | Cys | D-Iva |
| 58 | | | | | D-Nar | Cys | D-Iva |
| 59 | | | | | D-Nar | Cys | bAc5c |
| 60 | | | | | D-Nar | Cys | bAc5c |
| 61 | | | | | Beta-homoArg | Cys | bAc5c |
| 62 | | | | | Beta-homoArg | Cys | bAc5c |
| 63 | | | | | D-Nar | Cys | bAc4c |
| 64 | | | | | D-Nar | Cys | bAc4c |
| 65 | | | | | Beta-homoArg | Cys | bAc4c |
| 66 | | | | | Beta-homoArg | Cys | bAc4c |
| 67 | | | | | D-Nar | Cys | bAc3c |
| 68 | | | | | D-Nar | Cys | bAc3c |
| 69 | | | | | Beta-homoArg | Cys | bAc3c |
| 70 | | | | | Beta-homoArg | Cys | bAc3c |
| 71 | | | | | D-Nar | Cys | Ac3c |
| 72 | | | | | D-Nar | Cys | Ac4c |
| 73 | | | | | D-Nar | Cys | Ac6c |
| 74 | | | | | Beta-homoArg | Cys | Ac3c |
| 75 | | | | | Beta-homoArg | Cys | Ac4c |
| 76 | | | | | Beta-homoArg | Cys | Ac6c |
| 77 | | | | | D-Nar | Cys | Cyclo-Leu |
| 78 | | | | | D-Nar | Cys | Cyclo-Leu |
| 79 | | | | | Beta-homoArg | Cys | Cyclo-Leu |
| 80 | | | | | Beta-homoArg | Cys | Cyclo-Leu |
| 81 | | | | | Beta-homoArg | Cys | Cyclo-Leu |
| 82 | | | | | Beta-homoArg | Cys | Cyclo-Leu |
| 83 | | | | | D-Nar | Cys | Aib(O-cyclic) |
| 84 | | | | | D-Nar | Cys | D-aMeOrn |
| 85 | | | | | D-Nar | Cys | Phg |
| 86 | | | | | D-Nar | Cys | Aib(O-cyclic) |
| 87 | | | | | D-Nar | Cys | D-aMeOrn |
| 88 | | | | | D-Nar | Cys | Phg |
| 89 | | | | | D-Nar | Cys | Aib(O-cyclic) |
| 90 | | | | | D-Nar | Cys | D-aMeOrn |
| 91 | | | | | D-Nar | Cys | Phg |
| 92 | | | | | D-Nar | Cys | Aib(O-cyclic) |
| 93 | | | | | D-Nar | Cys | D-aMeOrn |
| 94 | | | | | D-Nar | Cys | Phg |
| 95 | | | | | D-Nar | Cys | D-aMeSer |
| 96 | | | | | D-Nar | Cys | D-aMeSer |
| 97 | | | | | D-Nar | Cys | D-aMeSer |
| 98 | | | | | D-Nar | Cys | D-aMeSer |
| 99 | | | | | D-Nar | Cys | D-aMeSer |
| 100 | | | | | Beta-homoArg | Cys | D-aMeSer |
| 101 | | | | | Beta-homoArg | Cys | D-aMeSer |
| 102 | | | | | D-Nar | Cys | L-aMeSer |
| 103 | | | | | D-Nar | Cys | L-aMeSer |
| 104 | | | | | D-Nar | Cys | L-aMeSer |
| 105 | | | | | D-Nar | Cys | L-aMeSer |
| 106 | | | | | D-Nar | Cys | L-aMeSer |
| 107 | | | | | Beta-homoArg | Cys | L-aMeSer |
| 108 | | | | | Beta-homoArg | Cys | L-aMeSer |
| 109 | | | | | D-Nar | Cys | D-aMeAsp |
| 110 | | | | | D-Nar | Cys | D-aMeSer |
| 111 | Lys* | Gly | Gly | Gly | D-Nar | Cys | Aib(O-cyclic) |
| 112 | | Lys* | Gly | Gly | D-Nar | Cys | Aib(O-cyclic) |
| 113 | | | Lys* | Gly | D-Nar | Cys | Aib(O-cyclic) |
| 114 | | | | Lys* | D-Nar | Cys | Aib(O-cyclic) |
| 123 | | Lys* | Gly | Gly | D-Nar | Cys | Phg |
| 124 | | | Lys* | Gly | D-Nar | Cys | Phg |
| 125 | | | | Lys* | D-Nar | Cys | Phg |
| 130 | | Lys* | Gly | Gly | D-Nar | Cys | D-aMeOrn |
| 131 | | | Lys* | Gly | D-Nar | Cys | D-aMeOrn |
| 132 | | | | Lys* | D-Nar | Cys | D-aMeOrn |
| 137 | | | | | Beta-homoArg | Cys | Aib(O-cyclic) |
| 138 | | | Lys* | Gly | D-Nar | Cys | Cyclo-Leu |
| 139 | | | Lys* | Gly | D-Nar | Cys | Aib(O-cyclic) |
| 140 | | | | Lys* | Arg | Cys | Aib(O-cyclic) |
| 141 | | | | Lys* | D-Nar | Cys | Aib(O-cyclic) |
| 142 | | | | Lys* | BetahomoArg | Cys | Aib(O-cyclic) |
| 143 | | | | Lys* | Arg | Cys | Aib(O-cyclic) |
| 144 | | | | Lys* | D-Nar | Cys | Aib(O-cyclic) |
| 145 | | | | Lys* | BetahomoArg | Cys | Aib(O-cyclic) |
| 146 | | | | Lys* | Arg | Cys | Aib(O-cyclic) |
| 147 | | | | Lys* | D-Nar | Cys | Aib(O-cyclic) |
| 148 | | | | Lys* | BetahomoArg | Cys | Aib(O-cyclic) |
| 149 | | | | Lys* | Arg | Cys | Aib(O-cyclic) |
| 150 | | | | Lys* | D-Nar | Cys | Aib(O-cyclic) |
| 151 | | | | Lys* | BetahomoArg | Cys | Aib(O-cyclic) |
| 152 | | | | | D-Nar | Cys | Aib(O-cyclic) |
| 153 | | | | | D-Nar | Cys | Aib(O-cyclic) |
| 154 | | | | | Beta-homoArg | Cys | D-aMeOrn |
| 155 | | | | | Beta-homoArg | Cys | D-aMeOrn |
| 156 | | | | | Beta-homoArg | Cys | D-aMeOrn |
| 157 | | | | | Beta-homoArg | Cys | Cyclo-Leu |
| 158 | | | | | D-Nar | Cys | Cyclo-Leu |
| 159 | | | | | D-Nar | Cys | D-Iva |
| 160 | | | | | D-Nar | Cys | D-Iva |
| 161 | | | | | Beta-homoArg | Cys | Cyclo-Leu |
| 162 | | | | | D-Nar | Cys | Cyclo-Leu |
| 163 | | | | | D-Nar | Cys | D-Iva |
| 164 | | | | | D-Nar | Cys | D-Iva |
| 165 | | | | Lys* | Arg | Cys | Aib |
| 166 | | | | Lys* | D-Nar | Pen | Aib(O-cyclic) |
| 167 | | | | Lys* | D-Nar | Pen | Aib(O-cyclic) |
| 168 | | | | | D-Nar | Pen | Aib(O-cyclic) |
| 169 | | | | | D-Nar | Pen | Aib(O-cyclic) |
| 170 | | | | | D-Nar | Cys | Aib(O-cyclic) |
| 171 | | | | | D-Nar | Cys | (3S)-3- |
| | | | | | | | Aminotetrahydro- |
| | | | | | | | 3-furancarboxylic acid |
| 172 | | | | | D-Nar | Cys | (3R)-3- |
| | | | | | | | Aminotetrahydro- |
| | | | | | | | 3-furancarboxylic acid |
| 173 | | | | | D-Nar | Cys | (3S)-3- |
| | | | | | | | Aminotetrahydro- |
| | | | | | | | 3-thiphenecarboxylic |
| | | | | | | | acid |
| 174 | | | | | D-Nar | Cys | (3R)-3- |
| | | | | | | | Aminotetrahydro- |
| | | | | | | | 3-thiophenecarboxylic |
| | | | | | | | acid |
| 175 | | | | | D-Nar | Cys | N-Boc-(3S)-3-amino- |
| | | | | | | | 1,3- |
| | | | | | | | pyrrolidinedicarboxylate |
| 176 | | | | | D-Nar | Cys | N-Boc-(3R)-3-amino- |
| | | | | | | | 1,3- |
| | | | | | | | pyrrolidinedicarboxylate |
| 177 | | | | | D-Nar | Cys | 3-Amino-3-thietane- |
| | | | | | | | carboxylic acid |
| 178 | | | | | D-Nar | Cys | 3-Aminothietane-3- |
| | | | | | | | carboxylic acid 1,1- |
| | | | | | | | dioxide |
| 179 | | | | | D-Nar | Cys | N-Boc-3-amino-1,3- |
| | | | | | | | azetidinedicarboxylate |
| 180 | | | | | D-Nar | Cys | 1-Amino-3,3- |
| | | | | | | | dimethylcyclobutane- |
| | | | | | | | carboxylic acid |
| 181 | | | | | D-Nar | Cys | 5- |
| | | | | | | | Aminospiro[2.3]hexane- |
| | | | | | | | 5-carboxylic acid |
| 182 | | | | | D-Nar | Cys | 6-Amino-2- |
| | | | | | | | oxaspiro[3.3]heptane- |
| | | | | | | | 6-carboxylic acid |
| 183 | | | | | D-Nar | Cys | 2-amino-2- |
| | | | | | | | ethylbutanoic acid |
| 184 | | | | | D-Nar | Cys | (1S)-1-Amino-2,3- |
| | | | | | | | dihydro- |
| | | | | | | | 1H-indene-1-carboxylic |
| | | | | | | | acid |
| 185 | | | | | D-Nar | Cys | (1R)-1-Amino-2,3- |
| | | | | | | | dihydro- |
| | | | | | | | 1H-indene-1-carboxylic |
| | | | | | | | acid |
| 186 | | | | | D-Nar | Cys | Aib(O-cyclic) |
| 187 | | | | | D-Nar | Cys | Aib(O-cyclic) |
| 188 | | | | | D-Nar | Cys | Aib(O-cyclic) |
| 189 | | | | | D-Nar | Cys | Aib(O-cyclic) |
| 190 | | | | | D-Nar | Cys | Aib(O-cyclic) |
| 191 | | | | | D-Nar | Cys | Aib(O-cyclic) |
| 192 | | | | | D-Nar | Cys | Aib(O-cyclic) |
| 193 | | | | | D-Nar | Cys | Aib(O-cyclic) |
| 194 | | | | | D-Nar | Cys | Aib(O-cyclic) |
| 195 | | | | | D-Nar | Cys | Aib(O-cyclic) |
| 196 | | | | | D-Nar | Cys | Aib(O-cyclic) |
| 197 | | | | | D-Nar | Cys | Aib(O-cyclic) |
| 198 | | | | | D-Nar | Cys | Aib(O-cyclic) |
| 199 | | | | | D-Nar | Cys | Aib(O-cyclic) |
| 200 | | | | | D-Nar | Cys | Aib(O-cyclic) |
| 201 | | | | | N-4-aminobutyl- | Cys | D-Ala |
| | | | | | Gly | | |
| 202 | | | | | Arg | Cys | D-Asp |
| 203 | | | | | Arg | Cys | D-Glu |
| 204 | | | | | Arg | Cys | D-Dab |
| 205 | | | | | Arg | Cys | D-Ala |
| 206 | | | | | Arg | Cys | Glu |
| 207 | | | | | Arg | Cys | D-Ser |
| 208 | | | | | Arg | Cys | D-Abu |
| 209 | | | | | Arg | Cys | Glu |
| 210 | | | | | Arg | Cys | D-Ala |
| 211 | | | | | Arg | Cys | D-Ala |
| 212 | | | | | Arg | Cys | D-Ala |
| 213 | | | | | Arg | Cys | D-Ala |
| 214 | | | | | Arg | Cys | D-Ala |
| 215 | | | | | Beta-homoArg | Cys | D-Ala |
| 216 | | | | | Arg | Cys | Ala(2-Me) |
| 1001 | | | | | Arg | Cys | Cyclo-Leu |
| 218 | | | | | Arg | Cys | D-Ala |
| 219 | | | | | Arg | Cys | D-Ala |
| 220 | | | | | Arg | Cys | D-Ala |
| 221 | | | | | Arg | Cys | D-Ala |
| 222 | | | | | Arg | Cys | D-Ala |
| 223 | | | | | Arg | Cys | D-Ala |
| 224 | | | | | Arg | Cys | D-Ala |
| 1002 | | | | | Arg | Cys | D-Ala |
| 226 | | | | | Arg | Cys | D-Ala |
| 227 | | | | | D-hArg | Cys | D-Ala |
| 228 | | | | | L-hArg | Cys | D-Ala |
| 229 | | | | | [delta- | Cys | D-Ala |
| | | | | | Guanidinovaleric | | |
| | | | | | acid | | |
| 230 | | | | | [4- | Cys | D-Ala |
| | | | | | Guanidinobutyric | | |
| | | | | | acid] | | |
| 1003 | | | | | Arg | Cys | L-aMeGlu |
| 232 | | | | | Arg | Cys | D-aMeAsp |
| 1004 | | | | | Arg | Cys | L-aMeAsp |
| 234 | | | | | Arg | Cys | D-aMeSer |
| 235 | | | | | Arg | Cys | L-aMeSer |
| 236 | | | | | Arg | Cys | Ac4c |
| 237 | | | | | Arg | Cys | Ac6c |
| 238 | | | | | Arg | Cys | 4-aminooxane-4- |
| | | | | | | | carboxylic acid |
| 239 | | | | | Arg | Cys | D-hSer |
| 240 | | | | | Arg | Cys | D-Nva |
| 241 | | | | | Arg | Cys | D-Ala |
| 242 | | | | | D-Arg | Cys | Ala(2-Me) |
| 1005 | | | | | D-Arg | Cys | Cyclo-Leu |
| 244 | | | | | Arg | Cys | Glu |
| 245 | | | | | Arg | Cys | Ala(2-Me) |
| 246 | | | | | D-Arg | Cys | D-Asp |
| 247 | | | | | Beta-homoArg | Cys | Glu |
| 248 | | | | | D-Arg | Cys | D-Ser |
| 249 | | | | | Arg | Cys | D-Asp |
| 250 | | | | | D-Arg | Cys | D-Ala |
| 251 | | | | | Arg | Cys | Ala(2-Me) |
| 252 | | | | | Arg | Cys | Glu |
| 253 | | | | | Beta-homoArg | Cys | Ala(2-Me) |
| 1006 | | | | | Beta-homoArg | Cys | Cyclo-Leu |
| 255 | | | | | D-Arg | Cys | D-Dab |
| 1007 | | | | | D-Arg | Cys | L-aMeGlu |
| 257 | | | | | Orn | Cys | D-Ala |
| 258 | | | | | D-Nar | Cys | D-Ala |
| 259 | | | | | Arg | Cys | 3-aminoazetidine-3- |
| | | | | | | | carboxylic acid |
| 260 | | | | | Arg | Cys | D-Lys |
| 261 | | | | | Arg | Cys | D-Orn |
| 262 | | | | | Arg | Cys | Ame-L-Abu |
| 263 | | | | | Arg | Cys | D-aMeLeu |
| 264 | | | | | Arg | Cys | Gln |
| 265 | | | | | Arg | Cys | D-Leu |
| 266 | | | | | Arg | Cys | D-Ala |
| 267 | | | | | Arg | Cys | D-Ala |
| 268 | | | | | Arg | Cys | D-Ala |
| 269 | | | | | Arg | Cys | D-Ala |
| 270 | | | | | Arg | Cys | D-Ala |
| 271 | | | | | Arg | Cys | D-Ala |
| 272 | | | | | Beta-homoArg | Cys | D-Dab |
| 273 | | | | | Arg | Cys | Ala(2-Me) |
| 274 | | | | | Beta-homoArg | Cys | D-Dap |
| 275 | | | | | Beta-homoArg | Cys | D-Dab |
| 276 | | | | | Beta-homoArg | Cys | Ala(2-Me) |
| 1008 | | | | | Beta-homoArg | Cys | Cyclo-Leu |
| 278 | | | | | D-Arg | Cys | D-Dab |
| 279 | | | | | D-Arg | Cys | Ala(2-Me) |
| 1009 | | | | | D-Arg | Cys | Cyclo-Leu |
| 287 | | | | gGlu | Arg | Cys | D-Ala |
| 288 | | | | gGlu | D-Arg | Cys | D-Ala |
| 289 | | | | gGlu | Beta-homoArg | Cys | D-Ala |
| 290 | | | Arg | Gly | Arg | Cys | D-Ala |
| 291 | | | Glu | Pro | Arg | Cys | D-Ala |
| 292 | | | | Inp | D-Arg | Cys | D-Ala |
| 293 | | | | Tyr | Arg | Cys | D-Ala |
| 294 | | | | D-homoPhe | Arg | Cys | D-Ala |
| 295 | | | | Beta- | Arg | Cys | D-Ala |
| | | | | homoArg | | | |
| 296 | | | Leu | Ala | Arg | Cys | D-Ala |
| 297 | | | Glu | Ala | Beta-homoArg | Cys | D-Ala |
| 298 | | | Arg | Gly | Beta-homoArg | Cys | D-Ala |
| 299 | | | Leu | Ala | Beta-homoArg | Cys | D-Ala |
| 300 | | | Glu | Pro | Beta-homoArg | Cys | D-Ala |
| 301 | | | Phe | Gly | Beta-homoArg | Cys | D-Ala |
| 345 | | | | | Arg | Cys | D-homoPhe |
| 346 | | | | | Arg | Cys | D-Ala |
| 347 | | | | D-Phe | Arg | Cys | D-Ala |
| 348 | | | | D-Ty | Arg | Cys | D-Ala |
| 349 | | | Ser | Tyr | Arg | Cys | D-Ala |
| 350 | | | | | Lys | Cys | D-Ala |
| 351 | | | | | Ser | Cys | D-Ala |
| 357 | | | | | Arg | Cys | D-Ala |
| 358 | | | | | Arg | Cys | D-Ala |
| 359 | | | | | Arg | Cys | D-Ala |
| 360 | | | | | Arg | Cys | D-Ala |
| 361 | | | | | Arg | Cys | D-Tyr |
| 362 | | | | | Arg | Cys | D-Ala |
| 363 | | | | Arg(Me) | Arg | Cys | D-Ala |
| 364 | | | | | Arg | Cys | D-Ala |
| 365 | | | | PEG1 | Arg | Cys | D-Ala |
| 366 | | | | | Nar | Cys | D-Ala |
| 367 | | | | | Arg | Cys | L-aMeOrn |
| 1010 | | | | | Arg | Cys | D-aMeOrn |
| 369 | | | | | Arg | Cys | beta-Ala(2Me) |
| 370 | | | | | Arg | Cys | D-Ala |
| 374 | | | | | Arg | Cys | L-aMeGly(allyl) |
| 375 | | | | | Arg | Cys | D-Ala |
| 376 | | | | | Arg | Cys | D-Ala |
| 377 | | | | | Arg | Cys | Nip(4-NH2) |
| 378 | | | | | D-Arg | Cys | Nip(4-NH2) |
| 379 | | | | | D-Arg | Cys | D-Dap |
| 380 | | | | Gaba | D-Arg | Cys | D-Ala |
| 381 | | | | | Arg | Cys | L-Dab |
| 382 | | | | | D-Arg | Cys | L-Dab |
| 383 | | | | | Arg | Cys | Orn |
| 385 | | | | | D-Arg | Cys | L-aMeVal |
| 386 | | | | | D-Arg | Cys | D-aMeVal |
| 387 | | | | gGlu | Me-D-Arg | Cys | D-Ala |
| 388 | | | Glu | Gly | Beta-homoArg | Cys | D-Ala |
| 390 | | | | Inp | Arg | Cys | D-Ala |
| 391 | | | | D-Arg | Arg | Cys | D-Ala |
| 392 | | | | Tranexamic | Arg | Cys | D-Ala |
| | | | | acid | | | |
| 393 | | | | homoPhe | Arg | Cys | D-Ala |
| 394 | | | | D-hArg | Arg | Cys | D-Ala |
| 395 | | | | Gaba | Arg | Cys | D-Ala |
| 396 | | | | Gaba | Beta-homoArg | Cys | D-Ala |
| 397 | | | Gln | Gly | Arg | Cys | D-Ala |
| 399 | | | Lys | Gly | Me-Arg | Cys | D-Ala |
| 400 | | | | gGlu | Me-Arg | Cys | D-Ala |
| 401 | | | | 2Nal | Arg | Cys | D-Ala |
| 1011 | | | | | Arg | Cys | D-Ala |
| 1012 | | | | | Arg | Cys | Cyclo-Leu |
| 407 | | | | | Arg | Cys | Ala(2-Me) |
| 1013 | | | | | Arg | Cys | Ala(2-Me) |
| 409 | | | | | Arg | Cys | D-Glu |
| 410 | | | | | Arg | Cys | D-Ala |
| 411 | | | | Tyr | Beta-homoArg | Cys | D-Ala |
| 412 | | | | | Arg | Cys | Nip(4-NH2) |
| 413 | | | | | Beta-homoArg | Cys | Nip(4-NH2) |
| 414 | | | | | Arg | Cys | Ala(2-Me) |
| 1014 | | | | | Beta-homoArg | Cys | D-Dab |
| 419 | | | | | Arg | Cys | D-Dab |
| 1015 | | | | | D-Arg | Cys | Cyclo-Leu |
| 421 | | | Lys | Gly | Me-D-Arg | Cys | D-Ala |
| 422 | | | | | Beta-homoArg | Cys | Ala(2-Me) |
| 424 | | | | PEG2 | Arg | Cys | D-Ala |
| 1016 | | | | | D-Arg | Cys | L-aMeGlu |
| 1017 | | | | | D-Arg | Cys | L-aMeGlu |
| 1018 | | | | Gly | D-Arg | Cys | L-aMeGlu |
| 428 | | | Lys* | Gly | D-Arg | Cys | L-aMeGlu |
| 1019 | | | | | D-Nar | Cys | L-aMeGlu |
| 1020 | | | | | D-Nar | Cys | L-aMeGlu |
| 1021 | | | | | D-Nar | Cys | L-aMeGlu |
| 1022 | | | | | Beta-homoArg | Cys | L-aMeGlu |
| 1023 | | | | | Beta-homoArg | Cys | L-aMeGlu |
| 1024 | | | | | Beta-homoArg | Cys | L-aMeGlu |
| 1025 | | | | Gly | Beta-homoArg | Cys | L-aMeGlu |
| 436 | | | Lys* | Gly | Beta-homoArg | Cys | L-aMeGlu |
| 437 | | | | | Beta-homoArg | Cys | D-hSer |
| 1026 | | | | | Beta-homoArg | Cys | L-aMeGlu |
| 439 | | | | | Beta-homoArg | Cys | 4-aminooxane-4- |
| | | | | | | | carboxylic acid |
| 1027 | | | | | Beta-homoArg | Cys | Cyclo-Leu |
| 441 | | | | | Beta-homoArg | Cys | D-aMeSer |
| 1028 | | | | | Beta-homoArg | Cys | L-aMeGlu |
| 1029 | | | | | L-hArg | Cys | L-aMeGlu |
| 444 | | | | | L-hArg | Cys | D-hSer |
| 1030 | | | | | Beta-homoArg | Cys | L-aMeGlu |
| 1031 | | | | | Beta-homoArg | Cys | L-aMeGlu |
| 1032 | | | | | Beta-homoArg | Cys | L-aMeGlu |
| 1033 | | | | | Beta-homoArg | Cys | bhGlu |
| 449 | | | | | Beta-homoArg | Cys | hGlu |
| 450 | | | | | Beta-homoArg | Cys | D-3Thi |
| 451 | | | | | Beta-homoArg | Cys | D-Iva |
| 452 | | | | | Beta-homoArg | Cys | bAc5c |
| 1034 | | | | | Beta-homoArg | Cys | Phg |
| 454 | | | | | Beta-homoArg | Cys | D-Phg |
| 455 | | | | | D-Nar | Cys | Cyclo-Leu(3-ene) |
| 456 | | | | | D-Nar | Cys | L-Apm |
| 1035 | | | | | D-Nar | Cys | L-aMeGlu |
| 1036 | | | | | D-Nar | Cys | D-aMeOrn |
| 1037 | | | | | D-Nar | Cys | Ala(2-Me) |
| 1038 | | | | | D-Nar | Cys | L-aMeGlu |
| 1039 | | | | | D-Nar | Cys | D-aMeSer |
| 1040 | | | | | D-Nar | Cys | L-aMeGlu |
| 463 | | | | | D-Nar | Cys | Ac3c |
| 1041 | | | | | D-Nar | Cys | L-aMeGlu |
| 1042 | | | | | D-Nar | Cys | L-aMeAsp |
| 1043 | | | | | D-Nar | Cys | L-aMeGlu |
| 467 | | | | | D-Nar | Cys | bAc4c |
| 468 | | | | | D-Nar | Cys | 4-aminooxane-4- |
| | | | | | | | carboxylic acid |
| 469 | | | | | D-Nar | Cys | Ala(2-Me) |
| 1044 | | | | | D-Nar | Cys | D-bhGlu |
| 1045 | | | | | D-Nar | Cys | L-aMeGlu |
| 1046 | | | | | D-Nar | Cys | L-aMeGlu |
| 1047 | | | | | D-Nar | Cys | L-aMeGlu |
| 1048 | | | | | D-Nar | Cys | L-aMeGlu |
| 475 | | | | | D-Nar | Cys | bhGlu |
| 1049 | | | | | D-Nar | Cys | hGlu |
| 1050 | | | | | D-Nar | Cys | L-aMeGlu |
| 1051 | | | | | D-Nar | Cys | L-aMeGlu |
| 1052 | | | | | Beta-homoArg | Cys | cyclo-Leu |
| 1053 | | | | | Arg | Cys | L-aMeGlu |
| 1054 | | | | | L-hArg | Cys | L-aMeGlu |
| 482 | | | | | D-Nar | Cys | Ac3c |
| 1055 | | | | | D-Arg | Cys | D-aMeOrn |
| 1056 | | | | | Arg | Cys | L-aMeGlu |
| 1057 | | | | | Arg | Cys | L-aMeGlu |
| 1058 | | | | | D-Nar | Cys | Aib(O-cyclic) |
| 487 | | | | | D-Nar | Cys | D-hSer |
| 1059 | | | | | Beta-homoArg | Cys | L-aMeGlu |
| 489 | | | | | Beta-homoArg | Cys | D-hSer |
| 1060 | | | | | D-Arg | Cys | L-aMeGlu |
| 1061 | | | | | L-hArg | Cys | L-aMeGlu |
| 1062 | | | | | D-Nar | Cys | L-aMeAsp |
| 1063 | | | | | D-Arg | Cys | L-aMeAsp |
| 1129 | | Lys* | Gly | D-Arg | D-Nar | Cys | L-aMeGlu |
| 1128 | | Lys* | Glu | PRO | D-Nar | Cys | L-aMeGlu |
| 1133 | | Lys* | Gly | gGlu | D-Nar | Cys | L-aMeGlu |
| 1064 | | | | | D-Nar | Cys | L-aMeAsp |
| 499 | | Lys* | Gly | Gly | D-Nar | Cys | L-aMeAsp |
| 1131 | | Lys* | PEG1 | PEG1 | D-Nar | Cys | L-aMeAsp |
| 1065 | | | | | beta-homoArg | Cys | L-aMeAsp |
| 1130 | | Lys* | Gly | Gly | beta-homoArg | Cys | L-aMeAsp |
| 504 | | Lys* | PEG1 | PEG1 | beta-homoArg | Cys | L-aMeAsp |
| 1135 | | Lys* | Gly | Gly | D-Arg | Cys | L-aMeAsp |
| 1066 | | | | | D-Nar | Glu | L-aMeGlu |
| 1067 | | | | | D-Nar | Asp | L-aMeGlu |
| 1068 | | | | | D-Nar | Glu | L-aMeAsp |
| 1069 | | | | | D-Nar | Asp | L-aMeAsp |
| 1070 | | | | | Arg | Cys | D-Ala |
| 1071 | | | | | D-Nar | Cys | L-aMeGlu |
| 1072 | | | | | D-Nar | Cys | L-aMeGlu |
| 1073 | | | | | D-Nar | Cys | L-aMeGlu |
| 1074 | | | | | D-Nar | Cys | L-aMeGlu |
| 1075 | | | | | D-Nar | Cys | L-aMeGlu |
| 1076 | | | | | D-Nar | Cys | L-aMeGlu |
| 1077 | | | | | D-Nar | Cys | L-aMeGlu |
| 1078 | | | | | D-Nar | Cys | L-aMeGlu |
| 1079 | | | | | D-Nar | Cys | L-aMeGlu |
| 1080 | | | | | D-Nar | Cys | L-aMeGlu |
| 1081 | | | | | D-Nar | Cys | L-aMeGlu |
| 1082 | | | | | D-Nar | Cys | L-aMeGlu |
| 1083 | | | | | D-Nar | Cys | L-aMeGlu |
| 1084 | | | | | D-Nar | Cys | L-aMeGlu |
| 1085 | | | | | D-Nar | Cys | L-aMeGlu |
| 1086 | | | | | D-Nar | Cys | L-aMeGlu |
| 1087 | | | | | D-Nar | Cys | L-aMeGlu |
| 1088 | | | | | D-Nar | Cys | L-aMeGlu |
| 1089 | | | | | D-Nar | Cys | L-aMeAsp |
| 532 | | | | | D-Nar | Cys | D-bhGlu |
| 533 | | | | | D-Nar | Cys | D-bhGlu |
| 534 | | | | | D-Nar | Cys | D-bhGlu |
| 535 | | | | | D-Nar | Cys | hGlu |
| 536 | | | | | D-Nar | Cys | hGlu |
| 537 | | | | | D-Nar | Cys | hGlu |
| 538 | | | | | D-Nar | Cys | hGlu |
| 539 | | | | | D-Nar | Cys | Apm |
| 540 | | | | | D-Nar | Cys | Apm |
| 541 | | | | | D-Nar | Cys | Apm |
| 542 | | | | | D-Nar | Cys | Apm |
| 1090 | | | | | D-Nar | Cys | L-aMeGlu |
| 1091 | | | | | D-Nar | Cys | hGlu |
| 1092 | | | | | D-Nar | Cys | Aib(O-cyclic) |
| 1093 | | | | | D-Nar | Cys | Aib(O-cyclic) |
| 1094 | | | | | D-Nar | Cys | D-aMeOrn |
| 1096 | | | | | D-Nar | Cys | D-aMeSer |
| 1097 | | | | | D-Nar | Cys | D-aMeSer |
| 1098 | | | | | D-Nar | Cys | bhGlu |
| 1099 | | | | | D-Nar | Cys | Aib |
| 1100 | | | | | D-Nar | Cys | D-aMeOrn |
| 1101 | | | | | D-Nar | Cys | D-aMeOrn |
| 1102 | | | | | D-Nar | Cys | Aib(O-cyclic) |
| 1103 | | | | | D-Nar | Cys | Aib(O-cyclic) |
| 1104 | | | | | D-Nar | Cys | L-aMeGlu |
| 1106 | | | | | D-Nar | Cys | Aib(O-cyclic) |
| 1107 | | | | | D-Nar | Cys | Aib(O-cyclic) |
| 1108 | | | | | D-Nar | Cys | Cyclo-Leu |
| 1109 | | | | | D-Nar | Cys | D-aMeOrn |
| 561 | Lys* | D-Arg | PEG1 | PEG1 | D-Nar | Cys | L-aMeGlu |
| 562 | Lys* | PEG1 | PEG1 | PEG1 | D-Nar | Cys | L-aMeGlu |
| 563 | Lys* | Glu | PEG1 | PEG1 | D-Nar | Cys | L-aMeGlu |
| 564 | Lys* | Glu | Glu | Pro | D-Nar | Cys | L-aMeGlu |
| 1141 | Lys* | Ser | Glu | Pro | D-Nar | Cys | L-aMeGlu |
| 566 | Lys* | Ser | Gly | D-Arg | D-Nar | Cys | L-aMeGlu |
| 567 | Lys* | D-Arg | Gly | D-Arg | D-Nar | Cys | L-aMeGlu |
| 568 | Lys* | D-Arg | Ser | γ-Glu | D-Nar | Cys | L-aMeGlu |
| 569 | Lys* | Glu | Gly | γ-Glu | D-Nar | Cys | L-aMeGlu |
| 1143 | Lys* | Gly | Gly | γ-Glu | D-Nar | Cys | L-aMeGlu |
| 571 | | Lys* | Gly | γ-Glu | Me-D-Arg | Cys | L-aMeGlu |
| 572 | | Lys* | Gly | D-Arg | D-Nar | Cys | L-aMeGlu |
| 573 | | Lys* | Glu | Pro | D-Nar | Cys | L-aMeGlu |
| 574 | | Lys* | Glu | γ-Glu | D-Nar | Cys | L-aMeGlu |
| 575 | | Lys* | D-Arg | γ-Glu | D-Nar | Cys | L-aMeGlu |
| 1110 | | | | | D-Nar | Cys | Phg |
| 1111 | | | | | D-Nar | Cys | Phg |
| 1112 | | | | | D-Nar | Cys | Phg |
| 1113 | | | | | D-Nar | Cys | Phg |
| 1114 | | | | | Nar | Cys | L-aMeGlu |
| 1115 | | | | | Nar | Cys | L-aMeGlu |
| 1116 | | | | | D-Nar | Cys | L-aMeGlu |
| 1118 | | | | | D-Nar | Cys | L-aMeGlu |
| 1119 | | | | | D-Nar | Cys | Phg |
| 1120 | | | | | D-Nar | Glu | L-aMeAsp |
| 1121 | | | | | D-Nar | Glu | L-aMeAsp |
| 1134 | Lys* | Glu | Glu | Pro | D-Nar | Cys | L-aMeGlu |
| 1132 | Lys* | Glu | PEG1 | PEG1 | D-Nar | Cys | L-aMeGlu |
| 589 | Lys* | Glu | Glu | Pro | D-Nar | Cys | D-bhGlu |
| 590 | Lys* | Glu | PEG1 | PEG1 | D-Nar | Cys | D-bhGlu |

| Molecule | | | | | | N- | C- | |
|---|---|---|---|---|---|---|---|---|
| Name | X₄ | X₅ | X₆ | X₇ | X₈ | term | term | Cyclic |
| 1146 | 3Pal | D-Phe(4-F) | Arg | Trp(6-F) | Cys | Ac | NH₂ | Disulfide |
| 1139 | 3Pal | D-Phe(4-F) | Arg | Trp(6-F) | Cys | Ac | NH₂ | Disulfide |
| 1145 | 3Pal | D-Phe(4-F) | Arg | Trp(6-F) | Cys | Ac | NH₂ | Disulfide |
| 1147 | 3Pal | D-Phe(4-F) | Arg | Trp(6-F) | Cys | Ac | NH₂ | Disulfide |
| 1148 | Gln | D-Phe | Arg | Trp(6-F) | Cys | Ac | NH₂ | Disulfide |
| 1149 | Gln | D-Phe | Arg | Trp(6-F) | Cys | Ac | NH₂ | Disulfide |
| 1137 | Gln | D-Phe | Arg | Trp(6-F) | Cys | Ac | NH₂ | Disulfide |
| 1136 | Gln | D-Phe | Arg | Trp(6-F) | Cys | Ac | NH₂ | Disulfide |
| 1150 | Gln | D-Phe(4-F) | Arg | Trp(6-F) | Cys | Ac | NH₂ | Disulfide |
| 1142 | Gln | D-Phe(4-F) | Arg | Trp(6-F) | Cys | Ac | NH₂ | Disulfide |
| 1144 | Gln | D-Phe(4-F) | Arg | Trp(6-F) | Cys | Ac | NH₂ | Disulfide |
| 1151 | Gln | D-Phe(4-F) | Arg | Trp(6-F) | Cys | Ac | NH₂ | Disulfide |
| 1152 | 3Pal | D-Phe(4-F) | Arg | Trp(6-F) | Cys | Ac | NH₂ | Disulfide |
| 1153 | Orn | D-Phe(4-F) | Arg | Trp(6-F) | Cys | Ac | NH₂ | Disulfide |
| 1154 | 3Pal | D-Phe(4-F) | Arg | Trp(6-F) | Cys | Ac | NH₂ | Disulfide |
| 1155 | Orn | D-Phe(4-F) | Arg | Trp(6-F) | Cys | Ac | NH₂ | Disulfide |
| 1156 | 3Pal | D-Phe | Arg | Trp(6-F) | Cys | Ac | NH₂ | Disulfide |
| 1157 | Orn | D-Phe | Arg | Trp(6-F) | Cys | Ac | NH₂ | Disulfide |
| 1122 | 3Pal | D-Phe(4-F) | Arg | Trp(6-F) | Cys | Ac | NH₂ | Disulfide |
| 1123 | Orn | D-Phe(4-F) | Arg | Trp(6-F) | Cys | Ac | NH₂ | Disulfide |
| 1124 | 3Pal | D-Phe(4-F) | Arg | Trp(6-F) | Cys | Ac | NH₂ | Disulfide |
| 1125 | Orn | D-Phe(4-F) | Arg | Trp(6-F) | Cys | Ac | NH₂ | Disulfide |
| 1126 | 3Pal | D-Phe | Arg | Trp(6-F) | Cys | Ac | NH₂ | Disulfide |
| 1127 | Orn | D-Phe | Arg | Trp(6-F) | Cys | Ac | NH₂ | Disulfide |
| 25 | Gln | D-Phe(4-F) | Arg | Trp(6-F) | Dap | Ac | NH₂ | Disulfide |
| 26 | Gln | D-Phe | Arg | Trp(6-F) | Dap | Ac | NH₂ | Disulfide |
| 27 | 3Pal | D-Phe(4-F) | Arg | Trp(6-F) | Dap | Ac | NH₂ | Disulfide |
| 28 | Gln | D-Phe(4-F) | Arg | Trp(6-F) | Cys | Ac | NH₂ | Disulfide |
| 29 | Gln | D-Phe | Arg | Trp(6-F) | Cys | Ac | NH₂ | Disulfide |
| 30 | 3Pal | D-Phe(4-F) | Arg | Trp(6-F) | Cys | Ac | NH₂ | Disulfide |
| 31 | Gln | D-Phe(4-F) | Arg | Trp(6-F) | Cys | Ac | NH₂ | Disulfide |
| 32 | Gln | D-Phe(4-F) | Arg | Trp(6-F) | Cys | Ac | NH₂ | Disulfide |
| 33 | Cit | D-Phe(4-F) | Arg | Trp(6-F) | Cys | Ac | NH₂ | Disulfide |
| 34 | hCit | D-Phe(4-F) | Arg | Trp(6-F) | Cys | Ac | NH₂ | Disulfide |
| 35 | 3Pal | D-Phe(4-F) | Arg | Trp(6-F) | Cys | Ac | NH₂ | Disulfide |
| 36 | 3Pal | D-Phe(4-F) | Arg | Trp(6-F) | Cys | Ac | NH₂ | Disulfide |
| 37 | Gln | D-Phe(4-F) | Arg | Trp(6-F) | Cys | Ac | NH₂ | Disulfide |
| 38 | Gln | D-Phe(4-F) | Arg | Trp(6-F) | Cys | Ac | NH₂ | Disulfide |
| 39 | Gln | D-Phe(4-F) | Arg | Trp(6-F) | Cys | Ac | NH₂ | Disulfide |
| 1158 | Gln | D-Phe(4-F) | Arg | Trp(6-F) | Pen | Ac | NH₂ | Disulfide |
| 41 | Gln | D-Phe | Arg | Trp(6-F) | Pen | Ac | NH₂ | Disulfide |
| 42 | 3Pal | D-Phe(4-F) | Arg | Trp(6-F) | Pen | Ac | NH₂ | Disulfide |
| 43 | Gln | D-Phe(4-F) | Arg | Trp(6-F) | Pen | Ac | NH₂ | Disulfide |
| 44 | Gln | D-Phe | Arg | Trp(6-F) | Pen | Ac | NH₂ | Disulfide |
| 45 | 3Pal | D-Phe(4-F) | Arg | Trp(6-F) | Pen | Ac | NH₂ | Disulfide |
| 46 | Gln | D-Phe | Arg | Trp | Cys | Ac | NH₂ | Disulfide |
| 47 | Gln | D-Phe | Arg | Trp | Cys | Ac | NH₂ | Disulfide |
| 48 | 3Pal | D-Phe | Arg | Trp | Cys | Ac | NH₂ | Disulfide |
| 49 | 3Pal | D-Phe | Arg | Trp | Cys | Ac | NH₂ | Disulfide |
| 50 | Gln | D-Phe | Arg | Trp | Cys | Ac | NH₂ | Disulfide |
| 51 | Gln | D-Phe | Arg | Trp | Cys | Ac | NH₂ | Disulfide |
| 52 | Gln | D-Phe | Arg | Trp | Cys | Ac | NH₂ | Disulfide |
| 53 | Gln | D-Phe | Arg | Trp | Pen | Ac | NH₂ | Disulfide |
| 54 | Orn | D-Phe(4-F) | Arg | Trp(6-F) | Cys | Ac | NH₂ | Disulfide |
| 55 | 3Pal | D-Phe(4-F) | Arg | Trp(6-F) | Cys | Ac | NH₂ | Disulfide |
| 56 | Gln | D-Phe(4-F) | Arg | Trp(6-F) | Cys | Ac | NH₂ | Disulfide |
| 57 | 3Pal | D-Phe(4-F) | Arg | Trp(6-F) | Cys | Ac | NH₂ | Disulfide |
| 58 | Gln | D-Phe(4-F) | Arg | Trp(6-F) | Cys | Ac | NH₂ | Disulfide |
| 59 | 3Pal | D-Phe(4-F) | Arg | Trp(6-F) | Cys | Ac | NH₂ | Disulfide |
| 60 | Gln | D-Phe(4-F) | Arg | Trp(6-F) | Cys | Ac | NH₂ | Disulfide |
| 61 | 3Pal | D-Phe(4-F) | Arg | Trp(6-F) | Cys | Ac | NH₂ | Disulfide |
| 62 | Gln | D-Phe(4-F) | Arg | Trp(6-F) | Cys | Ac | NH₂ | Disulfide |
| 63 | 3Pal | D-Phe(4-F) | Arg | Trp(6-F) | Cys | Ac | NH₂ | Disulfide |
| 64 | Gln | D-Phe(4-F) | Arg | Trp(6-F) | Cys | Ac | NH₂ | Disulfide |
| 65 | 3Pal | D-Phe(4-F) | Arg | Trp(6-F) | Cys | Ac | NH₂ | Disulfide |
| 66 | Gln | D-Phe(4-F) | Arg | Trp(6-F) | Cys | Ac | NH₂ | Disulfide |
| 67 | 3Pal | D-Phe(4-F) | Arg | Trp(6-F) | Cys | Ac | NH₂ | Disulfide |
| 68 | Gln | D-Phe(4-F) | Arg | Trp(6-F) | Cys | Ac | NH₂ | Disulfide |
| 69 | 3Pal | D-Phe(4-F) | Arg | Trp(6-F) | Cys | Ac | NH₂ | Disulfide |
| 70 | Gln | D-Phe(4-F) | Arg | Trp(6-F) | Cys | Ac | NH₂ | Disulfide |
| 71 | 3Pal | D-Phe(4-F) | Arg | Trp(6-F) | Cys | Ac | NH₂ | Disulfide |
| 72 | 3Pal | D-Phe(4-F) | Arg | Trp(6-F) | Cys | Ac | NH₂ | Disulfide |
| 73 | 3Pal | D-Phe(4-F) | Arg | Trp(6-F) | Cys | Ac | NH₂ | Disulfide |
| 74 | 3Pal | D-Phe(4-F) | Arg | Trp(6-F) | Cys | Ac | NH₂ | Disulfide |
| 75 | 3Pal | D-Phe(4-F) | Arg | Trp(6-F) | Cys | Ac | NH₂ | Disulfide |
| 76 | 3Pal | D-Phe(4-F) | Arg | Trp(6-F) | Cys | Ac | NH₂ | Disulfide |
| 77 | 3Pal | D-Phe(4-F) | Arg | Trp(6-F) | Cys | Ac | NH₂ | Disulfide |
| 78 | Gln | D-Phe(4-F) | Arg | Trp(6-F) | Cys | Ac | NH₂ | Disulfide |
| 79 | 3Pal | D-Phe(4-F) | Arg | Trp(6-F) | Cys | Ac | NH₂ | Disulfide |
| 80 | Gln | D-Phe(4-F) | Arg | Trp(6-F) | Cys | Ac | NH₂ | Disulfide |
| 81 | Cit | D-Phe(4-F) | Arg | Trp(6-F) | Cys | Ac | NH₂ | Disulfide |
| 82 | hCit | D-Phe(4-F) | Arg | Trp(6-F) | Cys | Ac | NH₂ | Disulfide |
| 83 | Thr | D-Phe(4-F) | Arg | Trp(6-F) | Cys | Ac | NH₂ | Disulfide |
| 84 | Thr | D-Phe | Arg | Trp(6-F) | Cys | Ac | NH₂ | Disulfide |
| 85 | Thr | D-Phe(4-F) | Arg | Trp(6-F) | Cys | Ac | NH₂ | Disulfide |
| 86 | Thr | D-Phe(4-F) | Arg | Trp(6-F) | Pen | Ac | NH₂ | Disulfide |
| 87 | Thr | D-Phe | Arg | Trp(6-F) | Pen | Ac | NH₂ | Disulfide |
| 88 | Thr | D-Phe(4-F) | Arg | Trp(6-F) | Pen | Ac | NH₂ | Disulfide |
| 89 | Ser | D-Phe(4-F) | Arg | Trp(6-F) | Cys | Ac | NH₂ | Disulfide |
| 90 | Ser | D-Phe | Arg | Trp(6-F) | Cys | Ac | NH₂ | Disulfide |
| 91 | Ser | D-Phe(4-F) | Arg | Trp(6-F) | Cys | Ac | NH₂ | Disulfide |
| 92 | Ser | D-Phe(4-F) | Arg | Trp(6-F) | Pen | Ac | NH₂ | Disulfide |
| 93 | Ser | D-Phe | Arg | Trp(6-F) | Pen | Ac | NH₂ | Disulfide |
| 94 | Ser | D-Phe(4-F) | Arg | Trp(6-F) | Pen | Ac | NH₂ | Disulfide |
| 95 | 3Pal | D-Phe(4-F) | Arg | Trp(6-F) | Cys | Ac | NH₂ | Disulfide |
| 96 | Thr | D-Phe(4-F) | Arg | Trp(6-F) | Cys | Ac | NH₂ | Disulfide |
| 97 | Ser | D-Phe(4-F) | Arg | Trp(6-F) | Cys | Ac | NH₂ | Disulfide |
| 98 | Orn | D-Phe(4-F) | Arg | Trp(6-F) | Cys | Ac | NH₂ | Disulfide |
| 99 | 3Pal | D-Phe | Arg | Trp(6-F) | Cys | Ac | NH₂ | Disulfide |
| 100 | 3Pal | D-Phe | Arg | Trp(6-F) | Cys | Ac | NH₂ | Disulfide |
| 101 | Gln | D-Phe | Arg | Trp(6-F) | Cys | Ac | NH₂ | Disulfide |
| 102 | 3Pal | D-Phe(4-F) | Arg | Trp(6-F) | Cys | Ac | NH₂ | Disulfide |
| 103 | Thr | D-Phe(4-F) | Arg | Trp(6-F) | Cys | Ac | NH₂ | Disulfide |
| 104 | Ser | D-Phe(4-F) | Arg | Trp(6-F) | Cys | Ac | NH₂ | Disulfide |
| 105 | Orn | D-Phe(4-F) | Arg | Trp(6-F) | Cys | Ac | NH₂ | Disulfide |
| 106 | 3Pal | D-Phe | Arg | Trp(6-F) | Cys | Ac | NH₂ | Disulfide |
| 107 | 3Pal | D-Phe | Arg | Trp(6-F) | Cys | Ac | NH₂ | Disulfide |
| 108 | Gln | D-Phe | Arg | Trp(6-F) | Cys | Ac | NH₂ | Disulfide |
| 109 | 3Pal | D-Phe(4-F) | Arg | Trp(6-F) | Cys | Ac | NH₂ | Disulfide |
| 110 | Gln | D-Phe(4-F) | Arg | Trp(5-Me) | Cys | Ac | NH₂ | Disulfide |
| 111 | Gln | D-Phe(4-F) | Arg | Trp(6-F) | Cys | Ac | NH₂ | Disulfide |
| 112 | Gln | D-Phe(4-F) | Arg | Trp(6-F) | Cys | Ac | NH₂ | Disulfide |
| 113 | Gln | D-Phe(4-F) | Arg | Trp(6-F) | Cys | Ac | NH₂ | Disulfide |
| 114 | Gln | D-Phe(4-F) | Arg | Trp(6-F) | Cys | Ac | NH₂ | Disulfide |
| 123 | 3Pal | D-Phe(4-F) | Arg | Trp(6-F) | Cys | Ac | NH₂ | Disulfide |
| 124 | 3Pal | D-Phe(4-F) | Arg | Trp(6-F) | Cys | Ac | NH₂ | Disulfide |
| 125 | 3Pal | D-Phe(4-F) | Arg | Trp(6-F) | Cys | Ac | NH₂ | Disulfide |
| 130 | Gln | D-Phe | Arg | Trp(6-F) | Cys | Ac | NH₂ | Disulfide |
| 131 | Gln | D-Phe | Arg | Trp(6-F) | Cys | Ac | NH₂ | Disulfide |
| 132 | Gln | D-Phe | Arg | Trp(6-F) | Cys | Ac | NH₂ | Disulfide |
| 137 | Cit | D-Phe(4-F) | Arg | Trp(6-F) | Cys | Ac | NH₂ | Disulfide |
| 138 | 3Pal | D-Phe | Arg | Trp(6-F) | Cys | Ac | NH₂ | Disulfide |
| 139 | Cit | D-Phe | Arg | Trp(6-F) | Cys | Ac | NH₂ | Disulfide |
| 140 | Gln | D-Phe(4-F) | Arg | Trp(6-F) | Pen | Ac | NH₂ | Disulfide |
| 141 | Gln | D-Phe(4-F) | Arg | Trp(6-F) | Pen | Ac | NH₂ | Disulfide |
| 142 | Gln | D-Phe(4-F) | Arg | Trp(6-F) | Pen | Ac | NH₂ | Disulfide |
| 143 | Gln | D-Phe | Arg | Trp | Pen | Ac | NH₂ | Disulfide |
| 144 | Gln | D-Phe | Arg | Trp | Pen | Ac | NH₂ | Disulfide |
| 145 | Gln | D-Phe | Arg | Trp | Pen | Ac | NH₂ | Disulfide |
| 146 | Gln | D-Phe | Arg | Trp(6-F) | Pen | Ac | NH₂ | Disulfide |
| 147 | Gln | D-Phe | Arg | Trp(6-F) | Pen | Ac | NH₂ | Disulfide |
| 148 | Gln | D-Phe | Arg | Trp(6-F) | Pen | Ac | NH₂ | Disulfide |
| 149 | Gln | D-Phe(4-F) | Arg | Trp | Pen | Ac | NH₂ | Disulfide |
| 150 | Gln | D-Phe(4-F) | Arg | Trp | Pen | Ac | NH₂ | Disulfide |
| 151 | Gln | D-Phe(4-F) | Arg | Trp | Pen | Ac | NH₂ | Disulfide |
| 152 | Gln | D-Phe | Arg | Trp(6-F) | Pen | Ac | NH₂ | Disulfide |
| 153 | Gln | D-Phe(4-F) | Arg | Trp | Pen | Ac | NH₂ | Disulfide |
| 154 | Gln | D-Phe | Arg | Trp(6-F) | Pen | Ac | NH₂ | Disulfide |
| 155 | Gln | D-Phe(4-F) | Arg | Trp(6-F) | Pen | Ac | NH₂ | Disulfide |
| 156 | Gln | D-Phe(4-F) | Arg | Trp | Pen | Ac | NH₂ | Disulfide |
| 157 | Gln | D-Phe(4-F) | Arg | Trp(6-F) | Pen | Ac | NH₂ | Disulfide |
| 158 | Gln | D-Phe(4-F) | Arg | Trp(6-F) | Pen | Ac | NH₂ | Disulfide |
| 159 | 3Pal | D-Phe(4-F) | Arg | Trp(6-F) | Pen | Ac | NH₂ | Disulfide |
| 160 | Gln | D-Phe(4-F) | Arg | Trp(6-F) | Pen | Ac | NH₂ | Disulfide |
| 161 | Gln | D-Phe | Arg | Trp(6-F) | Pen | Ac | NH₂ | Disulfide |
| 162 | Gln | D-Phe | Arg | Trp(6-F) | Pen | Ac | NH₂ | Disulfide |
| 163 | 3Pal | D-Phe | Arg | Trp(6-F) | Pen | Ac | NH₂ | Disulfide |
| 164 | Gln | D-Phe | Arg | Trp(6-F) | Pen | Ac | NH₂ | Disulfide |
| 165 | Gln | D-Phe(4-F) | Arg | Trp(6-F) | Pen | Ac | NH₂ | Disulfide |
| 166 | Gln | D-Phe(4-F) | Arg | Trp(6-F) | Pen | Ac | NH₂ | Disulfide |
| 167 | Gln | D-Phe(4-F) | Arg | Trp(6-F) | Cys | Ac | NH₂ | Disulfide |
| 168 | Gln | D-Phe(4-F) | Arg | Trp(6-F) | Pen | Ac | NH₂ | Disulfide |
| 169 | Gln | D-Phe(4-F) | Arg | Trp(6-F) | Cys | Ac | NH₂ | Disulfide |
| 170 | Lys | D-Phe(4-F) | Arg | Trp(6-F) | Pen | Ac | NH₂ | Disulfide |
| 171 | Gln | D-Phe(4-F) | Arg | Trp(6-F) | Pen | Ac | NH₂ | Disulfide |
| 172 | Gln | D-Phe(4-F) | Arg | Trp(6-F) | Pen | Ac | NH₂ | Disulfide |
| 173 | Gln | D-Phe(4-F) | Arg | Trp(6-F) | Pen | Ac | NH₂ | Disulfide |
| 174 | Gln | D-Phe(4-F) | Arg | Trp(6-F) | Pen | Ac | NH₂ | Disulfide |
| 175 | Gln | D-Phe(4-F) | Arg | Trp(6-F) | Pen | Ac | NH₂ | Disulfide |
| 176 | Gln | D-Phe(4-F) | Arg | Trp(6-F) | Pen | Ac | NH₂ | Disulfide |
| 177 | Gln | D-Phe(4-F) | Arg | Trp(6-F) | Pen | Ac | NH₂ | Disulfide |
| 178 | Gln | D-Phe(4-F) | Arg | Trp(6-F) | Pen | Ac | NH₂ | Disulfide |
| 179 | Gln | D-Phe(4-F) | Arg | Trp(6-F) | Pen | Ac | NH₂ | Disulfide |
| 180 | Gln | D-Phe(4-F) | Arg | Trp(6-F) | Pen | Ac | NH₂ | Disulfide |
| 181 | Gln | D-Phe(4-F) | Arg | Trp(6-F) | Pen | Ac | NH₂ | Disulfide |
| 182 | Gln | D-Phe(4-F) | Arg | Trp(6-F) | Pen | Ac | NH₂ | Disulfide |
| 183 | Gln | D-Phe(4-F) | Arg | Trp(6-F) | Pen | Ac | NH₂ | Disulfide |
| 184 | Gln | D-Phe(4-F) | Arg | Trp(6-F) | Pen | Ac | NH₂ | Disulfide |
| 185 | Gln | D-Phe(4-F) | Arg | Trp(6-F) | Pen | Ac | NH₂ | Disulfide |
| 186 | Gln | L-Methionine | Arg | Trp(6-F) | Pen | Ac | NH₂ | Disulfide |
| | | sulfoxide | | | | | | |
| 187 | Gln | L-Methionine | Arg | Trp(6-F) | Pen | Ac | NH₂ | Disulfide |
| | | sulfone | | | | | | |
| 188 | Gln | (2S)-2-Amino-4- | Arg | Trp(6-F) | Pen | Ac | NH₂ | Disulfide |
| | | cyanobutanoic acid | | | | | | |
| 189 | Gln | 3-(Acetylamino)-L- | Arg | Trp(6-F) | Pen | Ac | NH₂ | Disulfide |
| | | alanine | | | | | | |
| 190 | Gln | O-Carbamoyl-L- | Arg | Trp(6-F) | Pen | Ac | NH₂ | Disulfide |
| | | serine | | | | | | |
| 191 | Gln | 2-Hydroxy-L- | Arg | Trp(6-F) | Pen | Ac | NH₂ | Disulfide |
| | | tryptophan | | | | | | |
| 192 | Gln | 3-(Trimethylsilyl)-D- | Arg | Trp(6-F) | Pen | Ac | NH₂ | Disulfide |
| | | alanine | | | | | | |
| 193 | Gln | 5,5,5-Trifluoro-D- | Arg | Trp(6-F) | Pen | Ac | NH₂ | Disulfide |
| | | norvaline | | | | | | |
| 194 | Gln | 3-(Trifluoromethyl)- | Arg | Trp(6-F) | Pen | Ac | NH₂ | Disulfide |
| | | D-alanine | | | | | | |
| 195 | Gln | 3-Cyano-D-alanine | Arg | Trp(6-F) | Pen | Ac | NH₂ | Disulfide |
| 196 | Gln | 3-Cyclopropyl-D- | Arg | Trp(6-F) | Pen | Ac | NH₂ | Disulfide |
| | | alanine | | | | | | |
| 197 | Gln | (R)-2-Amino-4- | Arg | Trp(6-F) | Pen | Ac | NH₂ | Disulfide |
| | | cyclopropylbutanoic | | | | | | |
| | | acid | | | | | | |
| 198 | Gln | (αR)-α-Amino-2- | Arg | Trp(6-F) | Pen | Ac | NH₂ | Disulfide |
| | | pyridine- | | | | | | |
| | | propanoic acid | | | | | | |
| 199 | Gln | (αR)-α-Amino-3- | Arg | Trp(6-F) | Pen | Ac | NH₂ | Disulfide |
| | | pyridine- | | | | | | |
| | | propanoic acid | | | | | | |
| 200 | Gln | (αR)-α-Amino-4- | Arg | Trp(6-F) | Pen | Ac | NH₂ | Disulfide |
| | | pyridine- | | | | | | |
| | | propanoic acid | | | | | | |
| 201 | His | D-Phe | Arg | Trp | Cys | Ac | NH₂ | Disulfide |
| 202 | His | D-Phe | Arg | Trp | Cys | Ac | NH₂ | Disulfide |
| 203 | His | D-Phe | Arg | Trp | Cys | Ac | NH₂ | Disulfide |
| 204 | His | D-Phe | Arg | Trp | Cys | Ac | NH₂ | Disulfide |
| 205 | His | (aMe)D-Phe | Arg | Trp | Cys | Ac | NH₂ | Disulfide |
| 206 | His | D-Phe | Arg | Trp | Cys | Ac | NH₂ | Disulfide |
| 207 | His | D-Phe | Arg | Trp | Cys | Ac | NH₂ | Disulfide |
| 208 | His | D-Phe | Arg | Trp | Cys | Ac | NH₂ | Disulfide |
| 209 | Pro(4OH) | D-Phe | Arg | Trp | Cys | Ac | NH₂ | Disulfide |
| 210 | Pro(4OH) | D-Phe | Arg | Trp | Cys | Ac | NH₂ | Disulfide |
| 211 | His | D-Phe(4-Me) | Arg | Trp | Cys | Ac | NH₂ | Disulfide |
| 212 | His | D-Phe(3-Me) | Arg | Trp | Cys | Ac | NH₂ | Disulfide |
| 213 | His | D-homoPhe | Arg | Trp | Cys | Ac | NH₂ | Disulfide |
| 214 | His | D-phenylGly | Arg | Trp | Cys | Ac | NH₂ | Disulfide |
| 215 | His | D-Phe | Arg | Trp | Cys | Ac | NH₂ | Disulfide |
| 216 | His | D-Phe | Arg | Trp | Cys | Ac | NH₂ | Disulfide |
| 1001 | His | D-Phe | Arg | Trp | Cys | Ac | NH₂ | Disulfide |
| 218 | N-Me-His | D-Phe | Arg | Trp | Cys | Ac | NH₂ | Disulfide |
| 219 | His | homoPhe | Arg | Trp | Cys | Ac | NH₂ | Disulfide |
| 220 | His | Pro(4-phenyl) | Arg | Trp | Cys | Ac | NH₂ | Disulfide |
| 221 | His | Phe(4-Me) | Arg | Trp | Cys | Ac | NH₂ | Disulfide |
| 222 | His | Phe(3-Me) | Arg | Trp | Cys | Ac | NH₂ | Disulfide |
| 223 | His | Phe(3,4-diMe) | Arg | Trp | Cys | Ac | NH₂ | Disulfide |
| 224 | Indoline- | D-Phe | Arg | Trp | Cys | Ac | NH₂ | Disulfide |
| | COOH | | | | | | | |
| 1002 | His | D-Phe(3,4-diMe) | Arg | Trp | Cys | Ac | NH₂ | Disulfide |
| 226 | His | D-Phe | Arg | Trp(6-Me) | Cys | Ac | NH₂ | Disulfide |
| 227 | His | D-Phe | Arg | Trp | Cys | Ac | NH₂ | Disulfide |
| 228 | His | D-Phe | Arg | Trp | Cys | Ac | NH₂ | Disulfide |
| 229 | His | D-Phe | Arg | Trp | Cys | Ac | NH₂ | Disulfide |
| 230 | His | D-Phe | Arg | Trp | Cys | Ac | NH₂ | Disulfide |
| 1003 | His | D-Phe | Arg | Trp | Cys | Ac | NH₂ | Disulfide |
| 232 | His | D-Phe | Arg | Trp | Cys | Ac | NH₂ | Disulfide |
| 1004 | His | D-Phe | Arg | Trp | Cys | Ac | NH₂ | Disulfide |
| 234 | His | D-Phe | Arg | Trp | Cys | Ac | NH₂ | Disulfide |
| 235 | His | D-Phe | Arg | Trp | Cys | Ac | NH₂ | Disulfide |
| 236 | His | D-Phe | Arg | Trp | Cys | Ac | NH₂ | Disulfide |
| 237 | His | D-Phe | Arg | Trp | Cys | Ac | NH₂ | Disulfide |
| 238 | His | D-Phe | Arg | Trp | Cys | Ac | NH₂ | Disulfide |
| 239 | His | D-Phe | Arg | Trp | Cys | Ac | NH₂ | Disulfide |
| 240 | His | D-Phe | Arg | Trp | Cys | Ac | NH₂ | Disulfide |
| 241 | D-His | D-Phe | Arg | Trp | Cys | Ac | NH₂ | Disulfide |
| 242 | His | D-Phe | Arg | Trp | Cys | Ac | NH₂ | Disulfide |
| 1005 | His | D-Phe | Arg | Trp | Cys | Ac | NH₂ | Disulfide |
| 244 | His | D-Phe(4-Me) | Arg | Trp | Cys | Ac | NH₂ | Disulfide |
| 245 | His | D-Phe(4-Me) | Arg | Trp | Cys | Ac | NH₂ | Disulfide |
| 246 | His | D-Phe | Arg | Trp | Cys | Ac | NH₂ | Disulfide |
| 247 | His | D-Phe | Arg | Trp | Cys | Ac | NH₂ | Disulfide |
| 248 | His | D-Phe | Arg | Trp | Cys | Ac | NH₂ | Disulfide |
| 249 | His | D-Phe | Arg | Trp(6-Me) | Cys | Ac | NH₂ | Disulfide |
| 250 | His | D-Phe | Arg | Trp(6-Me) | Cys | Ac | NH₂ | Disulfide |
| 251 | His | D-Phe | Arg | Trp(6-Me) | Cys | Ac | NH₂ | Disulfide |
| 252 | His | D-Phe | Arg | Trp(6-Me) | Cys | Ac | NH₂ | Disulfide |
| 253 | His | D-Phe | Arg | Trp | Cys | Ac | NH₂ | Disulfide |
| 1006 | His | D-Phe | Arg | Trp | Cys | Ac | NH₂ | Disulfide |
| 255 | His | D-Phe | Arg | Trp | Cys | Ac | NH₂ | Disulfide |
| 1007 | His | D-Phe | Arg | Trp | Cys | Ac | NH₂ | Disulfide |
| 257 | His | D-Phe | Arg | Trp | Cys | Ac | NH₂ | Disulfide |
| 258 | His | D-Phe | Arg | Trp | Cys | Ac | NH₂ | Disulfide |
| 259 | His | D-Phe | Arg | Trp | Cys | Ac | NH₂ | Disulfide |
| 260 | His | D-Phe | Arg | Trp | Cys | Ac | NH₂ | Disulfide |
| 261 | His | D-Phe | Arg | Trp | Cys | Ac | NH₂ | Disulfide |
| 262 | His | D-Phe | Arg | Trp | Cys | Ac | NH₂ | Disulfide |
| 263 | His | D-Phe | Arg | Trp | Cys | Ac | NH₂ | Disulfide |
| 264 | His | D-Phe | Arg | Trp | Cys | Ac | NH₂ | Disulfide |
| 265 | His | D-Phe | Arg | Trp | Cys | Ac | NH₂ | Disulfide |
| 266 | 3-Me-His | D-Phe | Arg | Trp | Cys | Ac | NH₂ | Disulfide |
| 267 | Ala(2- | D-Phe | Arg | Trp | Cys | Ac | NH₂ | Disulfide |
| | furyl) | | | | | | | |
| 268 | His | D-Phe(4-Br) | Arg | Trp | Cys | Ac | NH₂ | Disulfide |
| 269 | His | D-Phe(4-F) | Arg | Trp | Cys | Ac | NH₂ | Disulfide |
| 270 | His | D-Phe(4-Cl) | Arg | Trp | Cys | Ac | NH₂ | Disulfide |
| 271 | His | D-Phe | Arg | Trp(6-F) | Cys | Ac | NH₂ | Disulfide |
| 272 | His | D-Phe | Arg | Trp | Cys | Ac | NH₂ | Disulfide |
| 273 | His | D-Phe(3-Me) | Arg | Trp | Cys | Ac | NH₂ | Disulfide |
| 274 | His | D-Phe | Arg | Trp | Cys | Ac | NH₂ | Disulfide |
| 275 | His | D-Phe | Arg | Trp(6-Me) | Cys | Ac | NH₂ | Disulfide |
| 276 | His | D-Phe | Arg | Trp(6-Me) | Cys | Ac | NH₂ | Disulfide |
| 1008 | His | D-Phe | Arg | Trp(6-Me) | Cys | Ac | NH₂ | Disulfide |
| 278 | His | D-Phe | Arg | Trp(6-Me) | Cys | Ac | NH₂ | Disulfide |
| 279 | His | D-Phe | Arg | Trp(6-Me) | Cys | Ac | NH₂ | Disulfide |
| 1009 | His | D-Phe | Arg | Trp(6-Me) | Cys | Ac | NH₂ | Disulfide |
| 287 | His | D-Phe | Arg | Trp | Cys | Ac | NH₂ | Disulfide |
| 288 | His | D-Phe | Arg | Trp | Cys | Ac | NH₂ | Disulfide |
| 289 | His | D-Phe | Arg | Trp | Cys | Ac | NH₂ | Disulfide |
| 290 | His | D-Phe | Arg | Trp | Cys | Ac | NH₂ | Disulfide |
| 291 | His | D-Phe | Arg | Trp | Cys | Ac | NH₂ | Disulfide |
| 292 | His | D-Phe | Arg | Trp | Cys | Ac | NH₂ | Disulfide |
| 293 | His | D-Phe | Arg | Trp | Cys | Ac | NH₂ | Disulfide |
| 294 | His | D-Phe | Arg | Trp | Cys | Ac | NH₂ | Disulfide |
| 295 | His | D-Phe | Arg | Trp | Cys | Ac | NH₂ | Disulfide |
| 296 | His | D-Phe | Arg | Trp | Cys | Ac | NH₂ | Disulfide |
| 297 | His | D-Phe | Arg | Trp | Cys | Ac | NH₂ | Disulfide |
| 298 | His | D-Phe | Arg | Trp | Cys | Ac | NH₂ | Disulfide |
| 299 | His | D-Phe | Arg | Trp | Cys | Ac | NH₂ | Disulfide |
| 300 | His | D-Phe | Arg | Trp | Cys | Ac | NH₂ | Disulfide |
| 301 | His | D-Phe | Arg | Trp | Cys | Ac | NH₂ | Disulfide |
| 345 | His | D-Phe | Arg | Trp | Cys | Ac | NH₂ | Disulfide |
| 346 | His | D-Phe(3-F) | Arg | Trp | Cys | Ac | NH₂ | Disulfide |
| 347 | His | D-Phe | Arg | Trp | Cys | Ac | NH₂ | Disulfide |
| 348 | His | D-Phe | Arg | Trp | Cys | Ac | NH₂ | Disulfide |
| 349 | His | D-Phe | Arg | Trp | Cys | Ac | NH₂ | Disulfide |
| 350 | His | D-Phe | Arg | Trp | Cys | Ac | NH₂ | Disulfide |
| 351 | His | D-Phe | Arg | Trp | Cys | Ac | NH₂ | Disulfide |
| 357 | His | D-Phe | Arg | Trp | hCys | Ac | NH₂ | Disulfide |
| 358 | His | D-Phe | Arg | Trp(5-Me) | Cys | Ac | NH₂ | Disulfide |
| 359 | His | D-Bpa | Arg | Trp | Cys | Ac | NH₂ | Disulfide |
| 360 | Dap | D-Phe | Arg | Trp | Cys | Ac | NH₂ | Disulfide |
| 361 | His | D-Phe | Arg | Trp | Cys | Ac | NH₂ | Disulfide |
| 362 | His | D-Tyr | Arg | Trp | Cys | Ac | NH₂ | Disulfide |
| 363 | His | D-Phe | Arg | Trp | Cys | Ac | NH₂ | Disulfide |
| 364 | His | D-Tyr | Arg | Trp(5-OH) | Cys | Ac | NH₂ | Disulfide |
| 365 | His | D-Phe | Arg | Trp | Cys | Ac | NH₂ | Disulfide |
| 366 | His | D-Phe | Arg | Trp | Cys | Ac | NH₂ | Disulfide |
| 367 | His | D-Phe | Arg | Trp | Cys | Ac | NH₂ | Disulfide |
| 1010 | His | D-Phe | Arg | Trp | Cys | Ac | NH₂ | Disulfide |
| 369 | His | D-Phe | Arg | Trp | Cys | Ac | NH₂ | Disulfide |
| 370 | His | D-Phe(3-Ph) | Arg | Trp | Cys | Ac | NH₂ | Disulfide |
| 374 | His | D-Phe | Arg | Trp | Cys | Ac | NH₂ | Disulfide |
| 375 | 2Pal | D-Phe | Arg | Trp | Cys | Ac | NH₂ | Disulfide |
| 376 | His | D-Phe | Arg | Trp(7-Me) | Cys | Ac | NH₂ | Disulfide |
| 377 | His | D-Phe | Arg | Trp | Cys | Ac | NH₂ | Disulfide |
| 378 | His | D-Phe | Arg | Trp | Cys | Ac | NH₂ | Disulfide |
| 379 | His | D-Phe | Arg | Trp | Cys | Ac | NH₂ | Disulfide |
| 380 | His | D-Phe | Arg | Trp | Cys | Ac | NH₂ | Disulfide |
| 381 | His | D-Phe | Arg | Trp(6-Me) | Cys | Ac | NH₂ | Disulfide |
| 382 | His | D-Phe(3-Me) | Arg | Trp | Cys | Ac | NH₂ | Disulfide |
| 383 | His | D-Phe | Arg | Trp(6-Me) | Cys | Ac | NH₂ | Disulfide |
| 385 | His | D-Phe | Arg | Trp | Cys | Ac | NH₂ | Disulfide |
| 386 | His | D-Phe | Arg | Trp | Cys | Ac | NH₂ | Disulfide |
| 387 | His | D-Phe | Arg | Trp | Cys | Ac | NH₂ | Disulfide |
| 388 | His | D-Phe | Arg | Trp | Cys | Ac | NH₂ | Disulfide |
| 390 | His | D-Phe | Arg | Trp | Cys | Ac | NH₂ | Disulfide |
| 391 | His | D-Phe | Arg | Trp | Cys | Ac | NH₂ | Disulfide |
| 392 | His | D-Phe | Arg | Trp | Cys | Ac | NH₂ | Disulfide |
| 393 | His | D-Phe | Arg | Trp | Cys | Ac | NH₂ | Disulfide |
| 394 | His | D-Phe | Arg | Trp | Cys | Ac | NH₂ | Disulfide |
| 395 | His | D-Phe | Arg | Trp | Cys | Ac | NH₂ | Disulfide |
| 396 | His | D-Phe | Arg | Trp | Cys | Ac | NH₂ | Disulfide |
| 397 | His | D-Phe | Arg | Trp | Cys | Ac | NH₂ | Disulfide |
| 399 | His | D-Phe | Arg | Trp | Cys | Ac | NH₂ | Disulfide |
| 400 | His | D-Phe | Arg | Trp | Cys | Ac | NH₂ | Disulfide |
| 401 | His | D-Phe | Arg | Trp | Cys | Ac | NH₂ | Disulfide |
| 1011 | Gln | D-Phe | Arg | Trp | Cys | Ac | NH₂ | Disulfide |
| 1012 | Gln | D-Phe | Arg | Trp | Cys | Ac | NH₂ | Disulfide |
| 407 | His | D-Phe(4-F) | Arg | Trp | Cys | Ac | NH₂ | Disulfide |
| 1013 | Gln | D-Phe | Arg | Trp | Cys | Ac | NH₂ | Disulfide |
| 409 | 3-Me-His | D-Phe | Arg | D-Trp | Cys | Ac | NH₂ | Disulfide |
| 410 | Ala(cPent) | D-Phe | Arg | Trp | Cys | Ac | NH₂ | Disulfide |
| 411 | His | D-Phe | Arg | Trp | Cys | Ac | NH₂ | Disulfide |
| 412 | His | D-Phe | Arg | Trp(6-Me) | Cys | Ac | NH₂ | Disulfide |
| 413 | His | D-Phe | Arg | Trp | Cys | Ac | NH₂ | Disulfide |
| 414 | His | D-Phe(3-F) | Arg | Trp | Cys | Ac | NH₂ | Disulfide |
| 1014 | Gln | D-Phe | Arg | Trp | Cys | Ac | NH₂ | Disulfide |
| 419 | His | D-Phe(4-Me) | Arg | Trp | Cys | Ac | NH₂ | Disulfide |
| 1015 | His | D-Phe(4-Me) | Arg | Trp(6-Me) | Cys | Ac | NH₂ | Disulfide |
| 421 | His | D-Phe | Arg | Trp | Cys | Ac | NH₂ | Disulfide |
| 422 | His | D-Phe | Arg | Trp(6-F) | Cys | Ac | NH₂ | Disulfide |
| 424 | His | D-Phe | Arg | Trp | Cys | Ac | NH₂ | Disulfide |
| 1016 | His | D-Phe | Arg | Trp(6-Me) | Cys | Ac | NH₂ | Disulfide |
| 1017 | His | D-Phe(4-Me) | Arg | Trp | Cys | Ac | NH₂ | Disulfide |
| 1018 | His | D-Phe | Arg | Trp | Cys | Ac | NH₂ | Disulfide |
| 428 | His | D-Phe | Arg | Trp | Cys | Ac | NH₂ | Disulfide |
| 1019 | His | D-Phe | Arg | Trp | Cys | Ac | NH₂ | Disulfide |
| 1020 | His | D-Phe | Arg | Trp(6-Me) | Cys | Ac | NH₂ | Disulfide |
| 1021 | His | D-Phe(4-Me) | Arg | Trp | Cys | Ac | NH₂ | Disulfide |
| 1022 | His | D-Phe | Arg | Trp | Cys | Ac | NH₂ | Disulfide |
| 1023 | His | D-Phe(4-Me) | Arg | Trp | Cys | Ac | NH₂ | Disulfide |
| 1024 | His | D-Phe | Arg | Trp(6-Me) | Cys | Ac | NH₂ | Disulfide |
| 1025 | His | D-Phe | Arg | Trp | Cys | Ac | NH₂ | Disulfide |
| 436 | His | D-Phe | Arg | Trp | Cys | Ac | NH₂ | Disulfide |
| 437 | His | D-Phe(4-Cl) | Arg | Trp(6-Me) | Cys | Ac | NH₂ | Disulfide |
| 1026 | His | D-Phe(4-Cl) | Arg | Trp(6-Me) | Cys | Ac | NH₂ | Disulfide |
| 439 | His | D-Phe | Arg | Trp(6-Me) | Cys | Ac | NH₂ | Disulfide |
| 1027 | His | D-Phe(4-Cl) | Arg | TRP | Cys | Ac | NH₂ | Disulfide |
| 441 | His | D-Phe | Arg | Trp(6-F) | Cys | Ac | NH₂ | Disulfide |
| 1028 | His | D-Phe | Arg | Trp(6-F) | Cys | Ac | NH₂ | Disulfide |
| 1029 | His | D-Phe | Arg | Trp(6-Me) | Cys | Ac | NH₂ | Disulfide |
| 444 | His | D-Phe | Arg | Trp(6-Me) | Cys | Ac | NH₂ | Disulfide |
| 1030 | His | D-Phe(3-CF3) | Arg | TRP | Cys | Ac | NH₂ | Disulfide |
| 1031 | His | D-Phe(3-Cl) | Arg | TRP | Cys | Ac | NH₂ | Disulfide |
| 1032 | His | D-Phe | Arg | Trp(6-Cl) | Cys | Ac | NH₂ | Disulfide |
| 1033 | His | D-Phe | Arg | TRP | Cys | Ac | NH₂ | Disulfide |
| 449 | His | D-Phe | Arg | TRP | Cys | Ac | NH₂ | Disulfide |
| 450 | His | D-Phe | Arg | TRP | Cys | Ac | NH₂ | Disulfide |
| 451 | His | D-Phe | Arg | TRP | Cys | Ac | NH₂ | Disulfide |
| 452 | His | D-Phe | Arg | TRP | Cys | Ac | NH₂ | Disulfide |
| 1034 | His | D-Phe | Arg | TRP | Cys | Ac | NH₂ | Disulfide |
| 454 | His | D-Phe | Arg | TRP | Cys | Ac | NH₂ | Disulfide |
| 455 | His | D-Phe | Arg | Trp(6-Me) | Cys | Ac | NH₂ | Disulfide |
| 456 | His | D-Phe | Arg | Trp(6-Me) | Cys | Ac | NH₂ | Disulfide |
| 1035 | His | D-Phe | Arg | Trp(6-F) | Cys | Ac | NH₂ | Disulfide |
| 1036 | His | D-Phe | Arg | Trp(6-F) | Cys | Ac | NH₂ | Disulfide |
| 1037 | His | D-Phe | Arg | Trp(6-F) | Cys | Ac | NH₂ | Disulfide |
| 1038 | His | D-Phe(4-F) | Arg | Trp(6-F) | Cys | Ac | NH₂ | Disulfide |
| 1039 | His | D-Phe | Arg | Trp(6-Me) | Cys | Ac | NH₂ | Disulfide |
| 1040 | His | D-Phe(3-F) | Arg | Trp(6-F) | Cys | Ac | NH₂ | Disulfide |
| 463 | His | D-Phe | Arg | Trp(6-Me) | Cys | Ac | NH₂ | Disulfide |
| 1041 | His | D-Phe(4-F) | Arg | Trp(6-Me) | Cys | Ac | NH₂ | Disulfide |
| 1042 | His | D-Phe | Arg | Trp(6-F) | Cys | Ac | NH₂ | Disulfide |
| 1043 | His | D-Phe | Arg | Trp(5-Me) | Cys | Ac | NH₂ | Disulfide |
| 467 | His | D-Phe | Arg | Trp(6-F) | Cys | Ac | NH₂ | Disulfide |
| 468 | His | D-Phe | Arg | Trp(6-Me) | Cys | Ac | NH₂ | Disulfide |
| 469 | His | D-Phe | Arg | Trp(6-Me) | Cys | Ac | NH₂ | Disulfide |
| 1044 | His | D-Phe | Arg | Trp(6-Me) | Cys | Ac | NH₂ | Disulfide |
| 1045 | His | D-Phe(4-F) | Arg | Trp(6-Cl) | Cys | Ac | NH₂ | Disulfide |
| 1046 | His | D-Phe(3,4,5-triF) | Arg | Trp(6-Me) | Cys | Ac | NH₂ | Disulfide |
| 1047 | His | D-Phe(3,4,5-triF) | Arg | Trp(6-F) | Cys | Ac | NH₂ | Disulfide |
| 1048 | His | D-Phe(3-Cl) | Arg | Trp(6-Me) | Cys | Ac | NH₂ | Disulfide |
| 475 | His | D-Phe | Arg | Trp(6-Me) | Cys | Ac | NH₂ | Disulfide |
| 1049 | His | D-Phe | Arg | Trp(6-Me) | Cys | Ac | NH₂ | Disulfide |
| 1050 | His | D-Phe(3-CF3) | Arg | Trp(6-Me) | Cys | Ac | NH₂ | Disulfide |
| 1051 | His | D-Phe(4-Cl) | Arg | Trp(6-F) | Cys | Ac | NH₂ | Disulfide |
| 1052 | His | D-Phe | Arg | Trp(6-F) | Cys | Ac | NH₂ | Disulfide |
| 1053 | His | D-Phe(4-F) | Arg | Trp(6-F) | Cys | Ac | NH₂ | Disulfide |
| 1054 | His | D-Phe(4-F) | Arg | Trp(6-F) | Cys | Ac | NH₂ | Disulfide |
| 482 | His | D-Phe | Arg | Trp(6-F) | Cys | Ac | NH₂ | Disulfide |
| 1055 | His | D-Phe | Arg | Trp(6-Me) | Cys | Ac | NH₂ | Disulfide |
| 1056 | His | D-Phe(4-F) | Arg | Trp(6-Me) | Cys | Ac | NH₂ | Disulfide |
| 1057 | His | D-Phe(3-F) | Arg | Trp(6-F) | Cys | Ac | NH₂ | Disulfide |
| 1058 | His | D-Phe | Arg | Trp(6-F) | Cys | Ac | NH₂ | Disulfide |
| 487 | His | D-Phe | Arg | Trp(6-F) | Cys | Ac | NH₂ | Disulfide |
| 1059 | His | D-Phe(4-F) | Arg | Trp(6-Me) | Cys | Ac | NH₂ | Disulfide |
| 489 | His | D-Phe | Arg | Trp(6-F) | Cys | Ac | NH₂ | Disulfide |
| 1060 | His | D-Phe(4-F) | Arg | Trp(6-Me) | Cys | Ac | NH₂ | Disulfide |
| 1061 | His | D-Phe(4-F) | Arg | Trp(6-Me) | Cys | Ac | NH₂ | Disulfide |
| 1062 | His | D-Phe(4-F) | Arg | Trp(6-Me) | Cys | Ac | NH₂ | Disulfide |
| 1063 | His | D-Phe(4-F) | Arg | Trp(6-Me) | Cys | Ac | NH₂ | Disulfide |
| 1129 | His | D-Phe | Arg | Trp(6-Me) | Cys | Ac | NH₂ | Disulfide |
| 1128 | His | D-Phe | Arg | Trp(6-Me) | Cys | Ac | NH₂ | Disulfide |
| 1133 | His | D-Phe | Arg | Trp(6-Me) | Cys | Ac | NH₂ | Disulfide |
| 1064 | His | D-Phe | Arg | Trp(6-Me) | Cys | Ac | NH₂ | Disulfide |
| 499 | His | D-Phe | Arg | Trp(6-Me) | Cys | Ac | NH₂ | Disulfide |
| 1131 | His | D-Phe | Arg | Trp(6-Me) | Cys | Ac | NH₂ | Disulfide |
| 1065 | His | D-Phe | Arg | Trp(6-Me) | Cys | Ac | NH₂ | Disulfide |
| 1130 | His | D-Phe | Arg | Trp(6-Me) | Cys | Ac | NH₂ | Disulfide |
| 504 | His | D-Phe | Arg | Trp(6-Me) | Cys | Ac | NH₂ | Disulfide |
| 1135 | His | D-Phe | Arg | Trp(6-Me) | Cys | Ac | NH₂ | Disulfide |
| 1066 | His | D-Phe | Arg | Trp(6-Me) | Dap | Ac | NH₂ | Disulfide |
| 1067 | His | D-Phe | Arg | Trp(6-Me) | Dap | Ac | NH₂ | Disulfide |
| 1068 | His | D-Phe | Arg | Trp(6-Me) | Dap | Ac | NH₂ | Disulfide |
| 1069 | His | D-Phe | Arg | Trp(6-Me) | Dap | Ac | NH₂ | Disulfide |
| 1070 | Gln | D-Phe | Arg | Trp | Cys | Ac | NH₂ | Disulfide |
| 1071 | His | D-Phe(3-F,4-Me) | Arg | Trp(6-F) | Cys | Ac | NH₂ | Disulfide |
| 1072 | His | D-Phe(4-CF3) | Arg | Trp(6-F) | Cys | Ac | NH₂ | Disulfide |
| 1073 | His | D-Phe(2-F,4-Cl) | Arg | Trp(6-F) | Cys | Ac | NH₂ | Disulfide |
| 1074 | His | D-Phe(3,4-diF) | Arg | Trp(6-F) | Cys | Ac | NH₂ | Disulfide |
| 1075 | His | D-Phe | Arg | Trp(6-CF3) | Cys | Ac | NH₂ | Disulfide |
| 1076 | His | D-Phe | Arg | Trp(4-F) | Cys | Ac | NH₂ | Disulfide |
| 1077 | His | D-Phe | Arg | Trp(5-F) | Cys | Ac | NH₂ | Disulfide |
| 1078 | His | D-Phe | Arg | Trp(7-F) | Cys | Ac | NH₂ | Disulfide |
| 1079 | His | D-Phe | Arg | Trp(5-Cl) | Cys | Ac | NH₂ | Disulfide |
| 1080 | His | D-Phe | Arg | Trp(6-Br) | Cys | Ac | NH₂ | Disulfide |
| 1081 | His | D-Phe(3-F) | Arg | Trp(5-F) | Cys | Ac | NH₂ | Disulfide |
| 1082 | His | D-Phe(2,4-diCl) | Arg | Trp(6-F) | Cys | Ac | NH₂ | Disulfide |
| 1083 | His | D-Phe(2,3-diF) | Arg | Trp(6-F) | Cys | Ac | NH₂ | Disulfide |
| 1084 | His | D-Phe(3-Cl) | Arg | Trp(6-F) | Cys | Ac | NH₂ | Disulfide |
| 1085 | His | D-Phe(3-F) | Arg | Trp(6-Me) | Cys | Ac | NH₂ | Disulfide |
| 1086 | His | D-Phe(3-Me) | Arg | Trp(6-F) | Cys | Ac | NH₂ | Disulfide |
| 1087 | His | D-Phe(2,4-diF) | Arg | Trp(6-F) | Cys | Ac | NH₂ | Disulfide |
| 1088 | His | D-Phe(2,4,5-triF) | Arg | Trp(6-F) | Cys | Ac | NH₂ | Disulfide |
| 1089 | His | D-Phe(3-CF3) | Arg | Trp(6-F) | Cys | Ac | NH₂ | Disulfide |
| 532 | His | D-Phe | Arg | Trp(6-F) | Cys | Ac | NH₂ | Disulfide |
| 533 | His | D-Phe(3-F) | Arg | Trp(6-F) | Cys | Ac | NH₂ | Disulfide |
| 534 | His | D-Phe | Arg | Trp(6-CF3) | Cys | Ac | NH₂ | Disulfide |
| 535 | His | D-Phe | Arg | Trp(6-F) | Cys | Ac | NH₂ | Disulfide |
| 536 | His | D-Phe(4-F) | Arg | Trp(6-F) | Cys | Ac | NH₂ | Disulfide |
| 537 | His | D-Phe(3,4,5-triF) | Arg | Trp(6-F) | Cys | Ac | NH₂ | Disulfide |
| 538 | His | D-Phe(3-F) | Arg | Trp(6-F) | Cys | Ac | NH₂ | Disulfide |
| 539 | His | D-Phe | Arg | Trp(6-F) | Cys | Ac | NH₂ | Disulfide |
| 540 | His | D-Phe | Arg | Trp(5-F) | Cys | Ac | NH₂ | Disulfide |
| 541 | His | D-Phe(3,4,5-triF) | Arg | Trp(6-F) | Cys | Ac | NH₂ | Disulfide |
| 542 | His | D-Phe(3-F) | Arg | Trp(6-F) | Cys | Ac | NH₂ | Disulfide |
| 1090 | Gln | D-Phe | Arg | Trp(6-F) | Cys | Ac | NH₂ | Disulfide |
| 1091 | Gln | D-Phe | Arg | Trp(6-F) | Cys | Ac | NH₂ | Disulfide |
| 1092 | Gln | D-Phe | Arg | Trp(6-F) | Cys | Ac | NH₂ | Disulfide |
| 1093 | Gln | D-Phe(4-F) | Arg | Trp(6-F) | Cys | Ac | NH₂ | Disulfide |
| 1094 | Gln | D-Phe | Arg | Trp(6-F) | Cys | Ac | NH₂ | Disulfide |
| 1096 | Gln | D-Phe | Arg | Trp(6-F) | Cys | Ac | NH₂ | Disulfide |
| 1097 | Gln | D-Phe(4-F) | Arg | Trp(6-F) | Cys | Ac | NH₂ | Disulfide |
| 1098 | Gln | D-Phe | Arg | Trp(6-F) | Cys | Ac | NH₂ | Disulfide |
| 1099 | Gln | D-Phe(4-F) | Arg | Trp(6-F) | Cys | Ac | NH₂ | Disulfide |
| 1100 | hGln | D-Phe | Arg | Trp(6-F) | Cys | Ac | NH₂ | Disulfide |
| 1101 | hGln | D-Phe(4-F) | Arg | Trp(6-F) | Cys | Ac | NH₂ | Disulfide |
| 1102 | hGln | D-Phe | Arg | Trp(6-F) | Cys | Ac | NH₂ | Disulfide |
| 1103 | Cit | D-Phe | Arg | Trp(6-F) | Cys | Ac | NH₂ | Disulfide |
| 1104 | Cit | D-Phe | Arg | Trp(6-F) | Cys | Ac | NH₂ | Disulfide |
| 1106 | Cit | D-Phe(4-F) | Arg | Trp(6-F) | Cys | Ac | NH₂ | Disulfide |
| 1107 | hCit | D-Phe(4-F) | Arg | Trp(6-F) | Cys | Ac | NH₂ | Disulfide |
| 1108 | 3Pal | D-Phe | Arg | Trp(6-F) | Cys | Ac | NH₂ | Disulfide |
| 1109 | 4Pal | D-Phe | Arg | Trp(6-F) | Cys | Ac | NH₂ | Disulfide |
| 561 | His | D-Phe | Arg | Trp(6-F) | Cys | Ac | NH₂ | Disulfide |
| 562 | His | D-Phe | Arg | Trp(6-F) | Cys | Ac | NH₂ | Disulfide |
| 563 | His | D-Phe | Arg | Trp(6-F) | Cys | Ac | NH₂ | Disulfide |
| 564 | His | D-Phe | Arg | Trp(6-F) | Cys | Ac | NH₂ | Disulfide |
| 1141 | His | D-Phe | Arg | Trp(6-F) | Cys | Ac | NH₂ | Disulfide |
| 566 | His | D-Phe | Arg | Trp(6-F) | Cys | Ac | NH₂ | Disulfide |
| 567 | His | D-Phe | Arg | Trp(6-F) | Cys | Ac | NH₂ | Disulfide |
| 568 | His | D-Phe | Arg | Trp(6-F) | Cys | Ac | NH₂ | Disulfide |
| 569 | His | D-Phe | Arg | Trp(6-F) | Cys | Ac | NH₂ | Disulfide |
| 1143 | His | D-Phe | Arg | Trp(6-F) | Cys | Ac | NH₂ | Disulfide |
| 571 | His | D-Phe | Arg | Trp(6-F) | Cys | Ac | NH₂ | Disulfide |
| 572 | His | D-Phe | Arg | Trp(6-F) | Cys | Ac | NH₂ | Disulfide |
| 573 | His | D-Phe | Arg | Trp(6-F) | Cys | Ac | NH₂ | Disulfide |
| 574 | His | D-Phe | Arg | Trp(6-F) | Cys | Ac | NH₂ | Disulfide |
| 575 | His | D-Phe | Arg | Trp(6-F) | Cys | Ac | NH₂ | Disulfide |
| 1110 | His | D-Phe | Arg | Trp(6-F) | Cys | Ac | NH₂ | Disulfide |
| 1111 | His | D-Phe(4-F) | Arg | Trp(6-F) | Cys | Ac | NH₂ | Disulfide |
| 1112 | His | D-Phe(4-F) | Arg | Trp(5-Me) | Cys | Ac | NH₂ | Disulfide |
| 1113 | His | D-Phe(4-F) | Arg | Trp(6-Me) | Cys | Ac | NH₂ | Disulfide |
| 1114 | His | D-Phe | Arg | Trp(6-F) | Cys | Ac | NH₂ | Disulfide |
| 1115 | His | D-Phe(4-F) | Arg | Trp(6-F) | Cys | Ac | NH₂ | Disulfide |
| 1116 | 3Pal | D-Phe | Arg | Trp(6-Me) | Cys | Ac | NH₂ | Disulfide |
| 1118 | 4Pal | D-Phe | Arg | Trp(6-Me) | Cys | Ac | NH₂ | Disulfide |
| 1119 | 3Pal | D-Phe(4-F) | Arg | Trp(6-F) | Cys | Ac | NH₂ | Disulfide |
| 1120 | His | D-Phe | Arg | Trp(6-F) | Dap | Ac | NH₂ | Disulfide |
| 1121 | His | D-Phe(4-F) | Arg | Trp(6-F) | Dap | Ac | NH₂ | Disulfide |
| 1134 | His | D-Phe(3,4,5-triF) | Arg | Trp(6-Me) | Cys | Ac | NH₂ | Disulfide |
| 1132 | His | D-Phe(3,4,5-triF) | Arg | Trp(6-Me) | Cys | Ac | NH₂ | Disulfide |
| 589 | His | D-Phe | Arg | Trp(6-Me) | Cys | Ac | NH₂ | Disulfide |
| 590 | His | D-Phe | Arg | Trp(6-Me) | Cys | Ac | NH₂ | Disulfide |

In embodiments, the peptide of formula (I) is selected from Table A1A.

In embodiments, the peptide of formula (I) is selected from Table A1A, wherein the N-terminal. C-terminal and/or cyclic structure are optional structure.

TABLE A1A
Exemplary peptides.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1093 | D- | Cys | Aib(O- | Gln | D-Phe(4- | Arg | Trp(6-F) | Cys | Ac | NH₂ | Disulfide |
| | Nar | | cyclic) | | F) | | | | | | |
| 1092 | D- | Cys | Aib(O- | Gln | D-Phe | Arg | Trp(6-F) | Cys | Ac | NH₂ | Disulfide |
| | Nar | | cyclic) | | | | | | | | |
| 1107 | D- | Cys | Aib(O- | hCit | D-Phe(4- | Arg | Trp(6-F) | Cys | Ac | NH₂ | Disulfide |
| | Nar | | cyclic) | | F) | | | | | | |
| 1106 | D-Nar | Cys | Aib(O | Cit | D-Phe(4- | Arg | Trp(6-F) | Cys | Ac | NH₂ | Disulfide |
| | | | cyclic) | | F | | | | | | |
| 1103 | D- | Cys | Aib(O- | Cit | D-Phe | Arg | Trp(6-F) | Cys | Ac | NH₂ | Disulfide |
| | Nar | | cyclic) | | | | | | | | |
| 1105 | D-Nar | Cys | Aib(O | Cit | D-Phe(4- | Arg | Trp(6-F) | Cys | Ac | NH₂ | Disulfide |
| | | | cyclic) | | Me) | | | | | | |
| 1095 | D- | Cys | Aib(O- | Gln | D-Phe(4- | Arg | Trp(6-F) | Cys | Ac | NH₂ | Disulfide |
| | Nar | | cyclic) | | Me) | | | | | | |
| 1122 | D- | Cys | Aib(O | 3- | D-Phe(4- | Arg | Trp(6-F) | Cys | Ac | NH₂ | Disulfide |
| | Nar | | cyclic) | Pal | F) | | | | | | |
| 1102 | D- | Cys | Aib(O- | hGln | D-Phe | Arg | Trp(6-F) | Cys | Ac | NH₂ | Disulfide |
| | Nar | | cyclic) | | | | | | | | |
| 1058 | D- | Cys | Aib(O- | His | D-Phe | Arg | Trp(6-F) | Cys | Ac | NH₂ | Disulfide |
| | Nar | | cyclic) | | | | | | | | |
| 1123 | D- | Cys | Aib(O | Orn | D-Phe(4- | Arg | Trp(6-F) | Cys | Ac | NH₂ | Disulfide |
| | Nar | | cyclic) | | F) | | | | | | |
| 1158 | D- | Cys | Aib(O- | Gln | D-Phe(4- | Arg | Trp(6-F) | Pen | Ac | NH₂ | Disulfide |
| | Nar | | cyclic) | | F) | | | | | | |

In embodiments, the peptide of formula (II) is selected from Table A2.

TABLE A2
Exemplary peptides.

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1150-2 | Lys* | D- | Gly | D- | D- | Cys | Aib(O- | Gln | D- | Arg | Trp(6- | Cys |
| | | Arg | | Arg | Nar | | cyclic) | | Phe(4- | | F) | |
| | | | | | | | | | F) | | | |
| 1142-2 | | Lys* | Gly | D- | D- | Cys | Aib(O- | Gln | D- | Arg | Trp(6- | Cys |
| | | | | Arg | Nar | | cyclic) | | Phe(4- | | F | |
| | | | | | | | | | F) | | | |
| 1144-2 | | Lys* | PEG1 | PEG1 | D- | Cys | Aib(O- | Gln | D- | Arg | Trp(6- | Cys |
| | | | | | Nar | | cyclic) | | Phe(4- | | F) | |
| | | | | | | | | | F) | | | |
| 1151-2 | Lys* | D- | PEG1 | D- | Beta- | Cys | Aib(O- | Gln | D- | Arg | Trp(6- | Cys |
| | | Arg | | Arg | homoArg | | cyclic) | | Phe(4- | | F) | |
| | | | | | | | | | F) | | | |
| 1152-2 | Lys* | D- | Gly | D- | D- | Cys | Aib(O- | 3Pal | D- | Arg | Trp(6- | Cys |
| | | Arg | | Arg | Nar | | cyclic) | | Phe(4- | | F) | |
| | | | | | | | | | F) | | | |
| 1153-2 | Lys* | D- | Gly | D- | D- | Cys | Aib(O- | Orn | D- | Arg | Trp(6- | Cys |
| | | Arg | | Arg | Nar | | cyclic) | | Phe(4- | | F) | |
| | | | | | | | | | F) | | | |

In embodiments, the peptide of formula (II) is selected from Table A2A.

In embodiments, the peptide of formula (II) is selected from Table A2A, wherein the N-terminal, C-terminal and/or cyclic structure are optional feature.

TABLE A2A
Exemplary lipidated molecules.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1150 | Lys* | D- | Gly | D- | D- | Cys | Aib(O- | Gln | D- | Arg | Trp(6- | Cys | Ac | NH₂ | Disulfide |
| | | Arg | | Arg | Nar | | cyclic) | | Phe(4- | | F) | | | | |
| | | | | | | | | | F) | | | | | | |
| 1142 | | Lys* | Gly | D- | D- | Cys | Aib(O- | Gln | D- | Arg | Trp(6- | Cys | Ac | NH₂ | Disulfide |
| | | | | Arg | Nar | | cyclic) | | Phe(4- | | F) | | | | |
| | | | | | | | | | F) | | | | | | |
| 1144 | | Lys* | PEG1 | PEG1 | D- | Cys | Aib(O- | Gln | D- | Arg | Trp(6- | Cys | Ac | NH₂ | Disulfide |
| | | | | | Nar | | cyclic) | | Phe(4- | | F) | | | | |
| | | | | | | | | | F) | | | | | | |
| 1151 | Lys* | D- | PEG1 | D- | Beta- | Cys | Aib(O- | Gln | D- | Arg | Trp(6- | Cys | Ac | NH₂ | Disulfide |
| | | Arg | | Arg | homoArg | | cyclic) | | Phe(4- | | F) | | | | |
| | | | | | | | | | F) | | | | | | |
| 1152 | Lys* | D- | Gly | D- | D- | Cys | Aib(O- | 3 | D- | Arg | Trp(6- | Cys | Ac | NH₂ | Disulfide |
| | | Arg | | Arg | Nar | | cyclic) | Pal | Phe | | F) | | | | |
| | | | | | | | | | 4-F) | | | | | | |
| 1153 | Lys* | D- | Gly | D- | D- | Cys | Aib(O- | Orn | D- | Arg | Trp(6- | Cys | Ac | NH₂ | Disulfide |
| | | Arg | | Arg | Nar | | cyclic) | | Phe(4- | | F) | | | | |
| | | | | | | | | | F) | | | | | | |

TABLE 2
Exemplary lipidated molecules. Cyclic peptides include
bridge (e.g. disulfide) between X₂ and X₈.

| Molecule | | | | | | | | |
|---|---|---|---|---|---|---|---|---|
| Name | X₋₄ | X₋₃ | X₋₂ | X₋₁ | X₁ | X₂ | X₃ | X₄ |
| 1146 | Lys* | D-Arg | Gly | D-Arg | D-Nar | Cys | Phg | 3Pal |
| 1139 | | Lys* | Gly | D-Arg | D-Nar | Cys | Phg | 3Pal |
| 1145 | | Lys* | PEG1 | PEG1 | D-Nar | Cys | Phg | 3Pal |
| 1147 | Lys* | D-Arg | PEG1 | D-Arg | Beta-homoArg | Cys | Phg | 3Pal |
| 1148 | Lys* | D-Arg | Gly | D-Arg | D-Nar | Cys | D-aMeOrn | Gln |
| 1149 | | Lys* | Gly | D-Arg | D-Nar | Cys | D-aMeOrn | Gln |
| 1137 | | Lys* | PEG1 | PEG1 | D-Nar | Cys | D-aMeOrn | Gln |
| 1136 | Lys* | D-Arg | PEG1 | D-Arg | Beta-homoArg | Cys | D-aMeOrn | Gln |
| 1150 | Lys* | D-Arg | Gly | D-Arg | D-Nar | Cys | Aib(O-cyclic) | Gln |
| 1142 | | Lys* | Gly | D-Arg | D-Nar | Cys | Aib(O-cyclic) | Gln |
| 1144 | | Lys* | PEG1 | PEG1 | D-Nar | Cys | Aib(O-cyclic) | Gln |
| 1151 | Lys* | D-Arg | PEG1 | D-Arg | Beta-homoArg | Cys | Aib(O-cyclic) | Gln |
| 1152 | Lys* | D-Arg | Gly | D-Arg | D-Nar | Cys | Aib(O-cyclic) | 3Pal |
| 1153 | Lys* | D-Arg | Gly | D-Arg | D-Nar | Cys | Aib(O-cyclic) | Orn |
| 1154 | Lys* | D-Arg | Gly | D-Arg | D-Nar | Cys | D-aMeOrn | 3Pal |
| 1155 | Lys* | D-Arg | Gly | D-Arg | D-Nar | Cys | D-aMeOrn | Orn |
| 1156 | Lys* | D-Arg | Gly | D-Arg | D-Nar | Cys | D-aMeOrn | 3Pal |
| 1157 | Lys* | D-Arg | Gly | D-Arg | D-Nar | Cys | D-aMeOrn | Orn |
| 111 | Lys* | Gly | Gly | Gly | D-Nar | Cys | Aib(O-cyclic) | Gln |
| 112 | | Lys* | Gly | Gly | D-Nar | Cys | Aib(O-cyclic) | Gln |
| 113 | | | Lys* | Gly | D-Nar | Cys | Aib(O-cyclic) | Gln |
| 114 | | | | Lys* | D-Nar | Cys | Aib(O-cyclic) | Gln |
| 123 | | Lys* | Gly | Gly | D-Nar | Cys | Phg | 3Pal |
| 124 | | | Lys* | Gly | D-Nar | Cys | Phg | 3Pal |
| 125 | | | | Lys* | D-Nar | Cys | Phg | 3Pal |
| 130 | | Lys* | Gly | Gly | D-Nar | Cys | D-aMeOrn | Gln |
| 131 | | | Lys* | Gly | D-Nar | Cys | D-aMeOrn | Gln |
| 132 | | | | Lys* | D-Nar | Cys | D-aMeOrn | Gln |
| 138 | | | Lys* | Gly | D-Nar | Cys | Cyclo-Leu | 3Pal |
| 139 | | | Lys* | Gly | D-Nar | Cys | Aib(O-cyclic) | Cit |
| 140 | | | | Lys* | Arg | Cys | Aib(O-cyclic) | Gln |
| 141 | | | | Lys* | D-Nar | Cys | Aib(O-cyclic) | Gln |
| 142 | | | | Lys* | BetahomoArg | Cys | Aib(O-cyclic) | Gln |
| 143 | | | | Lys* | Arg | Cys | Aib(O-cyclic) | Gln |
| 144 | | | | Lys* | D-Nar | Cys | Aib(O-cyclic) | Gln |
| 145 | | | | Lys* | BetahomoArg | Cys | Aib(O-cyclic) | Gln |
| 146 | | | | Lys* | Arg | Cys | Aib(O-cyclic) | Gln |
| 147 | | | | Lys* | D-Nar | Cys | Aib(O-cyclic) | Gln |
| 148 | | | | Lys* | BetahomoArg | Cys | Aib(O-cyclic) | Gln |
| 149 | | | | Lys* | Arg | Cys | Aib(O-cyclic) | Gln |
| 150 | | | | Lys* | D-Nar | Cys | Aib(O-cyclic) | Gln |
| 151 | | | | Lys* | BetahomoArg | Cys | Aib(O-cyclic) | Gln |
| 165 | | | | Lys* | Arg | Cys | Aib | Gln |
| 166 | | | | Lys* | D-Nar | Pen | Aib(O-cyclic) | Gln |
| 167 | | | | Lys* | D-Nar | Pen | Aib(O-cyclic) | Gln |
| 428 | | | Lys* | Gly | D-Arg | Cys | L-aMeGlu | His |
| 436 | | | Lys* | Gly | Beta-homoArg | Cys | L-aMeGlu | His |
| 1129 | | Lys* | Gly | D-Arg | D-Nar | Cys | L-aMeGlu | His |
| 1128 | | Lys* | Glu | PRO | D-Nar | Cys | L-aMeGlu | His |
| 1133 | | Lys* | Gly | gGlu | D-Nar | Cys | L-aMeGlu | His |
| 499 | | Lys* | Gly | Gly | D-Nar | Cys | L-aMeAsp | His |
| 1131 | | Lys* | PEG1 | PEG1 | D-Nar | Cys | L-aMeAsp | His |
| 1130 | | Lys* | Gly | Gly | beta-homoArg | Cys | L-aMeAsp | His |
| 504 | | Lys* | PEG1 | PEG1 | beta-homoArg | Cys | L-aMeAsp | His |
| 1135 | | Lys* | Gly | Gly | D-Arg | Cys | L-aMeAsp | His |
| 561 | Lys* | D-Arg | PEG1 | PEG1 | D-Nar | Cys | L-aMeGlu | His |
| 562 | Lys* | PEG1 | PEG1 | PEG1 | D-Nar | Cys | L-aMeGlu | His |
| 563 | Lys* | Glu | PEG1 | PEG1 | D-Nar | Cys | L-aMeGlu | His |
| 564 | Lys* | Glu | Glu | Pro | D-Nar | Cys | L-aMeGlu | His |
| 1141 | Lys* | Ser | Glu | Pro | D-Nar | Cys | L-aMeGlu | His |
| 566 | Lys* | Ser | Gly | D-Arg | D-Nar | Cys | L-aMeGlu | His |
| 567 | Lys* | D-Arg | Gly | D-Arg | D-Nar | Cys | L-aMeGlu | His |
| 568 | Lys* | D-Arg | Ser | γ-Glu | D-Nar | Cys | L-aMeGlu | His |
| 569 | Lys* | Glu | Gly | γ-Glu | D-Nar | Cys | L-aMeGlu | His |
| 1143 | Lys* | Gly | Gly | γ-Glu | D-Nar | Cys | L-aMeGlu | His |
| 571 | | Lys* | Gly | γ-Glu | Me-D-Arg | Cys | L-aMeGlu | His |
| 572 | | Lys* | Gly | D-Arg | D-Nar | Cys | L-aMeGlu | His |
| 573 | | Lys* | Glu | Pro | D-Nar | Cys | L-aMeGlu | His |
| 574 | | Lys* | Glu | γ-Glu | D-Nar | Cys | L-aMeGlu | His |
| 575 | | Lys* | D-Arg | γ-Glu | D-Nar | Cys | L-aMeGlu | His |
| 1134 | Lys* | Glu | Glu | Pro | D-Nar | Cys | L-aMeGlu | His |
| 1132 | Lys* | Glu | PEG1 | PEG1 | D-Nar | Cys | L-aMeGlu | His |
| 589 | Lys* | Glu | Glu | Pro | D-Nar | Cys | D-bhGlu | His |
| 590 | Lys* | Glu | PEG1 | PEG1 | D-Nar | Cys | D-bhGlu | His |
| 1146 | Lys* | D-Arg | Gly | D-Arg | D-Nar | Cys | Phg | 3Pal |

| Molecule | | | | | | | |
|---|---|---|---|---|---|---|---|
| Name | X₅ | X₆ | X₇ | X₈ | N-term | C-term | Cyclic |
| 1146 | D-Phe(4-F) | Arg | Trp(6-F) | Cys | Ac | NH2 | Disulfide |
| 1139 | D-Phe(4-F) | Arg | Trp(6-F) | Cys | Ac | NH2 | Disulfide |
| 1145 | D-Phe(4-F) | Arg | Trp(6-F) | Cys | Ac | NH2 | Disulfide |
| 1147 | D-Phe(4-F) | Arg | Trp(6-F) | Cys | Ac | NH2 | Disulfide |
| 1148 | D-Phe | Arg | Trp(6-F) | Cys | Ac | NH2 | Disulfide |
| 1149 | D-Phe | Arg | Trp(6-F) | Cys | Ac | NH2 | Disulfide |
| 1137 | D-Phe | Arg | Trp(6-F) | Cys | Ac | NH2 | Disulfide |
| 1136 | D-Phe | Arg | Trp(6-F) | Cys | Ac | NH2 | Disulfide |
| 1150 | D-Phe(4-F) | Arg | Trp(6-F) | Cys | Ac | NH2 | Disulfide |
| 1142 | D-Phe(4-F) | Arg | Trp(6-F) | Cys | Ac | NH2 | Disulfide |
| 1144 | D-Phe(4-F) | Arg | Trp(6-F) | Cys | Ac | NH2 | Disulfide |
| 1151 | D-Phe(4-F) | Arg | Trp(6-F) | Cys | Ac | NH2 | Disulfide |
| 1152 | D-Phe(4-F) | Arg | Trp(6-F) | Cys | Ac | NH2 | Disulfide |
| 1153 | D-Phe(4-F) | Arg | Trp(6-F) | Cys | Ac | NH2 | Disulfide |
| 1154 | D-Phe(4-F) | Arg | Trp(6-F) | Cys | Ac | NH2 | Disulfide |
| 1155 | D-Phe(4-F) | Arg | Trp(6-F) | Cys | Ac | NH2 | Disulfide |
| 1156 | D-Phe | Arg | Trp(6-F) | Cys | Ac | NH2 | Disulfide |
| 1157 | D-Phe | Arg | Trp(6-F) | Cys | Ac | NH2 | Disulfide |
| 111 | D-Phe(4-F) | Arg | Trp(6-F) | Cys | Ac | NH2 | Disulfide |
| 112 | D-Phe(4-F) | Arg | Trp(6-F) | Cys | Ac | NH2 | Disulfide |
| 113 | D-Phe(4-F) | Arg | Trp(6-F) | Cys | Ac | NH2 | Disulfide |
| 114 | D-Phe(4-F) | Arg | Trp(6-F) | Cys | Ac | NH2 | Disulfide |
| 123 | D-Phe(4-F) | Arg | Trp(6-F) | Cys | Ac | NH2 | Disulfide |
| 124 | D-Phe(4-F) | Arg | Trp(6-F) | Cys | Ac | NH2 | Disulfide |
| 125 | D-Phe(4-F) | Arg | Trp(6-F) | Cys | Ac | NH2 | Disulfide |
| 130 | D-Phe | Arg | Trp(6-F) | Cys | Ac | NH2 | Disulfide |
| 131 | D-Phe | Arg | Trp(6-F) | Cys | Ac | NH2 | Disulfide |
| 132 | D-Phe | Arg | Trp(6-F) | Cys | Ac | NH2 | Disulfide |
| 138 | D-Phe | Arg | Trp(6-F) | Cys | Ac | NH2 | Disulfide |
| 139 | D-Phe | Arg | Trp(6-F) | Cys | Ac | NH2 | Disulfide |
| 140 | D-Phe(4-F) | Arg | Trp(6-F) | Pen | Ac | NH2 | Disulfide |
| 141 | D-Phe(4-F) | Arg | Trp(6-F) | Pen | Ac | NH2 | Disulfide |
| 142 | D-Phe(4-F) | Arg | Trp(6-F) | Pen | Ac | NH2 | Disulfide |
| 143 | D-Phe | Arg | Trp | Pen | Ac | NH2 | Disulfide |
| 144 | D-Phe | Arg | Trp | Pen | Ac | NH2 | Disulfide |
| 145 | D-Phe | Arg | Trp | Pen | Ac | NH2 | Disulfide |
| 146 | D-Phe | Arg | Trp(6-F) | Pen | Ac | NH2 | Disulfide |
| 147 | D-Phe | Arg | Trp(6-F) | Pen | Ac | NH2 | Disulfide |
| 148 | D-Phe | Arg | Trp(6-F) | Pen | Ac | NH2 | Disulfide |
| 149 | D-Phe(4-F) | Arg | Trp | Pen | Ac | NH2 | Disulfide |
| 150 | D-Phe(4-F) | Arg | Trp | Pen | Ac | NH2 | Disulfide |
| 151 | D-Phe(4-F) | Arg | Trp | Pen | Ac | NH2 | Disulfide |
| 165 | D-Phe(4-F) | Arg | Trp(6-F) | Pen | Ac | NH2 | Disulfide |
| 166 | D-Phe(4-F) | Arg | Trp(6-F) | Pen | Ac | NH2 | Disulfide |
| 167 | D-Phe(4-F) | Arg | Trp(6-F) | Cys | Ac | NH2 | Disulfide |
| 428 | D-Phe | Arg | Trp | Cys | Ac | NH2 | Disulfide |
| 436 | D-Phe | Arg | Trp | Cys | Ac | NH2 | Disulfide |
| 1129 | D-Phe | Arg | Trp(6-Me) | Cys | Ac | NH2 | Disulfide |
| 1128 | D-Phe | Arg | Trp(6-Me) | Cys | Ac | NH2 | Disulfide |
| 1133 | D-Phe | Arg | Trp(6-Me) | Cys | Ac | NH2 | Disulfide |
| 499 | D-Phe | Arg | Trp(6-Me) | Cys | Ac | NH2 | Disulfide |
| 1131 | D-Phe | Arg | Trp(6-Me) | Cys | Ac | NH2 | Disulfide |
| 1130 | D-Phe | Arg | Trp(6-Me) | Cys | Ac | NH2 | Disulfide |
| 504 | D-Phe | Arg | Trp(6-Me) | Cys | Ac | NH2 | Disulfide |
| 1135 | D-Phe | Arg | Trp(6-Me) | Cys | Ac | NH2 | Disulfide |
| 561 | D-Phe | Arg | Trp(6-F) | Cys | Ac | NH2 | Disulfide |
| 562 | D-Phe | Arg | Trp(6-F) | Cys | Ac | NH2 | Disulfide |
| 563 | D-Phe | Arg | Trp(6-F) | Cys | Ac | NH2 | Disulfide |
| 564 | D-Phe | Arg | Trp(6-F) | Cys | Ac | NH2 | Disulfide |
| 1141 | D-Phe | Arg | Trp(6-F) | Cys | Ac | NH2 | Disulfide |
| 566 | D-Phe | Arg | Trp(6-F) | Cys | Ac | NH2 | Disulfide |
| 567 | D-Phe | Arg | Trp(6-F) | Cys | Ac | NH2 | Disulfide |
| 568 | D-Phe | Arg | Trp(6-F) | Cys | Ac | NH2 | Disulfide |
| 569 | D-Phe | Arg | Trp(6-F) | Cys | Ac | NH2 | Disulfide |
| 1143 | D-Phe | Arg | Trp(6-F) | Cys | Ac | NH2 | Disulfide |
| 571 | D-Phe | Arg | Trp(6-F) | Cys | Ac | NH2 | Disulfide |
| 572 | D-Phe | Arg | Trp(6-F) | Cys | Ac | NH2 | Disulfide |
| 573 | D-Phe | Arg | Trp(6-F) | Cys | Ac | NH2 | Disulfide |
| 574 | D-Phe | Arg | Trp(6-F) | Cys | Ac | NH2 | Disulfide |
| 575 | D-Phe | Arg | Trp(6-F) | Cys | Ac | NH2 | Disulfide |
| 1134 | D-Phe(3,4,5- | Arg | Trp(6-Me) | Cys | Ac | NH2 | Disulfide |
| | triF) | | | | | | |
| 1132 | D-Phe(3,4,5- | Arg | Trp(6-Me) | Cys | Ac | NH2 | Disulfide |
| | triF) | | | | | | |
| 589 | D-Phe | Arg | Trp(6-Me) | Cys | Ac | NH2 | Disulfide |
| 590 | D-Phe | Arg | Trp(6-Me) | Cys | Ac | NH2 | Disulfide |
| 1146 | D-Phe(4-F) | Arg | Trp(6-F) | Cys | Ac | NH2 | Disulfide |

In embodiments, one or more amino acid residues and/or linkers are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₁ have the sequential designation of X₋₁, X₋₂, X₋₃, X₋₄, X₋₅, and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2. In embodiments, one or more amino acid residues and/or linkers are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, at least 1, or at least 2, or at least 3, or at least 4, or at least 5 linkers and/or spacers are conjugated to X₁ of any one of the peptides of formula (I) or formula (II). In embodiments, the one or more amino acid residues and/or linkers conjugated to X₈ have the sequential designation of X₉, X₁₀, X₁₁, X₁₂, X₁₃, X₁₄ and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, the one or more amino acid residues and/or linkers are selected from Table 3. In embodiments, the linker is a PEG linker or lipid selected from Table 3.

In embodiments, a linker is an amino acid, including but not limited to modified amino acids. Non-limiting examples of modified amino acids include PEG groups (e.g., PEG-2), and lipids. In embodiments, the modified amino acid is Lys(AEEAc-AEEAc-L-γ-Glu-17-carboxyheptadecanoyl) (Lys*). In embodiments, the linker is Lys*.

In embodiments, the peptide further comprises one or more lipids conjugated to X₁ and/or X₈.

In embodiments, the peptide further comprises one or more PEG linkers conjugated to X₁ and/or X₈.

In embodiments, the peptide of formula (I) is a peptide of formula (Ia):

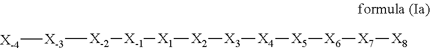

wherein in formula (Ia): X₋₁, X₋₂, X₋₃, X₋₄, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ib):

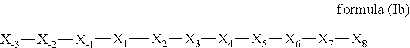

wherein in formula (Ib): X₋₁, X₋₂, X₋₃, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ic):

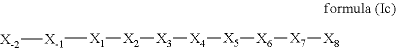

wherein in formula (Ic): X₋₁, X₋₂, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Id):

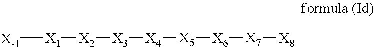

wherein in formula (Id): X₋₁, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ie):

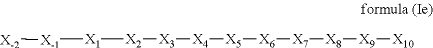

wherein in formula (Ie): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the peptide of formula (I) is a peptide of formula (If):

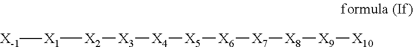

wherein in formula (Ie): X₋₁, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (I) is a cyclic peptide of any one of formula (Ia), formula (Ib), formula (Ic), formula (Id), formula (Ie), or formula (If), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide consists of the amino acid sequence as set forth in formula (Ia). In embodiments, the peptide consists of the amino acid sequence as set forth in formula (Ib). In embodiments, the peptide consists of the amino acid sequence as set forth in formula (Ic). In embodiments, the peptide consists of the amino acid sequence as set forth in formula (Id). In embodiments, the peptide consists of the amino acid sequence as set forth in formula (Ie). In embodiments, the peptide consists of the amino acid sequence as set forth in formula (If).

In embodiments, the API is a peptide, or a salt thereof, wherein the peptide comprises the amino acid sequence of formula (BI), formula (CI), formula (DI), formula (EI), or formula (FI).

In embodiments, in formula (BI), X₁, X₂, X₃, X₄, X₅, X₆, X₇, and X₈ are each independently an amino acid selected from Table 1, Table 2, Table 3, Table C1, Table CIA, Table C2, and Table C2A.

In embodiments, in formula (CI), X₁, X₂, X₃, X₄, X₅, X₆, X₇, and X₈ are each independently an amino acid selected from Table 1, Table 2, Table 3, Table C1, Table CIA, Table C2, and Table C2A.

In embodiments, in formula (DI), X₁, X₂, X₃, X₄, X₅, X₆, X₇, and X₈ are each independently an amino acid selected from Table 1, Table 2, Table 3, Table DI, Table DIA, Table D2, and Table D2A.

In embodiments, in formula (EI), X₁, X₂, X₃, X₄, X₅, X₆, X₇, and X₈ are each independently an amino acid selected from Table 1, Table 2, Table 3, Table E1, Table E1A, Table E2, and Table E2A.

In embodiments, in formula (FI), X₁, X₂, X₃, X₄, X₅, X₆, X₇, and X₈ are each independently an amino acid selected from Table 1, Table 2, Table 3, Table F1, Table F1A, Table F2, and Table F2A.

In embodiments, the peptide of formula (I) is a peptide of any one of formula (Ia), formula (Ib), formula (Ic), formula (Id), formula (Ie), or formula (If).

In embodiments, the peptide of formula (BI) is a peptide of any one of formula (BIa), formula (BIb), formula (BIc), formula (BId), formula (BIe), or formula (BIf).

In embodiments, in formula (BIa), formula (BIb), formula (BIc), formula (BId), formula (BIe), or formula (BIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (CI) is a peptide of any one of formula (CIa), formula (CIb), formula (CIc), formula (CId), formula (CIe), or formula (CIf).

In embodiments, in formula (CIa), formula (CIb), formula (CIc), formula (CId), formula (CIe), or formula (CIf), X₋₁, X₋₂, X₋₃, X₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (DI) is a peptide of any one of formula (DIa), formula (DIb), formula (DIc), formula (DId), formula (DIe), or formula (DIf).

In embodiments, in formula (DIa), formula (DIb), formula (DIc), formula (DId), formula (DIe), or formula (DIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (EI) is a peptide of any one of formula (EIa), formula (EIb), formula (EIc), formula (EId), formula (EIe), or formula (EIf).

In embodiments, in formula (EIa), formula (EIb), formula (EIc), formula (EId), formula (EIe), or formula (EIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (FI) is a peptide of any one of formula (FIa), formula (FIb), formula (FIc), formula (FId), formula (FIe), or formula (FIf).

In embodiments, in formula (FIa), formula (FIb), formula (FIc), formula (FId), formula (FIe), or formula (FIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the API is a peptide, or a salt thereof, wherein the peptide consists of the amino acid sequence as set forth in formula (BI). In embodiments, the peptide consists of the amino acid sequence as set forth in formula (BIa). In embodiments, the peptide consists of the amino acid sequence as set forth in formula (BIb). In embodiments, the peptide consists of the amino acid sequence as set forth in formula (BIc). In embodiments, the peptide consists of the amino acid sequence as set forth in formula (BId). In embodiments, the peptide consists of the amino acid sequence as set forth in formula (BIe). In embodiments, the peptide consists of the amino acid sequence as set forth in formula (BIf).

In embodiments, the API is a peptide, or a salt thereof, wherein the peptide consists of the amino acid sequence as set forth in formula (CI). In embodiments, the peptide consists of the amino acid sequence as set forth in formula (CIa). In embodiments, the peptide consists of the amino acid sequence as set forth in formula (CIb). In embodiments, the peptide consists of the amino acid sequence as set forth in formula (CIc). In embodiments, the peptide consists of the amino acid sequence as set forth in formula (CId). In embodiments, the peptide consists of the amino acid sequence as set forth in formula (CIe). In embodiments, the peptide consists of the amino acid sequence as set forth in formula (CIf).

In embodiments, the API is a peptide, or a salt thereof, wherein the peptide consists of the amino acid sequence as set forth in formula (DI). In embodiments, the peptide consists of the amino acid sequence as set forth in formula (DIa). In embodiments, the peptide consists of the amino acid sequence as set forth in formula (DIb). In embodiments, the peptide consists of the amino acid sequence as set forth in formula (DIc). In embodiments, the peptide consists of the amino acid sequence as set forth in formula (DId). In embodiments, the peptide consists of the amino acid sequence as set forth in formula (DIe). In embodiments, the peptide consists of the amino acid sequence as set forth in formula (DIf).

In embodiments, the API is a peptide, or a salt thereof, wherein the peptide consists of the amino acid sequence as set forth in formula (EI). In embodiments, the peptide consists of the amino acid sequence as set forth in formula (EIa). In embodiments, the peptide consists of the amino acid sequence as set forth in formula (EIb). In embodiments, the peptide consists of the amino acid sequence as set forth in formula (EIc). In embodiments, the peptide consists of the amino acid sequence as set forth in formula (EId). In embodiments, the peptide consists of the amino acid sequence as set forth in formula (EIe). In embodiments, the peptide consists of the amino acid sequence as set forth in formula (EIf).

In embodiments, the API is a peptide, or a salt thereof, wherein the peptide consists of the amino acid sequence as set forth in formula (FI). In embodiments, the peptide consists of the amino acid sequence as set forth in formula (FIa). In embodiments, the peptide consists of the amino acid sequence as set forth in formula (FIb). In embodiments, the peptide consists of the amino acid sequence as set forth in formula (FIc). In embodiments, the peptide consists of the amino acid sequence as set forth in formula (FId). In embodiments, the peptide consists of the amino acid sequence as set forth in formula (FIe). In embodiments, the peptide consists of the amino acid sequence as set forth in formula (FIf).

Amino acids described herein are construed as L-amino acids unless specified otherwise (e.g., D-amino acids, such as D-arginine (D-Arg)).

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers and/or spacers at the amino and/or carboxy terminus.

In embodiments, the peptides of formula (I) or formula (II) comprises an N-terminal and/or C-terminal modification. In embodiments, the N-terminal modification comprises acetylation, propionylation, methylation, myristovlation, palmitoylation, or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein RI and R2 are selected from H or alkyl.

In embodiments, the peptides of formula (I) or formula (II) comprises an N-terminal acetyl. In embodiments, the peptides of formula (I) or formula (II) comprises a C-terminal amide. In embodiments, the peptides of formula (I) or formula (II) comprises an N-terminal acetyl and C-terminal amide.

In embodiments, the peptide of formula (I) is selected from Table 1 and Table 2.

In embodiments, the peptide of formula (II) is selected from Table 1 and Table 2.

In embodiments, X₄ is Gln. In embodiments, X₄ is Cit. In embodiments, X₄ is hCit. In embodiments, X₄ is 3-Pal. In embodiments, X₄ is hGln. In embodiments, X₄ is His. In embodiments, X₄ is Orn.

In embodiments, X₃-X₄ is selected from Aib(O-cyclic)-Gln, Aib(O-cyclic)-hCit, Aib(O-cyclic)-Cit, Aib(O-cyclic)-3-Pal, Aib(O-cyclic)-hGln, Aib(O-cyclic)-His, and Aib(O-cyclic)-Orn.

In embodiments, X₅ is selected from 4-fluoro-D-phenylalanine (D-Phe(4-F)), D-phenylalanine (D-Phe), and 4-methyl-D-phenylalanine (D-Phe(4-Me)).

In embodiments, X₅ is D-Phe(4-F).

In embodiments, X₅ is D-Phe.

In embodiments, X₅ is D-Phe(4-Me).

In embodiments, X₆ is arginine (Arg).

In embodiments, X₇ is 6-fluoro-L-tryptophan (Trp(6-F)).

In embodiments, X₈ is cysteine (Cys).

In embodiments, X₈ is penicillamine (Pen).

In embodiments, when X₂ and X₈ are Cys, X₂ and X₈ are linked by a disulfide bridge (—S—S—). In embodiments, when one of X₂ and X₈ is Cys and the other of X₂ and X₈ is Pen, X₂ and X₈ are linked by a disulfide bridge. In embodiments, when X₂ is Cys and X₈ is Pen, X₂ and X₈ are linked by a disulfide bridge.

In embodiments, X₁ is selected from D-norarginine (D-Nar) and beta-homo-L-arginine (Beta-homoArg).

In embodiments, X₁ is D-Nar.

In embodiments, X₁ is beta-homoArg.

In embodiments, X₂ is Cys.

In embodiments, X₃ is selected from X₃ column in Table 9 and X₄ is selected from X₄ column in Table 9. In embodiments, X₃ is selected from X₃ column in Table 1 and X₄ is selected from X₄ column in Table 1.

In embodiments, X₃-X₄ is selected from Phg-3-Pal, D-aMeOrn-Gln, Aib(O-cyclic)-Gln, Aib(O-cyclic)-hCit, Aib(O-cyclic)-Cit, Cyclo-Leu-3-Pal, L-aMeGlu-His, hGlu-Gln, D-aMeSer-Gln, Cyclo-Leu-Gln, hGlu-His, Ala (2-Me)-Gln, L-aMeAsp-His, Ala (2-Me)-His, D-bhGlu-His, D-aMeSer-His, bhGlu-His, D-aMeOrn-3-Pal, and Aib(O-cyclic)-3-Pal.

In embodiments, X₃-X₄ is selected from Phg-3-Pal, D-aMeOrn-Gln, Cyclo-Leu-3-Pal, and Aib(O-cyclic)-Gln.

In embodiments, X₃-X₄ is Phg-3-Pal.

In embodiments, X₃-X₄ is D-aMeOrn-Gln.

In embodiments, X₃-X₄ is Aib(O-cyclic)-Gln.

In embodiments, X₃-X₄ is Cyclo-Leu-3-Pal.

TABLE 9
Exemplary combinations of X₃ and X₄ positions.

| X₃ | X₄ |
|---|---|
| Phg | 3-Pal |
| D-aMeOrn | Gln |
| Aib(O-cyclic) | Gln |
| Aib(O-cyclic) | hCit |
| Aib(O-cyclic) | Cit |
| L-aMeGlu | His |
| hGlu | Gln |
| D-aMeSer | Gln |
| Cyclo-Leu | Gln |
| hGlu | His |
| Ala(2-Me) | Gln |
| L-aMeAsp | His |
| Ala(2-Me) | His |
| Cyclo-Leu | 3-Pal |
| D-bhGlu | His |
| D-aMeSer | His |
| bhGlu | His |
| D-aMeOrn | 3-Pal |
| Aib(O-cyclic) | 3-Pal |

In embodiments, X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), 3-fluoro-D-phenylalanine (D-Phe(3-F)), 2,3-difluoro-D-phenylalanine (D-Phe(2,3-diF)), 2,4,5-trifluoro-D-phenylalanine (D-Phe(2,4,5-triF)), 2,4-dichloro-D-phenylalanine (D-Phe(2,4-diCl)), 2,4-difluoro-D-phenylalanine (D-Phe(2,4-diF)), 4-chloro-2-fluoro-D-phenylalanine (D-Phe(2-F,4-Cl)), 3,4,5-trifluoro-D-phenylalanine (D-Phe(3,4,5-triF)), 3,4-difluoro-D-phenylalanine (D-Phe(3,4-diF)), 3,4-dimethyl-D-phenylalanine (D-Phe(3,4-diMe)), 3-chloro-D-phenylalanine (D-Phe(3-Cl)), 4-methyl-3-fluoro-D-phenylalanine (D-Phe(3-F,4-Me)), 3-methyl-D-phenylalanine (D-Phe(3-Me)), 4-(trifluoromethyl)-D-phenylalanine (D-Phe(4-CF3)), and 4-chloro-D-phenylalanine (D-Phe(4-CI)).

In embodiments, X₅ is 4-fluoro-D-phenylalanine (D-Phe(4-F)) or D-phenylalanine (D-Phe). In embodiments, X₅ is 4-Fluoro-D-phenylalanine (D-Phe(4-F)). In embodiments, X₅ is D-phenylalanine (D-Phe).

In embodiments, X₆ is arginine (Arg).

In embodiments, X₇ is selected from 4-fluoro-L-tryptophan (Trp(4-F)), 5-chloro-L-tryptophan (Trp(5-Cl)), 5-fluoro-L-tryptophan (Trp(5-F)), 5-methyl-L-tryptophan (Trp(5-Me)), 6-bromo-L-tryptophan (Trp(6-Br)), 6-(trifluoromethyl)-L-tryptophan (Trp(6-CF3)), 6-chloro-L-tryptophan (Trp(6-Cl)), 6-fluoro-L-tryptophan (Trp(6-F)), 6-methyl-L-tryptophan (Trp(6-Me)), 7-fluoro-L-tryptophan (Trp(7-F)), and tryptophan (Trp).

In embodiments, X₇ is 4-fluoro-L-tryptophan (Trp(4-F)). In embodiments, X₇ is 6-fluoro-L-tryptophan (Trp(6-F)).

In embodiments, X₈ is selected from Cysteine (Cys), 3-amino-L-alanine (Dap), and penicillamine (Pen).

In embodiments, X₈ is cysteine (Cys). In embodiments, X₈ is penicillamine (Pen).

In embodiments, X₁ is selected from Arg, beta-homo-L-arginine (Beta-homoArg), D-arginine (D-Arg), D-norarginine (D-Nar), homo-L-arginine (L-hArg), and L-norarginine (Nar).

In embodiments, X₁ is selected from D-arginine (D-Arg).

In embodiments, X₂ is selected from aspartic acid (Asp), Cys, and L-glutamate (Glu).

In embodiments, X₂ is Cysteine (Cys).

In embodiments, when X₂ and X₈ are Cys, the amino acids are linked by a disulfide bridge. In embodiments, when one of X₂ and X₈ is Cys and the other of X₂ and X₈ is Pen, the amino acids are linked by a disulfide bridge. In embodiments, when X₂ is Cys and X₈ is Pen, the amino acids are linked by a disulfide bridge. In embodiments, when X₂ is Asp or Glu, and X₈ is Dap, the amino acids are linked by an amide bond.

In embodiments, X₁ is selected from D-Nar, Arg, Beta-homoArg, D-Arg, X₅ is selected from D-Phe(4-F), D-Phe, D-Phe(3-CF3), D-Phe(3-F), and X₇ is selected from Trp(6-F), Trp(6-Me), Trp(5-Me) and Trp.

In embodiments, X₁ is selected from X₁ column of Table 10. In embodiments, X₅ is selected from X₅ column of Table 10. In embodiments, X₇ is selected from X₇ column of Table 10.

TABLE 10
Exemplary X₁, X₅, and X₇ Residues

| X₁ | X₅ | X₇ |
|---|---|---|
| D-Nar | D-Phe(4-F) | Trp(6-F) |
| Arg | D-Phe | Trp(6-Me) |
| Beta-homoArg | D-Phe(3-CF3) | Trp(5-Me) |
| D-Arg | D-Phe(3-F) | Trp |

In embodiments, X₁, X₅, and X₇ are D-Nar, D-Phe(4-F), and Trp(6-F), respectively. In embodiments, X₁, X₅, and X₇ are D-Nar, D-Phe, and Trp(6-F), respectively.

In embodiments, X₁, X₅, and X₇ are D-Nar, D-Phe(4-F), and Trp(6-F), respectively, and X₈ is penicillamine (Pen). In embodiments, X₁, X₅, and X₇ are D-Nar, D-Phe, and Trp(6-F), respectively, and X₈ is penicillamine (Pen).

In embodiments, the API is a peptide, or a salt thereof, wherein the peptide consists of the amino acid sequence as set forth in formula (III):

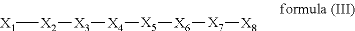

wherein in formula (III):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen).

In embodiments of formula (III), X₄ is glutamine (Gln). In embodiments of formula (III), X₄ is homocitrulline (hCit). In embodiments of formula (III), X₄ is citrulline (Cit). In embodiments of formula (III), X₅ is 4-fluoro-D-phenylalanine (D-Phe(4-F)). In embodiments of formula (III), X₅ is D-phenylalanine (D-Phe). In embodiments of formula (III), X₅ is 4-methyl-D-phenylalanine (D-Phe(4-Me)). In embodiments, the peptide of formula (III) is capped with N-terminal acetyl.

In embodiments, the API is a peptide, or a salt thereof, wherein the peptide consists of the amino acid sequence as set forth in formula (III):

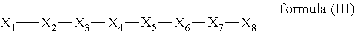

wherein in formula (III):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen).

In embodiments, the peptide of formula (III) is capped with N-terminal acetyl.

In embodiments, the cyclic peptide is a peptide consisting of the amino acid sequence as set forth in formula (IV):

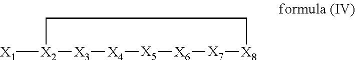

wherein in formula (IV):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen), wherein

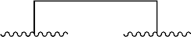 [SMILES: *CC*]

represents a disulfide bridge, and the peptide is capped with N-terminal acetyl.

In embodiments of formula (IV), X₄ is glutamine (Gln). In embodiments of formula (IV), X₄ is homocitrulline (hCit). In embodiments of formula (IV), X₄ is citrulline (Cit). In embodiments of formula (IV), X₅ is 4-fluoro-D-phenylalanine (D-Phe(4-F)). In embodiments of formula (IV), X₅ is D-phenylalanine (D-Phe). In embodiments of formula (IV), X₅ is 4-methyl-D-phenylalanine (D-Phe(4-Me)). In embodiments, the peptide of formula (IV) is capped with N-terminal acetyl.

In embodiments, the cyclic peptide is a peptide consisting of the amino acid sequence as set forth in formula (IV):

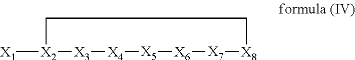

wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen), wherein

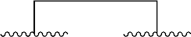 [SMILES: *CC*]

represents a disulfide bridge, and the peptide is capped with N-terminal acetyl.

### Cyclic Peptides

In embodiments, the API is a peptide, or a salt thereof, wherein the peptide is a cyclic peptide. In the embodiments, cyclic peptides include polypeptide chains taking cyclic ring structure. In embodiments, the ring structure is formed by linking one end of the peptide to its other end with an amide bond, or other chemically stable bonds such as lactam, ether, thioether, disulfide or via a stapled linkage. In embodiments, N-to-C (or head-to-tail) cyclization is amide bond formation between amino and carboxyl termini. In embodiments, the cyclic peptide comprises a bridge between amino acid at position X₂ and amino acid at position X₈ (e.g. as exemplified in formula II or formula IV).

In embodiments, the cyclic peptide comprises a disulfide bridge or a lactam bridge. In embodiments, the cyclic peptide comprises a disulfide bridge. In embodiments, the cyclic peptide comprises a lactam bridge (—NH—C(═O)—). In embodiments, the cyclic peptide comprises a bridge comprising one or more selected from amide, ether, disulfide, lactam, head-tail amidation, and Asp-Lys.

In embodiments, cyclic peptide comprises a bridge.

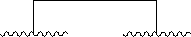 [SMILES: *CC*]

As used herein, the notation represents the linkage between two amino acids to form a cyclic peptide. In embodiments, the linkage is a bridge (e.g. a disulfide bridge —S—S—).

In embodiments, the cyclic peptide has the formula (II):

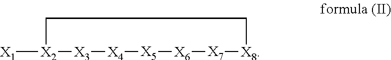

In embodiments, the peptide of formula (I) is a cyclic peptide of formula (II). In embodiments, the peptide of formula (I) is a peptide of formula (II) comprising a disulfide bridge. In embodiments, the peptide of formula (I) is a peptide of formula (II) comprising a lactam bridge. In embodiments, the peptide of molecules 1150, 1142, 1144, 1151, or 1158 is a cyclic peptide comprising a disulfide bridge. In embodiments, the peptide of molecules 1150, 1142, 1144, 1151, or 1158 is a cyclic peptide comprising a lactam bridge.

In embodiments, the peptide further comprises one or more lipids conjugated to X₁ and/or X₈. In embodiments, the peptide further comprises one or more amino acids conjugated to X₁ and/or X₈.

In embodiments, the one or more lipids are directly conjugated to X₁ and/or X₈. In embodiments, the one or more lipids are directly conjugated to the one or more amino acids, and the one or more amino acids are directly conjugated to X₁ and/or X₈.

In embodiments, the peptide further comprises one or more PEG linkers conjugated to X₁ and/or X₈.

In embodiments, the one or more PEG linkers are directly conjugated to X₁ and/or X₈. In embodiments, the one or more PEG linkers are directly conjugated to the one or more amino acids, and the one or more amino acids are directly conjugated to X₁ and/or X₈.

In embodiments, one or more amino acid residues and/or linkers are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₁ have the sequential designation of X₋₁, X₋₂, X₋₃, X₋₄, X₋₅, and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, one or more amino acid residues and/or linkers are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₈ have the sequential designation of X₉, X₁₀, X₁₁, X₁₂, X₁₃, X₁₄ and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers and/or spacers at the amino and/or carboxy terminus.

In embodiments, the cyclic peptide of formula (II) is a cyclic peptide of formula (IIa):

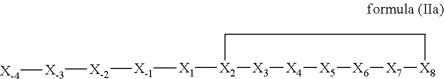

wherein in formula (IIa): X₋₁, X₋₂, X₋₃, X₋₄, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIb):

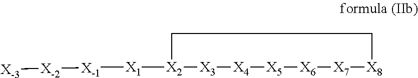

wherein in formula (IIb): X₋₁, X₋₂, X₋₃, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIc):

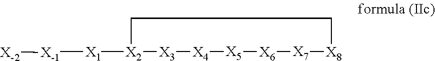

wherein in formula (IIc): X₋₁, X₋₂, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IId):

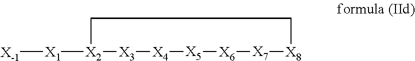

wherein in formula (IId): X₋₁, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIe):

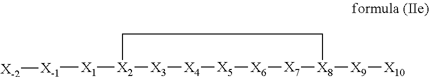

wherein in formula (IIe): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (II) is a cyclic peptide of formula (IIf):

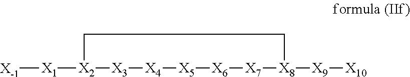

wherein in formula (IIf): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptides of formula (I) or formula (II) comprises an N-terminal acetyl (—C(═O) CH₃). In embodiments, the peptides of formula (I) or formula (II) comprises a C-terminal amide. In embodiments, the peptides of formula (I) or formula (II) comprises an N-terminal acetyl and C-terminal amide.

In embodiments, the peptide is capped with N-terminal acetyl and/or C-terminal amide groups.

In embodiments, the peptides of formula (I) or formula (II) comprises an N-terminal and/or C-terminal modification. In embodiments, the N-terminal modification comprises acetylation, propionylation, methylation, myristoylation, palmitoylation or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein RI and R2 are selected from H or alkyl.

In embodiments, the peptide of formula (I) or formula (II) is selected from Table 1 and Table 2.

In embodiments, the API is a peptide, or a salt thereof, wherein the peptide comprises the amino acid sequence of formula (I):

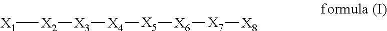

wherein in formula (I):
X₃ is 3-Aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

In embodiments, there is provided a composition comprising an API, wherein the API is a peptide, or a salt thereof, wherein the peptide is of any one of formula (Ia), formula (Ib), formula (Ic), formula (Id), formula (Ie), or formula (If):

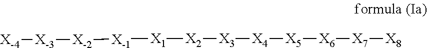

wherein in formula (Ia):
X₋₁, X₋₂, X₋₃, X₋₄, X₁, X₂, X₃, X₄, X₅, X₆, X₇, and X₈ are each independently an amino acid selected from Table A1; or Table A1 or a linker;

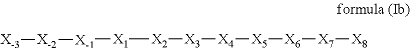

wherein in formula (Ib):
X₋₁, X₋₂, X₋₃, X₁, X₂, X₃, X₄, X₅, X₆, X₇, and X₈ are each independently an amino acid selected from Table A1; or Table A1 or a linker;

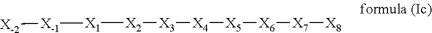

wherein in formula (Ic):
X₋₁, X₋₂, X₁, X₂, X₃, X₄, X₅, X₆, X₇, and X₈ are each independently an amino acid selected from Table A1; or Table A1 or a linker;

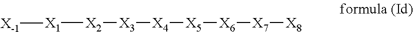

wherein in formula (Id):
X₋₁, X₁, X₂, X₃, X₄, X₅, X₆, X₇, and X₈ are each independently an amino acid selected from Table A1; or Table A1 or a linker;

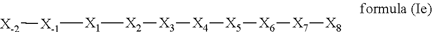

wherein in formula (Ie):
X₋₁, X₋₂, X₁, X₂, X₃, X₄, X₅, X₆, X₇, X₈, X₉, and X₁₀ are each independently an amino acid selected from Table A1; or Table A1 or a linker;

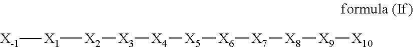

wherein in formula (If):
X₋₁, X₁, X₂, X₃, X₄, X₅, X₆, X₇, X₈, X₉, and X₁₀ are each independently an amino acid selected from Table A1; or Table A1 or a linker.

In embodiments, X₄ is Gln.

In embodiments, X₄ is Cit.

In embodiments, X₄ is hCit.

In embodiments, X₄ is 3-Pal.

In embodiments, X₄ is hGln.

In embodiments, X₄ is His.

In embodiments, X₄ is Om.

In embodiments, X₅ is selected from 4-fluoro-D-phenylalanine (D-Phe(4-F)), D-phenylalanine (D-Phe), and 4-methyl-D-phenylalanine (D-Phe(4-Me)).

In embodiments, X₅ is D-Phe(4-F).

In embodiments, X₅ is D-Phe.

In embodiments, X₅ is D-Phe(4-Me).

In embodiments, X₆ is arginine (Arg).

In embodiments, X₇ is 6-fluoro-L-tryptophan (Trp(6-F)).

In embodiments, X₈ is penicillamine (Pen) or cysteine (Cys).

In embodiments, X₁ is selected from D-norarginine (D-Nar) and beta-homo-L-arginine (Beta-homoArg).

In embodiments, X₁ is D-Nar.

In embodiments, X₂ is Cys.

In embodiments, the peptide of formula (I) is selected from Table A1, Table A1A, Table A2 and Table A2A.

In embodiments, the peptide is a cyclic peptide.

In embodiments, the cyclic peptide comprises a disulfide bridge or a lactam bridge.

In embodiments, there is provided a composition comprising an API, wherein the API is a peptide, or a salt thereof, wherein the peptide comprises the amino acid sequence of formula (II):

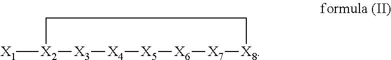

In embodiments, there is provided a composition comprising an API, wherein the API is a peptide, or a salt thereof, wherein the peptide comprises the amino acid sequence of any one of formula (IIa), formula (IIb), formula (IIc), formula (IId), formula (IIe), or formula (IIf):

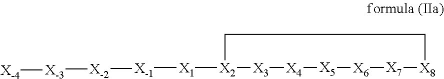

wherein in formula (IIa): X₋₁, X₋₂, X₋₃, X₋₄, X₁, X₂, X₃, X₄, X₅, X₆, X₇, and X₈ are each independently an amino acid selected from Table A1; or Table A1 or a linker;

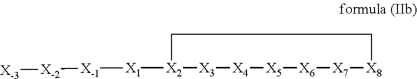

wherein in formula (IIb): X₋₁, X₋₂, X₋₃, X₁, X₂, X₃, X₄, X₅, X₆, X₇, and X₈ are each independently an amino acid selected from Table A1; or Table A1 or a linker;

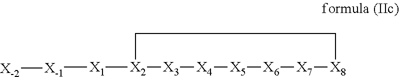

wherein in formula (IIc): X₋₁, X₋₂, X₁, X₂, X₃, X₄, X₅, X₆, X₇, and X₈ are each independently an amino acid selected from Table A1; or Table Alor a linker;

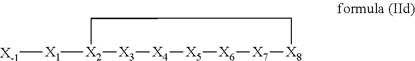

wherein in formula (IId): X₋₁, X₁, X₂, X₃, X₄, X₅, X₆, X₇, and X₈ are each independently an amino acid selected from Table A1; or Table Alor a linker;

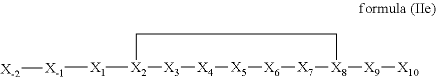

wherein in formula (IIe): X₋₁, X₋₂, X₁, X₂, X₃, X₄, X₅, X₆, X₇, X₈, X₉, and X₁₀ are each independently an amino acid selected from Table A1; or Table Alor a linker;

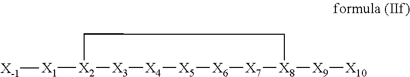

wherein in formula (IIf): X₋₁, X₋₂, X₁, X₂, X₃, X₄, X₅, X₆, X₇, X₈, X₉, and X₁₀ are each independently an amino acid selected from Table A1; or Table Alor a linker.

In embodiments, the peptide further comprises one or more amino acids conjugated to X₁ and/or X₈, optionally wherein the one or more amino acids are selected from D-Arginine (D-Arg), glycine (Gly), and L-Lys (AEEAc-AEEAc-L-γ-Glu-17-carboxyheptadecanoyl) (Lys*).

In embodiments, the peptide further comprises one or more lipids conjugated to X₁ and/or X₈.

In embodiments, the peptide is capped with N-terminal acetyl and/or C-terminal amide groups.

In embodiments, the peptide is selected from Table A1, Table A1A, Table A2 and Table A2A.

In embodiments, there is provided a composition comprising an API, wherein the API is a peptide, or a salt thereof, wherein the peptide consists of the amino acid sequence as set forth in formula (III):

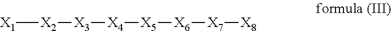

wherein in formula (III);
X₁ is D-norarginine (D-Nar);
X₂ is cysteine (Cys);
   X₃ is 3-Aminooxetane-3-carboxylic acid (Aib(O-cyclic));
   X₄ is glutamine (Gln);
X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen).

In embodiments, the peptide is a cyclic peptide.

In embodiments, the cyclic peptide comprises a disulfide bridge or a lactam bridge.

In embodiments, the cyclic peptide is a peptide consisting of the amino acid sequence as set forth in formula (IV):

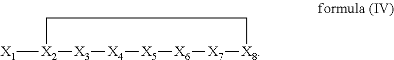

wherein in formula (IV):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen).

In embodiments, the peptide is capped with N-terminal acetyl and/or C-terminal amide groups.

In embodiments, the peptide is capped with N-terminal acetyl.

In embodiments, the API is a peptide or a salt thereof, wherein the peptide consists of the amino acid sequence as set forth in formula (IV):

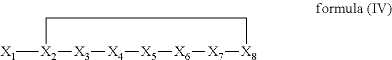

wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen),
wherein

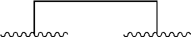 [SMILES: *CC*]

represents a disulfide bridge, and the peptide is capped with a N-terminal acetyl.

In embodiments, In embodiments, there is provided a composition comprising an API, wherein the API is a peptide, or a salt thereof, wherein the peptide comprises the amino acid sequence of formula (BI):

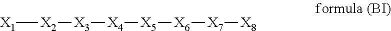

wherein in formula (BI):
X₃ is L-Phenylglycine (Phg);
X₄ is 3-(3-Pyridyl)-L-alanine (3-Pal); and
X₁, X₂, X₅, X₆, X₇, and X₈ are each independently a canonical or non-canonical amino acid.

In embodiments, In embodiments, there is provided a composition comprising an API, wherein the API is a peptide, or a salt thereof, wherein the peptide is of any one of formula (BIa), formula (BIb), formula (BIc), formula (BId), formula (BIe), or formula (BIf):

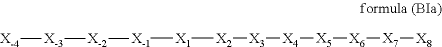

wherein in formula (BIa):
X₋₁, X₋₂, X₋₃, X₋₄, X₁, X₂, X₃, X₄, X₅, X₆, X₇, and X₈ are each independently an amino acid selected from Table B1; or Table B1 or a linker;

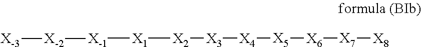

wherein in formula (Ib): X₋₁, X₋₂, X₋₃, X₁, X₂, X₃, X₄, X₅, X₆, X₇, and X₈ are each independently an amino acid selected from Table B1; or Table B1 or a linker;

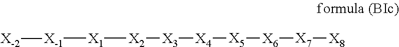

wherein in formula (Ic): X₋₁, X₋₂, X₁, X₂, X₃, X₄, X₅, X₆, X₇, and X₈ are each independently an amino acid selected from Table B1; or Table B1 or a linker;

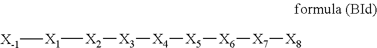

wherein in formula (Id): X₋₁, X₁, X₂, X₃, X₄, X₅, X₆, X₇, and X₈ are each independently an amino acid selected from Table B1; or Table B1 or a linker;

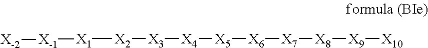

wherein in formula (Ie): X₋₁, X₋₂, X₁, X₂, X₃, X₄, X₅, X₆, X₁, X₈, X₉, and X₁₀ are each independently an amino acid selected from Table B1; or Table B1 or a linker;

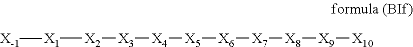

wherein in formula (BIf): X₋₁, X₁, X₂, X₃, X₄, X₅, X₆, X₇, X₈, X₉, and X₁₀ are each independently an amino acid selected from Table B1; or Table B1 or a linker.

In embodiments, there is provided a composition comprising an API, wherein the API is a peptide, or a salt thereof, wherein the peptide is of a cyclic peptide of formula (II):

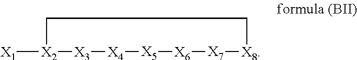

In embodiments, there is provided a composition comprising an API, wherein the API is a peptide, or a salt thereof, wherein the peptide is of any one of formula (BIIa), formula (BIIb), formula (BIIc), formula (BIId), formula (BIIe), or formula (BIIf):

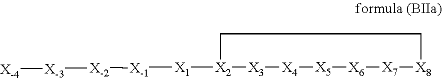

wherein in formula (BIIa): X₋₁, X₋₂, X₋₃, X₋₄, X₁, X₂, X₃, X₄, X₅, X₆, X₇, and X₈ are each independently an amino acid selected from Table B1; or Table B1 or a linker;

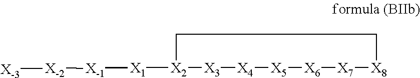

wherein in formula (IIb): X₋₁, X₋₂, X₋₃, X₁, X₂, X₃, X₄, X₅, X₆, X₇, and X₈ are each independently an amino acid selected from Table B1; or Table B1 or a linker;

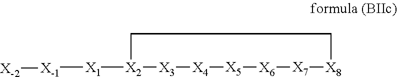

wherein in formula (BIIc): X₋₁, X₋₂, X₁, X₂, X₃, X₄, X₅, X₆, X₇, and X₈ are each independently an amino acid selected from Table B1; or Table B1 or a linker;

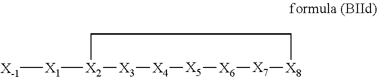

wherein in formula (IId): X₋₁, X₁, X₂, X₃, X₄, X₅, X₆, X₇, and X₈ are each independently an amino acid selected from Table B1; or Table B1 or a linker;

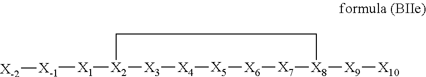

wherein in formula (IIe): X₋₁, X₋₂, X₁, X₂, X₃, X₄, X₅, X₆, X₁, X₈, X₉, and X₁₀ are each independently an amino acid selected from Table B1; or Table B1 or a linker;

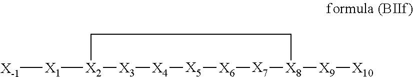

wherein in formula (IIf): X₋₁, X₋₂, X₁, X₂, X₃, X₄, X₅, X₆, X₇, X₈, X₉, and X₁₀ are each independently an amino acid selected from Table B1; or Table B1 or a linker.

In embodiments, there is provided a composition comprising an API, wherein the API is a peptide, or a salt thereof, wherein the peptide comprises the amino acid sequence of formula (I):

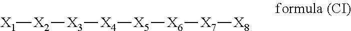

wherein in formula (CI):
X₃ is alpha-Methyl-D-Ornithine (D-aMeOrn);
X₄ is Glutamine (Gln); and
X₁, X₂, X₅, X₆, X₇, and X₈ are each independently a canonical or non-canonical amino acid.

In embodiments, there is provided a composition comprising an API, wherein the API is a peptide, or a salt thereof, wherein the peptide is of any one of formula (CIa), formula (CIb), formula (CIc), formula (CId), formula (CIe), or formula (CIf):

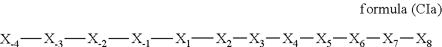

wherein in formula (CIa):
X₋₁, X₋₂, X₋₃, X₋₄, X₁, X₂, X₃, X₄, X₅, X₆, X₇, and X₈ are each independently an amino acid selected from Table C1; or Table C1 or a linker;

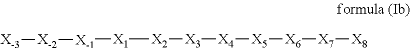

wherein in formula (Ib): X₋₁, X₋₂, X₋₃, X₁, X₂, X₃, X₄, X₅, X₆, X₇, and X₈ are each independently an amino acid selected from Table C1; or Table C1 or a linker;

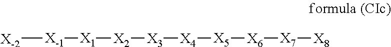

wherein in formula (Ic): X₋₁, X₋₂, X₁, X₂, X₃, X₄, X₃, X₆, X₇, and X₈ are each independently an amino acid selected from Table C1; or Table C1 or a linker;

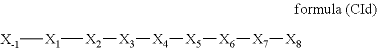

wherein in formula (Id): X₋₁, X₁, X₂, X₃, X₄, X₅, X₆, X₇, and X₈ are each independently an amino acid selected from Table C1; or Table C1 or a linker;

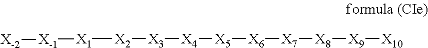

wherein in formula (Ie): X₋₁, X₋₂, X₁, X₂, X₃, X₄, X₅, X₆, X₁, X₈, X₉, and X₁₀ are each independently an amino acid selected from Table C1; or Table C1 or a linker;

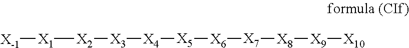

wherein in formula (If): X₋₁, X₁, X₂, X₃, X₄, X₅, X₆, X₁, X₈, X₉, and X₁₀ are each independently an amino acid selected from Table C1; or Table C1 or a linker.

In embodiments, there is provided a composition comprising an API, wherein the API is a peptide, or a salt thereof, wherein the peptide has the formula (CII):

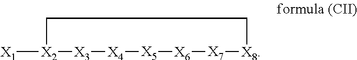

In embodiments, there is provided a composition comprising an API, wherein the API is a peptide, or a salt thereof, wherein the peptide is of any one of formula (CIIa), formula (CIIb), formula (CIIc), formula (CIId), formula (CIIe), or formula (CIIf):

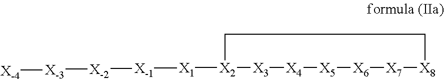

wherein in formula (IIa): X₋₁, X₋₂, X₋₃, X₋₄, X₁, X₂, X₃, X₄, X₅, X₆, X₇, and X₈ are each independently an amino acid selected from Table C1; or Table C1 or a linker;

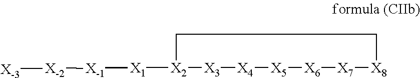

wherein in formula (IIb): X₋₁, X₋₂, X₋₃, X₁, X₂, X₃, X₄, X₅, X₆, X₇, and X₈ are each independently an amino acid selected from Table C1; or Table C1 or a linker;

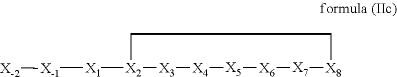

wherein in formula (IIc): X₋₁, X₋₂, X₁, X₂, X₃, X₄, X₅, X₆, X₇, and X₈ are each independently an amino acid selected from Table C1; or Table C1 or a linker;

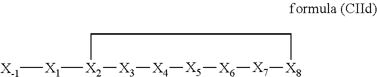

wherein in formula (IId): X₋₁, X₁, X₂, X₃, X₄, X₅, X₆, X₇, and X₈ are each independently an amino acid selected from Table C1; or Table C1 or a linker:

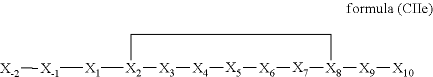

wherein in formula (IIe): X₋₁, X₋₂, X₁, X₂, X₃, X₄, X₅, X₆, X₇, X₈, X₉, and X₁₀ are each independently an amino acid selected from Table C1; or Table C1 or a linker:

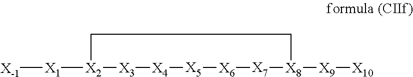

wherein in formula (IIf): X₋₁, X₋₂, X₁, X₂, X₃, X₄, X₅, X₆, X₇, X₈, X₉, and X₁₀ are each independently an amino acid selected from Table C1; or Table C1 or a linker.
acid selected from Table D1; or Table D1 or a linker;

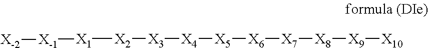

wherein in formula (Ie): X₋₁, X₋₂, X₁, X₂, X₃, X₄, X₅, X₆, X₇, X₈, X₉, and X₁₀ are each independently an amino acid selected from Table DI; or Table DI or a linker:

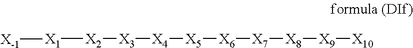

wherein in formula (DIf): X₋₁, X₁, X₂, X₃, X₄, X₅, X₆, X₇, X₈, X₉, and X₁₀ are each independently an amino acid selected from Table DI; or Table DI or a linker.

In embodiments, X₃ is selected from 2-methyl-alanine (Ala (2-Me)), 3-aminooxetane-3-carboxylic acid (Aib(O-cyclic)), alpha-methyl-D-aspartic acid (L-aMeAsp), alpha-methyl-D-ornithine (D-aMeOrn), alpha-methyl-D-serine (D-aMeSer), alpha-methyl-L-glutamic acid (L-aMeGlu), cycloleucine (Cyclo-Leu), beta-D-homoglutamic acid (D-bhGlu), beta-L-homoglutamic acid (bhGlu), L-homoglutamic acid (hGlu), and L-phenylglycine (Phg).

In embodiments, X₄ is selected from 3-(3-pyridyl)-L-alanine (3-Pal), glutamine (Gln), homocitrulline (hCit), citrulline (Cit), histidine (His), and L-ornithine (Orn).

In embodiments, X₃ is selected from X₃ column in Table DI and Table DIA and X₄ is selected from X₄ column in Table DI and Table DIA.

In embodiments, X₃-X₄ is selected from Phg-3-Pal, D-aMeOrn-Gln, Aib(O-cyclic)-Gln, Aib(O-cyclic)-hCit, Aib(O-cyclic)-Cit, hGlu-Gln, D-aMeSer-Gln, Cyclo-Leu-Gln, hGlu-His, Ala (2-Me)-Gln, L-aMeGlu-His, L-aMeAsp-His, and Ala (2-Me)-His, Phg-His, Cyclo-Leu-3-Pal, D-bhGlu-His, D-aMeSer-His, bhGlu-His, D-aMeOrn-3-Pal, and Aib(O-cyclic)-3-Pal.

In embodiments, X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), and 3-fluoro-D-phenylalanine (D-Phe(3-F)).

In embodiments, X₆ is arginine (Arg).

In embodiments, X₇ is selected from 6-fluoro-L-tryptophan (Trp(6-F)), 6-methyl-L-tryptophan (Trp(6-Me)), tryptophan (Trp), and 5-methyl-L-tryptophan (Trp(5-Me)).

In embodiments, X₈ is cysteine (Cys) or penicillamine (Pen).

In embodiments, X₁ is selected from D-norarginine (D-Nar), Arg, and beta-homo-L-arginine (Beta-homoArg).

In embodiments, X₂ is Cys.

In embodiments, there is provided a salt of a cyclic peptide having the formula (DII):

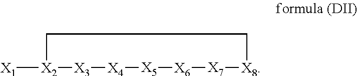

In embodiments, there is provided a composition comprising an API, wherein the API is a peptide, or a salt thereof, wherein the peptide is of any one of formula (DIIa), formula (DIIb), formula (DIIc), formula (DIId), formula (IIe), or formula (DIIf):

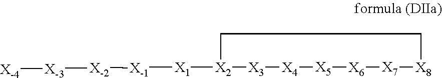

wherein in formula (DIIa): X₋₁, X₋₂, X₋₃, X₋₄, X₁, X₂, X₃, X₄, X₅, X₆, X₇, and X₈ are each independently an amino acid selected from Table DI; or Table DI or a linker:

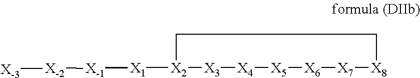

wherein in formula (DIIb): X₋₁, X₋₂, X₋₃, X₁, X₂, X₃, X₄, X₅, X₆, X₇, and X₈ are each independently an amino acid selected from Table D1; or Table D1 or a linker;

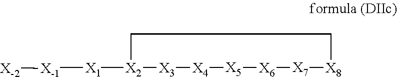

wherein in formula (DIIc): X₋₁, X₋₂, X₁, X₂, X₃, X₄, X₅, X₆, X₇, and X₈ are each independently an amino acid selected from Table D1; or Table D1 or a linker;

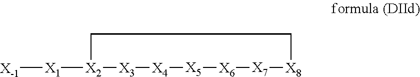

wherein in formula (DIId): X₋₁, X₁, X₂, X₃, X₄, X₅, X₆, X₇, and X₈ are each independently an amino acid selected from Table D1; or Table D1 or a linker;

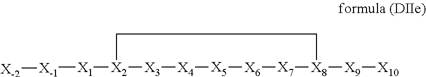

wherein in formula (DIIe): X₋₁, X₋₂, X₁, X₂, X₃, X₄, X₅, X₆, X₇, X₈, X₉, and X₁₀ are each independently an amino acid selected from Table D1; or Table D1 or a linker;

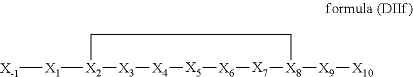

wherein in formula (DIIf): X₋₁, X₋₂, X₁, X₂, X₃, X₄, X₅, X₆, X₇, X₈, X₉, and X₁₀ are each independently an amino acid selected from Table D1; or Table D1 or a linker.

In embodiments, there is provided a composition comprising an API, wherein the API is a peptide, or a salt thereof, wherein the peptide comprises the amino acid sequence of formula (EI):

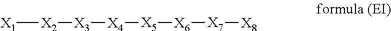

wherein in formula (EI):
X₁, X₂, X₃, X₄, X₅, X₆, X₇, and X₈ are each independently an amino acid selected from Table E1.

In embodiments, there is provided a composition comprising an API, wherein the API is a peptide, or a salt thereof, wherein the peptide is of any one of formula (EIa), formula (EIb), formula (EIc), formula (EId), formula (EIe), or formula (EIf):

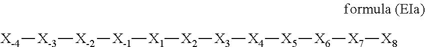

wherein in formula (EIa):
X₋₁, X₋₂, X₋₃, X₋₄, X₁, X₂, X₃, X₄, X₅, X₆, X₇, and X₈ are each independently an amino acid selected from Table E1; or Table E1 or a linker;

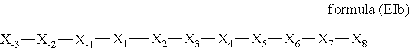

wherein in formula (Ib): X₋₁, X₋₂, X₋₃, X₁, X₂, X₃, X₄, X₅, X₆, X₇, and X₈ are each independently an amino acid selected from Table E1; or Table E1 or a linker;

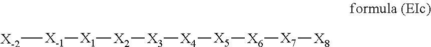

wherein in formula (Ic): X₋₁, X₋₂, X₁, X₂, X₃, X₄, X₅, X₆, X₇, and X₈ are each independently an amino acid selected from Table E1; or Table E1 or a linker;

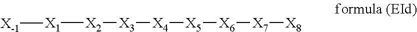

wherein in formula (Id): X₋₁, X₁, X₂, X₃, X₄, X₅, X₆, X₇, and X₈ are each independently an amino acid selected from Table E1; or Table E1 or a linker;

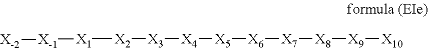

wherein in formula (Ie): X₋₁, X₋₂, X₁, X₂, X₃, X₄, X₅, X₆, X₇, X₈, X₉, and X₁₀ are each independently an amino acid selected from Table E1; or Table E1 or a linker;

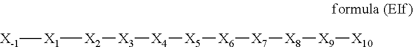

wherein in formula (EIf): X₋₁, X₁, X₂, X₃, X₄, X₅, X₆, X₇, X₈, X₉, and X₁₀ are each independently an amino acid selected from Table E1; or Table E1 or a linker.

In embodiments, X₃ is selected from 2-methyl-alanine (Ala (2-Me)), 3-aminooxetane-3-carboxylic acid (Aib(O-cyclic)), alpha-methyl-D-aspartic acid (L-aMeAsp), alpha-methyl-D-ornithine (D-aMeOrn), alpha-methyl-D-serine (D-aMeSer), alpha-methyl-L-glutamic acid (L-aMeGlu), cycloleucine (Cyclo-Leu), L-homoglutamic acid (hGlu), L-phenylglycine (Phg), beta-L-homoglutamic acid (bhGlu), D-alanine (D-ala), D-2,4-diaminobutyric acid (D-Dab), and L-homoglutamic acid (hGlu).

In embodiments, X₄ is selected from 3-(3-Pyridyl)-L-alanine (3-Pal), 3-(4-pyridyl)-L-alanine (4-Pal), glutamine (Gln), homocitrulline (hCit), citrulline (Cit), histidine (His), L-homoglutamine (hGln) and L-ornithine (Orn).

In embodiments, X₃ is selected from X₃ column in Table E1, and Table E1A and X₄ is selected from X₄ column in Table E1, and Table E1A.

In embodiments, X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), 3-fluoro-D-phenylalanine (D-Phe(3-F)), 2,3-difluoro-D-phenylalanine (D-Phe(2,3-diF)), 2,4,5-trifluoro-D-phenylalanine (D-Phe(2,4,5-triF)), 2,4-dichloro-D-phenylalanine (D-Phe(2,4-diCl)), 2,4-difluoro-D-phenylalanine (D-Phe(2,4-diF)), 4-Chloro-2-fluoro-D-phenylalanine (D-Phe(2-F,4-C1)), 3,4,5-trifluoro-D-phenylalanine (D-Phe(3,4,5-triF)), 3,4-difluoro-D-phenylalanine (D-Phe(3,4-diF)), 3,4-dimethyl-D-phenylalanine (D-Phe(3,4-diMe)), 3-chloro-D-phenylalanine (D-Phe(3-Cl)), 4-methyl-3-fluoro-D-phenylalanine (D-Phe(3-F,4-Me)), 3-methyl-D-phenylalanine (D-Phe(3-Me)), 4-(trifluoromethyl)-D-phenylalanine (D-Phe(4-CF3)), and 4-chloro-D-phenylalanine (D-Phe(4-Cl)).

In embodiments, X₆ is Arginine (Arg).

In embodiments, X₇ is selected from 4-fluoro-L-tryptophan (Trp(4-F)), 5-chloro-L-tryptophan (Trp(5-Cl)), 5-fluoro-L-tryptophan (Trp(5-F)), 5-methyl-L-tryptophan (Trp(5-Me)), 6-bromo-L-tryptophan (Trp(6-Br)), 6-(trifluoromethyl)-L-tryptophan (Trp(6-CF3)), 6-chloro-L-tryptophan (Trp(6-Cl)), 6-fluoro-L-tryptophan (Trp(6-F)), 6-methyl-L-tryptophan (Trp(6-Me)), 7-fluoro-L-tryptophan (Trp(7-F)), and tryptophan (Trp).

In embodiments, X₈ is selected from cysteine (Cys), 3-amino-L-alanine (Dap), and penicillamine (Pen).

In embodiments, X₁ is selected from Arg, beta-homo-L-arginine (Beta-homoArg), D-arginine (D-Arg), D-norarginine (D-Nar), homo-L-arginine (L-hArg), and L-norarginine (Nar).

In embodiments, X₂ is selected from aspartic acid (Asp), Cys, and L-glutamate (Glu).

In embodiments, there is provided a composition comprising an API, wherein the API is a peptide, or a salt thereof, wherein the peptide is of a cyclic peptide having the formula (EII):

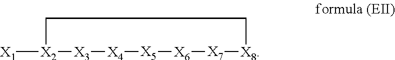

In embodiments, there is provided a composition comprising an API, wherein the API is a peptide, or a salt thereof, wherein the peptide is of any one of formula (EIIa), formula (EIIb), formula (EIIc), formula (EIId), formula (EIIe), or formula (EIIf):

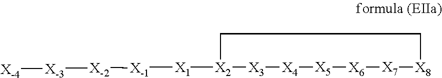

wherein in formula (EIIa): X₋₁, X₋₂, X₋₃, X₋₄, X₁, X₂, X₃, X₄, X₅, X₆, X₇, and X₈ are each independently an amino acid selected from Table E1; or Table E1 or a linker;

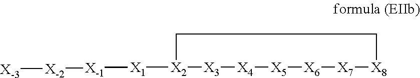

wherein in formula (EIIb): X₋₁, X₋₂, X₋₃, X₁, X₂, X₃, X₄, X₅, X₆, X₇, and X₈ are each independently an amino acid selected from Table E1; or Table E1 or a linker;

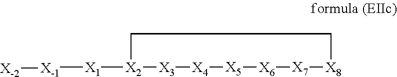

wherein in formula (EIIc): X₋₁, X₋₂, X₁, X₂, X₃, X₄, X₅, X₆, X₇, and X₈ are each independently an amino acid selected from Table E1; or Table E1 or a linker;

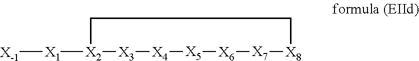

wherein in formula (IId): X₋₁, X₁, X₂, X₃, X₄, X₅, X₆, X₇, and X₈ are each independently an amino acid selected from Table E1; or Table E1 or a linker;

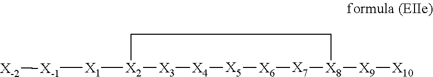

wherein in formula (EIIe): X₋₁, X₋₂, X₁, X₂, X₃, X₄, X₅, X₆, X₇, X₈, X₉, and X₁₀ are each independently an amino acid selected from Table E1; or Table E1 or a linker;

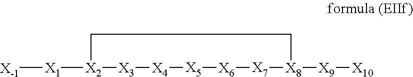

wherein in formula (EIIf): X₋₁, X₋₂, X₁, X₂, X₃, X₄, X₅, X₆, X₇, X₈, X₉, and X₁₀ are each independently an amino acid selected from Table E1; or Table E1 or a linker.

In embodiments, there is provided a composition comprising an API, wherein the API is a peptide, or a salt thereof, wherein the peptide comprises the amino acid sequence of formula (FI):

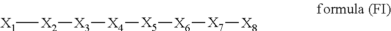

wherein in formula (FI):
X₃ is Cycloleucine (Cyclo-Leu);
X₄ is 3-(3-Pyridyl)-L-alanine (3Pal); and
X₁, X₂, X₅, X₆, X₇, and X₈ are each independently a canonical or non-canonical amino acid.

In embodiments, there is provided a composition comprising an API, wherein the API is a peptide, or a salt thereof, wherein the peptide is of any one of formula (FIa), formula (FIb), formula (FIc), formula (FId), formula (FIe), or formula (FIf):

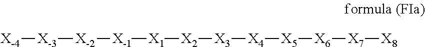

wherein in formula (FIa):
X₋₁, X₋₂, X₋₃, X₋₄, X₁, X₂, X₃, X₄, X₅, X₆, X₇, and X₈ are each independently an amino acid selected from Table F1; or Table F1 or a linker;

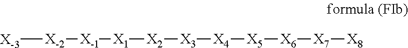

wherein in formula (Ib): X₋₁, X₋₂, X₋₃, X₁, X₂, X₃, X₄, X₅, X₆, X₇, and X₈ are each independently an amino acid selected from Table F1; or Table F1 or a linker;

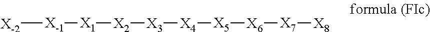

wherein in formula (Ic): X₋₁, X₁, X₂, X₃, X₄, X₅, X₆, X₇, and X₈ are each independently an amino acid selected from Table F1; or Table F1 or a linker;

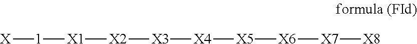

wherein in formula (Id): X₋₁, X₁, X₂, X₃, X₄, X₅, X₆, X₇, and X₈ are each independently an amino acid selected from Table F1; or Table F1 or a linker;

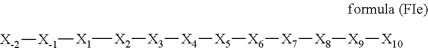

wherein in formula (Ie): X₋₁, X₋₂, X₁, X₂, X₃, X₄, X₅, X₆, X₇, X₈, X₉, and X₁₀ are each independently an amino acid selected from Table F1; or Table F1 or a linker;

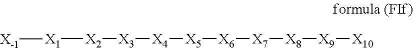

wherein in formula (Ie): X₋₁, X₁, X₂, X₃, X₄, X₅, X₆, X₇, X₈, X₉, and X₁₀ are each independently an amino acid selected from Table F1; or Table F1 or a linker.

In embodiments, X₅ is D-Phenylalanine (D-Phe).

In embodiments, X₆ is Arginine (Arg).

In embodiments, X₇ is 6-Fluoro-L-Tryptophan (Trp(6-F)).

In embodiments, X₈ is Cysteine (Cys).

In embodiments, X₁ is selected from D-Norarginine (D-Nar) and beta-homo-L-arginine (Beta-homoArg).

In embodiments, X₁ is D-Nar.

In embodiments, X₂ is Cys.

In embodiments, there is provided a salt of a cyclic peptide having the formula (FII):

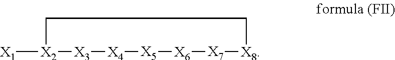

In embodiments, there is provided a composition comprising an API, wherein the API is a peptide, or a salt thereof, wherein the peptide is of any one of formula (FIIa), formula (FIIb), formula (FIIc), formula (FIId), formula (FIIe), or formula (IFIf):

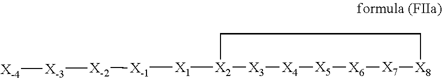

wherein in formula (FIIa): X₋₁, X₋₂, X₋₃, X₋₄, X₁, X₂, X₃, X₄, X₅, X₆, X₇, and X₈ are each independently an amino acid selected from Table F1; or Table F1 or a linker;

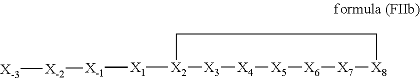

wherein in formula (FIIb): X₋₁, X₋₂, X₋₃, X₁, X₂, X₃, X₄, X₅, X₆, X₇, and X₈ are each independently an amino acid selected from Table F1; or Table F1 or a linker;

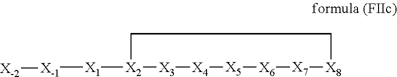

wherein in formula (FIIc): X₋₁, X₋₂, X₁, X₂, X₃, X₄, X₅, X₆, X₇, and X₈ are each independently an amino acid selected from Table F1; or Table F1 or a linker;

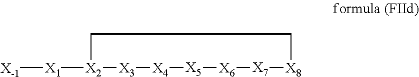

wherein in formula (IId): X₋₁, X₁, X₂, X₃, X₄, X₅, X₆, X₇, and X₈ are each independently an amino acid selected from Table F1; or Table F1 or a linker;

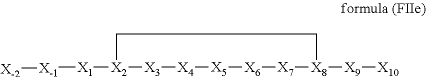

wherein in formula (FIIe): X₋₁, X₋₂, X₁, X₂, X₃, X₄, X₅, X₆, X₇, X₈, X₉, and X₁₀ are each independently an amino acid selected from Table F1; or Table F1 or a linker;

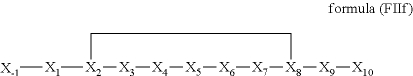

wherein in formula (FIIf): X₋₁, X₋₂, X₁, X₂, X₃, X₄, X₅, X₆, X₁, X₈, X₉, and X₁₀ are each independently an amino acid selected from Table F1; or Table F1 or a linker.

In embodiments, there is provided a composition comprising an API, wherein the API is a peptide, or a salt thereof, wherein the peptide comprises the amino acid sequence of formula (DI):

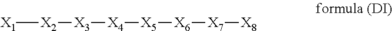

wherein in formula (DI):
X₁, X₂, X₃, X₄, X₅, X₆, X₇, and X₈ are each independently an amino acid selected from Table D1.

In embodiments, there is provided a composition comprising an API, wherein the API is a peptide, or a salt thereof, wherein the peptide is of any one of formula (Dla), formula (DIb), formula (DIc), formula (DId), formula (DIe), or formula (DIf):

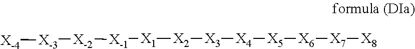

wherein in formula (DIa):
X₋₁, X₋₂, X₋₃, X₋₄, X₁, X₂, X₃, X₄, X₅, X₆, X₇, and X₈ are each independently an amino acid selected from Table D1; or Table DI or a linker;

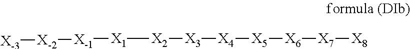

wherein in formula (Ib): X₋₁, X₋₂, X₋₃, X₁, X₂, X₃, X₄, X₅, X₆, X₇, and X₈ are each independently an amino acid selected from Table D1; or Table DI or a linker;

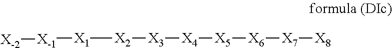

wherein in formula (Ic): X₋₁, X₋₂, X₁, X₂, X₃, X₄, X₃, X₆, X₇, and X₈ are each independently an amino acid selected from Table D1; or Table DI or a linker;

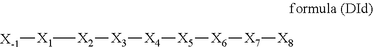

wherein in formula (Id): X₋₁, X₁, X₂, X₃, X₄, X₅, X₆, X₇, and X₈ are each independently an amino selected from Table D1; or Table DI or a linker.

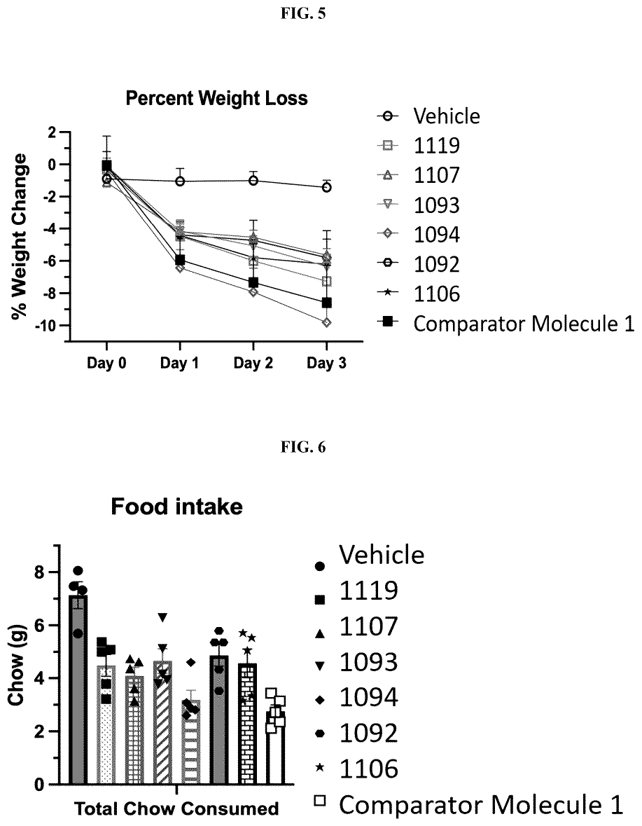

In embodiments, the API is a peptide, or a salt thereof, wherein the peptide is a peptide described in FIG. 5.

In embodiments, the API is a peptide, or a salt thereof, wherein the peptide is a peptide described in FIG. 6.

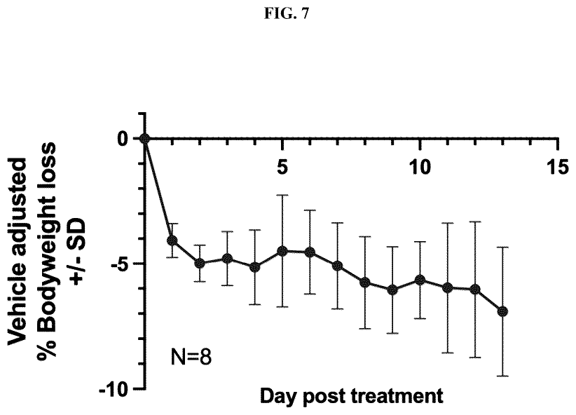

In embodiments, the API is a peptide, or a salt thereof, wherein the peptide is a peptide described in FIG. 7.

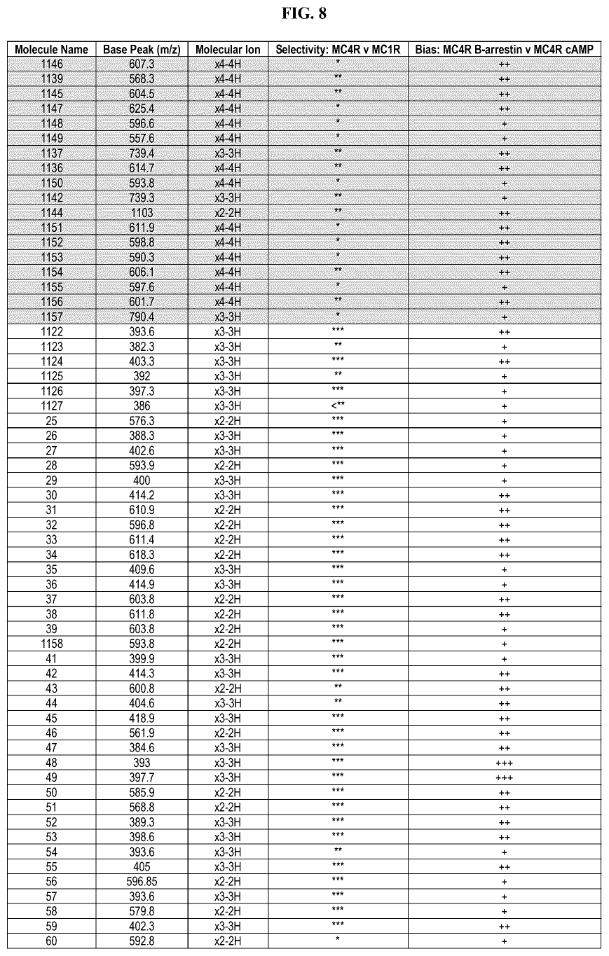

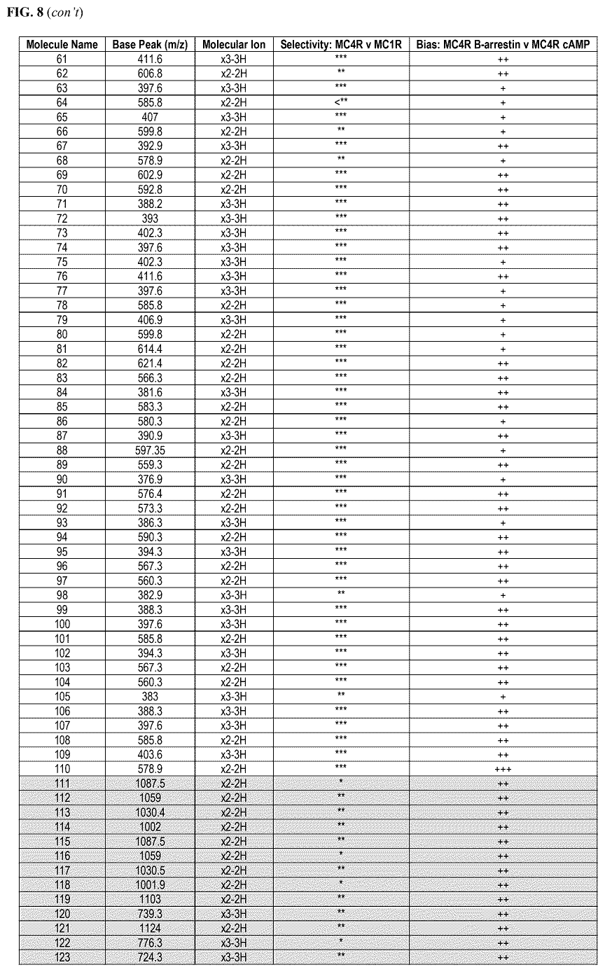

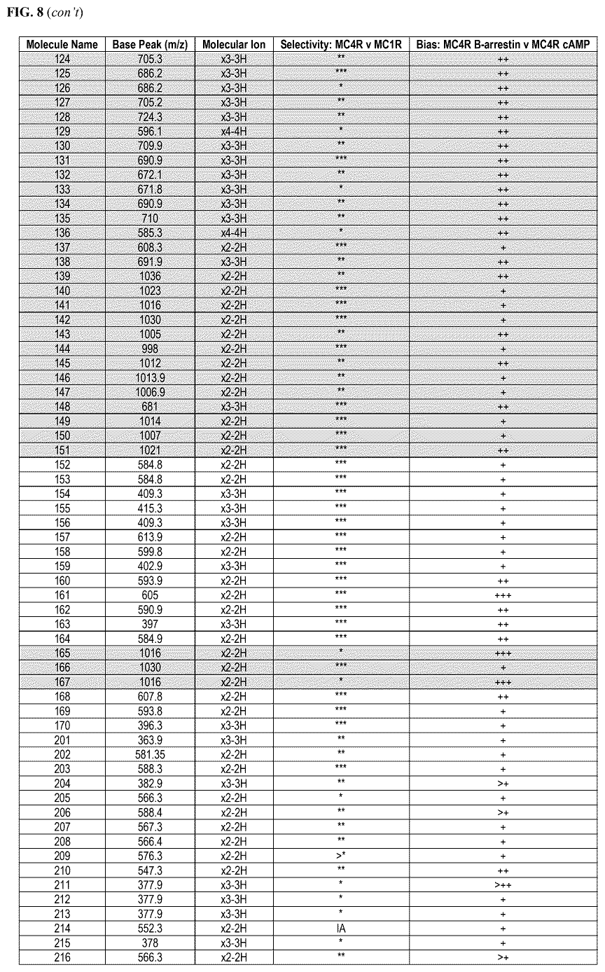

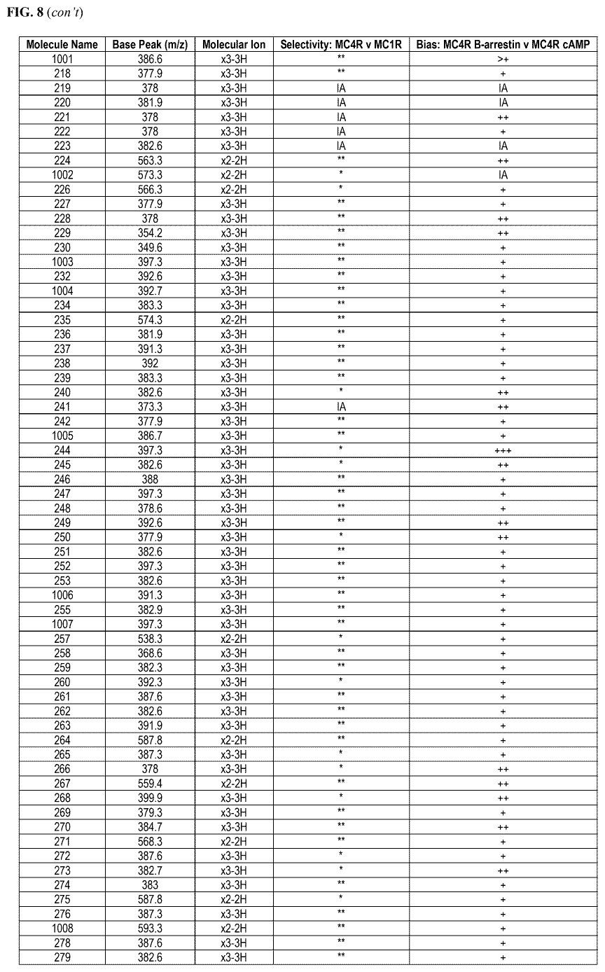

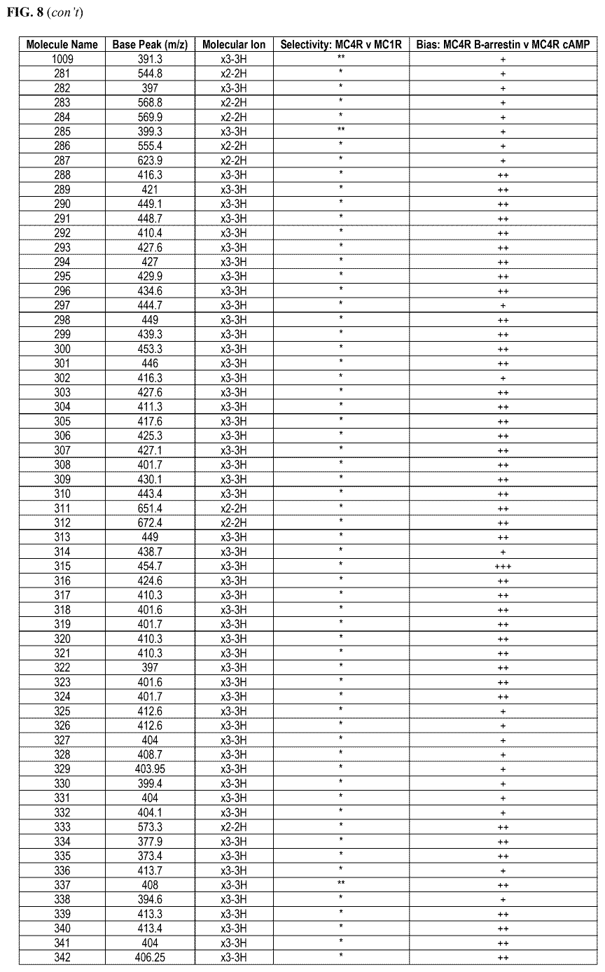

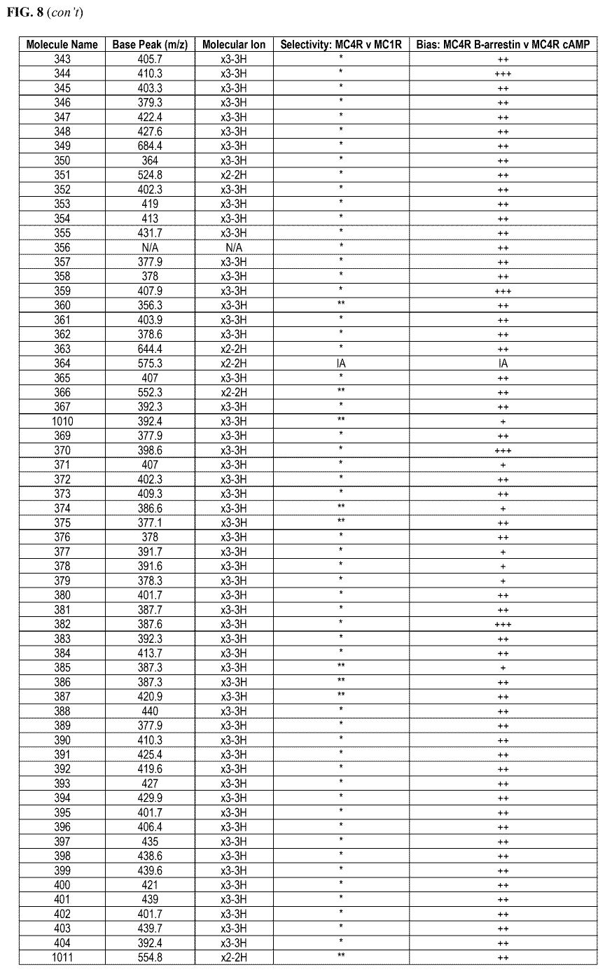

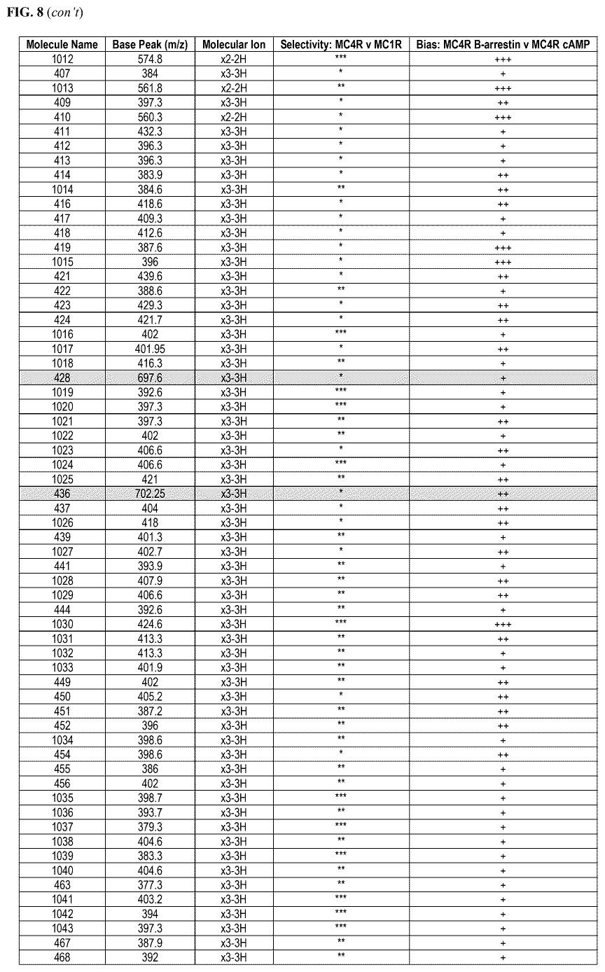

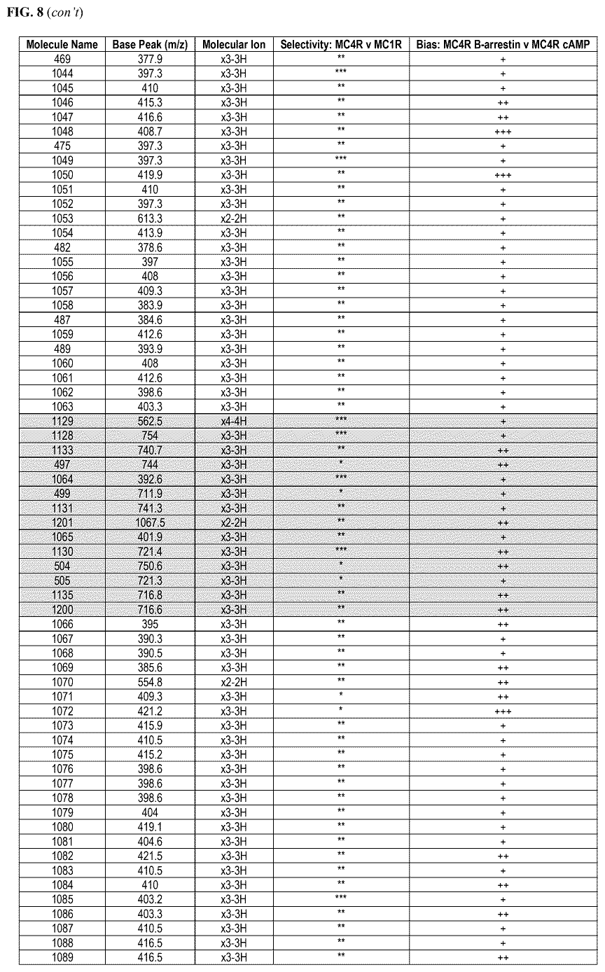

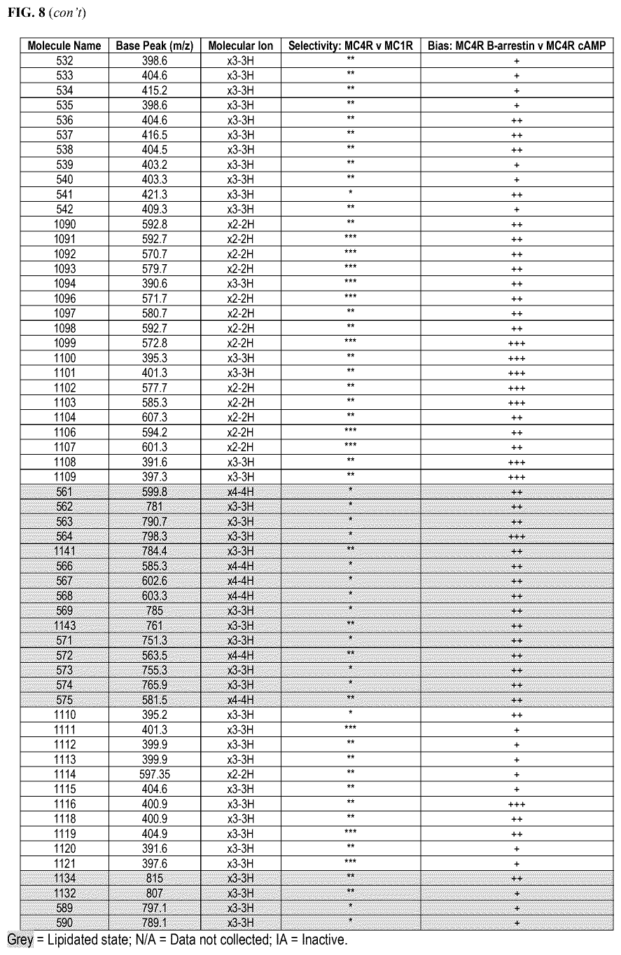

In embodiments, the API is a peptide, or a salt thereof, wherein the peptide is a peptide described in FIG. 8.

In embodiments, the API is a peptide, or a salt thereof, wherein the peptide is a peptide is molecule 1092:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1092 | D-Nar | Cys | Aib(O-cyclic) | Gln | D-Phe | Arg | Trp(6-F) | Cys | Ac | NH2 | Disulfide |

In embodiments, the API is a peptide, or a salt thereof, wherein the peptide is a peptide is molecule 1093:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1093 | D-Nar | Cys | Aib(O-cyclic) | Gln | D-Phe(4-F) | Arg | Trp(6-F) | Cys | Ac | NH2 | Disulfide |

In embodiments, the API is a peptide, or a salt thereof, wherein the peptide is a peptide is molecule 1094:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1094 | D-Nar | Cys | D-aMeOrn | Gln | D-Phe | Arg | Trp(6-F) | Cys | Ac | NH2 | Disulfide |

In embodiments, the API is a peptide, or a salt thereof, wherein the peptide is a peptide is molecule 1106:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1106 | D-Nar | Cys | Aib(O-cyclic) | Cit | D-Phe(4-F) | Arg | Trp(6-F) | Cys | Ac | NH2 | Disulfide |

In embodiments, the API is a peptide, or a salt thereof, wherein the peptide is a peptide is molecule 1107:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1107 | D-Nar | Cys | Aib(O-cyclic) | hCit | D-Phe(4-F) | Arg | Trp(6-F) | Cys | Ac | NH2 | Disulfide |

In embodiments, the API is a peptide, or a salt thereof, wherein the peptide is a peptide is molecule 1119:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1119 | D-Nar | Cys | Phg | 3Pal | D-Phe(4-F) | Arg | Trp(6-F) | Cys | Ac | NH2 | Disulfide |

In embodiments, the API is a peptide, or a salt thereof, wherein the peptide is a peptide is molecule 1158:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1158 | D-Nar | Cys | Aib(O-cyclic) | Gln | D-Phe(4-F) | Arg | Trp(6-F) | Pen | Ac | NH2 | Disulfide |

In embodiments, the API is an acetate salt of a peptide comprising the amino acid sequence as set forth in formula (III):

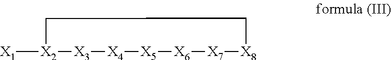

wherein in formula (III):
   X₁ is D-norarginine (D-Nar);
   X₂ is cysteine (Cys);
   X₃ is 3-Aminooxetane-3-carboxylic acid (Aib(O-cyclic);
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

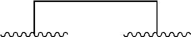 [SMILES: *CC*]

represents a disulfide bridge, and the peptide is capped with N-terminal acetyl.

In embodiments, the API is a trifluoroacetate salt of a peptide comprising the amino acid sequence as set forth in formula (III):

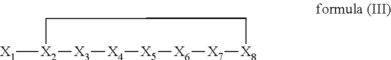

wherein in formula II):
   X₁ is D-norarginine (D-Nar);
   X₂ is cysteine (Cys);
   X₃ is 3-Aminooxetane-3-carboxylic acid (Aib(O-cyclic);
   X₄ is glutamine (Gln);
   X₅ is 4-fluoro-D-phenylalanine (D-Phe(4-F));
   X₆ is arginine (Arg);
   X₇ is 6-fluoro-L-tryptophan (Trp(6-F)); and
   X₈ is penicillamine (Pen), wherein

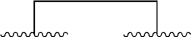 [SMILES: *CC*]

represents a disulfide bridge, and the peptide is capped with N-terminal acetyl.

In embodiments, the API is a bistrifluoroacetate salt of a peptide comprising the amino acid sequence as set forth in formula (III):

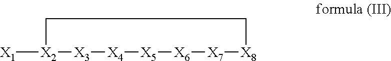

wherein in formula (III):
   X₁ is D-norarginine (D-Nar);
   X₂ is cysteine (Cys);
   X₃ is 3-Aminooxetane-3-carboxylic acid (Aib(O-cyclic);
   X₄ is glutamine (Gin);
   X₅ is 4-fluoro-D-phenylalanine (D-Pho (4-F);
   X₆ is arginine (Arg);
   X₇ is 6-fluoro-L-tryptophan (Trp(6-F); and
   X₈ is penidilamine (Pen), wherein

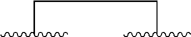 [SMILES: *CC*]

represents a disulfide bridge, and the peptide is capped with N-terminal acetyl.

In embodiments, the API is an acetate salt of peptide 1158:

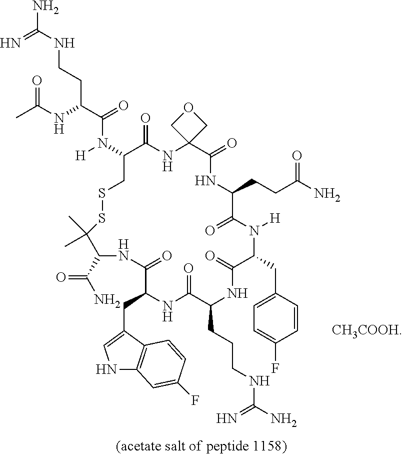

In embodiments, the API is a trifluoroacetate salt of peptide 1158:

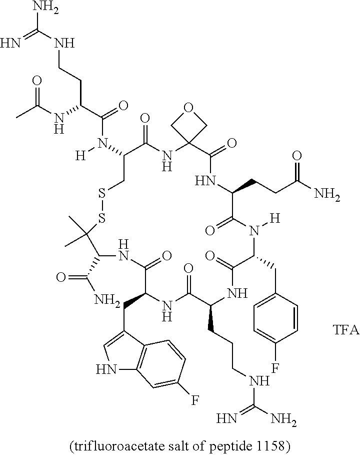

In embodiments, the API is a bistrifluoroacetate salt of peptide 1158:

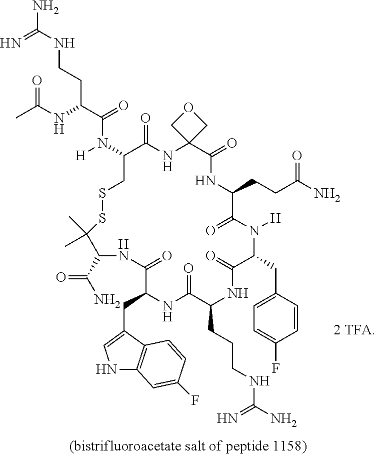

### Modified Peptides

In aspects, a method for achieving half-life extension is through chemical modification of molecules to extend their residence time in the serum. In embodiments, the API is a peptide, or a salt thereof, wherein the peptide is modified, for example, to achieve half-life extension. In embodiments, the peptides comprise a lipid-containing moiety grafted with linker fragments onto a peptide (e.g., without limitation the peptides of Table 1 and Table 2). In embodiments, the half-life of the peptides can be extended without substantially affecting the selectivity and efficacy parameters. In embodiments, the peptides are highly selective, B-arrestin biased MC4R agonists that can be modified for extended half-life in vivo.

In embodiments, the API is a peptide, or a salt thereof, wherein the peptide is lipidated. In a non-limiting example, a lipidated peptide further comprises one or more lipids conjugated to an amino acid of the peptide. In embodiments, the peptide of formula (I) comprises one or more lipids conjugated to one or more of X₁-X₈. In embodiments, the peptide of formula (I) comprises one or more lipids conjugated to X₁ and/or X₈. In embodiments, the one or more lipids comprise cysteine prenylation, N-terminal glycine myristylation, cysteine palmitoylation, and serine and/or lysine fatty acylation.

In embodiments, the peptide further comprises one or more amino acids conjugated to X₁ and/or X₈. In embodiments the one or more amino acids are selected from D-arginine (D-Arg), glycine (Gly), and L-Lys (AEEAc-AEEAc-L-γ-Glu-17-carboxyheptadecanoyl) (Lys*). In embodiments, the one or more amino acids are selected from Table 1 and Table 2. In embodiments, the one or more amino acid is modified. Non-limiting examples of modified amino acids include PEG groups (e.g., PEG-2), and lipids. In embodiments, the modified amino acid is Lys (AEEAc-AEEAc-L-γ-Glu-17-carboxyheptadecanoyl) (Lys*).

In embodiments, the peptide further comprises one or more lipids conjugated to X₁ and/or X₈. In embodiments, the peptide further comprises one or more lipids conjugated to the one or more amino acids conjugated to X₁ and/or X₈.

In embodiments, the peptide further comprises one or more PEG linkers conjugated to X₁ and/or X₈.

In embodiments, the peptide further comprises one or more dicarboxylic acid, e.g., a C12-C24 dicarboxylic acid.

In embodiments, one or more amino acid residues are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, the one or more amino acid residues conjugated to X₁ have the sequential designation of X₋₁, X₋₂, X₋₃, X₋₄, X₋₅, and so forth.

In embodiments, one or more amino acid residues are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, the one or more amino acid residues conjugated to X₈ have the sequential designation of X₉, X₁₀, X₁₁, X₁₂, X₁₃, X₁₄ and so forth.

In embodiments, the peptides of formula (I) or formula (II) comprises an N-terminal acetyl. In embodiments, the peptides of formula (I) or formula (II) comprises a C-terminal amide. In embodiments, the peptides of formula (I) or formula (II) comprises an N-terminal acetyl and C-terminal amide.

In embodiments, the peptide is capped with N-terminal acetyl and/or C-terminal amide groups.

In embodiments, the peptides of formula (I) or formula (II) comprises an N-terminal and/or C-terminal modification. In embodiments, the N-terminal modification comprises acetylation, propionylation, methylation, myristoylation, palmitoylation or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein R1 and R2 are selected from H or alkyl.

In embodiments, the peptide of formula (I) or formula (II) is selected from Table 1 and Table 2.

In embodiments, one or more amino acid residues are conjugated to X₈. In embodiments, the one or more amino acid residues conjugated to X₈ have the sequential designation of X₉, X₁₀, X₁₁, and X₁₂ as exemplified in Table 3, and/or cyclic structure.

In embodiments, the one or more amino acid residues conjugated to X₈ comprise an N-terminal acetyl and C-terminal amide and/or a cyclic structure as exemplified in Table 3.

TABLE 3
Exemplary peptides with of one or more amino acid residues conjugated to X₈. Cyclic peptides include bridge (e.g. disulfide) between X₂ and X₈.

| Molecule | | | | | | | | | | | | | | | Cyclic |
|---|---|---|---|---|---|---|---|---|---|---|---|---|---|---|---|
| Name | X₁ | X₂ | X₃ | X₄ | X₅ | X₆ | X₇ | X₈ | X₉ | X₁₀ | X₁₁ | X₁₂ | N-term | C-term | molecule |
| 115 | D-Nar | Cys | Aib(O-cyclic) | Gln | D-Phe(4-F) | Arg | Trp(6-F) | Cys | Gly | Gly | Gly | Lys* | Ac | NH2 | Disulfide |
| 116 | D-Nar | Cys | Aib(O-cyclic) | Gln | D-Phe(4-F) | Arg | Trp(6-F) | Cys | Gly | Gly | Lys* | | Ac | NH2 | Disulfide |
| 117 | D-Nar | Cys | Aib(O-cyclic) | Gln | D-Phe(4-F) | Arg | Trp(6-F) | Cys | Gly | Lys* | | | Ac | NH2 | Disulfide |
| 118 | D-Nar | Cys | Aib(O-cyclic) | Gln | D-Phe(4-F) | Arg | Trp(6-F) | Cys | Lys* | | | | Ac | NH2 | Disulfide |
| 119 | D-Nar | Cys | Aib(O-cyclic) | Gln | D-Phe(4-F) | Arg | Trp(6-F) | Cys | PEG1 | PEG1 | Lys* | | Ac | NH2 | Disulfide |
| 120 | D-Nar | Cys | Aib(O-cyclic) | Gln | D-Phe(4-F) | Arg | Trp(6-F) | Cys | D-Arg | Gly | Lys* | | Ac | NH2 | Disulfide |
| 121 | D-Nar | Cys | Aib(O-cyclic) | Gln | D-Phe(4-F) | Arg | Trp(6-F) | Cys | Pro | Phe | Lys* | | Ac | NH2 | Disulfide |
| 122 | D-Nar | Cys | Aib(O-cyclic) | Gln | D-Phe(4-F) | Arg | Trp(6-F) | Cys | Lys | Pro | Val | Lys* | Ac | NH2 | Disulfide |
| 126 | D-Nar | Cys | Phg | 3Pal | D-Phe(4-F) | Arg | Trp(6-F) | Cys | Lys* | | | | Ac | NH2 | Disulfide |
| 127 | D-Nar | Cys | Phg | 3Pal | D-Phe(4-F) | Arg | Trp(6-F) | Cys | Gly | Lys* | | | Ac | NH2 | Disulfide |
| 128 | D-Nar | Cys | Phg | 3Pal | D-Phe(4-F) | Arg | Trp(6-F) | Cys | Gly | Gly | Lys* | | Ac | NH2 | Disulfide |
| 129 | D-Nar | Cys | Phg | 3Pal | D-Phe(4-F) | Arg | Trp(6-F) | Cys | Lys | Pro | Val | Lys* | Ac | NH2 | Disulfide |
| 133 | D-Nar | Cys | D-aMeOrn | Gln | D-Phe | Arg | Trp(6-F) | Cys | Lys* | | | | Ac | NH2 | Disulfide |
| 134 | D-Nar | Cys | D-aMeOrn | Gln | D-Phe | Arg | Trp(6-F) | Cys | Gly | Lys* | | | Ac | NH2 | Disulfide |
| 135 | D-Nar | Cys | D-aMeOrn | Gln | D-Phe | Arg | Trp(6-F) | Cys | Gly | Gly | Lys* | | Ac | NH2 | Disulfide |
| 136 | D-Nar | Cys | D-aMeOrn | Gln | D-Phe | Arg | Trp(6-F) | Cys | Lys | Pro | Val | Lys* | Ac | NH2 | Disulfide |
| 281 | | A45 | Cys | His | D-Phe | Arg | Trp | Gaba | Cys | | | | Ac | NH2 | Disulfide |
| 282 | Arg | Gly | Cys | His | D-Phe | Arg | Trp | Gaba | Cys | | | | Ac | NH2 | Disulfide |
| 283 | | D-Phe | Cys | His | D-Phe | Arg | Trp | 5-Ava | Cys | | | | Ac | NH2 | Disulfide |
| 284 | | Arg | Asp | His | D-Phe | Arg | Trp | Ala | Lys | | | | Ac | NH2 | Lactam |
| 285 | Arg | Gly | Asp | His | D-Phe | Arg | Trp | Ala | Lys | | | | Ac | NH2 | Lactam |
| 286 | | A45 | Asp | His | D-Phe | Arg | Trp | Gaba | Lys | | | | Ac | NH2 | Lactam |
| 302 | Arg | Cys | D-Ala | His | D-Phe | Arg | Trp | Cys | Gln | | | | Ac | NH2 | Disulfide |
| 303 | Arg | Cys | D-Ala | His | D-Phe | Arg | Trp | Cys | Tyr | | | | Ac | NH2 | Disulfide |
| 304 | Arg | Cys | D-Ala | His | D-Phe | Arg | Trp | Cys | D-Orn | | | | Ac | NH2 | Disulfide |
| 305 | Arg | Cys | D-Ala | His | D-Phe | Arg | Trp | Cys | Phg | | | | Ac | NH2 | Disulfide |
| 306 | Arg | Cys | D-Ala | His | D-Phe | Arg | Trp | Cys | Arg | | | | Ac | NH2 | Disulfide |
| 307 | Arg | Cys | D-Ala | His | D-Phe | Arg | Trp | Cys | homoPhe | | | | Ac | NH2 | Disulfide |
| 308 | Arg | Cys | D-Ala | His | D-Phe | Arg | Trp | Cys | Gaba | | | | Ac | NH2 | Disulfide |
| 309 | Arg | Cys | D-Ala | His | D-Phe | Arg | Trp | Cys | Beta-homoArg | | | | Ac | NH2 | Disulfide |
| 310 | Arg | Cys | D-Ala | His | D-Phe | Arg | Trp | Cys | Ala | Chg | | | Ac | NH2 | Disulfide |
| 311 | Arg | Cys | D-Ala | His | D-Phe | Arg | Trp | Cys | Ala | Leu | | | Ac | NH2 | Disulfide |
| 312 | Arg | Cys | D-Ala | His | D-Phe | Arg | Trp | Cys | Pro | Glu | | | Ac | NH2 | Disulfide |
| 313 | Arg | Cys | D-Ala | His | D-Phe | Arg | Trp | Cys | Ala | Arg | | | Ac | NH2 | Disulfide |
| 314 | Arg | Cys | D-Ala | His | D-Phe | Arg | Trp | Cys | Gly | Gly(thien-3-yl) | | | Ac | NH2 | Disulfide |
| 315 | Arg | Cys | D-Ala | His | D-Phe | Arg | Trp | Cys | Pro | Phe | | | Ac | NH2 | Disulfide |
| 316 | Arg | Cys | D-Ala | His | D-Phe | Arg | Trp | Cys | Gly | Pro | | | Ac | NH2 | Disulfide |
| 317 | Arg | Pro | Cys | His | D-Phe | Arg | Trp | Gaba | Cys | | | | Ac | NH2 | Disulfide |
| 318 | Arg | Ala | Cys | His | D-Phe | Arg | Trp | Gaba | Cys | | | | Ac | NH2 | Disulfide |
| 319 | Arg | D-Ala | Cys | His | D-Phe | Arg | Trp | Gaba | Cys | | | | Ac | NH2 | Disulfide |
| 320 | Arg | D-Pro | Cys | His | D-Phe | Arg | Trp | Gaba | Cys | | | | Ac | NH2 | Disulfide |
| 321 | D-Arg | Pro | Cys | His | D-Phe | Arg | Trp | Gaba | Cys | | | | Ac | NH2 | Disulfide |
| 322 | D-Arg | Gly | Cys | His | D-Phe | Arg | Trp | Gaba | Cys | | | | Ac | NH2 | Disulfide |
| 323 | D-Arg | D-Ala | Cys | His | D-Phe | Arg | Trp | Gaba | Cys | | | | Ac | NH2 | Disulfide |
| 324 | D-Arg | Ala | Cys | His | D-Phe | Arg | Trp | Gaba | Cys | | | | Ac | NH2 | Disulfide |
| 325 | Arg | D-Pro | Asp | His | D-Phe | Arg | Trp | Ala | Lys | | | | Ac | NH2 | Lactam |
| 326 | Arg | Pro | Asp | His | D-Phe | Arg | Trp | Ala | Lys | | | | Ac | NH2 | Lactam |
| 327 | Arg | Ala | Asp | His | D-Phe | Arg | Trp | Ala | Lys | | | | Ac | NH2 | Lactam |
| 328 | Arg | Ala(2Me) | Asp | His | D-Phe | Arg | Trp | Ala | Lys | | | | Ac | NH2 | Lactam |
| 329 | Arg | D-Ala | Asp | His | D-Phe | Arg | Trp | Ala | Lys | | | | Ac | NH2 | Lactam |
| 330 | D-Arg | Gly | Asp | His | D-Phe | Arg | Trp | Ala | Lys | | | | Ac | NH2 | Lactam |
| 331 | D-Arg | Ala | Asp | His | D-Phe | Arg | Trp | Ala | Lys | | | | Ac | NH2 | Lactam |
| 332 | D-Arg | D-Ala | Asp | His | D-Phe | Arg | Trp | Ala | Lys | | | | Ac | NH2 | Lactam |
| 333 | | Arg | Cys | His | D-Phe | Arg | Trp | 5-Ava | Cys | | | | Ac | NH2 | Disulfide |
| 334 | | Arg | Cys | His | D-Phe | Arg | Trp | Gaba | Cys | | | | Ac | NH2 | Disulfide |
| 335 | | Arg | Cys | His | D-Phe | Arg | Trp | bAla | Cys | | | | Ac | NH2 | Disulfide |
| 336 | Arg | Gly | Asp | His | D-Phe | Arg | Trp | D-Orn | Lys | | | | Ac | NH2 | Lactam |
| 337 | Arg | Gly | Asp | His | D-Phe | Arg | Trp | Pro | Lys | | | | Ac | NH2 | Lactam |
| 338 | Arg | Gly | Asp | His | D-Phe | Arg | Trp | Gly | Lys | | | | Ac | NH2 | Lactam |
| 339 | Arg | Gly | Asp | His | D-Phe | Arg | Trp | Leu | Lys | | | | Ac | NH2 | Lactam |
| 340 | Arg | Gly | Asp | His | D-Phe | Arg | Trp | D-Leu | Lys | | | | Ac | NH2 | Lactam |
| 341 | Arg | Ala | Asp | His | D-Phe | Arg | Trp | Ala(2-Me) | Lys | | | | Ac | NH2 | Lactam |
| 342 | Arg | Gly | Cys | His | D-Phe | Arg | Trp | 4-amino-4- | Cys | | | | Ac | NH2 | Disulfide |
| | | | | | | | | methylpentanoic acid | | | | | | | |
| 343 | Arg | Gly | Cys | His | D-Phe | Arg | Trp | Inp | Cys | | | | Ac | NH2 | Disulfide |
| 344 | Arg | Gly | Cys | His | D-Phe | Arg | Trp | (1S,3R)-3- | Cys | | | | Ac | NH2 | Disulfide |
| | | | | | | | | aminocyclohexane- | | | | | | | |
| | | | | | | | | 1-carboxylic acid | | | | | | | |
| 352 | Arg | Cys | D-Ala | His | D-Phe | Arg | Trp | Cys | Ser | | | | Ac | NH2 | Disulfide |
| 353 | Arg | Cys | D-Ala | His | D-Phe | Arg | Trp | Cys | D-His | | | | Ac | NH2 | Disulfide |
| 354 | Arg | Cys | D-Ala | His | D-Phe | Arg | Trp | Cys | 0FU | | | | Ac | NH2 | Disulfide |
| 355 | Arg | Cys | D-Ala | His | D-Phe | Arg | Trp | Cys | Nva(Ph) | | | | Ac | NH2 | Disulfide |
| 356 | Arg | Cys | D-Ala | His | D-Phe | Arg | Trp | Cys | Ala(4-piperidyl) | | | | Ac | NH2 | Disulfide |
| 371 | Arg | Cys | D-Ala | His | D-Phe | Arg | Trp | Cys | PEG1 | | | | Ac | NH2 | Disulfide |
| 372 | Arg | Gly | Cys | His | D-Phe | Arg | Trp | PEG1 | Cys | | | | Ac | NH2 | Disulfide |
| 373 | Arg | Gly | Lys | His | D-Phe | Arg | Trp | PEG1 | Asp | | | | Ac | NH2 | Lactam |
| 384 | Arg | Gly | Asp | His | D-Phe | Arg | Trp | Orn | Lys | | | | Ac | NH2 | Lactam |
| 389 | | D-Arg | Cys | His | D-Phe | Arg | Trp | Gaba | Cys | | | | Ac | NH2 | Disulfide |
| 398 | Arg | Cys | D-Ala | His | D-Phe | Arg | Trp | Cys | Gly | Chg | | | Ac | NH2 | Disulfide |
| 402 | Arg | Gly | Cys | His | D-Phe | Arg | Trp | Me-Gaba | Cys | | | | Ac | NH2 | Disulfide |
| 403 | Arg | Cys | D-Ala | His | D-Phe | Arg | Trp | Cys | Gly | Nar | | | Ac | NH2 | Disulfide |
| 404 | Arg | Gly | Cys | His | D-Phe | Arg | Trp | bAla | Cys | | | | Ac | NH2 | Disulfide |
| 416 | Arg | Gly | Asp | His | D-Phe | Arg | Trp | D-Glu | Lys | | | | Ac | NH2 | Lactam |
| 417 | Arg | Ala | Asp | His | D-Phe | Arg | Trp | Ser | Lys | | | | Ac | NH2 | Lactam |
| 418 | Arg | D-Pro | Asp | His | D-Phe | Arg | Trp | Ala | Lys | | | | Ac | NH2 | Lactam |
| 423 | Arg | Cys | D-Ala | His | D-Phe | Arg | Trp | Cys | D-Ala | Pro | | | Ac | NH2 | Disulfide |
| 497 | D-Nar | Cys | L-aMeGlu | His | D-Phe | Arg | Trp(6-Me) | Cys | Gly | Gly(thien-3-yl) | Lys* | | Ac | NH2 | Disulfide |
| 1201 | D-Nar | Cys | L-aMeAsp | His | D-Phe | Arg | Trp(6-Me) | Cys | Gly | Gly | Lys* | | Ac | NH2 | Disulfide |
| 505 | beta-homoArg | Cys | L-aMeAsp | His | D-Phe | Arg | Trp(6-Me) | Cys | Gly | Gly | Lys* | | Ac | NH2 | Disulfide |
| 1200 | D-Arg | Cys | L-aMeAsp | His | D-Phe | Arg | Trp(6-Me) | Cys | Gly | Gly | Lys* | | Ac | NH2 | Disulfide |

In embodiments, the API is a peptide, or a salt thereof, wherein the peptide is a peptide listed in any of the tables disclosed herein.

In embodiments, the API is a peptide, or a salt thereof, wherein the peptide (e.g., without limitation, peptides of formula (I) or formula (II)) comprises additional substituents and/or functional groups that further modify the properties and/or function of the peptide.

In embodiments, the peptide comprises a half-life extending moiety. Non-limiting examples of half-life extending moieties include polyethylene glycol (PEG), recombinant PEG mimetics, glycosylation of carbohydrates, Fc-fusion proteins or conjugates, albumin fusion proteins or conjugates (including fusions with albumin-binding proteins and peptides that, in turn, recruit albumin molecules), polypropylene glycol (PPG), XTEN fusion protein or conjugates, or a combination thereof.

In embodiments, the peptides are pegylated. In some embodiments, attachment of the PEG moiety increases the half-life and/or reduces the immunogenicity of the peptide. In some embodiments, wherein the PEG-based moiety comprises one or more of poly(ethylene glycol) (PEG), an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker: a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG): a copper-free click chemistry PEG-based linker (e.g. DBCO-PEG, BCN-PEG): a Cu-catalyzed click chemistry PEG-based linker (e.g. propargyl-PEG): a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-pAsp, PEG-pGlu, PEG-b-PMPMC, and PEG-PGLA.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide further comprises a therapeutic, diagnostic, and/or imaging moiety. Non-limiting examples of therapeutic, diagnostic, and/or imaging moiety include a small molecule, a biological (e.g., without limitation, a biopolymer, a protein, a nucleic acid, a polysaccharide), or a radionuclide.

In embodiments, the therapeutic, diagnostic, and/or imaging moiety is the functional and clinically significant part of the active ingredient substance(s) present in a medicinal product. In embodiments, the medicinal product comprises orlistat, phentermine-topiramate, naltrexone-bupropion, liraglutide, retatrutide, tirzepatide, semaglutide, and setmelanotide.

In embodiments, the therapeutic, diagnostic, and/or imaging moiety comprises an antibody or antigen-binding fragment thereof, an aptamer, a peptide, a biological ligand (e.g., including a glycoconjugate), lipid, sterol, cholesterol or derivative thereof, integrin, RGD peptide, or cell-penetrating peptide (CPP). In embodiments, the moiety is selected from a single-domain antibody, a single chain antibody, a bi-specific antibody, a recombinant heavy-chain-only antibody (VHH), a single-chain antibody (scFv), a shark heavy-chain-only antibody (VNAR), a microprotein (cysteine knot protein, knottin), a DARPin, a tetranectin, an affibody, a transbody, an anticalin, an AdNectin, an affilin, a microbody, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′) 2, and a peptide mimetic molecule. Various non-limiting examples of ligand-binding platforms are described in US Patent Nos, or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties.

In embodiments, the disclosure provides conjugates comprising a peptide or pharmaceutical composition of the present disclosure (e.g., without limitation, a peptide of formula (I) or formula (II)) conjugated to or co-formulated with a therapeutic agent or therapeutic moiety).

In embodiments, the therapeutic moiety or therapeutic agent comprises incretin, an incretin analogue, or a modulator of an incretin receptor.

In embodiments, the modulator is an agonist. In embodiments, the agonist is an agonist of GLP-1, GIP, and/or glucagon receptor. In embodiments, the agonist is a GLP-1 analogue. In embodiments, the GLP-1 analogue comprises a non-canonical amino acid. In embodiments, the GLP-1 analogue comprises tirzepatide, liraglutide, retatrutide, exenatide, lixisenatide, semaglutide or a semaglutide derivative.

In embodiments, the agonist is an agonist of GIP receptor. In embodiments, the agonist is a dual GLP-1-GIP receptor co-agonist. In embodiments, the agonist is a triple hormone receptor agonist. In embodiments, the agonist comprises retatrutide.

In embodiments, the agonist is an agonist of the amylin receptor. In embodiments, the agonist is an amylin analogue. In embodiments, the amylin analogue is cagrlintide. In embodiments, the agonist is selected from a PYY (3-36) analogue, an orexin analogue, and a leptin analog.

In embodiments, the therapeutic agent is a clinically significant part of the active ingredient substance(s) present in a medicinal product. In embodiments, the medicinal product comprises orlistat, phentermine-topiramate, naltrexone-bupropion, liraglutide, retatrutide, semaglutide, tirzepatide, and setmelanotide.

In embodiments, the API is a peptide, or a salt thereof, wherein the peptide is labeled with a fluorescent compound. In a non-limiting example, when the fluorescently labeled peptide is exposed to light of the proper wavelength, its presence and/or amount can then be detected. In embodiments, fluorescent labeling compounds are selected from fluorescein isothiocyanate, rhodamine, phycoerythrin, phycocyanin, allophycocyanin, o-phthaldehyde and fluorescamine. In embodiments, the peptide can also be detectably labeled using fluorescence emitting metals such as 152Eu, or others of the lanthanide series. In embodiments, the metal is attached to the peptide using such metal chelating groups as diethylenetriaminepentacetic acid (DTPA) or ethylenediaminetetraacetic acid (EDTA). In embodiments, the peptide is detectably labeled by coupling to a chemiluminescent compound. In a non-limiting example, the presence of the chemiluminescent-tagged antibody coupled to the peptide is determined by detecting luminescence that arises during the course of a chemical reaction. In embodiments, chemiluminescent labeling compounds are selected from luminol, isoluminol, theromatic acridinium ester, imidazole, acridinium salt and oxalate ester. In embodiments, a bioluminescent compound may be used to label the peptide. In a non-limiting example, the presence of a bioluminescent protein is determined by detecting the presence of luminescence. In embodiments, bioluminescent compounds for purposes of labeling are luciferin, luciferase and aequorin.

In embodiments, the disclosure provides a protein comprising a peptide disclosed herein (e.g., without limitation, a peptide of formula (I) or a peptide of formula (II)).

In embodiments, the protein has a size of at least about 10 amino acid residues, or at least about 15 resides, or at least about 20 residues, or at least about 25 residues, or at least about 30) residues, or at least about 35 residues, or at least about 40) residues, or at least about 45 residues, or at least about 50 residues, or at least about 60 residues, or at least about 70) residues, or at least about 80) residues, or at least about 90 residues, or at least about 100 residues, or at least about 250) residues, or at least about 500 residues, at least about 750) residues, at least about 1,000 residues, at least about 1,250) residues, at least about 1,500 residues, at least about 1,750) residues, at least about 2,000 residues, at least about 3,000 residues, at least about 4,000 residues, or at least about 5,000 residues.

In embodiments, the disclosure provides a nucleic acid encoding the peptide disclosed herein, or the protein of disclosed herein. In embodiments, the disclosure provides a system for encoding one or more non-canonical amino acids. Site-specific incorporation of unnatural amino acids with orthogonal chemical reactivity into proteins enables the synthesis of structurally defined protein conjugates. Amino acids containing ketone, azide, alkyne, alkene, and tetrazine side chains can be genetically encoded in response to nonsense and frameshift codons. Kim, Chan Hyuk et al. “Protein conjugation with genetically encoded unnatural amino acids.” Current opinion in chemical biology vol. 17,3 (2013): 412-9.

In embodiments, the disclosure provides a solid synthesis device conjugated to a peptide disclosed herein (e.g., without limitation, a peptide of formula (I) or formula (II)), a protein disclosed herein, a nucleic acid disclosed herein, and/or an amino acid therein.

In embodiments, the solid synthesis device is conjugated to one or more amino acids, wherein the one or more amino acids are intermediates in the synthesis of a peptide of the present disclosure (e.g., without limitation, a peptide of formula (I) or formula (II)), a protein of the present disclosure, and/or a nucleic acid of the present disclosure. In embodiments, a non-limiting example of a solid synthesis device comprises covalently binding the peptide of the present disclosure onto a solid support material and synthesized step-by-step in a single reaction vessel utilizing selective protecting group chemistry. In embodiments, the peptides of the present disclosure are synthesized from the carbonyl group side (C-terminus) to amino group side (N-terminus) of the amino acid chain in the solid-phase peptide synthesis method.

In embodiments, the peptide is described from N-terminus to C-terminus unless stated otherwise.

In embodiments, the solid synthesis device is conjugated to about 1, about 2, about 3, about 4, about 5, about 6, about 7, about 8, or about 9, about 10, or about 10 or more amino acids, wherein the one or more amino acids are intermediates in the synthesis of a peptide of the present disclosure (e.g., without limitation, a peptide of formula (I) or formula (II)), a protein of the present disclosure, and/or a nucleic acid of the present disclosure.

In embodiments, the solid synthesis device comprises a solid phase material or resin. In embodiments, the resin is selected from core resin, Merrifield resin, hydroxymethyl resin, and amino core resin. In embodiments, the core resin comprises material selected from polystyrene polyacrylate, polyacrylamide, and polyethylene glycol. In embodiments, the polystyrene resin is crosslinked, uncrosslinked, or linear.

In embodiments, the solid synthesis device comprises aminomethyl resin or 4-methylbenzhydryl amine resin.

In embodiments, the solid synthesis device comprises polystyrene beads.

In embodiments, the size of the bead is about or at least about 1 micron, or about 5 microns, or about 10 microns, or about 20 microns, or about 30 microns, or about 40 microns, or about 50 microns, or about 75 microns, or about 100 microns, or about 200 microns, or about 300 microns, or about 400 microns, or about 500 microns, or about 600 microns, or about 700 microns, or about 800 microns in diameter.

In embodiments, the solid synthesis device comprises dosage forms such as pre-filled syringes, (including auto-injectors), patches, containers, solutions, suspensions, emulsion, drops, tablets, pills, pellets, capsules, capsules containing liquids, powders, sustained-release formulations, emulsions, aerosols, sprays, and suspensions.

### Salts of Peptides

In embodiments, API is a salt of a peptide, as disclosed herein. Salts of peptides and methods of designing and preparing thereof can be found in U.S. Provisional Application 63/926,069, the contents of which are hereby incorporated by reference herein in their entirety.

In embodiments, the salts of peptides described herein demonstrate enhanced MC4R function. In embodiments, the salts of peptides are selective melanocortin 4 receptor (MC4R) agonists. In embodiments, the salts of peptides described herein are MC4R agonists that display superior selectivity towards MC4R as compared with the other melanocortin receptors (such as MC1R).

In embodiments, the salts of peptides described herein display varying activity on G-protein coupled pathways stemming from the MC4R, namely one or more of Gs-coupled (e.g. CAMP), Gq-coupled, and B-arrestin dependent signaling pathways.

In embodiments, the salts of peptides of the present disclosure have increased in vitro selectivity and potency, in vivo effectiveness, pharmacokinetic attributes, and/or stability when compared to salts of other melanocortin receptor binding peptides or salts thereof.

Non-limiting examples of salts include acetate salts, trifluoroacetate salts, phosphate salts, phosphite salts, propionate salts, chloride salts, fumarate salts, citrate salts, tartrate salts, oxalate salts, succinate salts, mandelate salts, methanesulfonate salts, p-toluenesulfonate salts, bromide salts, iodide salts, hydroxide salts, sulfate salts, sulfite salts, nitrate salts, malate salts, maleate salts, aspartate salts, glutamate salts, lactate salts, gluconate salts, benzoate salts, salicylate salts, ethanesulfonate salts, naphthalenesulfonate salts, or camphorsulfonate salts.

In embodiments, the API is a salt, wherein the salt is a pharmaceutically acceptable salt. The phrase “pharmaceutically acceptable salt” as used herein, refers to pharmaceutically acceptable organic or inorganic salts of a parent compound (e.g. a peptide of the disclosure). Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. In embodiments, a pharmaceutically acceptable salt involves the inclusion of another molecule such as an acetate ion, a succinate ion or other counterion. In embodiments, the counterion is any organic or inorganic moiety that stabilizes the charge on the parent compound (e.g. peptide). In embodiments, a pharmaceutically acceptable salt has more than one charged atom in its structure. For example, instances where multiple charged atoms (e.g. multiple charged atoms on the peptide) are part of the pharmaceutically acceptable salt have multiple counterions. In embodiments, a pharmaceutically acceptable salt has one or more charged atoms and/or one or more counterions.

In embodiments each counterion is independently selected from an acetate ion, a trifluoroacetate ion, a phosphate ion, a phosphite ion, a propionate ion, a chloride ion, a fumarate ion, a citrate ion, a tartrate ion, an oxalate ion, a succinate ion, a mandelate ion, a methanesulfonate ion, a p-toluenesulfonate ion, a bromide ion, a iodide ion, a hydroxide ion, a sulfate ion, a sulfite ion, a nitrate ion, a malate ion, a maleate ion, a aspartate ion, a glutamate ion, a lactate ion, a gluconate ion, a benzoate ion, a salicylate ion, a ethanesulfonate ion, a naphthalenesulfonate ion, and a camphorsulfonate ion, optionally wherein each counterion is an acetate ion, optionally wherein each counterion is a trifluoroacetate ion.

In embodiments, each counterion is independently selected from a sulfate ion, a citrate ion, an acetate ion, an oxalate ion, a chloride ion, a bromide ion, an iodide ion, a nitrate ion, a bisulfate ion, a phosphate ion, an acid phosphate ion, an isonicotinate ion, a lactate ion, a salicylate ion, an acid citrate ion, a tartrate ion, an oleate ion, a tannate ion, a pantothenate ion, a bitartrate ion, a ascorbate ion, a succinate ion, a maleate ion, a gentisinate ion, a fumarate ion, a gluconate ion, a glucuronate ion, a saccharate ion, a formate ion, a benzoate ion, a glutamate ion, a methanesulfonate “mesylate” ion, an ethanesulfonate ion, a benzenesulfonate ion, a p-toluenesulfonate ion, and a pamoate (i.e., 1,1′-methylene-bis-(2-hydroxy-3-naphthoate)) ion.

In embodiments, a peptide of a salt of the present disclosure can have one or more charged atoms, and the salt has one or more counterions, e.g., acetate ions. In embodiments, the peptide comprises one, or two, or three, or four, or five, or six, or more charged atoms, and the salt comprises one, or two, or three, or four, or five, or six, or more counter ions. In embodiments, the counterions are acetate ions or trifluoroacetate ions. In embodiments, the counterions are acetates. In embodiments, the counterions are trifluoroacetates.

In embodiments, the salts of peptides of the disclosure are acetate salts. In embodiments, the peptide comprises one charged atom, and the salt comprises one acetate counterion. In embodiments, the peptide comprises two charged atoms, and the salt comprises two acetate counterions (e.g. bisacetate salt). In embodiments, the peptide comprises three charged atoms, and the salt comprises three acetate counterions (e.g. trisacetate salt). In embodiments, the peptide comprises four charged atoms, and the salt comprises four acetate counterions (e.g. tetraacetate salt).

In embodiments, the salts of peptides of the disclosure are trifluoroacetate salts. In embodiments, the peptide comprises one charged atom, and the salt comprises one trifluoroacetate counterion. In embodiments, the peptide comprises two charged atoms, and the salt comprises two trifluoroacetate counterions (e.g. bistrifluoroacetate salt). In embodiments, the peptide comprises three charged atoms, and the salt comprises three trifluoroacetate counterions (e.g. tristrifluoroacetate salt). In embodiments, the peptide comprises four charged atoms, and the salt comprises four trifluoroacetate counterions (e.g. tetrafluoroacetate salt).

### API (Peptide or Salt) Selectivity

Illustrative, non-limiting examples of MC4R agonist peptides can be found in PCT/US2025/030832, the contents of which are hereby incorporated by reference herein in their entirety.

In embodiments, the API is a peptide, or a salt thereof, wherein the peptide or the salt (e.g., a peptide of formula (I) or formula (II)) demonstrates increased selectivity for MC4R over MC1R when administered to a subject compared to a control.

In embodiments, the peptide or the salt demonstrates increased selectivity for MC4R over MC1R as measured by an in vitro, ex vivo, or in vivo assay when compared to a control.

In embodiments, the peptide or the salt demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide or the salt is administered, or to a pre-treatment or non-treatment state.

In embodiments, the peptide or the salt demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide or the salt is administered, or to a pre-treatment or non-treatment state.

In embodiments, the peptide or the salt demonstrates an increased ratio of MC4R intracellular signaling to MC1R intracellular signaling in a subject when administered to a subject compared to a control.

In embodiments, the peptide or the salt demonstrates an increased ratio of MC4R intracellular signaling to MC1R intracellular signaling as measured by an in vitro, ex vivo, or in vivo assay when compared to a control.

In embodiments, the peptide or the salt demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide or the salt is administered, or to a pre-treatment or non-treatment state.

In embodiments, the peptide or the salt demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is or the salt administered, or to a pre-treatment or non-treatment state.

In embodiments, the peptide or the salt demonstrates enhanced melanocortin 4 receptor (MC4R) function in a subject when compared to before the peptide or the salt is administered or to a pre-treatment or non-treatment state, or a subject treated with control.

In embodiments, the peptide or the salt demonstrates decreased melanocortin 1 receptor (MC1R) function in a subject when compared to before the peptide or the salt is administered or to a pre-treatment or non-treatment state, or a subject treated with control.

In embodiments, the peptide or the salt demonstrates enhanced melanocortin 4 receptor (MC4R) function as measured by an in vitro, ex vivo, or in vivo assay when compared to a control.

In embodiments, the peptide or the salt demonstrates decreased melanocortin 1 receptor (MC1R) function as measured by an in vitro, ex vivo, or in vivo assay when compared to a control.

In embodiments, the peptide or the salt demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide or the salt is administered, or to a pre-treatment or non-treatment state.

In embodiments, the peptide or the salt demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide or the salt is administered, or to a pre-treatment or non-treatment state.

In embodiments, the cyclic peptide or the salt thereof demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide or the salt thereof is administered, or to a pre-treatment or non-treatment state.

In embodiments, the cyclic peptide or the salt thereof demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide or the salt thereof is administered, or to a pre-treatment or non-treatment state.

In embodiments, the control is selected from LY2112688, setmelanotide, ring peptide compound from KR2002-0038400, NDP-aMSH, melanotan-II, a-MSH, and bremelanotide.

In embodiments, the control comprises a vehicle control such as saline or mineral oil.

In embodiments, the control comprises a subject that has not been administered the peptide or pharmaceutical composition of the present disclosure. In embodiments, the control comprises a subject that has been administered LY2112688, setmelanotide, Ring Peptide Compound from KR2002-0038400, NDP-aMSH, Melanotan-II, a-MSH, and/or Bremelanotide. In embodiments, the control is a subject that is in a pre-treatment or non-treatment state.

In embodiments, the in vitro, ex vivo, or in vivo assay that demonstrates increased selectivity for MC4R over MC1R is selected from, one or more of: CAMP assay, B-arrestin assay, ELISA, electro-chemiluminescence assays, radioimmunoassay (RIA), fluorescence immunoassay (FIA), thermal shift assay, LC-MS detection, surface plasmon resonance (SPR), and bio-layer interferometry (BLI), including combinations of the foregoing. In embodiments, other assays to test the functionality of the protein include chromatographic, electrophoretic and chemical techniques. In embodiments, exemplary assays and technique include, but are not limited to, luciferase assays, bimolecular fluorescence complementation, affinity electrophoresis, pull down assays, ELISA, western blots, immunoblotting, high-through screening of protein interaction, in vivo crosslinking of protein complexes, chemical cross linking, chemical cross linking followed by high mass MALDI mass spectrometry, quantitative immunoprecipitation combined with knock-down (QUICK), proximity ligation assay in situ, surface plasmon resonance (SPR), dual polarization interferometry (DPI), static light scattering (SLS), dynamic light scattering (DLS), flow-induced dispersion analysis (FIDA), fluorescence polarization/anisotropy, fluorescence resonance energy transfer (FRET), bio-layer interferometry (BLI), rotating cell-based ligand binding assay using radioactivity or fluorescence, single color reflectometry (SCORE), and protein NMR, including combinations of the foregoing.

Table 22 provides a list of residue shorthands and associated full residue name.

TABLE 22
Amino Acid Residues

| Residue Shorthand | Full Residue Name |
|---|---|
| (1S,3R)-3-aminocyclohexane-1-carboxylic acid | (1S,3R)-3-aminocyclohexane-1-carboxylic acid |
| (aMe)D-Phe | alpha-methyl-D-Phenylalanine |
| 0FU | 1,2-Phenylenedimethanamine |
| 2Nal | 3-(2-naphthyl)-L-Alanine |
| 2Pal | 3-(2-Pyridyl)-L-Alanine |
| 3-aminoazetidine-3-carboxylic acid | 3-aminoazetidine-3-carboxylic acid |
| 3-Pal | 3-(3-Pyridyl)-L-alanine |
| 4-amino-4-methylpentanoic acid | 4-amino-4-methylpentanoic acid |
| 4-aminooxane-4-carboxylic acid | 4-aminooxane-4-carboxylic acid |
| 4-Guanidinobutyric acid | 4-Guanidinobutyric acid |
| 4-Pal | 3-(4-pyridyl)-L-alanine |
| 5-Ava | 5-aminovaleric acid |
| Abu | L-2-Aminobutyric acid |
| Ac3c | 1-aminocyclopropane-1-carboxylic acid |
| Ac4c | 1-Aminocyclobutanecarboxylic acid |
| Ac6c | 1-aminocyclohexane-1-carboxylic acid |
| Aib | 2-Aminoisobutyric acid |
| Aib(O-cyclic) | 3-Aminooxetane-3-carboxylic acid |
| Ala(2-furyl) | 3-(2-Furyl)-L-Alanine |
| Ala(2Me) | 2-Methyl-Alanine |
| Ala(4-piperidyl) | 3-(4-Piperidinyl)-L-alanine |
| Ala(cPent) | 3-Cyclopentane-L-Alanine |
| Ame-L-Abu | Isovaline |
| aMeGly(allyl) | (S)-2-Amino-2-methylpent-4-enoic acid |
| aMeOrn | alpha-Methyl-L-Ornithine |
| Arg | Arginine |
| Arg(Me) | N-Monomethyl-L-Arginine, Tilarginine |
| Asp | Aspartic acid |
| bAc4c | 1-(Aminomethyl)cyclobutanecarboxylic acid |
| bAc5c | 1-(Aminomethyl)cyclopentanecarboxylic acid |
| bAla | beta-alanine |
| bDGLN | beta-D-Glutamine |
| beta-Ala(2-Me) | 3-amino-2,2-dimethyl-propionic acid |
| Beta-homoArg | beta-homo-L-arginine |
| bhDSER | beta-homo-D-Serine |
| bhGlu | beta-L-homoglutamic acid |
| Chg | L-Cyclohexylglycine |
| Cit | Citrulline |
| Cyclo-Leu | Cycloleucine |
| Cyclo-Leu(3-ene) | 1-Aminocyclopent-3-enecarboxylic acid |
| Cys | Cysteine |
| D-2Nal | 3-(2-Naphthyl)-D-Alanine |
| D-3Thi | 3-(3-Thienyl)-D-Alanine |
| D-Abu | D-2-Aminobutyric acid |
| D-Ala | D-Alanine |
| D-aMeAsp | alpha-methyl-D-aspartic acid |
| D-aMeLeu | Alpha-methyl-D-Leucine |
| D-aMeOrn | alpha-Methyl-D-Ornithine |
| D-aMeSer | Alpha-methyl-D-serine |
| D-aMeVal | alpha-Methyl-D-Valine |
| D-Arg | D-Arginine |
| D-Asp | D-Aspartic acid |
| D-bhGlu | beta-D-homoglutamic acid |
| D-Bpa | 4-Benzoyl-D-Phenylalanine |
| D-Dab | D-2,4-Diaminobutyric acid |
| D-Dap | 3-Amino-L-Alanine |
| D-Glu | D-Glutamic acid |
| D-hArg | Homo-D-Arginine |
| D-His | D-Histidine |
| D-homoPhe | Homo-D-Phenylalanine |
| D-hSer | Homo-D-Serine |
| D-Iva | D-Isovaline |
| D-Leu | D-Leucine |
| D-Lys | D-Lysine |
| D-Nar | D-Norarginine |
| D-Nva | D-Norvaline |
| D-Orn | D-Ornithine |
| D-Phe | D-Phenylalanine |
| D-Phe(2-F,4-Cl) | 4-Chloro-2-fluoro-D-phenylalanine |
| D-Phe(2,3-diF) | 2,3-difluoro-D-phenylalanine |
| D-Phe(2,4-diCl) | 2,4-dichloro-D-phenylalanine |
| D-Phe(2,4-diF) | 2,4-difluoro-D-phenylalanine |
| D-Phe(2,4,5-triF) | 2,4,5-trifluoro-D-phenylalanine |
| D-Phe(3-CF3) | 3-Trifluoromethyl-D-Phenylalanine |
| D-Phe(3-Cl) | 3-Chloro-D-Phenylalanine |
| D-Phe(3-F,4-Me) | 4-methyl-3-fluoro-D-phenylalanine |
| D-Phe(3-F) | 3-fluoro-D-phenylalanine |
| D-Phe(3-Me) | 3-methyl-D-phenylalanine |
| D-Phe(3-Ph) | 3-phenyl-D-phenylalanine |
| D-Phe(3,4-diF) | 3,4-difluoro-D-phenylalanine |
| D-Phe(3,4-diMe) | 3,4-Dimethyl-D-Phenylalanine |
| D-Phe(3,4,5-triF) | 3,4,5-trifluoro-D-phenylalanine |
| D-Phe(4-Br) | 4-Bromo-D-phenylalanine |
| D-Phe(4-CF3) | 4-(trifluoromethyl)-D-phenylalanine |
| D-Phe(4-Cl) | 4-Chloro-D-Phenylalanine |
| D-Phe(4-F) | 4-Fluoro-D-phenylalanine |
| D-Phe(4-Me) | 4-Methyl-D-Phenylalanine |
| D-Phg | D-Phenylglycine |
| D-Pro | D-Proline |
| D-Ser | D-Serine |
| D-Trp | D-Tryptophan |
| D-Tyr | D-Tyrosine |
| Dap | 3-Amino-L-Alanine |
| delta-Guanidinovaleric acid | 5-guanidinopentanoic acid |
| delta-Guanidinovaleric acid | delta-Guanidinovaleric acid |
| Gaba | gamma-aminobutyric-acid |
| Gln | Glutamine |
| Glu | L-glutamate |
| Gly | Glycine |
| Gly(thien-3-yl) | L-alpha-(3-Thienyl)glycine |
| hCit | Homocitrulline |
| hCys | Homo-L-Cysteine |
| hGln | L-Homoglutamine |
| hGlu | L-homoglutamic acid |
| His | Histidine |
| His(3-Me) | 3-Methyl-L-Histidine |
| homoPhe | Homo-L-Phenylalanine |
| Indoline-COOH | L-Indoline-2-carboxylic acid |
| Inp | Isonipecotic acid |
| L-aMeAsp | alpha-methyl-L-aspartic acid |
| L-aMeGlu | alpha-methyl-L-glutamic acid |
| L-aMeSer | Alpha-methyl-L-serine |
| L-aMeVal | alpha-Methyl-L-Valine |
| L-Apm | L-Aminopimelic acid |
| L-Dab | L-2,4-Diaminobutanoic acid |
| L-hArg | Homo-L-Arginine |
| Lys | Lysine |
| Lys* | L-Lys(AEEAc-AEEAc-L-γ-Glu-17-carboxyheptadecanoyl) |
| Me-Arg | N2-Methyl-L-Arginine |
| Me-D-Arg | N2-Methyl-D-Arginine |
| Me-Gaba | 4-(methylamino)butanoic acid |
| N-4-aminobutyl-Gly | N-(4-aminobutyl)-Glycine |
| N-Me-His | N-Methyl-L-Histidine |
| Nar | L-Norarginine |
| Nip(4-NH₂) | 4-Amino-4-piperidinecarboxylic acid |
| Nle | L-Norleucine |
| Nva(Ph) | 5-Phenyl-L-Norvaline |
| Orn | L-Ornithine |
| PEG1 | 2-(2-aminoethoxy)acetic Acid |
| PEG2 | PEG2 |
| Pen | Penicillamine |
| Phe(3-Me) | 3-Methyl-L-Phenylalanine |
| Phe(3,4-diMe) | 3,4-Dimethyl-L-Phenylalanine |
| Phe(4-Me) | 4-Methyl-L-Phenylalanine |
| Phg | L-Phenylglycine |
| Pro(4-OH) | 4-Hydroxy-L-Proline |
| Pro(4-phenyl) | trans-4-Phenyl-L-Proline |
| Sar | N-methyl-glycine |
| Ser | Serine |
| Thz | L-Thioproline |
| Tranexamic acid | Tranexamic acid |
| Trp | Tryptophan |
| Trp(4-F) | 4-Fluoro-L-tryptophan |
| Trp(5-Cl) | 5-Chloro-L-tryptophan |
| Trp(5-F) | 5-Fluoro-L-tryptophan |
| Trp(5-Me) | 5-Methyl-L-Tryptophan |
| Trp(5-OH) | 5-Hydroxy-L-Tryptophan |
| Trp(6-Br) | 6-Bromo-L-tryptophan |
| Trp(6-CF3) | 6-(trifluoromethyl)-L-tryptophan |
| Trp(6-Cl) | 6-Chloro-L-Tryptophan |
| Trp(6-F) | 6-Fluoro-L-Tryptophan |
| Trp(6-Me) | 6-Methyl-L-Tryptophan |
| Trp(7-F) | 7-Fluoro-L-tryptophan |
| Trp(7-Me) | 7-methyl-L-Tryptophan |

In embodiments, the API is a peptide or a pharmaceutically acceptable salt thereof and present in the composition at a concentration of about 0.1%-20% (W/W). In embodiments, the API is a peptide or a pharmaceutically acceptable salt thereof and present in the composition at a concentration of about 1%-15% (W/W). In embodiments, the API is a peptide or a pharmaceutically acceptable salt thereof and present in the composition at a concentration of about 1%-10% (W/W).

In embodiments, the composition comprises:
(a) about 20-60% (W/W) total phospholipids consisting of Phospholipon® 90 G and 1,2-dioleoyl-sn-glycero-3-phosphoethanolamine (DOPE) in a ratio of about: 80:20, 75:25, 70:30, 60:40, 50:50, or 40:60 by weight;
(b) about 10-60% (W/W) Miglyol® 812 N;
(c) about 5-30% (W/W) ethanol; and
(d) about 1-15% (W/W) of an active pharmaceutical ingredient (API), wherein the API is a peptide or a pharmaceutically acceptable salt thereof, and wherein the peptide comprises one or more non-canonical amino acids

In embodiments, the composition comprises:
(a) about 20-60% (W/W) total phospholipids consisting of Phospholipon® 90 G and 1,2-dioleoyl-sn-glycero-3-phosphoethanolamine (DOPE) in a ratio of about: 80:20, 75:25, 70:30, 60:40, 50:50, or 40:60 by weight;
(b) about 10-60% (W/W) Miglyol® 812 N;
(c) about 5-30% (W/W) ethanol; and
(d) about 1-15% (W/W) of an active pharmaceutical ingredient (API), wherein the API is peptide 1158 or a salt thereof, e.g., an acetate salt or a trifluoroacetate salt thereof.

In embodiments, the composition consists of:
(a) about 20-60% (W/W) total phospholipids consisting of Phospholipon® 90 G and 1,2-dioleoyl-sn-glycero-3-phosphoethanolamine (DOPE) in a ratio of about: 80:20, 75:25, 70:30, 60:40, 50:50, or 40:60 by weight;
(b) about 10-60% (W/W) Miglyol® 812 N;
(c) about 5-30% (W/W) ethanol; and
(d) about 1-15% (W/W) of an active pharmaceutical ingredient (API), wherein the API is a peptide or a pharmaceutically acceptable salt thereof, and wherein the peptide comprises one or more non-canonical amino acids.

In embodiments, the composition consists of:
(e) about 20-60% (W/W) total phospholipids consisting of Phospholipon® 90 G and 1,2-dioleoyl-sn-glycero-3-phosphoethanolamine (DOPE) in a ratio of about: 80:20, 75:25, 70:30, 60:40, 50:50, or 40:60 by weight;
(f) about 10-60% (W/W) Miglyol® 812 N;
(g) about 5-30% (W/W) ethanol; and
(h) about 1-15% (W/W) of an active pharmaceutical ingredient (API), wherein the API is peptide 1158 or a salt thereof, e.g., an acetate salt or a trifluoroacetate salt thereof.

In embodiments, the composition is an extended-release composition. In embodiments, the composition provides extended release of the API (e.g., a peptide or a pharmaceutically acceptable salt thereof). In embodiments, the composition comprises an API and, upon administration to a mammalian subject (e.g., by subcutaneous injection), exhibits an extended release of the API relative to a control composition as evaluated by, for example, maximum serum concentration (Cmax), steady-state concentration (Css), or flat exposure of the API. The term “Cmax” refers to the maximum concentration reached by a given dose of API in a biological sample (e.g. serum). The term “Css” refers to the steady state concentration such that (1) Css=total exposure of the API (AUC)/dosing interval or (2) active plasma concentrations of API observed over multiple days after single bolus subcutaneous injection. In embodiments, the control composition includes a composition that does not comprise the second phospholipid species. In embodiments, the extended release is up to about 24, or about 36, or about 48, or about 60, or about 72, or about 84, or about 96, or about 120, or about 144, or about 168 hours or more upon administration of the composition.

In embodiments, the composition comprises or consists of:
(a) about 22.8% (W/W) of Phosphatidylcholine;
(b) about 22.8% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 22.8% (W/W) of Phosphatidylcholine;
(b) about 22.8% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 22.8% (W/W) of PL 90 G;
(b) about 22.8% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 22.8% (W/W) of PL 90 G;
(b) about 22.8% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 25.1% (W/W) of PL 90 G;
(b) about 20.5% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 28.5% (W/W) of PL 90 G;
(b) about 17.1% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 34.2% (W/W) of PL 90 G;
(b) about 11.4% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 18.3% (W/W) of PL 90 G;
(b) about 27.4% (W/W) of DOPE;
(c) about 11.7% (W/W) of ethanol;
(d) about 39.8% (W/W) of Miglyol® 812 N; and
(e) about 2.9% (W/W) of API.

In embodiments, the composition comprises or consists of:
(a) about 26.2% (W/W) of PL 90 G;
(b) about 26.2% (W/W) of DOPE;
(c) about 11.7% (W/W) of ethanol;
(d) about 33% (W/W) of Miglyol® 812 N; and
(e) about 2.9% (W/W) of API.

In embodiments, the composition comprises or consists of:
(a) about 26.2% (W/W) of PL 90 G;
(b) about 26.2% (W/W) of DOPE;
(c) about 28.1% (W/W) of ethanol;
(d) about 16.5% (W/W) of Miglyol® 812 N; and
(e) about 2.9% (W/W) of API.

In embodiments, the composition comprises or consists of:
(a) about 25.5% (W/W) of PL 90 G;
(b) about 25.5% (W/W) of DOPE;
(c) about 11.3% (W/W) of ethanol;
(d) about 32% (W/W) of Miglyol® 812 N; and
(e) about 5.7% (W/W) of API.

In embodiments, the composition comprises or consists of:
(a) about 27.4% (W/W) of PL 90 G;
(b) about 27.4% (W/W) of DOPE;
(c) about 11.7% (W/W) of ethanol;
(d) about 30.7% (W/W) of Miglyol® 812 N; and
(e) about 2.9% (W/W) of API.

In embodiments, the composition comprises or consists of:
(a) about 27.4% (W/W) of PL 90 G;
(b) about 27.4% (W/W) of DOPE;
(c) about 27% (W/W) of ethanol;
(d) about 15.3% (W/W) of Miglyol® 812 N; and
(e) about 2.9% (W/W) of API.

In embodiments, the composition comprises or consists of:
(a) about 26.6% (W/W) of PL 90 G;
(b) about 26.6% (W/W) of DOPE;
(c) about 11.3% (W/W) of ethanol;
(d) about 29.8% (W/W) of Miglyol® 812 N; and
(e) about 5.7% (W/W) of API.

In embodiments, the composition comprises or consists of:
(a) about 29.1% (W/W) of PL 90 G;
(b) about 29.1% (W/W) of DOPE;
(c) about 11.7% (W/W) of ethanol;
(d) about 27.2% (W/W) of Miglyol® 812 N; and
(e) about 2.9% (W/W) of API.

In embodiments, the composition comprises or consists of:
(a) about 29.1% (W/W) of PL 90 G;
(b) about 29.1% (W/W) of DOPE;
(c) about 25.2% (W/W) of ethanol;
(d) about 13.6% (W/W) of Miglyol® 812 N; and
(e) about 2.9% (W/W) of API.

In embodiments, the composition comprises or consists of:
(a) about 28.3% (W/W) of PL 90 G;
(b) about 28.3% (W/W) of DOPE;
(c) about 11.3% (W/W) of ethanol;
(d) about 26.4% (W/W) of Miglyol® 812 N; and
(e) about 5.7% (W/W) of API.

In embodiments, the composition comprises or consists of:
(a) about 0.2-10.7% (W/W) of API;
(b) about 16.8-35.2% (W/W) of PL 90 G;
(c) about 10.5-29.9% (W/W) of DOPE;
(d) about 12.5-40.9% (W/W) of Miglyol® 812 N; and
(e) about 10.7-28.9% (W/W) of ethanol.

In embodiments, the composition comprises or consists of:
(a) about 0.2% (W/W) of API;
(b) about 23.4% (W/W) of PL 90 G;
(c) about 23.4% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of API;
(b) about 25.8% (W/W) of PL 90 G;
(c) about 21.1% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of API;
(b) about 29.3% (W/W) of PL 90 G;
(c) about 17.6% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of API;
(b) about 35.2% (W/W) of PL 90 G;
(c) about 11.7% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of API;
(b) about 18.8% (W/W) of PL 90 G;
(c) about 28.1% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of API;
(b) about 27% (W/W) of PL 90 G;
(c) about 27% (W/W) of DOPE;
(d) about 33.9% (W/W) of Miglyol® 812 N; and
(e) about 12% (W/W) of ethanol.

In embodiments, the composition comprises or consists of:
(a) about 0.2% (W/W) of API;
(b) about 27% (W/W) of PL 90 G;
(c) about 27% (W/W) of DOPE;
(d) about 16.9% (W/W) of Miglyol® 812 N; and
(e) about 28.9% (W/W) of ethanol.

In embodiments, the composition comprises or consists of:
(a) about 0.2% (W/W) of API;
(b) about 28.1% (W/W) of PL 90 G;
(c) about 28.1% (W/W) of DOPE;
(d) about 31.5% (W/W) of Miglyol® 812 N; and
(e) about 12% (W/W) of ethanol.

In embodiments, the composition comprises or consists of:
(a) about 0.2% (W/W) of API;
(b) about 28.1% (W/W) of PL 90 G;
(c) about 28.1% (W/W) of DOPE;
(d) about 15.8% (W/W) of Miglyol® 812 N; and
(e) about 27.7% (W/W) of ethanol.

In embodiments, the composition comprises or consists of:
(a) about 0.2% (W/W) of API;
(b) about 29.9% (W/W) of PL 90 G;
(c) about 29.9% (W/W) of DOPE;
(d) about 27.9% (W/W) of Miglyol® 812 N; and
(e) about 12% (W/W) of ethanol.

In embodiments, the composition comprises or consists of:
(a) about 0.2% (W/W) of API;
(b) about 29.9% (W/W) of PL 90 G;
(c) about 29.9% (W/W) of DOPE;
(d) about 14% (W/W) of Miglyol® 812 N; and
(e) about 25.9% (W/W) of ethanol.

In embodiments, the composition comprises or consists of:
(a) about 1% (W/W) of API;
(b) about 23.3% (W/W) of PL 90 G;
(c) about 23.3% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of API;
(b) about 25.6% (W/W) of PL 90 G;
(c) about 20.9% (W/W) of DOPE;
(d) about 40.6% (W/W) of Miglyol® 812 N; and
(e) about 11.9% (W/W) of ethanol.

In embodiments, the composition comprises or consists of:

(a) about 1% (W/W) of API;
(b) about 29.1% (W/W) of PL 90 G;
(c) about 17.5% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of API;
(b) about 34.9% (W/W) of PL 90 G;
(c) about 11.6% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of API;
(b) about 18.6% (W/W) of PL 90 G;
(c) about 27.9% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of API;
(b) about 26.8% (W/W) of PL 90 G;
(c) about 26.8% (W/W) of DOPE;
(d) about 33.6% (W/W) of Miglyol® 812 N; and
(e) about 11.9% (W/W) of ethanol.

In embodiments, the composition comprises or consists of:
(a) about 1% (W/W) of API;
(b) about 26.8% (W/W) of PL 90 G;
(c) about 26.8% (W/W) of DOPE;
(d) about 16.8% (W/W) of Miglyol® 812 N; and
(e) about 28.7% (W/W) of ethanol.

In embodiments, the composition comprises or consists of:
(a) about 1% (W/W) of API;
(b) about 27.9% (W/W) of PL 90 G;
(c) about 27.9% (W/W) of DOPE;
(d) about 31.3% (W/W) of Miglyol® 812 N; and
(e) about 11.9% (W/W) of ethanol.

In embodiments, the composition comprises or consists of:
(a) about 1% (W/W) of API;
(b) about 27.9% (W/W) of PL 90 G;
(c) about 27.9% (W/W) of DOPE;
(d) about 15.6% (W/W) of Miglyol® 812 N; and
(e) about 27.5% (W/W) of ethanol.

In embodiments, the composition comprises or consists of:
(a) about 1% (W/W) of API;
(b) about 29.7% (W/W) of PL 90 G;
(c) about 29.7% (W/W) of DOPE;
(d) about 27.8% (W/W) of Miglyol® 812 N; and
(e) about 11.9% (W/W) of ethanol.

In embodiments, the composition comprises or consists of:
(a) about 1% (W/W) of API;
(b) about 29.7% (W/W) of PL 90 G;
(c) about 29.7% (W/W) of DOPE;
(d) about 13.9% (W/W) of Miglyol® 812 N; and
(e) about 25.7% (W/W) of ethanol.

In embodiments, the composition comprises or consists of:
(a) about 2.9% (W/W) of API;
(b) about 22.8% (W/W) of PL 90 G;
(c) about 22.8% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of API;
(b) about 25.1% (W/W) of PL 90 G;
(c) about 20.5% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of API;
(b) about 28.5% (W/W) of PL 90 G;
(c) about 17.1% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of API;
(b) about 34.2% (W/W) of PL 90 G;
(c) about 11.4% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of API;
(b) about 18.3% (W/W) of PL 90 G;
(c) about 27.4% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of API;
(b) about 26.2% (W/W) of PL 90 G;
(c) about 26.2% (W/W) of DOPE;
(d) about 33% (W/W) of Miglyol® 812 N; and
(e) about 11.7% (W/W) of ethanol.

In embodiments, the composition comprises or consists of:
(a) about 2.9% (W/W) of API;
(b) about 26.2% (W/W) of PL 90 G;
(c) about 26.2% (W/W) of DOPE;
(d) about 16.5% (W/W) of Miglyol® 812 N; and
(e) about 28.1% (W/W) of ethanol.

In embodiments, the composition comprises or consists of:
(a) about 2.9% (W/W) of API;
(b) about 27.4% (W/W) of PL 90 G;
(c) about 27.4% (W/W) of DOPE;
(d) about 30.7% (W/W) of Miglyol® 812 N; and
(e) about 11.7% (W/W) of ethanol.

In embodiments, the composition comprises or consists of:
(a) about 2.9% (W/W) of API;
(b) about 27.4% (W/W) of PL 90 G;
(c) about 27.4% (W/W) of DOPE;
(d) about 15.3% (W/W) of Miglyol® 812 N; and
(e) about 27% (W/W) of ethanol.

In embodiments, the composition comprises or consists of:
(a) about 2.9% (W/W) of API;
(b) about 29.1% (W/W) of PL 90 G;
(c) about 29.1% (W/W) of DOPE;
(d) about 27.2% (W/W) of Miglyol® 812 N; and
(e) about 11.7% (W/W) of ethanol.

In embodiments, the composition comprises or consists of:
(a) about 2.9% (W/W) of API;
(b) about 29.1% (W/W) of PL 90 G;
(c) about 29.1% (W/W) of DOPE;
(d) about 13.6% (W/W) of Miglyol® 812 N; and
(e) about 25.2% (W/W) of ethanol.

In embodiments, the composition comprises or consists of:
(a) about 4.3% (W/W) of API;
(b) about 22.5% (W/W) of PL 90 G;
(c) about 22.5% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of API;
(b) about 24.7% (W/W) of PL 90 G;
(c) about 20.2% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of API;
(b) about 28.1% (W/W) of PL 90 G;
(c) about 16.9% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of API;
(b) about 33.7% (W/W) of PL 90 G;
(c) about 11.2% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of API;
(b) about 18% (W/W) of PL 90 G;
(c) about 27% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of API;
(b) about 25.9% (W/W) of PL 90 G;
(c) about 25.9% (W/W) of DOPE;
(d) about 32.5% (W/W) of Miglyol® 812 N; and
(e) about 11.5% (W/W) of ethanol.

In embodiments, the composition comprises or consists of:
(a) about 4.3% (W/W) of API;
(b) about 25.9% (W/W) of PL 90 G;
(c) about 25.9% (W/W) of DOPE;
(d) about 16.2% (W/W) of Miglyol® 812 N; and
(e) about 27.7% (W/W) of ethanol.

In embodiments, the composition comprises or consists of:
(a) about 4.3% (W/W) of API;
(b) about 27% (W/W) of PL 90 G;
(c) about 27% (W/W) of DOPE;
(d) about 30.2% (W/W) of Miglyol® 812 N; and
(e) about 11.5% (W/W) of ethanol.

In embodiments, the composition comprises or consists of:
(a) about 4.3% (W/W) of API;
(b) about 27% (W/W) of PL 90 G;
(c) about 27% (W/W) of DOPE;
(d) about 15.1% (W/W) of Miglyol® 812 N; and
(e) about 26.6% (W/W) of ethanol.

In embodiments, the composition comprises or consists of:
(a) about 4.3% (W/W) of API;
(b) about 28.7% (W/W) of PL 90 G;
(c) about 28.7% (W/W) of DOPE;
(d) about 26.8% (W/W) of Miglyol® 812 N; and
(e) about 11.5% (W/W) of ethanol.

In embodiments, the composition comprises or consists of:
(a) about 4.3% (W/W) of API;
(b) about 28.7% (W/W) of PL 90 G;
(c) about 28.7% (W/W) of DOPE;
(d) about 13.4% (W/W) of Miglyol® 812 N; and
(e) about 24.9% (W/W) of ethanol.

In embodiments, the composition comprises or consists of:
(a) about 5.7% (W/W) of API;
(b) about 22.2% (W/W) of PL 90 G;
(c) about 22.2% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of API;
(b) about 24.4% (W/W) of PL 90 G;
(c) about 20% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of API;
(b) about 27.7% (W/W) of PL 90 G;
(c) about 16.6% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of API;
(b) about 33.3% (W/W) of PL 90 G;
(c) about 11.1% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of API;
(b) about 17.7% (W/W) of PL 90 G;
(c) about 26.6% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of API;
(b) about 25.5% (W/W) of PL 90 G;
(c) about 25.5% (W/W) of DOPE;
(d) about 32% (W/W) of Miglyol® 812 N; and
(e) about 11.3% (W/W) of ethanol.

In embodiments, the composition comprises or consists of:
(a) about 5.7% (W/W) of API;
(b) about 25.5% (W/W) of PL 90 G;
(c) about 25.5% (W/W) of DOPE;
(d) about 16% (W/W) of Miglyol® 812 N; and
(e) about 27.3% (W/W) of ethanol.

In embodiments, the composition comprises or consists of:
(a) about 5.7% (W/W) of API;
(b) about 26.6% (W/W) of PL 90 G;
(c) about 26.6% (W/W) of DOPE;
(d) about 29.8% (W/W) of Miglyol® 812 N; and
(e) about 11.3% (W/W) of ethanol.

In embodiments, the composition comprises or consists of:
(a) about 5.7% (W/W) of API;
(b) about 26.6% (W/W) of PL 90 G;
(c) about 26.6% (W/W) of DOPE;
(d) about 14.9% (W/W) of Miglyol® 812 N; and
(e) about 26.2% (W/W) of ethanol.

In embodiments, the composition comprises or consists of:
(a) about 5.7% (W/W) of API;
(b) about 28.3% (W/W) of PL 90 G;
(c) about 28.3% (W/W) of DOPE;
(d) about 26.4% (W/W) of Miglyol® 812 N; and
(e) about 11.3% (W/W) of ethanol.

In embodiments, the composition comprises or consists of:
(a) about 5.7% (W/W) of API;
(b) about 28.3% (W/W) of PL 90 G;
(c) about 28.3% (W/W) of DOPE;
(d) about 13.2% (W/W) of Miglyol® 812 N; and
(e) about 24.5% (W/W) of ethanol.

In embodiments, the composition comprises or consists of:
(a) about 10.7% (W/W) of API;
(b) about 21% (W/W) of PL 90 G;
(c) about 21% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of API;
(b) about 23.1% (W/W) of PL 90 G;
(c) about 18.9% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of API;
(b) about 26.2% (W/W) of PL 90 G;
(c) about 15.7% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of API;
(b) about 31.5% (W/W) of PL 90 G;
(c) about 10.5% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of API;
(b) about 16.8% (W/W) of PL 90 G;
(c) about 25.2% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of API;
(b) about 24.1% (W/W) of PL 90 G;
(c) about 24.1% (W/W) of DOPE;
(d) about 30.3% (W/W) of Miglyol® 812 N; and
(e) about 10.7% (W/W) of ethanol.

In embodiments, the composition comprises or consists of:
(a) about 10.7% (W/W) of API;
(b) about 24.1% (W/W) of PL 90 G;
(c) about 24.1% (W/W) of DOPE;
(d) about 15.2% (W/W) of Miglyol® 812 N; and
(e) about 25.9% (W/W) of ethanol.

In embodiments, the composition comprises or consists of:
(a) about 10.7% (W/W) of API;
(b) about 25.2% (W/W) of PL 90 G;
(c) about 25.2% (W/W) of DOPE;
(d) about 28.2% (W/W) of Miglyol® 812 N; and
(e) about 10.7% (W/W) of ethanol.

In embodiments, the composition comprises or consists of:
(a) about 10.7% (W/W) of API;
(b) about 25.2% (W/W) of PL 90 G;
(c) about 25.2% (W/W) of DOPE;
(d) about 14.1% (W/W) of Miglyol® 812 N; and
(e) about 24.8% (W/W) of ethanol.

In embodiments, the composition comprises or consists of:
(a) about 10.7% (W/W) of API;
(b) about 26.8% (W/W) of PL 90 G;
(c) about 26.8% (W/W) of DOPE;
(d) about 25% (W/W) of Miglyol® 812 N; and
(e) about 10.7% (W/W) of ethanol.

In embodiments, the composition comprises or consists of:
(a) about 10.7% (W/W) of API;
(b) about 26.8% (W/W) of PL 90 G;
(c) about 26.8% (W/W) of DOPE;
(d) about 12.5% (W/W) of Miglyol® 812 N; and
(e) about 23.2% (W/W) of ethanol.

In embodiments, the composition comprises or consists of:
(a) about 25.1% (W/W) of PL 90 G;
(b) about 20.5% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 28.5% (W/W) of PL 90 G;
(b) about 17.1% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 34.2% (W/W) of PL 90 G;
(b) about 11.4% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 18.3% (W/W) of PL 90 G;
(b) about 27.4% (W/W) of DOPE;
(c) about 11.7% (W/W) of ethanol;
(d) about 39.8% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 26.2% (W/W) of PL 90 G;
(b) about 26.2% (W/W) of DOPE;
(c) about 11.7% (W/W) of ethanol;
(d) about 33% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 26.2% (W/W) of PL 90 G;
(b) about 26.2% (W/W) of DOPE;
(c) about 28.1% (W/W) of ethanol;
(d) about 16.5% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 25.5% (W/W) of PL 90 G;
(b) about 25.5% (W/W) of DOPE;
(c) about 11.3% (W/W) of ethanol;
(d) about 32% (W/W) of Miglyol® 812 N; and
(e) about 5.7% (W/W) of peptide 1158 or a salt thereof, e.g., an acetate salt or a trifluoroacetate salt thereof.

In embodiments, the composition comprises or consists of:
(a) about 27.4% (W/W) of PL 90 G;
(b) about 27.4% (W/W) of DOPE;
(c) about 11.7% (W/W) of ethanol;
(d) about 30.7% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 27.4% (W/W) of PL 90 G;
(b) about 27.4% (W/W) of DOPE;
(c) about 27% (W/W) of ethanol;
(d) about 15.3% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 26.6% (W/W) of PL 90 G;
(b) about 26.6% (W/W) of DOPE;
(c) about 11.3% (W/W) of ethanol;
(d) about 29.8% (W/W) of Miglyol® 812 N; and
(e) about 5.7% (W/W) of peptide 1158 or a salt thereof, e.g., an acetate salt or a trifluoroacetate salt thereof.

In embodiments, the composition comprises or consists of:
(a) about 29.1% (W/W) of PL 90 G;
(b) about 29.1% (W/W) of DOPE;
(c) about 11.7% (W/W) of ethanol;
(d) about 27.2% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 29.1% (W/W) of PL 90 G;
(b) about 29.1% (W/W) of DOPE;
(c) about 25.2% (W/W) of ethanol;
(d) about 13.6% (W/W) of Miglyol® 812 N; and
(e) about 2.9% (W/W) of peptide 1158 or a salt thereof, e.g., an acetate salt or a trifluoroacetate salt thereof.

In embodiments, the composition comprises or consists of:
(a) about 28.3% (W/W) of PL 90 G;
(b) about 28.3% (W/W) of DOPE;
(c) about 11.3% (W/W) of ethanol;
(d) about 26.4% (W/W) of Miglyol® 812 N; and
(e) about 5.7% (W/W) of peptide 1158 or a salt thereof, e.g., an acetate salt or a trifluoroacetate salt thereof.

In embodiments, the composition comprises or consists of:
(a) about 0.2-10.7% (W/W) of peptide 1158 or a salt thereof, e.g., an acetate salt or a trifluoroacetate salt thereof;
(b) about 16.8-35.2% (W/W) of PL 90 G;
(c) about 10.5-29.9% (W/W) of DOPE;
(d) about 12.5-40.9% (W/W) of Miglyol® 812 N; and
(e) about 10.7-28.9% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 23.4% (W/W) of PL 90 G;
(c) about 23.4% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 25.8% (W/W) of PL 90 G;
(c) about 21.1% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 29.3% (W/W) of PL 90 G;
(c) about 17.6% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 35.2% (W/W) of PL 90 G;
(c) about 11.7% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 18.8% (W/W) of PL 90 G;
(c) about 28.1% (W/W) of DOPE;
(d) about 40.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 27% (W/W) of PL 90 G;
(c) about 27% (W/W) of DOPE;
(d) about 33.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 27% (W/W) of PL 90 G;
(c) about 27% (W/W) of DOPE;
(d) about 16.9% (W/W) of Miglyol® 812 N; and
(e) about 28.9% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 28.1% (W/W) of PL 90 G;
(c) about 28.1% (W/W) of DOPE;
(d) about 31.5% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 28.1% (W/W) of PL 90 G;
(c) about 28.1% (W/W) of DOPE;
(d) about 15.8% (W/W) of Miglyol® 812 N; and
(e) about 27.7% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 29.9% (W/W) of PL 90 G;
(c) about 29.9% (W/W) of DOPE;
(d) about 27.9% (W/W) of Miglyol® 812 N; and
(e) about 12% (W/W) of ethanol.

In embodiments, the composition comprises or consists of:
(a) about 0.2% (W/W) of peptide 1158 or a salt thereof, e.g., an acetate salt or a trifluoroacetate salt thereof;
(b) about 29.9% (W/W) of PL 90 G;
(c) about 29.9% (W/W) of DOPE;
(d) about 14% (W/W) of Miglyol® 812 N; and
(e) about 25.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 23.3% (W/W) of PL 90 G;
(c) about 23.3% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 25.6% (W/W) of PL 90 G;
(c) about 20.9% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 29.1% (W/W) of PL 90 G;
(c) about 17.5% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 34.9% (W/W) of PL 90 G;
(c) about 11.6% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 18.6% (W/W) of PL 90 G;
(c) about 27.9% (W/W) of DOPE;
(d) about 40.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 26.8% (W/W) of PL 90 G;
(c) about 26.8% (W/W) of DOPE;
(d) about 33.6% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 26.8% (W/W) of PL 90 G;
(c) about 26.8% (W/W) of DOPE;
(d) about 16.8% (W/W) of Miglyol® 812 N; and
(e) about 28.7% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 27.9% (W/W) of PL 90 G;
(c) about 27.9% (W/W) of DOPE;
(d) about 31.3% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 27.9% (W/W) of PL 90 G;
(c) about 27.9% (W/W) of DOPE;
(d) about 15.6% (W/W) of Miglyol® 812 N; and
(e) about 27.5% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 29.7% (W/W) of PL 90 G;
(c) about 29.7% (W/W) of DOPE;
(d) about 27.8% (W/W) of Miglyol® 812 N; and
(e) about 11.9% (W/W) of ethanol.

In embodiments, the composition comprises or consists of:
(a) about 1% (W/W) of peptide 1158 or a salt thereof, e.g., an acetate salt or a trifluoroacetate salt thereof;
(b) about 29.7% (W/W) of PL 90 G;
(c) about 29.7% (W/W) of DOPE;
(d) about 13.9% (W/W) of Miglyol® 812 N; and
(e) about 25.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 22.8% (W/W) of PL 90 G;
(c) about 22.8% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 25.1% (W/W) of PL 90 G;
(c) about 20.5% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 28.5% (W/W) of PL 90 G;
(c) about 17.1% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 34.2% (W/W) of PL 90 G;
(c) about 11.4% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 18.3% (W/W) of PL 90 G;
(c) about 27.4% (W/W) of DOPE;
(d) about 39.8% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 26.2% (W/W) of PL 90 G;
(c) about 26.2% (W/W) of DOPE;
(d) about 33% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 26.2% (W/W) of PL 90 G;
(c) about 26.2% (W/W) of DOPE;
(d) about 16.5% (W/W) of Miglyol® 812 N; and
(e) about 28.1% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 27.4% (W/W) of PL 90 G;
(c) about 27.4% (W/W) of DOPE;
(d) about 30.7% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 27.4% (W/W) of PL 90 G;
(c) about 27.4% (W/W) of DOPE;
(d) about 15.3% (W/W) of Miglyol® 812 N; and
(e) about 27% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 29.1% (W/W) of PL 90 G;
(c) about 29.1% (W/W) of DOPE;
(d) about 27.2% (W/W) of Miglyol® 812 N; and
(e) about 11.7% (W/W) of ethanol.

In embodiments, the composition comprises or consists of:
(a) about 2.9% (W/W) of peptide 1158 or a salt thereof, e.g., an acetate salt or a trifluoroacetate salt thereof;
(b) about 29.1% (W/W) of PL 90 G;
(c) about 29.1% (W/W) of DOPE;
(d) about 13.6% (W/W) of Miglyol® 812 N; and
(e) about 25.2% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 22.5% (W/W) of PL 90 G;
(c) about 22.5% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 24.7% (W/W) of PL 90 G;
(c) about 20.2% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 28.1% (W/W) of PL 90 G;
(c) about 16.9% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 33.7% (W/W) of PL 90 G;
(c) about 11.2% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 18% (W/W) of PL 90 G;
(c) about 27% (W/W) of DOPE;
(d) about 39.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 25.9% (W/W) of PL 90 G;
(c) about 25.9% (W/W) of DOPE;
(d) about 32.5% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 25.9% (W/W) of PL 90 G;
(c) about 25.9% (W/W) of DOPE;
(d) about 16.2% (W/W) of Miglyol® 812 N; and
(e) about 27.7% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 27% (W/W) of PL 90 G;
(c) about 27% (W/W) of DOPE;
(d) about 30.2% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 27% (W/W) of PL 90 G;
(c) about 27% (W/W) of DOPE;
(d) about 15.1% (W/W) of Miglyol® 812 N; and
(e) about 26.6% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 28.7% (W/W) of PL 90 G;
(c) about 28.7% (W/W) of DOPE;
(d) about 26.8% (W/W) of Miglyol® 812 N; and
(e) about 11.5% (W/W) of ethanol.

In embodiments, the composition comprises or consists of:
(a) about 4.3% (W/W) of peptide 1158 or a salt thereof, e.g., an acetate salt or a trifluoroacetate salt thereof;
(b) about 28.7% (W/W) of PL 90 G;
(c) about 28.7% (W/W) of DOPE;
(d) about 13.4% (W/W) of Miglyol® 812 N; and
(e) about 24.9% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 22.2% (W/W) of PL 90 G;
(c) about 22.2% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 24.4% (W/W) of PL 90 G;
(c) about 20% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 27.7% (W/W) of PL 90 G;
(c) about 16.6% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 33.3% (W/W) of PL 90 G;
(c) about 11.1% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 17.7% (W/W) of PL 90 G;
(c) about 26.6% (W/W) of DOPE;
(d) about 38.7% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 25.5% (W/W) of PL 90 G;
(c) about 25.5% (W/W) of DOPE;
(d) about 32% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 25.5% (W/W) of PL 90 G;
(c) about 25.5% (W/W) of DOPE;
(d) about 16% (W/W) of Miglyol® 812 N; and
(e) about 27.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 26.6% (W/W) of PL 90 G;
(c) about 26.6% (W/W) of DOPE;
(d) about 29.8% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 26.6% (W/W) of PL 90 G;
(c) about 26.6% (W/W) of DOPE;
(d) about 14.9% (W/W) of Miglyol® 812 N; and
(e) about 26.2% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 28.3% (W/W) of PL 90 G;
(c) about 28.3% (W/W) of DOPE;
(d) about 26.4% (W/W) of Miglyol® 812 N; and
(e) about 11.3% (W/W) of ethanol.

In embodiments, the composition comprises or consists of:
(a) about 5.7% (W/W) of peptide 1158 or a salt thereof, e.g., an acetate salt or a trifluoroacetate salt thereof;
(b) about 28.3% (W/W) of PL 90 G;
(c) about 28.3% (W/W) of DOPE;
(d) about 13.2% (W/W) of Miglyol® 812 N; and
(e) about 24.5% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 21% (W/W) of PL 90 G;
(c) about 21% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 23.1% (W/W) of PL 90 G;
(c) about 18.9% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 26.2% (W/W) of PL 90 G;
(c) about 15.7% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 31.5% (W/W) of PL 90 G;
(c) about 10.5% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 16.8% (W/W) of PL 90 G;
(c) about 25.2% (W/W) of DOPE;
(d) about 36.6% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 24.1% (W/W) of PL 90 G;
(c) about 24.1% (W/W) of DOPE;
(d) about 30.3% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 24.1% (W/W) of PL 90 G;
(c) about 24.1% (W/W) of DOPE;
(d) about 15.2% (W/W) of Miglyol® 812 N; and
(e) about 25.9% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 25.2% (W/W) of PL 90 G;
(c) about 25.2% (W/W) of DOPE;
(d) about 28.2% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 25.2% (W/W) of PL 90 G;
(c) about 25.2% (W/W) of DOPE;
(d) about 14.1% (W/W) of Miglyol® 812 N; and
(e) about 24.8% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 26.8% (W/W) of PL 90 G;
(c) about 26.8% (W/W) of DOPE;
(d) about 25% (W/W) of Miglyol® 812 N; and
(e) about 10.7% (W/W) of ethanol.

In embodiments, the composition comprises or consists of:
(a) about 10.7% (W/W) of peptide 1158 or a salt thereof, e.g., an acetate salt or a trifluoroacetate salt thereof;
(b) about 26.8% (W/W) of PL 90 G;
(c) about 26.8% (W/W) of DOPE;
(d) about 12.5% (W/W) of Miglyol® 812 N; and
(e) about 23.2% (W/W) of ethanol.

In embodiments, the composition is a composition selected from any one of Table BBB, Table CCC, or Table DDD.

In aspects, the present disclosure provides articles of manufacture comprising a composition of the present disclosure.

In embodiments, the article comprises a vial or a prefilled medical device. In embodiments, the article comprises a syringe. In embodiments, the syringe is made of glass, cyclic olefin polymer (COP), or cyclic olefin copolymer (COC). In embodiments, the syringe has a closed stopper and/or plunger. In embodiments, the article comprises a cartridge.

In aspects, the present disclosure provides methods comprising administering an effective amount of the composition of the present disclosure to a subject. In embodiments, the method comprises administering an effective amount of the composition of the present disclosure via the article of the present disclosure.

In embodiments, the compositions disclosed herein are administered by injection. In embodiments, the compositions disclosed herein are administered by subcutaneous injection, intravenous injection, intramuscular injection, or depot injection. In embodiments, the compositions disclosed herein are administered by subcutaneous injection. In embodiments, the compositions disclosed herein are administered by injection by syringe.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. In embodiments, pharmaceutically acceptable compositions of the present disclosure further comprise preservatives. Non-limiting examples of preservatives include m-cresol, phenol, benzyl alcohol, tonicifiers, such as glycerin, mannitol, sucrose, and buffering agents such as phosphate, Tris, acetate, citrate.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions of the present disclosure may be administered orally, parenterally, by inhalation spray, topically, rectally, nasally, buccally, sub-lingually, vaginally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this disclosure are aqueous or oleaginous suspension. These suspensions are formulated according to techniques known in the art using suitable dispersing or wetting agents and suspending agents. The sterile injectable preparation is a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

In embodiments, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation or in a suitable enema formulation. In embodiments, the compositions of the present disclosure are topically-transdermal patches. In embodiments, the composition of the present disclosure are smart pills designed for targeted drug delivery to the GI tract and systemic, needle-free delivery.

For topical applications, provided compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In certain embodiments, compositions of this disclosure are formulated for oral administration. Such formulations may be administered with or without food. In some embodiments, compositions of this disclosure are administered without food. In other embodiments, compositions of this disclosure are administered with food.

Compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the disease being treated. In certain embodiments, the compositions of the disclosure may be administered orally or parenterally at dosage levels of about 0.01 mg/kg to about 50 mg/kg and preferably from about 1 mg/kg to about 25 mg/kg of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In embodiments, the capsules, tablets, and pills can be enterically coated.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

One or more compositions of the disclosure can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a composition of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin.

In embodiments, the method comprises administering an effective amount of the composition of the present disclosure via a syringe.

In embodiments, the method comprises administering an effective amount of the composition of the present disclosure via a syringe made of glass, cyclic olefin polymer (COP), or cyclic olefin copolymer (COC) and optionally with a closed stopper and/or plunger.

### Methods of Preparation

In aspects, the present disclosure provides methods of preparing the compositions of the present disclosure.

In embodiments, the method comprises a step of mixing a slow diffusing solvent with a fast diffusing solvent to obtain a solution. In embodiments, the fast diffusing solvent is added to the slow diffusing solvent. In embodiments, the solution is mixed for about 1 min, or about 5 min, or about 10 min, or about 15 min, or more. In embodiments, the solution is mixed for about 5-15 minutes.

In embodiments, the method comprises a step of adding a first species of phospholipid to a solution. In embodiments, the solution comprises the slow diffusing solvent and the fast diffusing solvent. In embodiments, the first species of phospholipid is added while mixing the solution.

In embodiments, the method comprises a step of mixing the solution following the addition of the first species of phospholipid. In embodiments, the solution comprises the slow diffusing solvent and the fast diffusing solvent. In embodiments, the solution is mixed for about 15 to about 90 minutes following the addition of the first species of phospholipid. In embodiments, the solution is mixed for about 1 min, or about 5 min, or about 10 min, or about 15 min, or about 20 min, or about 30 min, or about 40 min, or about 50 min, or about 60 min, or about 70 min, or about 80 min, or about 90 min, or about 100 min, or 100 min or more following the addition of the first species of phospholipid.

In embodiments, the method comprises a step of adding a second species of phospholipid to a solution. In embodiments, the solution comprises one or more of the slow diffusing solvent, the fast diffusing solvent, and the first species of phospholipid. In embodiments, the solution comprises the slow diffusing solvent, the fast diffusing solvent, and the first species of phospholipid. In embodiments, the second species of phospholipid is added while mixing the solution.

In embodiments, the method comprises a step of mixing a solution following the addition of the second species of phospholipid to the solution. In embodiments, the solution comprises one or more of the slow diffusing solvent, the fast diffusing solvent, and the first species of phospholipid. In embodiments, the solution comprises the slow diffusing solvent, the fast diffusing solvent, and the first species of phospholipid. In embodiments, the solution is mixed for about 20 minutes to about 8 hours following the addition of the second species of phospholipid. In embodiments, the solution is mixed for about 1 min, or about 5 min, or about 10 min, or about 15 min, or about 20 min, or about 30 min, or about 40 min, or about 1 hour, or about 2 hours, or about 3 hours, or about 4 hours, or about 5 hours, or about 6 hours, or about 7 hours, or about 8 hours, or about 9 hours, or about 10 hours, or about 10 hours or more following the addition of the second species of phospholipid.

In embodiments, the method comprises a step of adding an API to a solution. In embodiments, the solution comprises one or more of the slow diffusing solvent, the fast diffusing solvent, the first species of phospholipid, and the second species of phospholipid. In embodiments, the solution comprises the slow diffusing solvent, the fast diffusing solvent, the first species of phospholipid, and the second species of phospholipid. In embodiments, the API is added while mixing the solution.

In embodiments, the method comprises a step of mixing a solution following the addition of the API. In embodiments, the solution comprises one or more of the slow diffusing solvent, the fast diffusing solvent, the first species of phospholipid, and the second species of phospholipid. In embodiments, the solution comprises the slow diffusing solvent, the fast diffusing solvent, the first species of phospholipid, and the second species of phospholipid. In embodiments, the solution is mixed for about 5 minutes to about 2 hours following the addition of the API. In embodiments, the solution is mixed for about 1 min, or about 5 min, or about 10 min, or about 20 min, or about 30 min, or about 40 min, or about 50 min, or about 60 min, or about 90 min, or about 120 min, or about 150 min, or 150 min or more following the addition of the API.

In embodiments, one or more steps of the method are performed at about room temperature (e.g., about 15-25° C.). In embodiments, one or more steps of the method include a heading step (e.g. the method is performed at a temperature above about 25° C.). In embodiments, one or more steps of the method are performed at about 25° C. to about 50° C., about 25° C. to about 30° C., about 30° C. to about 40° C., or about 30° C. to about 50° C., optionally while mixing a solution. In embodiments, the solution is heated to a temperature above room temperature while mixing the solution. In embodiments, the solution is heated to a temperature at about 28° C., or about 29° C., or about 30° C., or about 31° C., or about 32° C., or about 33° C., or about 34° C., or about 35° C., or about 36° C., or about 37° C., or about 38° C., or about 38° C., or more. In embodiments, one or more steps of the method comprise heating a solution to a temperature of about 30° C. to about 35° C. In embodiments, one or more steps of the method comprise a step of cooling a solution to about room temperature (e.g., after heating the solution to a temperature above room temperature e.g., greater than about 25° C.).

In embodiments, the method comprises a step of filtering a solution through a filter. In embodiments, the filter is sterile. In embodiments, the sterile filter is a 0.22 μm filter, or a 0.8/0.22 μm filter, or a 0.4/0.22 μm filter, or a 0.22/0.4 μm filter.

In embodiments, the method comprises a step of titrating a solution (e.g., after filtering) to a target weight of the solution.

In embodiments, the step of titrating is performed using the fast diffusing solvent (e.g., by adding the fast diffusing solvent to the solution (e.g., after filtering). In embodiments, the fast diffusing solvent is itself filtered through a sterile filter before adding to the solution for titrating. In embodiments, the fast diffusing solvent is filtered through the same filter used for filtering the solution. In embodiments, the fast diffusing solvent is filtered through a 0.22 μm filter, or a 0.8/0.22 μm filter, or a 0.4/0.22 μm filter, or a 0.22/0.4 μm filter for titrating.

In embodiments, the method comprises a step of filling an amount of the solution (e.g., after filtering and/or titrating) into a container. In embodiments, the container is a vial, a pre-syringe, or a cartridge. In embodiments, the vial, pre-syringe, or cartridge is sterile. In embodiments, the amount is about 0.1 mL, or about 0.5 mL, or about 1 mL, or about 2 mL, or about 3 mL, or about 4 mL, or about 5 mL, or about 6 mL, or about 7 mL, or about 8 mL, or about 10) mL, or about 20 mL or more. In embodiments, the amount is about 1-10 mL.

The steps of the methods of present disclosure may be performed in different orders and/or simultaneously.

In embodiments, the step of adding the first species of phospholipid (and the step of mixing the solution following the addition of the first species of phospholipid) is performed prior to the step of adding the second species of phospholipid to the solution (and the step of mixing the solution following the addition of the second species of phospholipid). In embodiments, the step of adding the second species of phospholipid (and the step of mixing the solution following the addition of the second species of phospholipid) is performed prior to the step of adding the first species of phospholipid to a solution (and the step of mixing the solution after following the addition of the first species of phospholipid).

In embodiments, a heating step is performed prior the steps of adding the first species of phospholipid, the second species of phospholipid, and/or the API to a solution. In embodiments, a heating step is performed after the step of adding the first species of phospholipid to the solution and prior to the steps of adding the second species of phospholipid and/or the API to the solution. In embodiments, a heating step is performed after the steps of adding the first species of phospholipid and the second species of phospholipid to the solution and prior the step of adding the API to the solution.

In embodiments, a cooling step is performed after the steps of adding the steps of adding the first species of phospholipid, the second species of phospholipid, and/or the API to the solution. In embodiments, a cooling step is performed after the steps of adding the first species of phospholipid and the second species of phospholipid to the solution and before the steps of adding the API to the solution.

In embodiments, the method comprises the steps of:
(a) adding the slow diffusing solvent (e.g., to a container);
(b) adding the fast diffusing solvent to the slow diffusing solvent while mixing (e.g., mixing for about 5-15 min) to obtain a solution;
(c) adding the first species of phospholipid while mixing the solution of (b);
(d) mixing the solution of (c) (e.g., mixing for about 5-90 min) following the addition of the first species of phospholipid;
(e) heating the solution of (d) (e.g., heating the solution to about 30-35° C.) while mixing the solution of (d);
(f) adding the second species of phospholipid while mixing the solution of (e);
(g) mixing the solution of (f) (e.g., mixing the solution for about 20 min to about 8 hours) following the addition of the second species of phospholipid;
(h) adding the API to the solution of (g) while mixing;
(i) mixing the solution of (h) (e.g., mixing for about 5 min to about 2 hours) following the addition of the API;
(j) cooling the solution of (i) to room temperature (e.g., about 15-25° C.);
(k) filtering the solution of (j) through a sterile filter (e.g., a 0.22 μm sterile filter, or a 0.8/0.22 μm sterile filter, or a 0.4/0.22 μm sterile filter, or a 0.22/0.4 μm sterile filter); and
(l) filling an amount (e.g., 1-10 mL) of the solution of (k) into a sterile vial, pre-syringe, or cartridge.

In embodiments, the first species of phospholipid is Phospholipon® 90 G. In embodiments, the second species of phospholipid is 1,2-dioleoyl-sn-glycero-3-phosphoethanolamine (DOPE). In embodiments, the slow diffusing solvent is Miglyol® 812 N. In embodiments, the fast diffusing solvent is ethanol. In embodiments, the API is a peptide or a pharmaceutically acceptable salt thereof. In embodiments, the API is a MC4R agonist peptide or a pharmaceutically acceptable salt thereof.

In embodiments, the method comprises the steps of:
(a) adding the slow diffusing solvent (e.g., to a container);
(b) adding the fast diffusing solvent to the slow diffusing solvent while mixing (e.g., mixing for about 5-15 min) to obtain a solution;
(c) adding the first species of phospholipid while mixing the solution of (b);
(d) mixing the solution of (c) (e.g., mixing for about 5-90 min) after adding the first species of phospholipid;
(e) adding the second species of phospholipid to the solution of (d) while mixing the solution;
(f) heating the solution of (e) (e.g., heating to about 30-35° C.) while mixing the solution:
(g) mixing the solution of (f) (e.g., mixing for about 20 min to about 8 hours);
(h) cooling the solution of (g) to room temperature (e.g., about 15-25° C.);
(i) adding the API to the solution of (h) while mixing the solution;
(j) mixing the solution of (i) (e.g., mixing for about 5 min to about 2 hours) following the addition of the API;
(k) filtering the solution of (j) through a sterile filter (e.g., a 0.22 μm sterile filter, or a 0.8/0.22 μm sterile filter, or a 0.4/0.22 μm sterile filter, or a 0.22/0.4 μm sterile filter); and
(l) filling an amount (e.g., 1-10 mL) of the solution of (k) into a sterile vial, pre-syringe, or cartridge.

In embodiments, the first species of phospholipid is Phospholipon® 90 G. In embodiments, the second species of phospholipid is 1,2-dioleoyl-sn-glycero-3-phosphoethanolamine (DOPE). In embodiments, the slow diffusing solvent is Miglyol® 812 N. In embodiments, the fast diffusing solvent is ethanol. In embodiments, the API is a peptide or a pharmaceutically acceptable salt thereof. In embodiments, the API is a MC4R agonist peptide or a pharmaceutically acceptable salt thereof.

In embodiments, the method comprises the steps of:
(a) adding the slow diffusing solvent (e.g., to a container);
(b) adding the fast diffusing solvent to the slow diffusing solvent while mixing (e.g., mixing for about 5-15 min) to obtain a solution;
(c) adding the first species of phospholipid to the solution of (b) while mixing the solution;
(d) mixing the solution of (c) (e.g., mixing for about 5-90 min) following the addition of the first species of phospholipid;
(e) adding the second species of phospholipid to the solution of (d) while mixing the solution;
(f) mixing the solution of (e) (e.g., mixing for about 20 min to about 8 hours) following the addition of the second species of phospholipid;
(g) adding the API to the solution of (f) while mixing the solution;
(h) mixing the solution of (g) (e.g., mixing for about 5 min to about 2 hours) following the addition of the API;
(i) filtering the solution of (h) through a sterile filter (e.g., a 0.22 μm sterile filter, or a 0.8/0.22 μm sterile filter, or a 0.4/0.22 μm sterile filter, or a 0.22/0.4 μm sterile filter); and
(j) filling an amount (e.g., 1-10 mL) of the solution of (i) into a sterile vial, pre-syringe, or cartridge.

In embodiments, the first species of phospholipid is Phospholipon® 90 G. In embodiments, the second species of phospholipid is 1,2-dioleoyl-sn-glycero-3-phosphoethanolamine (DOPE). In embodiments, the slow diffusing solvent is Miglyol® 812 N. In embodiments, the fast diffusing solvent is ethanol. In embodiments, the API is a peptide or a pharmaceutically acceptable salt thereof. In embodiments, the API is a MC4R agonist peptide or a pharmaceutically acceptable salt thereof

In embodiments, the method comprises the steps of:
(a) adding the slow diffusing solvent (e.g., to a container);
(b) adding the fast diffusing solvent to the slow diffusing solvent while mixing (e.g., mixing for about 5-15 min) to obtain a solution;
(c) adding the first species of phospholipid to the solution of (b) while mixing the solution;
(d) mixing the solution of (c) (e.g., mixing for about 5-90 min) following the addition of the first species of phospholipid;
(e) heating the solution of (d) (e.g., heating to about 30-35° C.) while mixing the solution;
(f) adding the second species of phospholipid to the solution of (e) while mixing the solution;
(g) mixing the solution of (f) (e.g., mixing for about 20 min to about 8 hours) following the addition of the second species of phospholipid;
(h) adding the API to the solution of (g) while mixing the solution;
(i) mixing the solution of (h) (e.g., mixing for about 5 min to about 2 hours) following the addition of the API;
(j) cooling the solution of (i) to room temperature (e.g., about 15-25° C.);
(k) filtering the solution of (j) through a sterile filter (e.g., a 0.22 μm sterile filter, or a 0.8/0.22 μm sterile filter, or a 0.4/0.22 μm sterile filter, or a 0.22/0.4 μm sterile filter);
(l) adding ethanol to the solution of (k) following filtration to obtain a target weight of the solution by passing the ethanol through the same filter of step (k); and
(m) filling an amount (e.g., 1-10 mL) of the solution of (1) into a sterile vial, pre-syringe, or cartridge.

In embodiments, the first species of phospholipid is Phospholipon® 90 G. In embodiments, the second species of phospholipid is 1,2-dioleoyl-sn-glycero-3-phosphoethanolamine (DOPE). In embodiments, the slow diffusing solvent is Miglyol® 812 N. In embodiments, the fast diffusing solvent is ethanol. In embodiments, the API is a peptide or a pharmaceutically acceptable salt thereof. In embodiments, the API is a MC4R agonist peptide or a pharmaceutically acceptable salt thereof

In embodiments, the method comprises the steps of:
(a) adding the slow diffusing solvent (e.g., to a container);
(b) adding the fast diffusing solvent to the slow diffusing solvent while mixing (e.g., mixing for about 5-15 min) to obtain a solution;
(c) heating the solution of (b) (e.g., heating to about 30-35° C.) while mixing the solution;
(d) adding the second species of phospholipid to the solution of (c) while mixing the solution;
(e) mixing the solution of (d) (e.g., mixing for about 20 min to about 8 hours) following the addition of the second species of phospholipid;
(f) adding the first species of phospholipid to the solution of (e) while mixing the solution;
(g) mixing the solution of (f) (e.g., mixing for about 5-90 min) following the addition of the first species of phospholipid;
(h) adding the API to the solution of (g) while mixing the solution;
(i) mixing the solution of (h) (e.g., mixing for about 5 min to about 2 hours) following the addition of the API;
(j) cooling the solution of (i) to room temperature (e.g., about 15-25° C.);
(k) filtering the solution of (j) through a sterile filter (e.g., a 0.22 μm sterile filter, or a 0.8/0.22 μm sterile filter, or a 0.4/0.22 μm sterile filter, or a 0.22/0.4 μm sterile filter); and
(l) filling an amount (e.g., 1-10 mL) of the solution of (k) into a sterile vial, pre-syringe, or cartridge.

In embodiments, the first species of phospholipid is Phospholipon® 90 G. In embodiments, the second species of phospholipid is 1,2-dioleoyl-sn-glycero-3-phosphoethanolamine (DOPE). In embodiments, the slow diffusing solvent is Miglyol® 812 N. In embodiments, the fast diffusing solvent is ethanol. In embodiments, the API is a peptide or a pharmaceutically acceptable salt thereof. In embodiments, the API is a MC4R agonist peptide or a pharmaceutically acceptable salt thereof.

In embodiments, the compositions of present disclosure may be prepared using any of the Processes I, II, III, IV, and V in Table EEE.

Features of the compositions or methods of using them can include one or more of the following enumerated embodiments.

Embodiment 1. A composition comprising
a. about: 20-60% (W/W) total phospholipids, wherein the phospholipids comprise a first and a second species of phospholipid in a ratio of about: 80:20, 70:30, 60:40, 50:50, and 40:60 of the first species to the second species, wherein the second species is a phospholipid comprising a lipid with dioleoyl and glycero group and/or phosphoethanolamine group;
b. about: 10-60% (W/W) of a slow diffusing solvent;
c. about: 5-30% (W/W) of a fast diffusing solvent or solvent that is highly miscible with water such as an alcohol (such as ethanol), NMP, DMSO or a combination thereof, and
d. optionally an active pharmaceutical ingredient (API), optionally wherein the API is a peptide, optionally wherein the peptide comprises one or more non-canonical amino acids.

Embodiment 2. The composition of embodiment 1, wherein the second species of phospholipid is DOPE.

Embodiment 3. The composition of embodiment 1 or 2, wherein the first species of phospholipid is phosphatidyl choline.

Embodiment 4. The composition of any one of the preceding embodiments, wherein the first species of phospholipid is phosphatidyl choline, the second species of phospholipid is DOPE, and the first and second species are in a ratio of about: 80:20, 75:25, 70:30, 60:40; 50:50; 40:60; optionally wherein the total phospholipid content is at least about: 30, 35, 36, 37, 38, 39, 40, 41, 42, 43, 44, 45, 46, 47, 48, 49, 50, 51, 52, 53, 54, 55, 60%; e.g., about: 40-60%.

Embodiment 5. The composition of any one of the preceding embodiments, wherein the slow diffusing solvent comprises one or more of medium or long chain triglyceride, Miglyol® 812 N, sesame oil, castor oil, polyoxyl 35 castor oil, soybean oil, PEG-60 hydrogenated castor oil, peanut oil, cottonseed oil, corn oil, glycerin, monothioglycerol, glyceryl palmitostearate, glycerol dioleate, including combinations of the foregoing.

Embodiment 6. The composition of embodiment 5, wherein the slow diffusing solvent is a medium chain triglyceride.

Embodiment 7. The composition of embodiment 5 or 6 wherein the slow diffusing solvent is about: 14, 15, 16, 17, 20, 25, 28, 30, 31, 33, 34, 35, 37, 38, 39, 40, 41, 42, 43, 44, 52, 53, 55, 58, or 60% (W/W).

Embodiment 8. The composition of any one of the preceding embodiments wherein the fast diffusing solvent is ethanol, optionally at a concentration of about: 6, 7, 8, 9, 10, 11, 12, 13, 14, 15, 16, 20, 25, 26, 27, 28, 29, or 30% (W/W).

Embodiment 9. The composition of any one of the preceding embodiments wherein the composition comprises an API.

Embodiment 10. The composition of embodiment 9, wherein the API is present at a concentration of at least about: 1, 2, 3, 4, 5, 6, 7, 8, 9, 10% (W/W), or more.

Embodiment 11. The composition of embodiment 9 or 10, wherein the API is a peptide comprising at least about: 5, 6, 7, 8, 9, or 10 amino acids.

Embodiment 12. The composition of any one of embodiments 9-11, wherein the API is a peptide comprising one or more non-canonical amino acids.

Embodiment 13. The composition of any one of embodiments 9-12, wherein the API is a G protein coupled receptor (GPCR) modulator, such as an agonist, antagonist, or biased signaling molecule: e.g., wherein the GPCR is a melanocortin receptor, such as a MC4R.

Embodiment 14. The composition of any one of the preceding embodiments wherein the composition comprises an API and, upon administration to a mammalian subject (e.g., by subcutaneous injection), exhibits and extended release of the API, relative to a control composition, such as a control composition not comprising the second phospholipid species, as evaluated by, for example Cmax, Css, or flat exposure, e.g., at up to 24, 36, 48. 60, 72, 84, or 96 hours.

Embodiment 15. The composition of any one of the preceding embodiments, which is an extended release composition.

Embodiment 16. An article of manufacture comprising the composition of any one of the preceding embodiments.

Embodiment 17. The article of embodiment 16, wherein the article comprises a prefilled medical device, such as a syringe.

Embodiment 18. A method comprising administering an effective amount of the composition of any one of embodiments 1-15 to a subject, optionally via the article of embodiment 16 or 17.

### EXAMPLES

### Example 1: Extended Release of API

The combination of lipids (PL 90 G and DOPE) along with Solvent system (Miglyol® 812 N and Ethanol) when used for a peptide with high clearance rate or short half-life provides a sustained plasma concentrations for up to or more than 7 days when administered subcutaneously.

In addition, above the composition also helps to maintain low Cmax to Css state or flat exposure up to 36 h to 72 h in mini pigs and more than 96 h in rats.

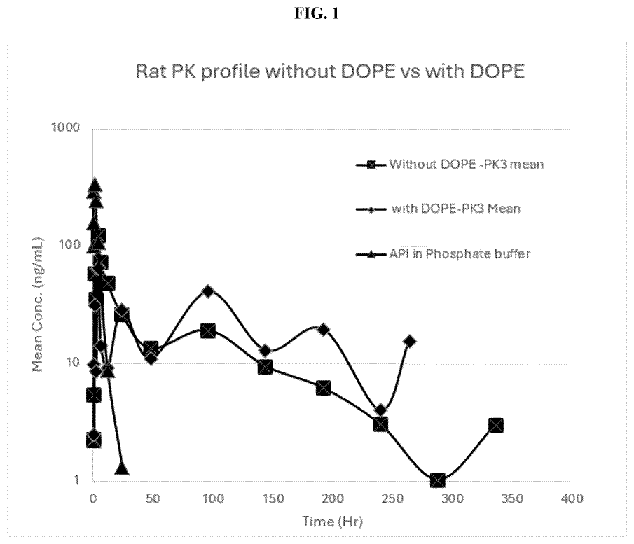

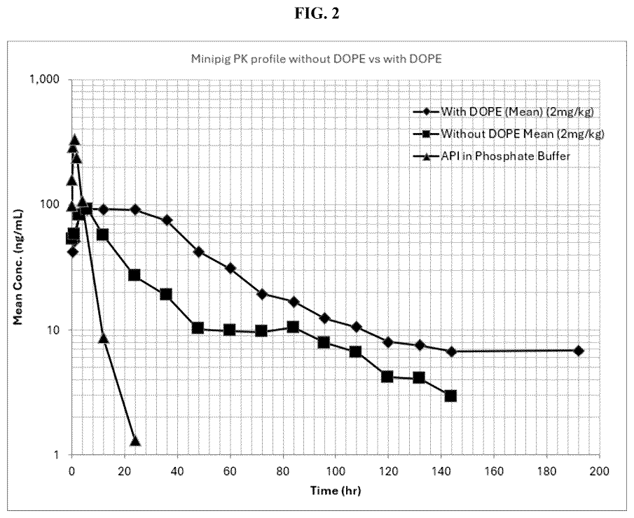

Without the DOPE in the system this cannot be achieved. Illustrative results are shown in FIGS. 1 and 2. Respective, formulations with API were prepared and administered subcutaneously to rats (FIG. 1) and mini pigs (FIG. 2). Exemplary APIs in the compositions include, e.g., peptide 1158 or a salt thereof, such as an acetate salt or a trifluoroacetate salt thereof. Post administration, multiple blood samples were collected at various timepoints ranging from 0.5 hours to 336 hours. Collected samples were stored at −70° C. until analysis. The samples were then tested using LCMS/MS method (Method indicated below as shown in Table AAA) to measure plasma concentrations of API at respective time points.

TABLE AAA
Summary of Analysis Methods
BioAnalysis Method Summary
LC Conditions:

| Injection Volume | 10 uL | | | |
|---|---|---|---|---|
| | | Time | % | Flow |
| | | (min) | MPB | (mL/min) |
| Column Id, & | Acquity UPLC HSS T3, 2.1 × 50 mm, 1.8 um | | | |
| Dimensions | | | | |
| Temperature (° C.) | 55 | 0.00 | 10.0 | 0.800 |
| Mobile Phase A | 100:0.2 (v:v) Water:Formic Acid | 1.00 | 55.0 | 0.800 |
| Mobile Phase B | 100:0.2 (v:v) Acetonitrile:Formic Acid | 1.01 | 95.0 | 0.800 |
| Needle Rinse 1 | 50:25:25 Isopropanol:Acetone:Methanol | 1.50 | 95.0 | 0.800 |
| Needle Rinse 2 | 80:10:10:0.1 | 1.51 | 10.0 | 0.800 |
| | Water:Methanol:Acetonitrile:Formic Acid | | | |
| | | 2.00 | 10.0 | 0.800 |

MS Conditions

| | |
|---|---|
| MS/MS: | API6500+ |
| Ionization Method: | ESI |
| Positive/Negative | Positive |
| Ion: | |
| Resolution: | Unit/Unit |
| Source | 500 |
| Temperature (° C.): | |

Data Analysis

| | |
|---|---|
| Acceptance Criteria | ±25%(±30% LLOQ) |
| Regression Type | Linear (1/(x * x)) |
| Accepted Curve | 1.00-5000 ng/mL |
| Range | |
| Carryover | <0.10% |

### Example 2: Formulation Compositions

This example provides illustrative, non-limiting formulation compositions in accordance with exemplary embodiments of the present disclosure, as shown in Table BBB, and Table CCC, and Table DDD. Exemplary APIs in the compositions include. e.g., peptide 1158 or a salt thereof, such as an acetate salt or a trifluoroacetate salt thereof.

TABLE BBB
Composition of Formulation V0

| Ingredients | % w/w | Conc.(mg/mL) |
|---|---|---|
| Phosphatidylcholine | 22.8 | 235 |
| DOPE | 22.8 | 235 |
| Ethanol | 11.7 | 120 |
| Miglyol ® 812 N | 39.8 | 410 |
| API | 2.9 | 30 |

TABLE CCC
Composition of Formulations V0-V7.2

| | | Concentration (mg/mL) | | | | | |
|---|---|---|---|---|---|---|---|
| | | API | | | Solvent System | | Final |
| S. | Formu- | Pep- | Lipid/Polymer | | Miglyol ® | | weight |
| No | lation | tide | PL 90 G | DOPE | 812N | Ethanol | (mg) |
| 1 | V0 | 30 | 235 | 235 | 410 | 120 | 1030 |
| 2 | V1 | 30 | 258.5 | 211.5 | 410 | 120 | 1030 |
| 3 | V2 | 30 | 293.75 | 176.25 | 410 | 120 | 1030 |
| 4 | V3 | 30 | 352.5 | 117.5 | 410 | 120 | 1030 |
| 5 | V4 | 30 | 188 | 282 | 410 | 120 | 1030 |
| 6 | V5 | 30 | 270.25 | 270.25 | 339.5 | 120 | 1030 |
| 7 | V5.1 | 30 | 270.25 | 270.25 | 169.75 | 289.75 | 1030 |
| 8 | V5.2 | 60 | 270.25 | 270.25 | 339.5 | 120 | 1060 |
| 9 | V6 | 30 | 282 | 282 | 316 | 120 | 1030 |
| 10 | V6.1 | 30 | 282 | 282 | 158 | 278 | 1030 |
| 11 | V6.2 | 60 | 282 | 282 | 316 | 120 | 1060 |
| 12 | V7 | 30 | 300 | 300 | 280 | 120 | 1030 |
| 13 | V7.1 | 30 | 300 | 300 | 140 | 260 | 1030 |
| 14 | V7.2 | 60 | 300 | 300 | 280 | 120 | 1060 |

TABLE DDD
Composition of Formulations with Varying API Concentrations

| | | Concentration (mg/mL) | | | | | |
|---|---|---|---|---|---|---|---|
| | | API | | | Solvent System | | Final |
| S. | Formu- | Pep- | Lipid/Polymer | | Miglyol ® | | weight |
| No | lation | tide | PL 90 G | DOPE | 812N | Ethanol | (mg) |
| 1 | P5 | 2.5- | 235 | 235 | 410 | 120 | 1002.5- |
| | | 120 | | | | | 1120 |
| 2 | P6 | 2.5- | 258.5 | 211.5 | 410 | 120 | 1002.5- |
| | | 120 | | | | | 1120 |
| 3 | P7 | 2.5- | 293.75 | 176.25 | 410 | 120 | 1002.5- |
| | | 120 | | | | | 1120 |
| 4 | P8 | 2.5- | 352.5 | 117.5 | 410 | 120 | 1002.5- |
| | | 120 | | | | | 1120 |
| 5 | P9 | 2.5- | 188 | 282 | 410 | 120 | 1002.5- |
| | | 120 | | | | | 1120 |
| 6 | P10 | 2.5- | 270.25 | 270.25 | 339.5 | 120 | 1002.5- |
| | | 120 | | | | | 1120 |
| 7 | P11 | 2.5- | 270.25 | 270.25 | 169.75 | 289.75 | 1002.5- |
| | | 120 | | | | | 1120 |
| 8 | P13 | 2.5- | 282 | 282 | 316 | 120 | 1002.5- |
| | | 120 | | | | | 1120 |
| 9 | P14 | 2.5- | 282 | 282 | 158 | 278 | 1002.5- |
| | | 120 | | | | | 1120 |
| 12 | P16 | 2.5- | 300 | 300 | 280 | 120 | 1002.5- |
| | | 120 | | | | | 1120 |
| 13 | P17 | 2.5- | 300 | 300 | 140 | 260 | 1002.5- |
| | | 120 | | | | | 1120 |

### Example 3: Process for Preparing

This example provides illustrative, non-limiting processes for preparing the formulations in accordance with exemplary embodiment of the present disclosure, as shown in Table EEE. Exemplary APIs in the compositions include, e.g., peptide 1158 or a salt thereof, such as an acetate salt or a trifluoroacetate salt thereof.

TABLE EEE
Process for Preparing Formulations
Process

| Process I | Process II | Process III | Process IV | Process V |
|---|---|---|---|---|
| Add Miglyol ® | Add Miglyol ® | Add Miglyol ® | Add Miglyol ® | Add Miglyol ® |
| 812 (MCT) | 812 (MCT) | 812 (MCT) | 812 (MCT) | 812 (MCT) |
| Add Ethanol | Add Ethanol | Add Ethanol | Add Ethanol | Add Ethanol |
| while mixing | while mixing | while mixing | while mixing | while mixing |
| for 5 to 15 min | for 5 to 15 min | for 5 to 15 min | for 5 to 15 min | for 5 to 15 min |
| Add | Add | Add | Add | Heat the |
| Phospholipon ® | Phospholipon ® | Phospholipon ® | Phospholipon ® | solution to |
| 90 G while | 90 G while | 90 G while | 90 G while | 30-35 C. while |
| mixing | mixing | mixing | mixing | Mixing |
| Mixing for | Mixing for | Mixing for | Mixing for | Add DOPE |
| 15-90 min | 15-90 min | 15-90 min | 15-90 min | while mixing |
| Heat the | Add DOPE while | Add DOPE while | Heat the | Mixing time 20 |
| solution to | mixing | mixing | solution to | min to 8 h |
| 30-35 C. while | | | 30-35 C. while | |
| Mixing | | | Mixing | |
| Add DOPE | Heat the solution | Mixing time 20 | Add DOPE | Add |
| while mixing | to 30-35 C. | min to 8 h | while mixing | Phospholipon ® |
| | while Mixing | | | 90 G while |
| | | | | mixing |
| Mixing time 20 | Mixing time 20 | Add API or Drug | Mixing time 20 | Mixing for |
| min to 8 h | min to 8 h | substance while | min to 8 h | 15-90 min |
| | | mixing | | |
| Add API or | Cool Down the | Mixing time 5 | Add API or | Add API or |
| Drug substance | Solution to Room | min to 2 h | Drug substance | Drug substance |
| while mixing | temperature | | while mixing | while mixing |
| Mixing time 5 | Add API or Drug | Filter through | Mixing time 5 | Mixing time 5 |
| min to 2 h | substance while | 0.22μ or | min to 2 h | min to 2 h |
| | Mixing | 0.8/0.22μ or | | |
| | | 04/0.22μ or | | |
| | | 022/0.4μ Sterile | | |
| | | filter | | |
| Cool Down the | Mixing time 5 | Fill 1-10 mL | Cool Down the | Cool Down the |
| Solution to | min to 2 h | into sterile Vials | Solution to | Solution to |
| Room | | or Pre-Syringe or | Room | Room |
| temperature | | Cartridge. | temperature | temperature |
| Filter through | Filter through | | Filter through | Filter through |
| 0.22μ or | 0.22μ or | | 0.22μ or | 0.22μ or |
| 0.8/0.22μ or | 0.8/0.22μ or | | 0.8/0.22μ or | 0.8/0.22μ or |
| 04/0.22μ or | 04/0.22μ or | | 04/0.22μ or | 04/0.22μ or |
| 022/0.4μ Sterile | 022/0.4μ Sterile | | 022/0.4μ Sterile | 022/0.4μ Sterile |
| filter | filter | | filter | filter |
| Fill 1-10 mL | Fill 1-10 mL | | QS the | Fill 1-10 mL |
| into sterile | into sterile | | formulation to | into sterile |
| Vials or Pre- | Vials or Pre- | | target weight | Vials or Pre- |
| Syringe or | Syringe or | | with Ethanol by | Syringe or |
| Cartridge. | Cartridge. | | passing the | Cartridge. |
| | | | ethanol through | |
| | | | the same filter. | |
| | | | Fill 1-10 mL | |
| | | | into sterile | |
| | | | Vials or Pre- | |
| | | | Syringe or | |
| | | | Cartridge. | |

### Example 4: Reliability of the Methods of Preparing

This example illustrates the reliability of the various methods of preparing the compositions of present disclosure. Briefly, compositions comprising a peptide API prepared by different processes (see, Table EEE) were administered subcutaneously to dogs at a location between the shoulder blades at least 25 mm from the spine. Operators create a “tent” shaped fold in the scruff of the neck by grasping the skin between fingers and thumb and lifted the skin up from the animal's neck to create a triangle shape on the scruff. An 18G-30G gauge needle was inserted into the center of the triangle, with the tip of the needle being at least 5-8 mm below the skin surface while ensuring the needle tip is not pushed through the other side of the skin. The compositions were gently inverted prior to dosing. The syringe plunger was pressed at a uniform rate, and the needle was held in place for at least 3 seconds after full volume delivered to ensure no material ejection upon needle withdrawal. Post injection, samples were collected up to 480 h to measure plasma concentration of the API and to monitor the PK profile.

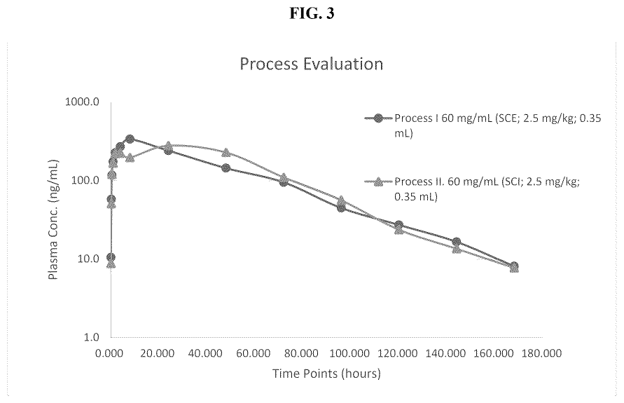

As shown in FIG. 3, compositions comprising a peptide API prepared by two different processes yield similar PK characteristics after administration. Exemplary APIs in the compositions include, e.g., peptide 1158 or a salt thereof, such as an acetate salt or a trifluoroacetate salt thereof.

### Example 5: API Concentration Study

This example illustrates the effect of API concentrations in the compositions on the PK profile of the compositions after administration. Compositions with different API concentrations were administered to mini pigs in manners similar to Example 4. Exemplary APIs in the compositions include, e.g., peptide 1158 or a salt thereof, such as an acetate salt or a trifluoroacetate salt thereof.

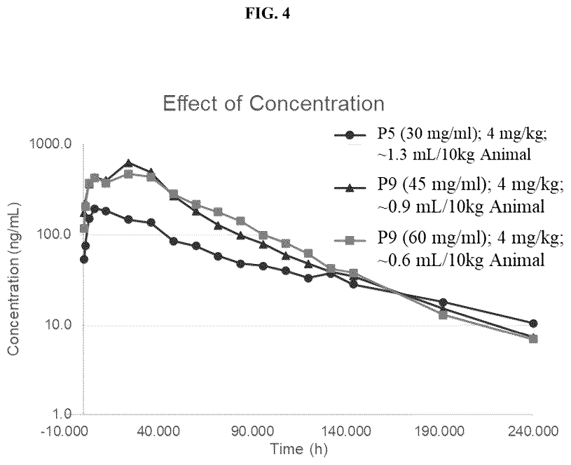

FIG. 4 shows the PK profiles of compositions with three different peptide API concentrations (30 mg/mL, 45 mg/mL, and 60 mg/mL) examined. The compositions with 30 mg/mL API, 45 mg/mL API, and 60 mg/mL API were prepared using formulations P5, P9, and P9, shown in Table DDD, respectively. Compositions with 45 mg/mL API and 60 mg/mL API, both of which were prepared using formulations P9, exhibited similar PK profiles after administration.

### Example 6: Exemplary, Non-Limiting Peptides

This example provides exemplary, non-limiting peptides in accordance with exemplary embodiments of present disclosure.

Table 7 shown below lists families of molecules that have the X₃ position (e.g., Aib(O-cyclic) in common. The X₃ residue chosen for investigation was based on X₃ and X₄ combinatorial pairings that elicited the greatest selectivity between MC4R and MC1R. Without being bound to a particular theory, throughout this Table, the peptide sequences illustrated how selectivity between the MC4R and MC1R receptors increased when a specific X₃ and X₄ pairing was identified. The X₁, X₅, and X₇ positions contributed to selectivity, as was seen in Table 7 through the contribution of the X₅ position to improvements in selectivity (Table 7, Molecule 1092, Molecule 1093, and Molecule 1158). However, the substitution interplay was most evident in analogues where certain X₃ and X₄ pairings lead to retention of high MC4R functional potency, with significant decrement of MC1R potency (to generate selectivity).

Without being bound to a particular theory, the family of peptides that contained Aib(O-cyclic) demonstrated that certain pairings produced improved selectivity, such as the Gln at X₄. This data also illustrated how the identity of the X₅ position contributes to selectivity. When comparing the selectivity of MC4R v MC1R, larger values indicate selectivity towards MC4R. When comparing the bias of MC4R B-arrestin v MC4R CAMP, larger values indicate bias towards B-arrestin.

TABLE 7
Compounds with Aib(O-cyclic) at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.
Table 7: Compounds with Aib(O-cyclic) at X₃

| | | | | | | | | | | | | | | | | | | Bias: MC4R |
|---|---|---|---|---|---|---|---|---|---|---|---|---|---|---|---|---|---|---|
| Molecule | | | | | | | | | | | | | | | | | Selectivity: | B-arrestin v |
| name | X₋₄ | X₋₃ | X₋₂ | X₋₁ | X₁ | X₂ | X₃ | X₄ | X₅ | X₆ | X₇ | X₈ | X₉ | X₁₀ | X₁₁ | X₁₂ | MC4R v MC1R | MC4R cAMP |
| 1150 | Lys* | D-Arg | Gly | D-Arg | D-Nar | Cys | Aib(O-cyclic) | Gln | D-Phe(4-F) | Arg | Trp(6-F) | Cys | | | | | * | + |
| 1142 | | Lys* | Gly | D-Arg | D-Nar | Cys | Aib(O-cyclic) | Gln | D-Phe(4-F) | Arg | Trp(6-F) | Cys | | | | | ** | + |
| 1144 | | Lys* | PEG1 | PEG1 | D-Nar | Cys | Aib(O-cyclic) | Gln | D-Phe(4-F) | Arg | Trp(6-F) | Cys | | | | | ** | ++ |
| 1151 | Lys* | D-Arg | PEG1 | D-Arg | Beta-homoArg | Cys | Aib(O-cyclic) | Gln | D-Phe(4-F) | Arg | Trp(6-F) | Cys | | | | | * | ++ |
| 1152 | Lys* | D-Arg | Gly | D-Arg | D-Nar | Cys | Aib(O-cyclic) | 3Pal | D-Phe(4-F) | Arg | Trp(6-F) | Cys | | | | | * | ++ |
| 1153 | Lys* | D-Arg | Gly | D-Arg | D-Nar | Cys | Aib(O-cyclic) | Orn | D-Phe(4-F) | Arg | Trp(6-F) | Cys | | | | | * | ++ |
| 1122 | | | | | D-Nar | Cys | Aib(O-cyclic) | 3Pal | D-Phe(4-F) | Arg | Trp(6-F) | Cys | | | | | *** | ++ |
| 1123 | | | | | D-Nar | Cys | Aib(O-cyclic) | Orn | D-Phe(4-F) | Arg | Trp(6-F) | Cys | | | | | ** | + |
| 25 | | | | | D-Nar | Glu | Aib(O-cyclic) | Gln | D-Phe(4-F) | Arg | Trp(6-F) | Dap | | | | | *** | + |
| 28 | | | | | Beta-homoArg | Cys | Aib(O-cyclic) | Gln | D-Phe(4-F) | Arg | Trp(6-F) | Cys | | | | | *** | + |
| 1158 | | | | | D-Nar | Cys | Aib(O-cyclic) | Gln | D-Phe(4-F) | Arg | Trp(6-F) | Pen | | | | | *** | + |
| 43 | | | | | D-Nar | hCys | Aib(O-cyclic) | Gln | D-Phe(4-F) | Arg | Trp(6-F) | Pen | | | | | ** | ++ |
| 46 | | | | | D-Nar | Cys | Aib(O-cyclic) | Gln | D-Phe | Arg | Trp | Cys | | | | | *** | ++ |
| 51 | | | | | Arg | Cys | Aib(O-cyclic) | Gln | D-Phe | Arg | Trp | Cys | | | | | *** | ++ |
| 83 | | | | | D-Nar | Cys | Aib(O-cyclic) | Thr | D-Phe(4-F) | Arg | Trp(6-F) | Cys | | | | | *** | ++ |
| 86 | | | | | D-Nar | Cys | Aib(O-cyclic) | Thr | D-Phe(4-F) | Arg | Trp(6-F) | Pen | | | | | *** | + |
| 89 | | | | | D-Nar | Cys | Aib(O-cyclic) | Ser | D-Phe(4-F) | Arg | Trp(6-F) | Cys | | | | | *** | ++ |
| 92 | | | | | D-Nar | Cys | Aib(O-cyclic) | Ser | D-Phe(4-F) | Arg | Trp(6-F) | Pen | | | | | *** | ++ |
| 111 | Lys* | Gly | Gly | Gly | D-Nar | Cys | Aib(O-cyclic) | Gln | D-Phe(4-F) | Arg | Trp(6-F) | Cys | | | | | * | ++ |
| 112 | | Lys* | Gly | Gly | D-Nar | Cys | Aib(O-cyclic) | Gln | D-Phe(4-F) | Arg | Trp(6-F) | Cys | | | | | ** | ++ |
| 113 | | | Lys* | Gly | D-Nar | Cys | Aib(O-cyclic) | Gln | D-Phe(4-F) | Arg | Trp(6-F) | Cys | | | | | ** | ++ |
| 114 | | | | Lys* | D-Nar | Cys | Aib(O-cyclic) | Gln | D-Phe(4-F) | Arg | Trp(6-F) | Cys | | | | | ** | ++ |
| 115 | | | | | D-Nar | Cys | Aib(O-cyclic) | Gln | D-Phe(4-F) | Arg | Trp(6-F) | Cys | Gly | Gly | Gly | Lys* | ** | ++ |
| 116 | | | | | D-Nar | Cys | Aib(O-cyclic) | Gln | D-Phe(4-F) | Arg | Trp(6-F) | Cys | Gly | Gly | Lys* | | * | ++ |
| 117 | | | | | D-Nar | Cys | Aib(O-cyclic) | Gln | D-Phe(4-F) | Arg | Trp(6-F) | Cys | Gly | Lys* | | | ** | ++ |
| 118 | | | | | D-Nar | Cys | Aib(O-cyclic) | Gln | D-Phe(4-F) | Arg | Trp(6-F) | Cys | Lys* | | | | * | ++ |
| 119 | | | | | D-Nar | Cys | Aib(O-cyclic) | Gln | D-Phe(4-F) | Arg | Trp(6-F) | Cys | PEG1 | PEG1 | Lys* | | ** | ++ |
| 120 | | | | | D-Nar | Cys | Aib(O-cyclic) | Gln | D-Phe(4-F) | Arg | Trp(6-F) | Cys | D-Arg | Gly | Lys* | | ** | ++ |
| 121 | | | | | D-Nar | Cys | Aib(O-cyclic) | Gln | D-Phe(4-F) | Arg | Trp(6-F) | Cys | Pro | Phe | Lys* | | ** | ++ |
| 122 | | | | | D-Nar | Cys | Aib(O-cyclic) | Gln | D-Phe(4-F) | Arg | Trp(6-F) | Cys | Lys | Pro | Val | Lys* | * | ++ |
| 137 | | | | | Beta-homoArg | Cys | Aib(O-cyclic) | Cit | D-Phe(4-F) | Arg | Trp(6-F) | Cys | | | | | *** | + |
| 139 | | | Lys* | Gly | D-Nar | Cys | Aib(O-cyclic) | Cit | D-Phe | Arg | Trp(6-F) | Cys | | | | | ** | ++ |
| 140 | | | | Lys* | Arg | Cys | Aib(O-cyclic) | Gln | D-Phe(4-F) | Arg | Trp(6-F) | Pen | | | | | *** | + |
| 141 | | | | Lys* | D-Nar | Cys | Aib(O-cyclic) | Gln | D-Phe(4-F) | Arg | Trp(6-F) | Pen | | | | | *** | + |
| 142 | | | | Lys* | BetahomoArg | Cys | Aib(O-cyclic) | Gln | D-Phe(4-F) | Arg | Trp(6-F) | Pen | | | | | *** | + |
| 143 | | | | Lys* | Arg | Cys | Aib(O-cyclic) | Gln | D-Phe | Arg | Trp | Pen | | | | | ** | ++ |
| 144 | | | | Lys* | D-Nar | Cys | Aib(O-cyclic) | Gln | D-Phe | Arg | Trp | Pen | | | | | *** | + |
| 145 | | | | Lys* | BetahomoArg | Cys | Aib(O-cyclic) | Gln | D-Phe | Arg | Trp | Pen | | | | | ** | ++ |
| 146 | | | | Lys* | Arg | Cys | Aib(O-cyclic) | Gln | D-Phe | Arg | Trp(6-F) | Pen | | | | | ** | + |
| 147 | | | | Lys* | D-Nar | Cys | Aib(O-cyclic) | Gln | D-Phe | Arg | Trp(6-F) | Pen | | | | | ** | + |
| 148 | | | | Lys* | BetahomoArg | Cys | Aib(O-cyclic) | Gln | D-Phe | Arg | Trp(6-F) | Pen | | | | | *** | ++ |
| 149 | | | | Lys* | Arg | Cys | Aib(O-cyclic) | Gln | D-Phe(4-F) | Arg | Trp | Pen | | | | | *** | + |
| 150 | | | | Lys* | D-Nar | Cys | Aib(O-cyclic) | Gln | D-Phe(4-F) | Arg | Trp | Pen | | | | | *** | + |
| 151 | | | | Lys* | BetahomoArg | Cys | Aib(O-cyclic) | Gln | D-Phe(4-F) | Arg | Trp | Pen | | | | | *** | ++ |
| 152 | | | | | D-Nar | Cys | Aib(O-cyclic) | Gln | D-Phe | Arg | Trp(6-F) | Pen | | | | | *** | + |
| 153 | | | | | D-Nar | Cys | Aib(O-cyclic) | Gln | D-Phe(4-F) | Arg | Trp | Pen | | | | | *** | + |
| 166 | | | | Lys* | D-Nar | Pen | Aib(O-cyclic) | Gln | D-Phe(4-F) | Arg | Trp(6-F) | Pen | | | | | *** | + |
| 167 | | | | Lys* | D-Nar | Pen | Aib(O-cyclic) | Gln | D-Phe(4-F) | Arg | Trp(6-F) | Cys | | | | | * | +++ |
| 168 | | | | | D-Nar | Pen | Aib(O-cyclic) | Gln | D-Phe(4-F) | Arg | Trp(6-F) | Pen | | | | | *** | ++ |
| 169 | | | | | D-Nar | Pen | Aib(O-cyclic) | Gln | D-Phe(4-F) | Arg | Trp(6-F) | Cys | | | | | *** | + |
| 170 | | | | | D-Nar | Cys | Aib(O-cyclic) | Lys | D-Phe(4-F) | Arg | Trp(6-F) | Pen | | | | | *** | + |
| 186 | | | | | D-Nar | Cys | Aib(O-cyclic) | Gln | L-Methionine | Arg | Trp(6-F) | Pen | | | | | N/A | N/A |
| | | | | | | | | | sulfoxide | | | | | | | | | |
| 187 | | | | | D-Nar | Cys | Aib(O-cyclic) | Gln | L-Methionine | Arg | Trp(6-F) | Pen | | | | | N/A | N/A |
| | | | | | | | | | sulfone | | | | | | | | | |
| 188 | | | | | D-Nar | Cys | Aib(O-cyclic) | Gln | (2S)-2-Amino-4- | Arg | Trp(6-F) | Pen | | | | | N/A | N/A |
| | | | | | | | | | cyanobutanoic acid | | | | | | | | | |
| 189 | | | | | D-Nar | Cys | Aib(O-cyclic) | Gln | 3-(Acetylamino)-L- | Arg | Trp(6-F) | Pen | | | | | N/A | N/A |
| | | | | | | | | | alanine | | | | | | | | | |
| 190 | | | | | D-Nar | Cys | Aib(O-cyclic) | Gln | O-Carbamoyl-L- | Arg | Trp(6-F) | Pen | | | | | N/A | N/A |
| | | | | | | | | | serine | | | | | | | | | |
| 191 | | | | | D-Nar | Cys | Aib(O-cyclic) | Gln | 2-Hydroxy-L- | Arg | Trp(6-F) | Pen | | | | | N/A | N/A |
| | | | | | | | | | tryptophan | | | | | | | | | |
| 192 | | | | | D-Nar | Cys | Aib(O-cyclic) | Gln | 3-(Trimethylsilyl)-D- | Arg | Trp(6-F) | Pen | | | | | N/A | N/A |
| | | | | | | | | | alanine | | | | | | | | | |
| 193 | | | | | D-Nar | Cys | Aib(O-cyclic) | Gln | 5,5,5-Trifluoro-D- | Arg | Trp(6-F) | Pen | | | | | N/A | N/A |
| | | | | | | | | | norvaline | | | | | | | | | |
| 194 | | | | | D-Nar | Cys | Aib(O-cyclic) | Gln | 3-(Trifluoromethyl)- | Arg | Trp(6-F) | Pen | | | | | N/A | N/A |
| | | | | | | | | | D-alanine | | | | | | | | | |
| 195 | | | | | D-Nar | Cys | Aib(O-cyclic) | Gln | 3-Cyano-D-alanine | Arg | Trp(6-F) | Pen | | | | | N/A | N/A |
| 196 | | | | | D-Nar | Cys | Aib(O-cyclic) | Gln | 3-Cyclopropyl-D- | Arg | Trp(6-F) | Pen | | | | | N/A | N/A |
| | | | | | | | | | alanine | | | | | | | | | |
| 197 | | | | | D-Nar | Cys | Aib(O-cyclic) | Gln | (R)-2-Amino-4- | Arg | Trp(6-F) | Pen | | | | | N/A | N/A |
| | | | | | | | | | cyclopropylbutanoic | | | | | | | | | |
| | | | | | | | | | acid | | | | | | | | | |
| 198 | | | | | D-Nar | Cys | Aib(O-cyclic) | Gln | (αR)-α-Amino-2- | Arg | Trp(6-F) | Pen | | | | | N/A | N/A |
| | | | | | | | | | pyridine- | | | | | | | | | |
| | | | | | | | | | propanoic acid | | | | | | | | | |
| 199 | | | | | D-Nar | Cys | Aib(O-cyclic) | Gln | (αR)-α-Amino-3- | Arg | Trp(6-F) | Pen | | | | | N/A | N/A |
| | | | | | | | | | pyridine- | | | | | | | | | |
| | | | | | | | | | propanoic acid | | | | | | | | | |
| 200 | | | | | D-Nar | Cys | Aib(O-cyclic) | Gln | (αR)-α-Amino-4- | Arg | Trp(6-F) | Pen | | | | | N/A | N/A |
| | | | | | | | | | pyridine- | | | | | | | | | |
| | | | | | | | | | propanoic acid | | | | | | | | | |
| 1058 | | | | | D-Nar | Cys | Aib(O-cyclic) | His | D-Phe | Arg | Trp(6-F) | Cys | | | | | ** | + |
| 1092 | | | | | D-Nar | Cys | Aib(O-cyclic) | Gln | D-Phe | Arg | Trp(6-F) | Cys | | | | | *** | ++ |
| 1093 | | | | | D-Nar | Cys | Aib(O-cyclic) | Gln | D-Phe(4-F) | Arg | Trp(6-F) | Cys | | | | | *** | ++ |
| 1102 | | | | | D-Nar | Cys | Aib(O-cyclic) | hGln | D-Phe | Arg | Trp(6-F) | Cys | | | | | ** | +++ |
| 1103 | | | | | D-Nar | Cys | Aib(O-cyclic) | Cit | D-Phe | Arg | Trp(6-F) | Cys | | | | | ** | +++ |
| 1106 | | | | | D-Nar | Cys | Aib(O-cyclic) | Cit | D-Phe(4-F) | Arg | Trp(6-F) | Cys | | | | | *** | ++ |
| 1107 | | | | | D-Nar | Cys | Aib(O-cyclic) | hCit | D-Phe(4-F) | Arg | Trp(6-F) | Cys | | | | | *** | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Tables 12-17 below show families of molecules that have the X₃ position in common. The X₃ residues chosen for investigation were based on X₃ and X₄ combinatorial pairings identified in Table 9 that elicited the greatest selectivity between MC4R and MC1R. Without being bound to a particular theory, throughout these Tables, the peptide sequences illustrated how selectivity between the MC4R and MC1R receptors increased when a specific X₃ and X₄ pairing was identified. The X₁, X₅, and X₇ positions contributed to selectivity, as was seen in Table 15 through the contribution of the X₅ position to improvements in selectivity (Table 14, molecule 1092, molecule 1093 and molecule 1158, and in Table 13, molecule 1101 and molecule 1100). However, the substitution interplay was most evident in analogues where certain X₃ and X₄ pairings lead to retention of high MC4R functional potency, with significant decrement of MC1R potency (to generate selectivity). When comparing the selectivity of MC4R v MC1R, larger values indicate selectivity towards MC4R. When comparing the bias of MC4R B-arrestin v MC4R cAMP, larger values indicate bias towards B-arrestin.

TABLE 12
Compounds with Phg at X₃. All peptides are N-acetylated, have a disulfide linkage and contain a C-terminal amide.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1119 | D-Nar | Cys | Phg | 3-Pal | D-Phe(4-F) | Arg | Trp(6-F) | Cys | *** | ++ |
| 1111 | D-Nar | Cys | Phg | His | D-Phe(4-F) | Arg | Trp(6-F) | Cys | ** | + |
| 1112 | D-Nar | Cys | Phg | His | D-Phe(4-F) | Arg | Trp(5-Me) | Cys | ** | + |
| 1113 | D-Nar | Cys | Phg | His | D-Phe(4-F) | Arg | Trp(6-Me) | Cys | ** | + |
| 1034 | Beta- | Cys | Phg | His | D-Phe | Arg | Trp | Cys | ** | + |
| | homoArg | | | | | | | | | |
| 1110 | D-Nar | Cys | Phg | His | D-Phe | Arg | Trp(6-F) | Cys | * | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Another example of the non-predictable pairing of X₃ and X₄ necessary to generate selectivity was the difference between the peptide Molecule 1094 and related peptides that vary in the X₄ position, namely Molecule 1036, or Molecule 1084, or Molecule 1100. Without being bound to a particular theory, the structures showed how the correct pairing of the X₃ and X₄ position was important in generating a transition from no selectivity to ˜60× selectivity between MC4R and MC1R. When comparing the selectivity of MC4R v MC1R, larger values indicate selectivity towards MC4R. When comparing the bias of MC4R B-arrestin v MC4R CAMP, larger values indicate bias towards B-arrestin.

TABLE 13
Compounds with D-aMeOrn at X₃. All peptides are N-acetylated,
have a disulfide linkage and contain a C-terminal amide.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1094 | D-Nar | Cys | D-aMeOrn | Gln | D-Phe | Arg | Trp(6-F) | Cys | *** | ++ |
| 1124 | D-Nar | Cys | D-aMeOrn | 3-Pal | D-Phe(4-F) | Arg | Trp(6-F) | Cys | *** | ++ |
| 1036 | D-Nar | Cys | D-aMeOrn | His | D-Phe | Arg | Trp(6-F) | Cys | ** | + |
| 1101 | D-Nar | Cys | D-aMeOrn | hGln | D-Phe(4-F) | Arg | Trp(6-F) | Cys | ** | +++ |
| 1125 | D-Nar | Cys | D-aMeOrn | Orn | D-Phe(4-F) | Arg | Trp(6-F) | Cys | ** | + |
| 1100 | D-Nar | Cys | D-aMeOrn | hGln | D-Phe | Arg | Trp(6-F) | Cys | ** | +++ |
| 1055 | D-Arg | Cys | D-aMeOrn | His | D-Phe | Arg | Trp(6-Me) | Cys | ** | + |
| 1109 | D-Nar | Cys | D-aMeOrn | 4-Pal | D-Phe | Arg | Trp(6-F) | Cys | ** | +++ |
| 1010 | Arg | Cys | D-aMeOrn | His | D-Phe | Arg | Trp | Cys | ** | + |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, the family of peptides that contained Aib(O-cyclic) demonstrated that certain pairings produced improved selectivity, such as the Gln at X₄. This data also illustrated how the identity of the X₅ position contributes to selectivity.

TABLE 14
Compounds with Aib(O-cyclic) at X₃. All peptides are N-acetylated, have a disulfide linkage and contain a C-terminal
amide. When comparing the selectivity of MC4R v MC1R, larger values indicate selectivity towards MC4R. When
comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1106 | D-Nar | Cys | Aib(O-cyclic) | Cit | D-Phe(4-F) | Arg | Trp(6-F) | Cys | *** | ++ |
| 1122 | D-Nar | Cys | Aib(O-cyclic) | 3-Pal | D-Phe(4-F) | Arg | Trp(6-F) | Cys | *** | ++ |
| 1107 | D-Nar | Cys | Aib(O-cyclic) | hCit | D-Phe(4-F) | Arg | Trp(6-F) | Cys | *** | ++ |
| 1093 | D-Nar | Cys | Aib(O-cyclic) | Gln | D-Phe(4-F) | Arg | Trp(6-F) | Cys | *** | ++ |
| 1092 | D-Nar | Cys | Aib(O-cyclic) | Gln | D-Phe | Arg | Trp(6-F) | Cys | *** | ++ |
| 1103 | D-Nar | Cys | Aib(O-cyclic) | Cit | D-Phe | Arg | Trp(6-F) | Cys | ** | +++ |
| 1102 | D-Nar | Cys | Aib(O-cyclic) | hGln | D-Phe | Arg | Trp(6-F) | Cys | ** | +++ |
| 1058 | D-Nar | Cys | Aib(O-cyclic) | His | D-Phe | Arg | Trp(6-F) | Cys | ** | + |
| 1123 | D-Nar | Cys | Aib(O-cyclic) | Orn | D-Phe(4-F) | Arg | Trp(6-F) | Cys | ** | + |
| 1158 | D-Nar | Cys | Aib(O-cyclic) | Gln | D-Phe(4-F) | Arg | Trp(6-F) | Pen | *** | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 15
Table 15: Compounds with L-aMeGlu at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | | Bias: MC4R |
|---|---|---|---|---|---|---|---|---|---|---|---|
| Molecule | | | | | | | | | | Selectivity: | B-arrestin v |
| Name | X₋₁ | X₁ | X₂ | X₃ | X₄ | X₅ | X₆ | X₇ | X₈ | MC4R v MC1R | MC4R cAMP |
| 1020 | | D-Nar | Cys | L-aMeGlu | His | D-Phe | Arg | Trp(6-Me) | Cys | *** | + |
| 1035 | | D-Nar | Cys | L-aMeGlu | His | D-Phe | Arg | Trp(6-F) | Cys | *** | + |
| 1043 | | D-Nar | Cys | L-aMeGlu | His | D-Phe | Arg | Trp(5-Me) | Cys | *** | + |
| 1041 | | D-Nar | Cys | L-aMeGlu | His | D-Phe(4-F) | Arg | Trp(6-Me) | Cys | *** | + |
| 1030 | | Beta-homoArg | Cys | L-aMeGlu | His | D-Phe(3-CF3) | Arg | TRP | Cys | *** | +++ |
| 1024 | | Beta-homoArg | Cys | L-aMeGlu | His | D-Phe | Arg | Trp(6-Me) | Cys | *** | + |
| 1019 | | D-Nar | Cys | L-aMeGlu | His | D-Phe | Arg | Trp | Cys | *** | + |
| 1085 | | D-Nar | Cys | L-aMeGlu | His | D-Phe(3-F) | Arg | Trp(6-Me) | Cys | *** | + |
| 1016 | | D-Arg | Cys | L-aMeGlu | His | D-Phe | Arg | Trp(6-Me) | Cys | *** | + |
| 1083 | | D-Nar | Cys | L-aMeGlu | His | D-Phe(2,3-diF) | Arg | Trp(6-F) | Cys | ** | + |
| 1057 | | Arg | Cys | L-aMeGlu | His | D-Phe(3-F) | Arg | Trp(6-F) | Cys | ** | + |
| 1040 | | D-Nar | Cys | L-aMeGlu | His | D-Phe(3-F) | Arg | Trp(6-F) | Cys | ** | + |
| 1046 | | D-Nar | Cys | L-aMeGlu | His | D-Phe(3,4,5-triF) | Arg | Trp(6-Me) | Cys | ** | ++ |
| 1088 | | D-Nar | Cys | L-aMeGlu | His | D-Phe(2,4,5-triF) | Arg | Trp(6-F) | Cys | ** | + |
| 1038 | | D-Nar | Cys | L-aMeGlu | His | D-Phe(4-F) | Arg | Trp(6-F) | Cys | ** | + |
| 1079 | | D-Nar | Cys | L-aMeGlu | His | D-Phe | Arg | Trp(5-Cl) | Cys | ** | + |
| 1003 | | Arg | Cys | L-aMeGlu | His | D-Phe | Arg | Trp | Cys | ** | + |
| 1090 | | D-Nar | Cys | L-aMeGlu | Gln | D-Phe | Arg | Trp(6-F) | Cys | ** | ++ |
| 1050 | | D-Nar | Cys | L-aMeGlu | His | D-Phe(3-CF3) | Arg | Trp(6-Me) | Cys | ** | +++ |
| 1007 | | D-Arg | Cys | L-aMeGlu | His | D-Phe | Arg | Trp | Cys | ** | + |
| 1022 | | Beta-homoArg | Cys | L-aMeGlu | His | D-Phe | Arg | Trp | Cys | ** | + |
| 1080 | | D-Nar | Cys | L-aMeGlu | His | D-Phe | Arg | Trp(6-Br) | Cys | ** | + |
| 1067 | | D-Nar | Asp | L-aMeGlu | His | D-Phe | Arg | Trp(6-Me) | Dap | ** | + |
| 1104 | | D-Nar | Cys | L-aMeGlu | Cit | D-Phe | Arg | Trp(6-F) | Cys | ** | ++ |
| 1084 | | D-Nar | Cys | L-aMeGlu | His | D-Phe(3-Cl) | Arg | Trp(6-F) | Cys | ** | ++ |
| 1115 | | Nar | Cys | L-aMeGlu | His | D-Phe(4-F) | Arg | Trp(6-F) | Cys | ** | + |
| 1028 | | Beta-homoArg | Cys | L-aMeGlu | His | D-Phe | Arg | Trp(6-F) | Cys | ** | ++ |
| 1087 | | D-Nar | Cys | L-aMeGlu | His | D-Phe(2,4-diF) | Arg | Trp(6-F) | Cys | ** | + |
| 1054 | | L-hArg | Cys | L-aMeGlu | His | D-Phe(4-F) | Arg | Trp(6-F) | Cys | ** | + |
| 1029 | | L-hArg | Cys | L-aMeGlu | His | D-Phe | Arg | Trp(6-Me) | Cys | ** | ++ |
| 1066 | | D-Nar | Glu | L-aMeGlu | His | D-Phe | Arg | Trp(6-Me) | Dap | ** | ++ |
| 1078 | | D-Nar | Cys | L-aMeGlu | His | D-Phe | Arg | Trp(7-F) | Cys | ** | + |
| 1025 | Gly | Beta-homoArg | Cys | L-aMeGlu | His | D-Phe | Arg | Trp | Cys | ** | ++ |
| 1031 | | Beta-homoArg | Cys | L-aMeGlu | His | D-Phe(3-Cl) | Arg | TRP | Cys | ** | ++ |
| 1053 | | Arg | Cys | L-aMeGlu | His | D-Phe(4-F) | Arg | Trp(6-F) | Cys | ** | + |
| 1060 | | D-Arg | Cys | L-aMeGlu | His | D-Phe(4-F) | Arg | Trp(6-Me) | Cys | ** | + |
| 1077 | | D-Nar | Cys | L-aMeGlu | His | D-Phe | Arg | Trp(5-F) | Cys | ** | + |
| 1114 | | Nar | Cys | L-aMeGlu | His | D-Phe | Arg | Trp(6-F) | Cys | ** | + |
| 1018 | Gly | D-Arg | Cys | L-aMeGlu | His | D-Phe | Arg | Trp | Cys | ** | + |
| 1056 | | Arg | Cys | L-aMeGlu | His | D-Phe(4-F) | Arg | Trp(6-Me) | Cys | ** | + |
| 1047 | | D-Nar | Cys | L-aMeGlu | His | D-Phe(3,4,5-triF) | Arg | Trp(6-F) | Cys | ** | ++ |
| 1081 | | D-Nar | Cys | L-aMeGlu | His | D-Phe(3-F) | Arg | Trp(5-F) | Cys | ** | + |
| 1075 | | D-Nar | Cys | L-aMeGlu | His | D-Phe | Arg | Trp(6-CF3) | Cys | ** | + |
| 1086 | | D-Nar | Cys | L-aMeGlu | His | D-Phe(3-Me) | Arg | Trp(6-F) | Cys | ** | ++ |
| 1061 | | L-hArg | Cys | L-aMeGlu | His | D-Phe(4-F) | Arg | Trp(6-Me) | Cys | ** | + |
| 1076 | | D-Nar | Cys | L-aMeGlu | His | D-Phe | Arg | Trp(4-F) | Cys | ** | + |
| 1116 | | D-Nar | Cys | L-aMeGlu | 3-Pal | D-Phe | Arg | Trp(6-Me) | Cys | ** | +++ |
| 1048 | | D-Nar | Cys | L-aMeGlu | His | D-Phe(3-Cl) | Arg | Trp(6-Me) | Cys | ** | +++ |
| 1074 | | D-Nar | Cys | L-aMeGlu | His | D-Phe(3,4-diF) | Arg | Trp(6-F) | Cys | ** | + |
| 1118 | | D-Nar | Cys | L-aMeGlu | 4-Pal | D-Phe | Arg | Trp(6-Me) | Cys | ** | ++ |
| 1051 | | D-Nar | Cys | L-aMeGlu | His | D-Phe(4-Cl) | Arg | Trp(6-F) | Cys | ** | + |
| 1059 | | Beta-homoArg | Cys | L-aMeGlu | His | D-Phe(4-F) | Arg | Trp(6-Me) | Cys | ** | + |
| 1045 | | D-Nar | Cys | L-aMeGlu | His | D-Phe(4-F) | Arg | Trp(6-Cl) | Cys | ** | + |
| 1032 | | Beta-homoArg | Cys | L-aMeGlu | His | D-Phe | Arg | Trp(6-Cl) | Cys | ** | + |
| 1082 | | D-Nar | Cys | L-aMeGlu | His | D-Phe(2,4-diCl) | Arg | Trp(6-F) | Cys | ** | ++ |
| 1073 | | D-Nar | Cys | L-aMeGlu | His | D-Phe(2-F, 4-Cl) | Arg | Trp(6-F) | Cys | ** | + |
| 1021 | | D-Nar | Cys | L-aMeGlu | His | D-Phe(4-Me) | Arg | Trp | Cys | ** | ++ |
| 1026 | | Beta-homoArg | Cys | L-aMeGlu | His | D-Phe(4-Cl) | Arg | Trp(6-Me) | Cys | * | ++ |
| 1017 | | D-Arg | Cys | L-aMeGlu | His | D-Phe(4-Me) | Arg | Trp | Cys | * | ++ |
| 1023 | | Beta-homoArg | Cys | L-aMeGlu | His | D-Phe(4-Me) | Arg | Trp | Cys | * | ++ |
| 1071 | | D-Nar | Cys | L-aMeGlu | His | D-Phe(3-F, 4-Me) | Arg | Trp(6-F) | Cys | * | ++ |
| 1072 | | D-Nar | Cys | L-aMeGlu | His | D-Phe(4-CF3) | Arg | Trp(6-F) | Cys | * | +++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 16
Table 16: Compounds with Cyclo-Leu at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1012 | Arg | Cys | Cyclo-Leu | Gln | D-Phe | Arg | Trp | Cys | *** | +++ |
| 1108 | D-Nar | Cys | Cyclo-Leu | 3-Pal | D-Phe | Arg | Trp(6-F) | Cys | ** | +++ |
| 1006 | Beta-homoArg | Cys | Cyclo-Leu | His | D-Phe | Arg | Trp | Cys | ** | + |
| 1005 | D-Arg | Cys | Cyclo-Leu | His | D-Phe | Arg | Trp | Cys | ** | + |
| 1052 | Beta-homoArg | Cys | Cyclo-Leu | His | D-Phe | Arg | Trp(6-F) | Cys | ** | + |
| 1008 | Beta-homoArg | Cys | Cyclo-Leu | His | D-Phe | Arg | Trp(6-Me) | Cys | ** | + |
| 1001 | Arg | Cys | Cyclo-Leu | His | D-Phe | Arg | Trp | Cys | ** | + |
| 1009 | D-Arg | Cys | Cyclo-Leu | His | D-Phe | Arg | Trp(6-Me) | Cys | ** | + |
| 1027 | Beta-homoArg | Cys | Cyclo-Leu | His | D-Phe(4-Cl) | Arg | TRP | Cys | * | ++ |
| 1015 | D-Arg | Cys | Cyclo-Leu | His | D-Phe(4-Me) | Arg | Trp(6-Me) | Cys | * | +++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 17
Table 17: Compounds with L-aMeAsp at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1121 | D-Nar | Glu | L-aMeAsp | His | D-Phe(4-F) | Arg | Trp(6-F) | Dap | *** | + |
| 1042 | D-Nar | Cys | L-aMeAsp | His | D-Phe | Arg | Trp(6-F) | Cys | *** | + |
| 1064 | D-Nar | Cys | L-aMeAsp | His | D-Phe | Arg | Trp(6-Me) | Cys | *** | + |
| 1062 | D-Nar | Cys | L-aMeAsp | His | D-Phe(4-F) | Arg | Trp(6-Me) | Cys | ** | + |
| 1068 | D-Nar | Glu | L-aMeAsp | His | D-Phe | Arg | Trp(6-Me) | Dap | ** | + |
| 1065 | beta-homoArg | Cys | L-aMeAsp | His | D-Phe | Arg | Trp(6-Me) | Cys | ** | + |
| 1004 | Arg | Cys | L-aMeAsp | His | D-Phe | Arg | Trp | Cys | ** | + |
| 1089 | D-Nar | Cys | L-aMeAsp | His | D-Phe(3-CF3) | Arg | Trp(6-F) | Cys | ** | ++ |
| 1063 | D-Arg | Cys | L-aMeAsp | His | D-Phe(4-F) | Arg | Trp(6-Me) | Cys | ** | + |
| 1120 | D-Nar | Glu | L-aMeAsp | His | D-Phe | Arg | Trp(6-F) | Dap | ** | + |
| 1069 | D-Nar | Asp | L-aMeAsp | His | D-Phe | Arg | Trp(6-Me) | Dap | ** | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, the data demonstrates that holding X₃ constant demonstrated that certain X₃ positions were amenable to retaining MC4R potency while losing MC1R potency. The data of Tables 8, 18-20 illustrates how the X₄ position itself was not sufficient to generate the selectivity that the combination of X₃ and X₄ positions generated, even with the X₁, X₅, and/or X₇ positions modified to improve selectivity/potency on MC4R.

TABLE 18
Peptides with 3-Pal at X₄. All peptides are N-acetylated,
have a disulfide linkage and contain a C-terminal amide.

| Molecule Name | X₁ | X₂ | X₃ | X₄ | X₅ | X₆ | X₇ | X₈ |
|---|---|---|---|---|---|---|---|---|
| 1119 | D-Nar | Cys | Phg | 3-Pal | D-Phe(4-F) | Arg | Trp(6-F) | Cys |
| 1122 | D-Nar | Cys | Aib(O-cyclic) | 3-Pal | D-Phe(4-F) | Arg | Trp(6-F) | Cys |
| 1124 | D-Nar | Cys | D-aMeOrn | 3-Pal | D-Phe(4-F) | Arg | Trp(6-F) | Cys |
| 1108 | D-Nar | Cys | Cyclo-Leu | 3-Pal | D-Phe | Arg | Trp(6-F) | Cys |
| 1116 | D-Nar | Cys | L-aMeGlu | 3-Pal | D-Phe | Arg | Trp(6-Me) | Cys |

TABLE 19
Molecule 1119, Molecule 1122, and Molecule 1124 retain high
cAMP potency at MC4R, while other analogues with 3-Pal at X₄ do
not. Molecule 1108 retains B-arrestin potency at MC4R but loses
cAMP potency, and Molecule 1116 loses potency across both cAMP
and B-arrestin. When comparing the selectivity of MC4R v MC1R,
larger values indicate selectivity towards MC4R. When comparing
the bias of MC4R B-arrestin v MC4R cAMP, larger values indicate
bias towards B-arrestin. Table 19:

| | Selectivity: | Bias: MC4R |
|---|---|---|
| Compound | MC4R v | B-arrestin v |
| Name | MC1R | MC4R cAMP |
| 1119 | *** | ++ |
| 1122 | *** | ++ |
| 1124 | *** | ++ |
| 1108 | ** | +++ |
| 1116 | ** | +++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, pairing Gln at X₄ with either D-aMeOm or Aib(O-cyclic) at X₃ outperformed the next best substitution at X₃ by >2× on MC4R vs MC1R selectivity. When comparing the selectivity of MCAR v MC1R, larger values indicate selectivity towards MCAR. When comparing the bias of MCAR B-arrestin v MC4R CAMP, larger values indicate bias towards B-arrestin.

TABLE 20
Compounds with Gln at X₄

| | Selectivity: | Bias: MC4R |
|---|---|---|
| Compound | MC4R v | B-arrestin v |
| Name | MC1R | MC4R cAMP |
| 1094 | *** | ++ |
| 1093 | *** | ++ |
| 1092 | *** | ++ |
| 1091 | *** | ++ |
| 1096 | *** | ++ |
| 1012 | *** | +++ |
| 1099 | *** | +++ |
| 1070 | ** | ++ |
| 1013 | ** | +++ |
| 1098 | ** | ++ |
| 1011 | ** | ++ |
| 1090 | ** | ++ |
| 1097 | ** | ++ |
| 1014 | ** | ++ |
| 1158 | *** | + |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Table 8 shown below lists families of molecules that have the X₄ position (e.g., Gln) in common.

The data of Table 8 illustrates how the X₄ position itself was not sufficient to generate the selectivity that the combination of X₃ and X₄ positions generated, even with the X₁, X₅, and/or X₇ positions modified to improve selectivity/potency on MC4R.

Without being bound to a particular theory, pairing Gln at X₄ with Aib(O-cyclic) at X₃ generally outperformed the next best substitution at X₃ by >2× on MC4R vs MC1R selectivity. When comparing the selectivity of MC4R v MC1R, larger values indicate selectivity towards MC4R. When comparing the bias of MC4R B-arrestin v MC4R CAMP, larger values indicate bias towards B-arrestin.

TABLE 8
Compounds with Gln at X₄

| | | | | | | | | | | | | | | | | | | Bias: MC4R |
|---|---|---|---|---|---|---|---|---|---|---|---|---|---|---|---|---|---|---|
| Molecule | | | | | | | | | | | | | | | | | Selectivity: | B-arrestin v |
| name | X₋₄ | X₋₃ | X₋₂ | X₋₁ | X₁ | X₂ | X₃ | X₄ | X₅ | X₆ | X₇ | X₈ | X₉ | X₁₀ | X₁₁ | X₁₂ | MC4R v MC1R | MC4R cAMP |
| 1148 | Lys* | D-Arg | Gly | D-Arg | D-Nar | Cys | D-aMeOrn | Gln | D-Phe | Arg | Trp(6-F) | Cys | | | | | * | + |
| 1149 | | Lys* | Gly | D-Arg | D-Nar | Cys | D-aMeOrn | Gln | D-Phe | Arg | Trp(6-F) | Cys | | | | | * | + |
| 1137 | | Lys* | PEG1 | PEG1 | D-Nar | Cys | D-aMeOrn | Gln | D-Phe | Arg | Trp(6-F) | Cys | | | | | ** | ++ |
| 1136 | Lys* | D-Arg | PEG1 | D-Arg | Beta-homoArg | Cys | D-aMeOrn | Gln | D-Phe | Arg | Trp(6-F) | Cys | | | | | ** | ++ |
| 1150 | Lys* | D-Arg | Gly | D-Arg | D-Nar | Cys | Aib(O-cyclic) | Gln | D-Phe(4-F) | Arg | Trp(6-F) | Cys | | | | | * | + |
| 1142 | | Lys* | Gly | D-Arg | D-Nar | Cys | Aib(O-cyclic) | Gln | D-Phe(4-F) | Arg | Trp(6-F) | Cys | | | | | ** | + |
| 1144 | | Lys* | PEG1 | PEG1 | D-Nar | Cys | Aib(O-cyclic) | Gln | D-Phe(4-F) | Arg | Trp(6-F) | Cys | | | | | ** | ++ |
| 1151 | Lys* | D-Arg | PEG1 | D-Arg | Beta-homoArg | Cys | Aib(O-cyclic) | Gln | D-Phe(4-F) | Arg | Trp(6-F) | Cys | | | | | * | ++ |
| 25 | | | | | D-Nar | Glu | Aib(O-cyclic) | Gln | D-Phe(4-F) | Arg | Trp(6-F) | Dap | | | | | *** | + |
| 26 | | | | | D-Nar | Glu | D-aMeOrn | Gln | D-Phe | Arg | Trp(6-F) | Dap | | | | | *** | + |
| 28 | | | | | Beta-homoArg | Cys | Aib(O-cyclic) | Gln | D-Phe(4-F) | Arg | Trp(6-F) | Cys | | | | | *** | + |
| 29 | | | | | Beta-homoArg | Cys | D-aMeOrn | Gln | D-Phe | Arg | Trp(6-F) | Cys | | | | | *** | + |
| 31 | | | | | Beta-homoArg | Cys | Phg | Gln | D-Phe(4-F) | Arg | Trp(6-F) | Cys | | | | | *** | ++ |
| 32 | | | | | D-Nar | Cys | Phg | Gln | D-Phe(4-F) | Arg | Trp(6-F) | Cys | | | | | *** | ++ |
| 37 | | | | | D-Nar | Cys | Phe | Gln | D-Phe(4-F) | Arg | Trp(6-F) | Cys | | | | | *** | ++ |
| 38 | | | | | D-Nar | Cys | Tyr | Gln | D-Phe(4-F) | Arg | Trp(6-F) | Cys | | | | | *** | ++ |
| 39 | | | | | D-Nar | Cys | D-Phe | Gln | D-Phe(4-F) | Arg | Trp(6-F) | Cys | | | | | *** | + |
| 1158 | | | | | D-Nar | Cys | Aib(O-cyclic) | Gln | D-Phe(4-F) | Arg | Trp(6-F) | Pen | | | | | *** | + |
| 41 | | | | | D-Nar | Cys | D-aMeOrn | Gln | D-Phe | Arg | Trp(6-F) | Pen | | | | | *** | + |
| 43 | | | | | D-Nar | hCys | Aib(O-cyclic) | Gln | D-Phe(4-F) | Arg | Trp(6-F) | Pen | | | | | ** | ++ |
| 44 | | | | | D-Nar | hCys | D-aMeOrn | Gln | D-Phe | Arg | Trp(6-F) | Pen | | | | | ** | ++ |
| 46 | | | | | D-Nar | Cys | Aib(O-cyclic) | Gln | D-Phe | Arg | Trp | Cys | | | | | *** | ++ |
| 47 | | | | | D-Nar | Cys | D-aMeOrn | Gln | D-Phe | Arg | Trp | Cys | | | | | *** | ++ |
| 50 | | | | | Arg | Cys | Phg | Gln | D-Phe | Arg | Trp | Cys | | | | | *** | ++ |
| 51 | | | | | Arg | Cys | Aib(O-cyclic) | Gln | D-Phe | Arg | Trp | Cys | | | | | *** | ++ |
| 52 | | | | | Arg | Cys | D-aMeOrn | Gln | D-Phe | Arg | Trp | Cys | | | | | *** | ++ |
| 53 | | | | | Arg | Cys | D-aMeOrn | Gln | D-Phe | Arg | Trp | Pen | | | | | *** | ++ |
| 56 | | | | | D-Nar | Cys | D-Phg | Gln | D-Phe(4-F) | Arg | Trp(6-F) | Cys | | | | | *** | + |
| 58 | | | | | D-Nar | Cys | D-Iva | Gln | D-Phe(4-F) | Arg | Trp(6-F) | Cys | | | | | *** | + |
| 60 | | | | | D-Nar | Cys | bAc5c | Gln | D-Phe(4-F) | Arg | Trp(6-F) | Cys | | | | | * | + |
| 62 | | | | | Beta-homoArg | Cys | bAc5c | Gln | D-Phe(4-F) | Arg | Trp(6-F) | Cys | | | | | ** | ++ |
| 64 | | | | | D-Nar | Cys | bAc4c | Gln | D-Phe(4-F) | Arg | Trp(6-F) | Cys | | | | | <** | + |
| 66 | | | | | Beta-homoArg | Cys | bAc4c | Gln | D-Phe(4-F) | Arg | Trp(6-F) | Cys | | | | | ** | + |
| 68 | | | | | D-Nar | Cys | bAc3c | Gln | D-Phe(4-F) | Arg | Trp(6-F) | Cys | | | | | ** | + |
| 70 | | | | | Beta-homoArg | Cys | bAc3c | Gln | D-Phe(4-F) | Arg | Trp(6-F) | Cys | | | | | *** | ++ |
| 78 | | | | | D-Nar | Cys | Cyclo-Leu | Gln | D-Phe(4-F) | Arg | Trp(6-F) | Cys | | | | | *** | + |
| 80 | | | | | Beta-homoArg | Cys | Cyclo-Leu | Gln | D-Phe(4-F) | Arg | Trp(6-F) | Cys | | | | | *** | + |
| 101 | | | | | Beta-homoArg | Cys | D-aMeSer | Gln | D-Phe | Arg | Trp(6-F) | Cys | | | | | *** | ++ |
| 108 | | | | | Beta-homoArg | Cys | L-aMeSer | Gln | D-Phe | Arg | Trp(6-F) | Cys | | | | | *** | ++ |
| 110 | | | | | D-Nar | Cys | D-aMeSer | Gln | D-Phe(4-F) | Arg | Trp(5-Me) | Cys | | | | | *** | +++ |
| 111 | Lys* | Gly | Gly | Gly | D-Nar | Cys | Aib(O-cyclic) | Gln | D-Phe(4-F) | Arg | Trp(6-F) | Cys | | | | | * | ++ |
| 112 | | Lys* | Gly | Gly | D-Nar | Cys | Aib(O-cyclic) | Gln | D-Phe(4-F) | Arg | Trp(6-F) | Cys | | | | | ** | ++ |
| 113 | | | Lys* | Gly | D-Nar | Cys | Aib(O-cyclic) | Gln | D-Phe(4-F) | Arg | Trp(6-F) | Cys | | | | | ** | ++ |
| 114 | | | | Lys* | D-Nar | Cys | Aib(O-cyclic) | Gln | D-Phe(4-F) | Arg | Trp(6-F) | Cys | | | | | ** | ++ |
| 115 | | | | | D-Nar | Cys | Aib(O-cyclic) | Gln | D-Phe(4-F) | Arg | Trp(6-F) | Cys | Gly | Gly | Gly | Lys* | ** | ++ |
| 116 | | | | | D-Nar | Cys | Aib(O-cyclic) | Gln | D-Phe(4-F) | Arg | Trp(6-F) | Cys | Gly | Gly | Lys* | | * | ++ |
| 117 | | | | | D-Nar | Cys | Aib(O-cyclic) | Gln | D-Phe(4-F) | Arg | Trp(6-F) | Cys | Gly | Lys* | | | ** | ++ |
| 118 | | | | | D-Nar | Cys | Aib(O-cyclic) | Gln | D-Phe(4-F) | Arg | Trp(6-F) | Cys | Lys* | | | | * | ++ |
| 119 | | | | | D-Nar | Cys | Aib(O-cyclic) | Gln | D-Phe(4-F) | Arg | Trp(6-F) | Cys | PEG1 | PEG1 | Lys* | | ** | ++ |
| 120 | | | | | D-Nar | Cys | Aib(O-cyclic) | Gln | D-Phe(4-F) | Arg | Trp(6-F) | Cys | D-Arg | Gly | Lys* | | ** | ++ |
| 121 | | | | | D-Nar | Cys | Aib(O-cyclic) | Gln | D-Phe(4-F) | Arg | Trp(6-F) | Cys | Pro | Phe | Lys* | | ** | ++ |
| 122 | | | | | D-Nar | Cys | Aib(O-cyclic) | Gln | D-Phe(4-F) | Arg | Trp(6-F) | Cys | Lys | Pro | Val | Lys* | * | ++ |
| 130 | | Lys* | Gly | Gly | D-Nar | Cys | D-aMeOrn | Gln | D-Phe | Arg | Trp(6-F) | Cys | | | | | ** | ++ |
| 131 | | | Lys* | Gly | D-Nar | Cys | D-aMeOrn | Gln | D-Phe | Arg | Trp(6-F) | Cys | | | | | *** | ++ |
| 132 | | | | Lys* | D-Nar | Cys | D-aMeOrn | Gln | D-Phe | Arg | Trp(6-F) | Cys | | | | | ** | ++ |
| 133 | | | | | D-Nar | Cys | D-aMeOrn | Gln | D-Phe | Arg | Trp(6-F) | Cys | Lys* | | | | * | ++ |
| 134 | | | | | D-Nar | Cys | D-aMeOrn | Gln | D-Phe | Arg | Trp(6-F) | Cys | Gly | Lys* | | | ** | ++ |
| 135 | | | | | D-Nar | Cys | D-aMeOrn | Gln | D-Phe | Arg | Trp(6-F) | Cys | Gly | Gly | Lys* | | ** | ++ |
| 136 | | | | | D-Nar | Cys | D-aMeOrn | Gln | D-Phe | Arg | Trp(6-F) | Cys | Lys | Pro | Val | Lys* | * | ++ |
| 140 | | | | Lys* | Arg | Cys | Aib(O-cyclic) | Gln | D-Phe(4-F) | Arg | Trp(6-F) | Pen | | | | | *** | + |
| 141 | | | | Lys* | D-Nar | Cys | Aib(O-cyclic) | Gln | D-Phe(4-F) | Arg | Trp(6-F) | Pen | | | | | *** | + |
| 142 | | | | Lys* | BetahomoArg | Cys | Aib(O-cyclic) | Gln | D-Phe(4-F) | Arg | Trp(6-F) | Pen | | | | | *** | + |
| 143 | | | | Lys* | Arg | Cys | Aib(O-cyclic) | Gln | D-Phe | Arg | Trp | Pen | | | | | ** | ++ |
| 144 | | | | Lys* | D-Nar | Cys | Aib(O-cyclic) | Gln | D-Phe | Arg | Trp | Pen | | | | | *** | + |
| 145 | | | | Lys* | BetahomoArg | Cys | Aib(O-cyclic) | Gln | D-Phe | Arg | Trp | Pen | | | | | ** | ++ |
| 146 | | | | Lys* | Arg | Cys | Aib(O-cyclic) | Gln | D-Phe | Arg | Trp(6-F) | Pen | | | | | ** | + |
| 147 | | | | Lys* | D-Nar | Cys | Aib(O-cyclic) | Gln | D-Phe | Arg | Trp(6-F) | Pen | | | | | ** | + |
| 148 | | | | Lys* | BetahomoArg | Cys | Aib(O-cyclic) | Gln | D-Phe | Arg | Trp(6-F) | Pen | | | | | *** | ++ |
| 149 | | | | Lys* | Arg | Cys | Aib(O-cyclic) | Gln | D-Phe(4-F) | Arg | Trp | Pen | | | | | *** | + |
| 150 | | | | Lys* | D-Nar | Cys | Aib(O-cyclic) | Gln | D-Phe(4-F) | Arg | Trp | Pen | | | | | *** | + |
| 151 | | | | Lys* | BetahomoArg | Cys | Aib(O-cyclic) | Gln | D-Phe(4-F) | Arg | Trp | Pen | | | | | *** | ++ |
| 152 | | | | | D-Nar | Cys | Aib(O-cyclic) | Gln | D-Phe | Arg | Trp(6-F) | Pen | | | | | *** | + |
| 153 | | | | | D-Nar | Cys | Aib(O-cyclic) | Gln | D-Phe(4-F) | Arg | Trp | Pen | | | | | *** | + |
| 154 | | | | | Beta-homoArg | Cys | D-aMeOrn | Gln | D-Phe | Arg | Trp(6-F) | Pen | | | | | *** | + |
| 155 | | | | | Beta-homoArg | Cys | D-aMeOrn | Gln | D-Phe(4-F) | Arg | Trp(6-F) | Pen | | | | | *** | + |
| 156 | | | | | Beta-homoArg | Cys | D-aMeOrn | Gln | D-Phe(4-F) | Arg | Trp | Pen | | | | | *** | + |
| 157 | | | | | Beta-homoArg | Cys | Cyclo-Leu | Gln | D-Phe(4-F) | Arg | Trp(6-F | Pen | | | | | *** | + |
| 158 | | | | | D-Nar | Cys | Cyclo-Leu | Gln | D-Phe(4-F) | Arg | Trp(6-F) | Pen | | | | | *** | + |
| 160 | | | | | D-Nar | Cys | D-Iva | Gln | D-Phe(4-F) | Arg | Trp(6-F) | Pen | | | | | *** | ++ |
| 161 | | | | | Beta-homoArg | Cys | Cyclo-Leu | Gln | D-Phe | Arg | Trp(6-F) | Pen | | | | | *** | +++ |
| 162 | | | | | D-Nar | Cys | Cyclo-Leu | Gln | D-Phe | Arg | Trp(6-F) | Pen | | | | | *** | ++ |
| 164 | | | | | D-Nar | Cys | D-Iva | Gln | D-Phe | Arg | Trp(6-F) | Pen | | | | | *** | ++ |
| 165 | | | | Lys* | Arg | Cys | Aib | Gln | D-Phe(4-F) | Arg | Trp(6-F) | Pen | | | | | * | +++ |
| 166 | | | | Lys* | D-Nar | Pen | Aib(O-cyclic) | Gln | D-Phe(4-F) | Arg | Trp(6-F) | Pen | | | | | *** | + |
| 167 | | | | Lys* | D-Nar | Pen | Aib(O-cyclic) | Gln | D-Phe(4-F) | Arg | Trp(6-F) | Cys | | | | | * | +++ |
| 168 | | | | | D-Nar | Pen | Aib(O-cyclic) | Gln | D-Phe(4-F) | Arg | Trp(6-F) | Pen | | | | | *** | ++ |
| 169 | | | | | D-Nar | Pen | Aib(O-cyclic) | Gln | D-Phe(4-F) | Arg | Trp(6-F) | Cys | | | | | *** | + |
| 171 | | | | | D-Nar | Cys | (3S)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-furancarboxylic | | | | | | | | | | | |
| | | | | | | | acid | | | | | | | | | | | |
| 172 | | | | | D-Nar | Cys | (3R)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-furancarboxylic | | | | | | | | | | | |
| | | | | | | | acid | | | | | | | | | | | |
| 173 | | | | | D-Nar | Cys | (3S)-3- | Gln | D-Phe(4-F) | Arg | Trp(6-F) | Pen | | | | | N/A | N/A |
| | | | | | | | Aminotetrahydro-3- | | | | | | | | | | | |
| | | | | | | | thiphenecarboxylic | | | | | | | | | | | |
| | | | | | | | acid | | | | | | | | | | | |
| 174 | | | | | D-Nar | Cys | (3R)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-thiophene | | | | | | | | | | | |
| | | | | | | | carboxylic | | | | | | | | | | | |
| | | | | | | | acid | | | | | | | | | | | |
| 175 | | | | | D-Nar | Cys | N-Boc-(3S)-3- | Gln | D-Phe(4-F) | Arg | Trp(6-F) | Pen | | | | | N/A | N/A |
| | | | | | | | amino-1,3- | | | | | | | | | | | |
| | | | | | | | pyrrolidine- | | | | | | | | | | | |
| | | | | | | | dicarboxylate | | | | | | | | | | | |
| 176 | | | | | D-Nar | Cys | N-Boc-(3R)-3- | Gln | D-Phe(4-F) | Arg | Trp(6-F) | Pen | | | | | N/A | N/A |
| | | | | | | | amino-1,3- | | | | | | | | | | | |
| | | | | | | | pyrrolidine- | | | | | | | | | | | |
| | | | | | | | dicarboxylate | | | | | | | | | | | |
| 177 | | | | | D-Nar | Cys | 3-Amino-3- | Gln | D-Phe(4-F) | Arg | Trp(6-F) | Pen | | | | | N/A | N/A |
| | | | | | | | thietane- | | | | | | | | | | | |
| | | | | | | | carboxylic | | | | | | | | | | | |
| | | | | | | | acid | | | | | | | | | | | |
| 178 | | | | | D-Nar | Cys | 3-Aminothietane- | Gln | D-Phe(4-F) | Arg | Trp(6-F) | Pen | | | | | N/A | N/A |
| | | | | | | | 3-carboxylic | | | | | | | | | | | |
| | | | | | | | acid 1,1- | | | | | | | | | | | |
| | | | | | | | dioxide | | | | | | | | | | | |
| 179 | | | | | D-Nar | Cys | N-Boc-3-amino- | Gln | D-Phe(4-F) | Arg | Trp(6-F) | Pen | | | | | N/A | N/A |
| | | | | | | | 1,3-azetidine | | | | | | | | | | | |
| | | | | | | | dicarboxylate | | | | | | | | | | | |
| 180 | | | | | D-Nar | Cys | 1-Amino-3,3- | Gln | D-Phe(4-F) | Arg | Trp(6-F) | Pen | | | | | N/A | N/A |
| | | | | | | | dimethyl- | | | | | | | | | | | |
| | | | | | | | cyclobutane- | | | | | | | | | | | |
| | | | | | | | carboxylic | | | | | | | | | | | |
| | | | | | | | acid | | | | | | | | | | | |
| 181 | | | | | D-Nar | Cys | 5-Amino- | Gln | D-Phe(4-F) | Arg | Trp(6-F) | Pen | | | | | N/A | N/A |
| | | | | | | | spiro[2.3]hex- | | | | | | | | | | | |
| | | | | | | | ane-5- | | | | | | | | | | | |
| | | | | | | | carboxylic | | | | | | | | | | | |
| | | | | | | | acid | | | | | | | | | | | |
| 182 | | | | | D-Nar | Cys | 6-Amino-2-oxa- | Gln | D-Phe(4-F) | Arg | Trp(6-F) | Pen | | | | | N/A | N/A |
| | | | | | | | spiro[3.3]hep- | | | | | | | | | | | |
| | | | | | | | tane-6- | | | | | | | | | | | |
| | | | | | | | carboxylic | | | | | | | | | | | |
| | | | | | | | acid | | | | | | | | | | | |
| 183 | | | | | D-Nar | Cys | 2-amino-2- | Gln | D-Phe(4-F) | Arg | Trp(6-F) | Pen | | | | | N/A | N/A |
| | | | | | | | ethylbutanoic | | | | | | | | | | | |
| | | | | | | | acid | | | | | | | | | | | |
| 184 | | | | | D-Nar | Cys | (1S)-1-Amino- | Gln | D-Phe(4-F) | Arg | Trp(6-F) | Pen | | | | | N/A | N/A |
| | | | | | | | 2,3-dihydro- | | | | | | | | | | | |
| | | | | | | | 1H-indene-1- | | | | | | | | | | | |
| | | | | | | | carboxylic | | | | | | | | | | | |
| | | | | | | | acid | | | | | | | | | | | |
| 185 | | | | | D-Nar | Cys | (1R)-1-Amino- | Gln | D-Phe(4-F) | Arg | Trp(6-F) | Pen | | | | | N/A | N/A |
| | | | | | | | 2,3-dihydro- | | | | | | | | | | | |
| | | | | | | | 1H-indene-1- | | | | | | | | | | | |
| | | | | | | | carboxylic | | | | | | | | | | | |
| | | | | | | | acid | | | | | | | | | | | |
| 186 | | | | | D-Nar | Cys | Aib(O-cyclic) | Gln | L-Methionine | Arg | Trp(6-F) | Pen | | | | | N/A | N/A |
| | | | | | | | | | sulfoxide | | | | | | | | | |
| 187 | | | | | D-Nar | Cys | Aib(O-cyclic) | Gln | L-Methionine | Arg | Trp(6-F) | Pen | | | | | N/A | N/A |
| | | | | | | | | | sulfone | | | | | | | | | |
| 188 | | | | | D-Nar | Cys | Aib(O-cyclic) | Gln | (2S)-2- | Arg | Trp(6-F) | Pen | | | | | N/A | N/A |
| | | | | | | | | | Amino-4- | | | | | | | | | |
| | | | | | | | | | cyanobutanoic | | | | | | | | | |
| | | | | | | | | | acid | | | | | | | | | |
| 189 | | | | | D-Nar | Cys | Aib(O-cyclic) | Gln | 3-(Acetylamino)- | Arg | Trp(6-F) | Pen | | | | | N/A | N/A |
| | | | | | | | | | L-alanine | | | | | | | | | |
| 190 | | | | | D-Nar | Cys | Aib(O-cyclic) | Gln | O-Carbamoyl- | Arg | Trp(6-F) | Pen | | | | | N/A | N/A |
| | | | | | | | | | L-serine | | | | | | | | | |
| 191 | | | | | D-Nar | Cys | Aib(O-cyclic) | Gln | 2-Hydroxy- | Arg | Trp(6-F) | Pen | | | | | N/A | N/A |
| | | | | | | | | | L-tryptophan | | | | | | | | | |
| 192 | | | | | D-Nar | Cys | Aib(O-cyclic) | Gln | 3-(Trimethylsilyl)- | Arg | Trp(6-F) | Pen | | | | | N/A | N/A |
| | | | | | | | | | D-alanine | | | | | | | | | |
| 193 | | | | | D-Nar | Cys | Aib(O-cyclic) | Gln | 5,5,5-Trifluoro- | Arg | Trp(6-F) | Pen | | | | | N/A | N/A |
| | | | | | | | | | D-norvaline | | | | | | | | | |
| 194 | | | | | D-Nar | Cys | Aib(O-cyclic) | Gln | 3-(Trifluoromethyl)- | Arg | Trp(6-F) | Pen | | | | | N/A | N/A |
| | | | | | | | | | D-alanine | | | | | | | | | |
| 195 | | | | | D-Nar | Cys | Aib(O-cyclic) | Gln | 3-Cyano- | Arg | Trp(6-F) | Pen | | | | | N/A | N/A |
| | | | | | | | | | D-alanine | | | | | | | | | |
| 196 | | | | | D-Nar | Cys | Aib(O-cyclic) | Gln | 3-Cyclopropyl- | Arg | Trp(6-F) | Pen | | | | | N/A | N/A |
| | | | | | | | | | D-alanine | | | | | | | | | |
| 197 | | | | | D-Nar | Cys | Aib(O-cyclic) | Gln | (R)-2-Amino-4- | Arg | Trp(6-F) | Pen | | | | | N/A | N/A |
| | | | | | | | | | cyclopropylbutanoic | | | | | | | | | |
| | | | | | | | | | acid | | | | | | | | | |
| 198 | | | | | D-Nar | Cys | Aib(O-cyclic) | Gln | (αR)-α-Amino- | Arg | Trp(6-F) | Pen | | | | | N/A | N/A |
| | | | | | | | | | 2-pyridine- | | | | | | | | | |
| | | | | | | | | | propanoic | | | | | | | | | |
| | | | | | | | | | acid | | | | | | | | | |
| 199 | | | | | D-Nar | Cys | Aib(O-cyclic) | Gln | (αR)-α-Amino- | Arg | Trp(6-F) | Pen | | | | | N/A | N/A |
| | | | | | | | | | 3-pyridine- | | | | | | | | | |
| | | | | | | | | | propanoic | | | | | | | | | |
| | | | | | | | | | acid | | | | | | | | | |
| 200 | | | | | D-Nar | Cys | Aib(O-cyclic) | Gln | (αR)-α-Amino- | Arg | Trp(6-F) | Pen | | | | | N/A | N/A |
| | | | | | | | | | 4-pyridine- | | | | | | | | | |
| | | | | | | | | | propanoic | | | | | | | | | |
| | | | | | | | | | acid | | | | | | | | | |
| 1011 | | | | | Arg | Cys | D-Ala | Gln | D-Phe | Arg | Trp | Cys | | | | | ** | ++ |
| 1012 | | | | | Arg | Cys | Cyclo-Leu | Gln | D-Phe | Arg | Trp | Cys | | | | | *** | +++ |
| 1013 | | | | | Arg | Cys | Ala(2-Me) | Gln | D-Phe | Arg | Trp | Cys | | | | | ** | +++ |
| 1014 | | | | | Beta-homoArg | Cys | D-Dab | Gln | D-Phe | Arg | Trp | Cys | | | | | ** | ++ |
| 1070 | | | | | Arg | Cys | D-Ala | Gln | D-Phe | Arg | Trp | Cys | | | | | ** | ++ |
| 1090 | | | | | D-Nar | Cys | L-aMeGlu | Gln | D-Phe | Arg | Trp(6-F) | Cys | | | | | ** | ++ |
| 1091 | | | | | D-Nar | Cys | hGlu | Gln | D-Phe | Arg | Trp(6-F) | Cys | | | | | *** | ++ |
| 1092 | | | | | D-Nar | Cys | Aib(O-cyclic) | Gln | D-Phe | Arg | Trp(6-F) | Cys | | | | | *** | ++ |
| 1093 | | | | | D-Nar | Cys | Aib(O-cyclic) | Gln | D-Phe(4-F) | Arg | Trp(6-F) | Cys | | | | | *** | ++ |
| 1094 | | | | | D-Nar | Cys | D-aMeOrn | Gln | D-Phe | Arg | Trp(6-F) | Cys | | | | | *** | ++ |
| 1096 | | | | | D-Nar | Cys | D-aMeSer | Gln | D-Phe | Arg | Trp(6-F) | Cys | | | | | *** | ++ |
| 1097 | | | | | D-Nar | Cys | D-aMeSer | Gln | D-Phe(4-F) | Arg | Trp(6-F) | Cys | | | | | ** | ++ |
| 1098 | | | | | D-Nar | Cys | bhGlu | Gln | D-Phe | Arg | Trp(6-F) | Cys | | | | | ** | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Table 11 lists the calculated MC4R vs MC1R selectivity of some exemplary peptides tested. Setmelanotide has a selectivity of 1.06, and Melanotan II has a selectivity of 0.08. When comparing the selectivity of MCAR v MC1R, larger values indicate selectivity towards MC4R. When comparing the bias of MCAR B-arrestin v MC4R CAMP, larger values indicate bias towards B-arrestin.

TABLE 11
The in vitro assay activity and calculated selectivity and
calculated bias of the foregoing exemplary molecules that
are selective for MC4R vs MC1R in cAMP functional screening
and may also be biased for B-arrestin over cAMP signal.

| | Selectivity: | Bias: MC4R |
|---|---|---|
| Peptide | MC4R v | B-arrestin v |
| Name | MC1R | MC4R cAMP |
| 1119 | *** | ++ |
| 1094 | *** | ++ |
| 1106 | *** | ++ |
| 1122 | *** | ++ |
| 1107 | *** | ++ |
| 1093 | *** | ++ |
| 1092 | *** | ++ |
| 1124 | *** | ++ |
| 1015 | *** | + |
| 1035 | *** | + |
| 1091 | *** | ++ |
| 1096 | *** | ++ |
| 1043 | *** | + |
| 1012 | *** | +++ |
| 1049 | *** | + |
| 1041 | *** | + |
| 1099 | *** | +++ |
| 1030 | *** | +++ |
| 1121 | *** | + |
| 1042 | *** | + |
| 1024 | *** | + |
| 1064 | *** | + |
| 1037 | *** | + |
| 1019 | *** | + |
| 1085 | *** | + |
| 1016 | *** | + |
| 1158 | *** | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

While positions at X₁, X₃, X₅, and X₇ were initially chosen based on the properties of computer aided designs and functional testing of individual and combinatorial substitutions, the improvements in selectivity were unexpectedly based on specific combinations of amino acids at the X₃ and X₄ positions in combination with X₁, X₅, and/or X₇ positional changes.

In addition, it was observed that when X₈ is Pen, selectivity was unexpectedly improved compared to other X₈ groups (e.g. when compared to Cys at X₈). Peptides with X₈=Pen were found to be 2×-10× more selective for MC4R v MC1R compared to peptides with X₈=Cys.

FIG. 8 is a table listing various exemplary peptides and their related results when assessing the selectivity of MC4R versus MC1R as well as the bias of MC4R B-arrestin versus MC4R cAMP. When comparing the selectivity of MC4R v MC1R, larger values indicate selectivity towards MC4R. When comparing the bias of MC4R B-arrestin v MC4R CAMP, larger values indicate bias towards B-arrestin. * denotes MC4R vs MC1R selectivity having a range of 0.01 to <1.00, ** denotes MC4R vs MC1R selectivity having a range of ≥1.00 to ≤7.40, *** denotes MC4R vs MC1R selectivity of about >7.40. + denotes MC4R B-arrestin v MC4R cAMP bias having a range of >0.00 to ≤2.00, ++ denotes MC4R B-arrestin v MC4R CAMP bias having a range of 2.00 to ≤10.00, +++ denotes MC4R B-arrestin v MC4R CAMP bias having a value of >10.00. Grey color denotes peptides with a lipidated state. N/A denotes peptides with no data collected. IA denotes an inactive state.

Murine Weight Loss Data with Various Peptides

3-day efficacy study was conducted in DIO mice to evaluate the effect on murine weight of the MC4R agonistic peptides described herein. In summary, highly selective peptides demonstrated efficacy in DIO mouse models (FIG. 5).

### 3-Day Murine Acute Feeding and Weight Loss Assay, Once Daily Dosing

In a 3-day acute feeding and weight loss assay with daily subcutaneous dosing, peptides described herein with selectivity for MC4R vs MC1R generated weight loss similar to comparator molecule 1, setmelanotide (FIG. 6).

14-Day Murine Weight Loss Assay with Daily MC4R Agonist Dosing

In a 14-day assay, efficacy studies were performed using diet-induced obesity (DIO) C57BL/6 male mice (aged 18-20 weeks) to evaluate the weight loss effects of MC4R agonist peptides (e.g., molecule 1158). Mice were obtained from Gem Pharmatech, having been maintained on a 60% high-fat diet for 18-20 weeks prior to arrival. Upon delivery, mice were acclimated to the vivarium for two weeks to re-establish baseline weight and adjust to their environment. To enable accurate food intake measurements, mice were single-housed, which minimized stress as they had already been singly housed before arrival. On Day-1, mice were weighed and randomized into cohorts matched by weight and age (within a two-week age range). On Day 0), mice were weighed, and each mouse received a subcutaneous injection between the scapulae of 10 mg/kg test peptide in a formulation to support daily dosing. Post-injection, mice were observed cageside for 30 minutes. This procedure was repeated daily through Day 13. The study concluded on Day 13 with final measurements of body weight. Weight loss was reported as mean±SD percent change from Day 0 relative to a vehicle-treated group. Each treatment group included eight mice (N=8/group) (FIG. 7).

### Lipidated Variants of Peptides

Lipidated variants of Molecule 1119, Molecule 1094, and Molecule 1093 were generated. Additional variants of these 3 parent compounds with 3-Pal and Orn at X₄ for Molecule 1094 and Molecule 1093 were generated. Finally, the X₅ position for Molecule 1094 was edited to D-Phe(4-F) to modulate selectivity (Table 21).

Table 2 provided elsewhere herein, provides a detailed list of various lipidated peptides.

Tables 9 and 21, as listed below herein, provide an exemplary listing of lipidated variant peptides.

TABLE 9
Exemplary Lipidated Variant Peptides.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | Term | term | Cyclic |
| 1150 | Lys* | D-Arg | Gly | D-Arg | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1142 | | Lys* | Gly | D-Arg | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1144 | | Lys* | PEG1 | PEG1 | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1151 | Lys* | D-Arg | PEG1 | D-Arg | Beta- | Cys | Aib(O- | Gln | D-Phe(4-F) | Arg | Trp(6-F) | Cys | Ac | NH₂ | Disulfide |
| | | | | | homoArg | | cyclic) | | | | | | | | |
| 1152 | Lys* | D-Arg | Gly | D-Arg | D-Nar | Cys | Aib(O- | 3-Pal | D-Phe(4-F) | Arg | Trp(6-F) | Cys | Ac | NH₂ | Disulfide |
| | | | | | | | cyclic) | | | | | | | | |
| 1153 | Lys* | D-Arg | Gly | D-Arg | D-Nar | Cys | Aib(O- | Orn | D-Phe(4-F) | Arg | Trp(6-F) | Cys | 1153 | Lys* | D-Arg |
| | | | | | | | cyclic) | | | | | | | | |

Lys* = L-Lys(AEEAc-AEEAc-L-γ-Glu-17-carboxyheptadecanoyl)

TABLE 21
Lipidated Variant Peptides.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | Term | term | Cyclic |
| 1146 | Lys* | D-Arg | Gly | D-Arg | D-Nar | Cys | Phg | 3-Pal | D-Phe(4-F) | Arg | Trp(6-F) | Cys | Ac | NH₂ | Disulfide |
| 1139 | | Lys* | Gly | D-Arg | D-Nar | Cys | Phg | 3-Pal | D-Phe(4-F) | Arg | Trp(6-F) | Cys | Ac | NH₂ | Disulfide |
| 1145 | | Lys* | PEG1 | PEG1 | D-Nar | Cys | Phg | 3-Pal | D-Phe(4-F) | Arg | Trp(6-F) | Cys | Ac | NH₂ | Disulfide |
| 1147 | Lys* | D-Arg | PEG1 | D-Arg | Beta- | Cys | Phg | 3-Pal | D-Phe(4-F) | Arg | Trp(6-F) | Cys | Ac | NH₂ | Disulfide |
| | | | | | homoArg | | | | | | | | | | |
| 1148 | Lys* | D-Arg | Gly | D-Arg | D-Nar | Cys | D-aMeOrn | Gln | D-Phe | Arg | Trp(6-F) | Cys | Ac | NH₂ | Disulfide |
| 1149 | | Lys* | Gly | D-Arg | D-Nar | Cys | D-aMeOrn | Gln | D-Phe | Arg | Trp(6-F) | Cys | Ac | NH₂ | Disulfide |
| 1137 | | Lys* | PEG1 | PEG1 | D-Nar | Cys | D-aMeOrn | Gln | D-Phe | Arg | Trp(6-F) | Cys | Ac | NH₂ | Disulfide |
| 1136 | Lys* | D-Arg | PEG1 | D-Arg | Beta- | Cys | D-aMeOrn | Gln | D-Phe | Arg | Trp(6-F) | Cys | Ac | NH₂ | Disulfide |
| | | | | | homoArg | | | | | | | | | | |
| 1150 | Lys* | D-Arg | Gly | D-Arg | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1142 | | Lys* | Gly | D-Arg | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1144 | | Lys* | PEG1 | PEG1 | D-Nar | Cys | Aib(O- | Gln | D-Phe(4-F) | Arg | Trp(6-F) | Cys | Ac | NH₂ | Disulfide |
| | | | | | | | cyclic) | | | | | | | | |
| 1151 | Lys* | D-Arg | PEG1 | D-Arg | Beta- | Cys | Aib(O- | Gln | D-Phe(4-F) | Arg | Trp(6-F) | Cys | Ac | NH₂ | Disulfide |
| | | | | | homoArg | | cyclic) | | | | | | | | |
| 1152 | Lys* | D-Arg | Gly | D-Arg | D-Nar | Cys | Aib(O- | 3-Pal | D-Phe(4-F) | Arg | Trp(6-F) | Cys | Ac | NH₂ | Disulfide |
| | | | | | | | cyclic) | | | | | | | | |
| 1153 | Lys* | D-Arg | Gly | D-Arg | D-Nar | Cys | Aib(O- | Orn | D-Phe(4-F) | Arg | Trp(6-F) | Cys | Ac | NH₂ | Disulfide |
| | | | | | | | cyclic) | | | | | | | | |
| 1154 | Lys* | D-Arg | Gly | D-Arg | D-Nar | Cys | D-aMeOrn | 3-Pal | D-Phe(4-F) | Arg | Trp(6-F) | Cys | Ac | NH₂ | Disulfide |
| 1155 | Lys* | D-Arg | Gly | D-Arg | D-Nar | Cys | D-aMeOrn | Orn | D-Phe(4-F) | Arg | Trp(6-F) | Cys | Ac | NH₂ | Disulfide |
| 1156 | Lys* | D-Arg | Gly | D-Arg | D-Nar | Cys | D-aMeOrn | 3-Pal | D-Phe | Arg | Trp(6-F) | Cys | Ac | NH₂ | Disulfide |
| 1157 | Lys* | D-Arg | Gly | D-Arg | D-Nar | Cys | D-aMeOrn | Orn | D-Phe | Arg | Trp(6-F) | Cys | Ac | NH₂ | Disulfide |

Lys* = L-Lys(AEEAc-AEEAc-L-γ-Glu-17-carboxyheptadecanoyl)

Unless defined otherwise, all technical and scientific terms used herein have the meaning commonly understood by a person skilled in the art to which this disclosure belongs.

As used herein, the term “room temperature” refers to a temperature that is about 15-25° C.

As used herein, the term “W/W” refers to the concentration of a component of a composition by weight relative to the total weight of the composition.

As used herein, the terms “comprise” and “comprising” are inclusive and open-ended. Although the inclusive and open-ended terms “comprise” and “comprising” are used herein to describe and claim the disclosure, the present disclosure, or embodiments thereof, may alternatively be described using alternative terms such as “consisting of” or “consisting essentially of.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

While the disclosure has been described in connection with embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to embodiments described specifically herein. Such equivalents are intended to be encompassed in the scope of the following claims.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

The publications discussed herein are provided solely for their disclosure prior to the filing date of the present application. Nothing herein is to be construed as an admission that the present disclosure is not entitled to antedate such publication by virtue of prior disclosure.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed disclosure by, inter alia, a negative proviso or disclaimer of the feature or combination of features.

1. A composition comprising:
(a) about: 20-60% (W/W) total phospholipids, wherein the total phospholipids comprise a first and a second species of phospholipid in a ratio of about;
80:20, 75:25, 70:30, 60:40, 50:50, or 40:60 of the first species to the second species, wherein the second species is a phospholipid comprising a lipid with a dioleoyl and glycero group and/or a phosphoethanolamine group;
(b) about: 10-60% (W/W) of a slow diffusing solvent;
(c) about: 5-30% (W/W) of a fast diffusing solvent or solvent that is highly miscible with water such as an alcohol (such as ethanol), NMP, DMSO, or a combination thereof; and
(d) an active pharmaceutical ingredient (API).

2. The composition of claim 1, wherein;
(a) the second species of phospholipid is 1,2-dioleoyl-sn-glycero-3-phosphoethanolamine (DOPE);
(b) the first species of phospholipid is phosphatidylcholine;
(c) the first and second species are in a ratio of about: 80:20, 75:25, 70:30, 60:40, 50:50, or 40:60; and/or
(d) the composition comprises at least about: 30, 35, 36, 37, 38, 39, 40, 41, 42, 43, 44, 45, 46, 47, 48, 49, 50, 51, 52, 53, 54, 55, or 60% (W/W) total phospholipids.

3.-4. (canceled)

5. The composition of claim 1, wherein the slow diffusing solvent comprises one or more of medium or long chain triglycerides, Miglyol® 812 N and variants thereof (e.g., Miglyol® 810 N, Miglyol® 840), capric triglycerides, caprylic triglycerides, caproic triglycerides, lauric triglycerides, MYRITOL® 318 and variants thereof (e.g., MYRITOL® 312, MYRITOL® 331 N), NEOBEE® Caprylic Triglyceride (e.g., NEOBEE® 1053 MB), CAPTEX® medium chain triglycerides (e.g., CAPTEX® 200P, CAPTEX® 300 EP/NF, CAPTEX® 8000), medium chain triglycerides oil, sesame oil, castor oil, polyoxyl 35 castor oil, soybean oil, PEG-60 hydrogenated castor oil, peanut oil, cottonseed oil, corn oil, coconut oil, glycerin, monothioglycerol, glyceryl palmitostearate, glycerol dioleate, including combinations of the foregoing, and/or wherein the slow diffusing solvent is present at a concentration of about: 14, 15, 16, 17, 20, 25, 28, 30, 31, 33, 34, 35, 37, 38, 39, 40, 41, 42, 43, 44, 52, 53, 55, 58, or 60% (W/W).

6.-7. (canceled)

8. The composition of claim 1, wherein the fast diffusing solvent is ethanol, and/or wherein the fast diffusing solvent is at a concentration of about: 6, 7, 8, 9, 10, 11, 12, 13, 14, 15, 16, 20, 25, 26, 27, 28, 29, or 30% (W/W).

9. (canceled)

10. The composition of claim 1, wherein the API is present at a concentration of at least about: 0.1, 0.5, 1, 2, 3, 4, 5, 6, 7, 8, 9, 10, 12, 15% (W/W), or more.

11.-12. (canceled)

13. The composition of claim 1, wherein the API is a G protein coupled receptor (GPCR) modulator, and/or wherein the API is a MC4R agonist peptide or a salt thereof.

14. The composition of claim 1, wherein the API is a peptide or a salt thereof.

15. The composition of claim 14, wherein the peptide comprises the amino acid sequence of formula (I): 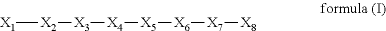
wherein in formula (I):
X₃ is 3-Aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

16. The composition of claim 15, wherein;
(a) X₄ is Gln;
(b) X₅ is selected from 4-fluoro-D-phenylalanine (D-Phe(4-F)), D-phenylalanine (D-Phe), and 4-methyl-D-phenylalanine (D-Phe(4-Me));
(c) X₆ is arginine (Arg);
(d) X₇ is 6-fluoro-L-tryptophan (Trp(6-F));
(e) X₈ is penicillamine (Pen) or cysteine (Cys);
(f) X₁ is selected from D-norarginine (D-Nar) and beta-homo-L-arginine (Beta-homoArg);
(g) X₂ is Cys;
(h) the peptide is a cyclic peptide;
(i) the peptide comprises a disulfide bridge or a lactam bridge; and/or
(j) the peptide is capped with N-terminal acetyl and/or C-terminal amide groups.

17.-24. (canceled)

25. The composition of claim 15, wherein the peptide is a cyclic peptide having the amino acid sequence of formula (II): 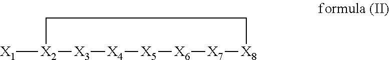
wherein: 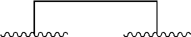 [SMILES: *CC*]
represents a disulfide bridge or a lactam bridge.

26.-28. (canceled)

29. The composition of claim 15, wherein the peptide of formula (I) is a peptide of any one of formula (Ia), formula (Ib), formula (Ic), formula (Id), formula (Ie), or formula (If): 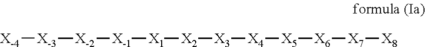
wherein in formula (Ia):
X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker; 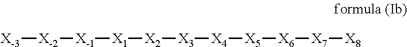
wherein in formula (Ib):
X₋₁, X₋₂, X₋₃, X₁, X₂, X₃, X₄, X₅, X₆, X₇, and X₈ are each independently an amino acid selected from Table 1, Table 2, Table 3, Table A1, Table A1A, Table A2, and Table A2A or a linker; 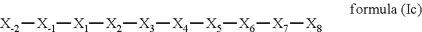
wherein in formula (Ic):
X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker; 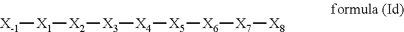
wherein in formula (Id);
X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker; 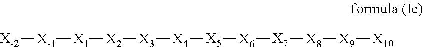
wherein in formula (Ie):
X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker; 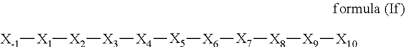
wherein in formula (If):
X₋₁, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

30. The composition of claim 25, wherein the cyclic peptide of formula (II) is a cyclic peptide of any one of formula (IIa), formula (IIb), formula (IIc), formula (IId), formula (IIe), or formula (IIf): 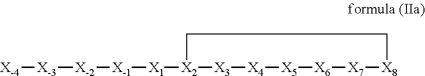
wherein in formula (IIa):
X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker; 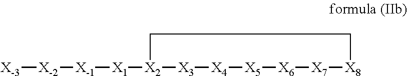
wherein in formula (IIb):
X₋₁, X₋₂, X₋₃, X₁, X₂, X₃, X₄, X₅, X₆, X₇, and X₈ are each independently an amino acid selected from Table 1, Table 2, Table 3, Table A1, Table A1A, Table A2, and Table A2A or a linker; 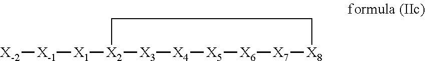
wherein in formula (IIc):
X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker; 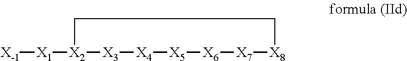
wherein in formula (IId):
X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker; 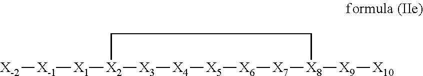
wherein in formula (IIe):
X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker; 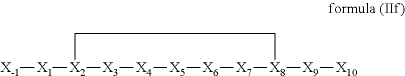
wherein in formula (IIf):
X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

31. The composition of claim 15, wherein the peptide is selected from Table A1, Table A1A, Table A2, Table A2A, Table 1, and Table 2.

32. The composition of claim 1, wherein the API is a salt of a peptide.

33. The composition of claim 32, wherein the salt is an acetate salt, a trifluoroacetate salt, a phosphate salt, a phosphite salt, a propionate salt, a chloride salt, a fumarate salt, a citrate salt, a tartrate salt, an oxalate salt, a succinate salt, a mandelate salt, a methanesulfonate salt, a p-toluenesulfonate salt, a bromide salt, an iodide salt, a hydroxide salt, a sulfate salt, a sulfite salt, a nitrate salt, a malate salt, a maleate salt, an aspartate salt, a glutamate salt, a lactate salt, a gluconate salt, a benzoate salt, a salicylate salt, an ethanesulfonate salt, a naphthalenesulfonate salt, or a camphorsulfonate salt.

34.-45. (canceled)

46. The composition of claim 15, wherein the peptide is peptide 1158 of the structure:

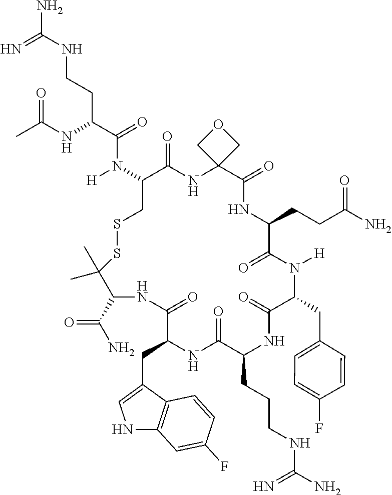

47. The composition of claim 15, wherein the peptide or salt thereof demonstrates one or more of (a)-(h):
(a) increased selectivity for MC4R over MC1R when administered to a subject compared to a control;
(b) increased selectivity for MC4R over MC1R when administered to a subject as measured by an in vitro, ex vivo, or in vivo assay when compared to a control;
(c) an increased ratio of MC4R intracellular signaling to MC1R intracellular signaling when administered to a subject compared to a control;
(d) increased selectivity for MC4R intracellular signaling to MC1R intracellular signaling as measured by an in vitro, ex vivo, or in vivo assay when compared to a control;
(e) enhanced melanocortin 4 receptor (MC4R) function in a subject when compared to before the salt is administered or to a pre-treatment or non-treatment state, or a subject treated with control;
(f) decreased melanocortin 1 receptor (MC1R) function in a subject when compared to before the salt is administered or to a pre-treatment or non-treatment state, or a subject treated with control;
(g) enhanced melanocortin 4 receptor (MC4R) function as measured by an in vitro, ex vivo, or in vivo assay when compared to a control; and
(h) decreased melanocortin 1 receptor (MC1R) function as measured by an in vitro, ex vivo, or in vivo assay when compared to a control.

48. The composition of claim 1, wherein the composition comprises an API and, upon administration to a mammalian subject, exhibits an extended release of the API, relative to a control composition, as evaluated by maximum serum concentration (Cmax), steady-state concentration (Css), and/or flat exposure of the API, at up to 24, 36, 48, 60, 72, 84, or 96 hours or more.

49.-51. (canceled)

52. A composition selected from any one of Table BBB, Table CCC, or Table DDD.

53.-66. (canceled)

67. The composition of claim 46, wherein the salt is: 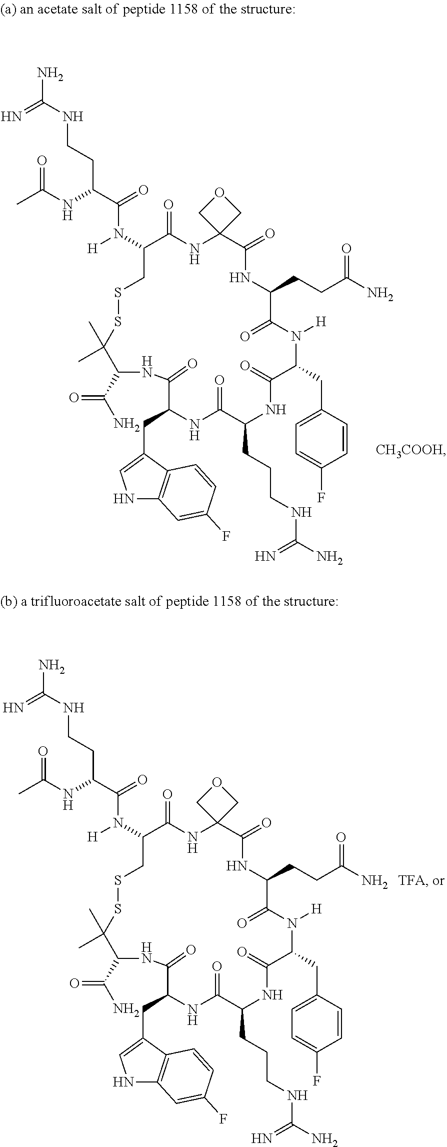 [chemical structure]
